(19) United States
(12) Patent Application Publication
(10) Pub. No.: US 2023/0391752 A1
(43) Pub. Date: Dec. 7, 2023

(54) PYRROLIDINE-3-CARBOXAMIDE DERIVATIVES AND RELATED USES

(71) Applicant: Chulalongkorn University, Bangkok (TH)

(72) Inventors: Tanachote RUENGSATRA, Bangkok (TH); Eakkaphon RATTANANGKOOL, Bangkok (TH); Pongkorn CHAIYAKUNVAT, Bangkok (TH); Sirikan DEESIRI, Bangkok (TH); Wilasinee DUNKOKSUNG, Bangkok (TH); Udomsak UDOMNILOBOL, Bangkok (TH); Natthaya CHUAYPEN, Bangkok (TH); Pisit TANGKIJVANICH, Bangkok (TH); Khanitha PUDHOM, Bangkok (TH); Thomayant PRUEKSARITANONT, Bangkok (TH)

(21) Appl. No.: 18/250,012

(22) PCT Filed: Oct. 22, 2021
(86) PCT No.: PCT/US2021/056267

§ 371 Date: Apr. 21, 2023

Related U.S. Application Data
(60) Provisional application No. 63/194,497, filed on May 28, 2021.
(60) Provisional application No. 63/104,103, filed on Oct. 22, 2020.

(51) Int. Cl. C07D 403/06; C07D 409/06; C07D 495/04; C07D 401/14; C07D 491/048; A61P 31/20; C07D 471/04; C07D 487/04; A61K 45/06; C07D 403/14; C07D 417/14

(52) U.S. Cl. CPC C07D 403/06; C07D 409/06; C07D 495/04; C07D 401/14; C07D 491/048; A61P 31/20; C07D 471/04; C07D 487/04; A61K 45/06; C07D 403/14; C07D 417/14

(57) ABSTRACT

The present disclosure relates to compounds of Formula (I′):

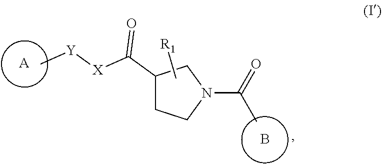

and to their prodrugs, pharmaceutically acceptable salts, pharmaceutical compositions, methods of use, and methods for their preparation. The compounds disclosed herein are useful for the treatment of a viral infection (e.g., hepatitis B virus or a Flaviviridae virus).

### RELATED APPLICATIONS

This application claims priority to, and the benefit of, U.S. Provisional Application Nos. 63/104,103, filed Oct. 22, 2020, and 63/194,497, filed May 28, 2021, the entire contents of each of which are incorporated herein by reference.

### BACKGROUND

The present disclosure relates to small molecule antiviral agents, designed for the treatment of hepatitis B virus (HBV) infection, inhibition of HBV viral replication, inhibition of the protein(s) encoded by HBV or interference with the function of the HBV replication cycle.

Chronic HBV infection is a significant global health problem, affecting over 5% of the world population (over 350 million people worldwide and 1.25 million individuals in the US). Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Problems that HBV direct acting antivirals may encounter are toxicity, mutagenicity, lack of selectivity, poor efficacy, poor bioavailability; low solubility and difficulty of synthesis. There is thus a need for additional inhibitors for the treatment, amelioration or prevention of HBV that may overcome at least one of these disadvantages or that have additional advantages such as increased potency or an increased safety window.

Administration of such therapeutic agents to an HBV infected patient, either as monotherapy or in combination with other HBV treatments or ancillary treatments, will lead to significantly reduced virus burden, improved prognosis, diminished progression of the disease and/or enhanced seroconversion rates.

The disclosure arises from a need to provide further compounds for treating HBV or inhibiting HBV viral replication, compositions comprising such compounds, and the process for making the compounds.

### SUMMARY

In some aspects, the present disclosure provides, inter alia, a compound of Formula (I′):

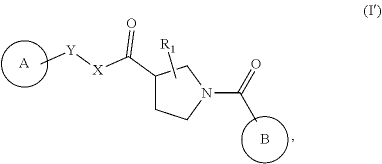

or a prodrug, solvate, or pharmaceutically acceptable salt thereof, wherein:
X is —N(Rₓ)— or —O—;
Y is absent or —C(R_{Y})₂—
Rₓ is H, C₁-C₆ alkyl, C₂-C₆ alkenyl, C₂-C₆ alkynyl, or C₁-C₆ haloalkyl;
each R_{Y} independently is H, C₁-C₆ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl;
Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A};
Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B};
R₁ is H, C₁-C₆ alkyl, C₂-C₆ alkenyl, C₂-C₆ alkynyl, or C₁-C₆ haloalkyl;
each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B} independently is halogen, —CN, —(CH₂)ₙ—OR_{B1}, —(CH₂)ₙN(R_{B1})(R_{B2}), —(CH₂)ₙ—S(R_{B1}), C₁-C₆ alkyl, C₂-C₆ alkenyl, C₁-C₆ haloalkyl, C₁-C₆ alkoxy, —(CH₂)ₙ—(C₆-C₁₀ aryl), (5- to 10-membered heteroaryl), (C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4};
each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or
R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₆ cycloalkyl;
each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′};
each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy;
each R_{B4} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₙ—C(O)R_{B4′}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4}″);
each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₁-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH;
n is 0, 1, 2, 3, 4, or 5; and
m is 0, 1, 2, 3, 4, or 5,
provided that when R_{B} is a substituted or unsubstituted alkyl, Ring A is substituted by at least one R_{A}.

In some aspects, the present disclosure provides, inter alia, a compound of Formula (I):

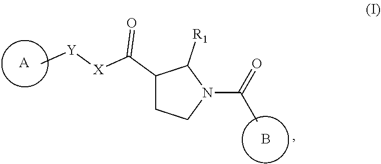

or a prodrug, solvate, or pharmaceutically acceptable salt thereof, wherein:
X is —N(Rₓ)— or —O—;
Y is absent or —C(R_{Y})₂—
Rₓ is H, C₁-C₆ alkyl, C₂-C₆ alkenyl, C₂-C₆ alkynyl, or C₁-C₆ haloalkyl;
each R_{Y} independently is H, C₁-C₆ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl;
Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A};
Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B};
R₁ is H, C₁-C₆ alkyl, C₂-C₆ alkenyl, C₂-C₆ alkynyl, or C₁-C₆ haloalkyl;
each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B} independently is halogen, —CN, —(CH₂)ₙ—OR_{B1}, —(CH₂)ₙ—N(R_{B1})(R_{B2}), —(CH₂)ₙ—S(R_{B1}), C₁-C₆ alkyl, C₂-C₆ alkenyl, C₁-C₆ haloalkyl, C₁-C₆ alkoxy, —(CH₂)ₙ—(C₆-C₁₀ aryl), (5- to 10-membered heteroaryl), (C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4};
each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or
R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′};
each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy;
each R_{B4} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₘ—C(O)R_{B4′}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4″});
each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH;
n is 0, 1, 2, 3, 4, or 5; and
m is 0, 1, 2, 3, 4, or 5,
provided that when R_{B} is a substituted or unsubstituted alkyl, Ring A is substituted by at least one R_{A}.

In some aspects, the present disclosure provides a compound obtainable by, or obtained by, a method for preparing a compound as described herein (e.g., a method comprising one or more steps described in Schemes I-V).

In some aspects, the present disclosure provides a pharmaceutical composition comprising a compound of the present disclosure, or a pharmaceutically acceptable salt thereof, and a pharmaceutically acceptable diluent or carrier.

In some aspects, the present disclosure provides an intermediate as described herein, being suitable for use in a method for preparing a compound as described herein (e.g., the intermediate is selected from the intermediates described in Examples 1-164).

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some embodiments, the disease or disorder is a viral infection. In some embodiments, the viral infection is hepatitis B virus (HBV).

In some aspects, the present disclosure provides a method of preparing a compound of the present disclosure.

In some aspects, the present disclosure provides a method of preparing a compound, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE DRAWINGS

FIG. 1A-FIG. 1E depict the electron micrographs of solvent control (2% DMSO) (FIG. 1A), BAY 41-4109 (FIG. 1B), NVR 3-778 (FIG. 1C), Example 42 (FIG. 1D), and Example 104 (FIG. 1E) for HBV capsid assembly determination.

FIG. 2 depicts the effects of Example 42, Example 104, GLS4 (class 1 compound), and entecavir (ETV) on HBV capsid, intracellular core protein, and encapsidated DNA and RNA levels in the stably HBV expressed HepG2.2.15 cell line.

FIG. 3A and FIG. 3B depict the inhibitory activities of Example 42, Example 149, and entecavir (ETV) on the cccDNA establishment in HBV-infected primary human hepatocytes in treatment scheme 1 (FIG. 3A) and treatment scheme 2 (FIG. 3B).

### DETAILED DESCRIPTION

The present disclosure relates to pyrrolidine-3-carboxamide derivatives, prodrugs, and pharmaceutically acceptable salts thereof, which may modulate the HBV replication cycle and are accordingly useful in methods of treatment of the human or animal body. The present disclosure also relates to processes for the preparation of these compounds, to pharmaceutical compositions comprising them and to their use in the viral infections, such as hepatitis B virus (HBV).

### Definitions

Unless otherwise stated, the following terms used in the specification and claims have the following meanings set out below.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, such depiction also refers to the various neutral forms, cationic forms, and anionic forms of the compound. For another example, when a compound the present disclosure is depicted in an anionic form, such depiction also refers to various salts (e.g., sodium salt) of the anionic form of the compound. In some embodiments, the amine of a compound of the present disclosure is protonated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₁-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₅). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, isothiazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a RM, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons. New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognise that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

It is to be understood that, unless otherwise stated, any description of a method of treatment includes use of the compounds to provide such treatment as is described herein. It is to be further understood, unless otherwise stated, any description of a method of treatment includes use of the compounds to prepare a medicament to treat such condition. The treatment includes treatment of human or non-human animals including rodents and other disease models used herein.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein. Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

As used here, the term “cure” or “curing” describes relieving a subject of development of the disease or condition at or below the level of detection. As used herein, the level of detection refers to levels of active virus. A cure may refer to the removal of active virus and/or inactivation of virus.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, the term “therapeutically effective amount”, refers to an amount of a pharmaceutical agent to treat or ameliorate an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognise the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition. An ordinarily skilled physician or veterinarian can readily determine and prescribe the effective amount of the drug required to counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

As use herein, the phrase “compound of the disclosure” refers to those compounds which are disclosed herein, both generically and specifically.

### Compounds of the Present Disclosure

In some aspects, the present disclosure provides, inter alia, a compound of Formula (I′):

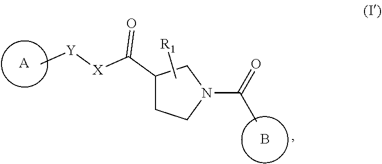

or a prodrug, solvate, or pharmaceutically acceptable salt thereof, wherein:
X is —N(Rₓ)— or —O—;
Y is absent or —C(R_{Y})₂—
Rₓ is H, C₁-C₆ alkyl, C₂-C₆ alkenyl, C₂-C₆ alkynyl, or C₁-C₆ haloalkyl;
each R_{Y} independently is H, C₁-C₆ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl;
Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A};
Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B};
R₁ is H, C₁-C₆ alkyl, C₂-C₆ alkenyl, C₂-C₆ alkynyl, or C₁-C₆ haloalkyl;
each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B} independently is halogen, —CN, —(CH₂)ₙ—OR_{B1}, —(CH₂)ₙN(R_{B1})(R_{B2}), —(CH₂)ₙ—S(R_{B1}), C₁-C₆ alkyl, C₂-C₆ alkenyl, C₁-C₆ haloalkyl, C₁-C₆ alkoxy, —(CH₂)ₙ—(C₆-C₁₀ aryl), (5- to 10-membered heteroaryl), (C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4};
each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or
R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆, alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′};
each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy;
each R_{B4} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₙ—C(O)R_{B4′}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4}″);
each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH;
n is 0, 1, 2, 3, 4, or 5; and
m is 0, 1, 2, 3, 4, or 5,
provided that when R_{B} is a substituted or unsubstituted alkyl, Ring A is substituted by at least one R_{A}.

In some aspects, the present disclosure provides, inter alia, a compound of Formula (I):

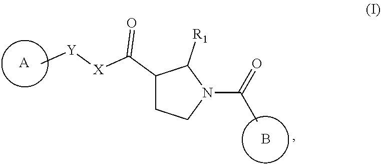

or a prodrug, solvate, or pharmaceutically acceptable salt thereof, wherein:
X is —N(Rₓ)— or —O—;
Y is absent or —C(R_{Y})₂—
Rₓ is H, C₁-C₆ alkyl, C₂-C₆ alkenyl, C₂-C₆ alkynyl, or C₁-C₆ haloalkyl;
each R_{Y} independently is H, C₁-C₆ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl;
Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A};
Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B};
R₁ is H, C₁-C₆ alkyl, C₂-C₆ alkenyl, C₂-C₆ alkynyl, or C₁-C₆ haloalkyl;
each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B} independently is halogen, —CN, —(CH₂)ₙ—OR_{B1}, —(CH₂)ₙN(R_{B1})(R_{B2}), —(CH₂)ₙ—S(R_{B1}), C₁-C₆ alkyl, C₂-C₆ alkenyl, C₁-C₆ haloalkyl, C₁-C₆ alkoxy, —(CH₂)ₙ(C₆-C₁₀ aryl), (5- to 10-membered heteroaryl), (C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4};
each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or
R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′};
each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy;
each R_{B4} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₘ—C(O)R_{B4}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4″});
each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₃-C₆ cycloalkyl, or 3- to 7-membered heterocycloalkyl, wherein the alkyl, alkenyl, alkynyl, cycloalkyl, and heterocycloalkyl are optionally substituted with one or more oxo or —OH;
n is 0, 1, 2, 3, 4, or 5; and
m is 0, 1, 2, 3, 4, or 5,
provided that when R_{B} is a substituted or unsubstituted alkyl, Ring A is substituted by at least one R_{A}.

It is understood that, for a compound of Formula (I) or (I′), X, Y, R_{X}, R_{Y}, Ring A, Ring B, R₁, R_{A}, R_{B}, R_{B1}, R_{B2}, R_{B3}, R_{B3′}, R_{B4}, R_{B4′}, R_{B4″}, n, and m can each be, where applicable, selected from the groups described herein, and any group described herein for any of X, Y, R_{X}, R_{Y}, Ring A, Ring B, R₁, R_{A}, R_{B}, R_{B1}, R_{B2}, R_{B3}, R_{B3′}, R_{B4}, R_{B4′}, R_{4″}, n, and m can be combined, where applicable, with any group described herein for one or more of the remainder of X, Y, R_{X}, R_{Y}, Ring A, Ring B, R₁, R_{A}, R_{B}, R_{B1}, R_{B2}, R_{B3}, R_{B3′}, R_{B4}, R_{B4′}, R_{4″}, n, and m.

In some aspects, the present disclosure provides a compound of Formula (I), Formula (I′), or a pharmaceutically acceptable salt thereof, wherein:
X is —N(Rₓ)—;
Y is absent;
Rₓ is H or C₁-C₆ alkyl;
Ring A is C₆-C₁₀ aryl or 5- to 10-membered heteroaryl, wherein the aryl and heteroaryl are optionally substituted with one or more R_{A};
Ring B is C₆-C₁₀ aryl or 5- to 10-membered heteroaryl, wherein the aryl and heteroaryl are optionally substituted with one or more R_{B}; provided that when R_{B} is a substituted or unsubstituted alkyl, Ring A is substituted by at least on R_{A}.

In some aspects, the present disclosure provides a compound of Formula (I), Formula (I′), or a pharmaceutically acceptable salt thereof, wherein:
X is —N(Rₓ)—;
Y is absent;
Rₓ is H or C₁-C₆ alkyl;
Ring A is C₆-C₁₀ aryl or 5- to 10-membered heteroaryl, wherein the aryl and heteroaryl are optionally substituted with one or more R_{A};
Ring B is 5- to 10-membered heteroaryl or 3- to 7-membered heterocycloalkyl, wherein the heteroaryl and heterocycloalkyl are optionally substituted with one or more R_{B}; and
R₁ is H or C₁-C₆ alkyl;
provided that when R_{B} is a substituted or unsubstituted alkyl, Ring A is substituted by at least on R_{A}.

In some aspects, the present disclosure provides a compound of Formula (I), Formula (I′), or a pharmaceutically acceptable salt thereof, wherein:
X is —N(Rₓ)—;
Y is absent;
Rₓ is H or C₁-C₆ alkyl;
Ring A is C₁-C₁₀ aryl or 5- to 10-membered heteroaryl, wherein the aryl and heteroaryl are optionally substituted with one or more R_{A};
Ring B is 5- to 10-membered heteroaryl optionally substituted with one or more R_{B}; and
R₁ is H or C₁-C₆ alkyl;
provided that when R_{B} is a substituted or unsubstituted alkyl, Ring A is substituted by at least on R_{A}.

In some embodiments, X is —N(Rₓ)— or —O—.

In some embodiments, X is —N(Rₓ)—. In some embodiments, X is —NH—. In some embodiments, X is —O—.

In some embodiments, Y is absent or —C(R_{Y})₂—.

In some embodiments, Y is absent. In some embodiments, Y is —C(R_{Y})₂—. In some embodiments, Y is —CH₂—.

In some embodiments, Rₓ is H, C₁-C₆ alkyl, C₂-C₆ alkenyl, C₂-C₆ alkynyl, or C₁-C₆ haloalkyl.

In some embodiments, Rₓ is H.

In some embodiments, Rₓ is C₁-C₆ alkyl, C₂-C₆ alkenyl, C₂-C₆ alkynyl, or C₁-C₆ haloalkyl.

In some embodiments, Rₓ is C₁-C₆ alkyl, C₂-C₆ alkenyl, or C₂-C₆ alkynyl.

In some embodiments, R_{T} is C₁-C₆ alkyl.

In some embodiments, Rₓ is methyl. In some embodiments, Rₓ is ethyl. In some embodiments, Rₓ is propyl. In some embodiments, Rₓ is butyl. In some embodiments, R_{T} is pentyl. In some embodiments, Rₓ is hexyl. In some embodiments, Rₓ is isopropyl. In some embodiments, Rₓ is isobutyl. In some embodiments, Rₓ is isopentyl. In some embodiments, Rₓ is isohexyl. In some embodiments, Rₓ is secbutyl. In some embodiments, Rₓ is secpentyl. In some embodiments, Rₓ is sechexyl. In some embodiments, Rₓ is tertbutyl.

In some embodiments, Rₓ is C₂-C₆ alkenyl. In some embodiments, Rₓ is C₂ alkenyl. In some embodiments, Rₓ is C₃ alkenyl. In some embodiments, Rₓ is C₄ alkenyl. In some embodiments, Rₓ is C₅ alkenyl. In some embodiments, Rₓ is C₆ alkenyl.

In some embodiments, Rₓ is C₂-C₆ alkynyl. In some embodiments, Rₓ is C₂ alkynyl. In some embodiments, Rₓ is C₃ alkynyl. In some embodiments, Rₓ is C₄ alkynyl. In some embodiments, Rₓ is C₅ alkynyl. In some embodiments, Rₓ is C₆ alkynyl.

In some embodiments, Rₓ is C₁-C₆ haloalkyl. In some embodiments, Rₓ is halomethyl. In some embodiments, Rₓ is haloethyl. In some embodiments, Rₓ is halopropyl. In some embodiments, Rₓ is halobutyl. In some embodiments, Rₓ is halopentyl. In some embodiments, Rₓ is halohexyl.

In some embodiments, each R_{Y} independently is H, C₁-C₆ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl.

In some embodiments, each R_{Y} independently is H, C₁-C₆ alkyl, C₂-C₆ alkenyl, C₂-C₆ alkynyl, or C₁-C₆ haloalkyl.

In some embodiments, R_{Y} is H.

In some embodiments, R_{Y} is C₁-C₆ alkyl, C₂-C₆ alkenyl, C₂-C₆ alkynyl, or C₁-C₅ haloalkyl.

In some embodiments, R_{Y} is C₁-C₆ alkyl, C₂-C₆ alkenyl, or C₂-C₆ alkynyl.

In some embodiments, R_{Y} is C₁-C₆ alkyl. In some embodiments, R_{Y} is methyl. In some embodiments, R_{Y} is ethyl. In some embodiments, R_{Y} is propyl. In some embodiments, R_{Y} is butyl. In some embodiments, R_{Y} is pentyl. In some embodiments, R_{Y} is hexyl. In some embodiments, R_{Y} is isopropyl. In some embodiments, R_{Y} is isobutyl. In some embodiments, R_{Y} is isopentyl. In some embodiments, R_{Y} is isohexyl. In some embodiments, R_{Y} is secbutyl. In some embodiments, R_{Y} is secpentyl. In some embodiments, R_{Y} is sechexyl. In some embodiments, R_{Y} is tertbutyl.

In some embodiments, R_{Y} is C₂-C₆ alkenyl. In some embodiments, R_{Y} is C₂ alkenyl. In some embodiments, R_{Y} is C₃ alkenyl. In some embodiments, R_{Y} is C₄ alkenyl. In some embodiments, R_{Y} is C₅ alkenyl. In some embodiments, R_{Y} is C₆ alkenyl.

In some embodiments, R_{Y} is C₂-C₆ alkynyl. In some embodiments, R_{Y} is C₂ alkynyl. In some embodiments, R_{Y} is C₃ alkynyl. In some embodiments, R_{Y} is C₄ alkynyl. In some embodiments, R_{Y} is C₅ alkynyl. In some embodiments, R_{Y} is C₆ alkynyl.

In some embodiments, R_{Y} is C₁-C₆ haloalkyl. In some embodiments, R_{Y} is halomethyl. In some embodiments, R_{Y} is haloethyl. In some embodiments, R_{Y} is halopropyl. In some embodiments, R_{Y} is halobutyl. In some embodiments, R_{Y} is halopentyl. In some embodiments, R_{Y} is halohexyl.

In some embodiments, two R_{Y} together with the atom to which they are attached form a 3- to 7-membered heterocycloalkyl or C₃-C₇ cycloalkyl.

In some embodiments, two R_{Y} together with the atom to which they are attached form a 3- to 7-membered heterocycloalkyl.

In some embodiments, two R_{Y} together with the atom to which they are attached form a 3-membered heterocycloalkyl. In some embodiments, two R_{Y} together with the atom to which they are attached form a 4-membered heterocycloalkyl. In some embodiments, two R_{Y} together with the atom to which they are attached form a 5-membered heterocycloalkyl. In some embodiments, two R_{Y} together with the atom to which they are attached form a 6-membered heterocycloalkyl. In some embodiments, two R_{Y} together with the atom to which they are attached form a 7-membered heterocycloalkyl.

In some embodiments, two R_{Y} together with the atom to which they are attached form a C₃-C₇ cycloalkyl.

In some embodiments, two R_{Y} together with the atom to which they are attached form a C cycloalkyl. In some embodiments, two R_{Y} together with the atom to which they are attached form a C₄ cycloalkyl. In some embodiments, two R_{Y} together with the atom to which they are attached form a C₅ cycloalkyl. In some embodiments, two R_{Y} together with the atom to which they are attached form a C₆ cycloalkyl. In some embodiments, two R_{Y} together with the atom to which they are attached form a C₇ cycloalkyl.

In some embodiments, Ring A is C₆-C₁₀ aryl, 5- to 10-membered heteroaryl, C₃-C₇ cycloalkyl, or 3- to 7-membered heterocycloalkyl.

In some embodiments, Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A}.

In some embodiments, Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{A}.

In some embodiments, Ring A is C₆-C₁₀ aryl or 5- to 10-membered heteroaryl.

In some embodiments, Ring A is C₆-C₁₀ aryl or 5- to 10-membered heteroaryl, wherein the aryl and heteroaryl are optionally substituted with one or more R_{A}.

In some embodiments, Ring A is C₆-C₁₀ aryl or 5- to 10-membered heteroaryl, wherein the aryl and heteroaryl are substituted with one or more R_{A}.

In some embodiments, Ring A is C₆-C₁₀ aryl. In some embodiments, Ring A is C₆-C₁₀ aryl, optionally substituted with one or more R_{A}. In some embodiments, Ring A is C₆-C₁₀ aryl, substituted with one or more R_{A}.

In some embodiments, Ring A is C₆ aryl (e.g., phenyl). In some embodiments, Ring A is C aryl (e.g., phenyl), optionally substituted with one or more R_{A}. In some embodiments, Ring A is C₆ aryl (e.g., phenyl), substituted with one or more R_{A}.

In some embodiments, Ring A is phenyl. In some embodiments, Ring A is phenyl, optionally substituted with one or more R_{A}. In some embodiments, Ring A is phenyl, substituted with one or more R_{A}. In some embodiments, Ring A is phenyl, substituted with one R_{A}. In some embodiments, Ring A is phenyl, substituted with two R_{A}. In some embodiments, Ring A is phenyl, substituted with three R_{A}.

In some embodiments, Ring A is C₅ aryl. In some embodiments, Ring A is C₅ aryl, optionally substituted with one or more R_{A}. In some embodiments, Ring A is C₅ aryl, substituted with one or more R_{A}.

In some embodiments, Ring A is C₁₀ aryl. In some embodiments, Ring A is C₁₀ aryl, optionally substituted with one or more R_{A}. In some embodiments, Ring A is C₁₀ aryl, substituted with one or more R_{A}.

In some embodiments, Ring A is 5- to 10-membered heteroaryl. In some embodiments, Ring A is 5- to 10-membered heteroaryl, optionally substituted with one or more R_{A}. In some embodiments, Ring A is 5- to 10-membered heteroaryl, substituted with one or more R_{A}.

In some embodiments, Ring A is 5-membered heteroaryl. In some embodiments, Ring A is 5-membered heteroaryl, optionally substituted with one or more R_{A}. In some embodiments, Ring A is 5-membered heteroaryl, substituted with one or more R_{A}.

In some embodiments, Ring A is 6-membered heteroaryl. In some embodiments, Ring A is 6-membered heteroaryl, optionally substituted with one or more R_{A}. In some embodiments, Ring A is 6-membered heteroaryl, substituted with one or more R_{A}.

In some embodiments, Ring A is 7-membered heteroaryl. In some embodiments, Ring A is 7-membered heteroaryl, optionally substituted with one or more R_{A}. In some embodiments, Ring A is 7-membered heteroaryl, substituted with one or more R_{A}.

In some embodiments, Ring A is 8-membered heteroaryl. In some embodiments, Ring A is 8-membered heteroaryl, optionally substituted with one or more R_{A}. In some embodiments, Ring A is 8-membered heteroaryl, substituted with one or more R_{A}.

In some embodiments, Ring A is 9-membered heteroaryl. In some embodiments, Ring A is 9-membered heteroaryl, optionally substituted with one or more R_{A}. In some embodiments, Ring A is 9-membered heteroaryl, substituted with one or more R_{A}.

In some embodiments, Ring A is 10-membered heteroaryl. In some embodiments, Ring A is 10-membered heteroaryl, optionally substituted with one or more R_{A}. In some embodiments, Ring A is 10-membered heteroaryl, substituted with one or more R_{A}.

In some embodiments, Ring A is C₃-C₇ cycloalkyl or 3- to 7-membered heterocycloalkyl. In some embodiments, Ring A is C₃-C₇ cycloalkyl or 3- to 7-membered heterocycloalkyl, wherein the cycloalkyl and heterocycloalkyl are optionally substituted with one or more R_{A}. In some embodiments, Ring A is C₃-C₇ cycloalkyl or 3- to 7-membered heterocycloalkyl, wherein the cycloalkyl and heterocycloalkyl are substituted with one or more R_{A}.

In some embodiments, Ring A is C₃-C₇ cycloalkyl. In some embodiments, Ring A is C₁-C₇ cycloalkyl, optionally substituted with one or more R_{A}. In some embodiments, Ring A is C₃-C₇ cycloalkyl, substituted with one or more R_{A}.

In some embodiments, Ring A is C₃ cycloalkyl. In some embodiments, Ring A is C₃ cycloalkyl, optionally substituted with one or more R_{A}. In some embodiments, Ring A is C₃ cycloalkyl, substituted with one or more R_{A}.

In some embodiments, Ring A is C₄ cycloalkyl. In some embodiments, Ring A is C₄ cycloalkyl, optionally substituted with one or more R_{A}. In some embodiments, Ring A is C₄ cycloalkyl, substituted with one or more R_{A}.

In some embodiments, Ring A is C₅ cycloalkyl. In some embodiments, Ring A is C₅ cycloalkyl, optionally substituted with one or more R_{A}. In some embodiments, Ring A is C₅ cycloalkyl, substituted with one or more R_{A}.

In some embodiments, Ring A is C₆ cycloalkyl. In some embodiments, Ring A is C₆ cycloalkyl, optionally substituted with one or more R_{A}. In some embodiments, Ring A is C₆ cycloalkyl, substituted with one or more R_{A}.

In some embodiments, Ring A is C₇ cycloalkyl. In some embodiments, Ring A is C₇ cycloalkyl, optionally substituted with one or more R_{A}. In some embodiments, Ring A is C₇ cycloalkyl, substituted with one or more R_{A}.

In some embodiments, Ring A is 3- to 7-membered heterocycloalkyl. In some embodiments, Ring A is 3- to 7-membered heterocycloalkyl, optionally substituted with one or more R_{A}. In some embodiments, Ring A is 3- to 7-membered heterocycloalkyl, substituted with one or more R_{A}.

In some embodiments, Ring A is 3-membered heterocycloalkyl. In some embodiments, Ring A is 3-membered heterocycloalkyl, optionally substituted with one or more R_{A}. In some embodiments, Ring A is 3-membered heterocycloalkyl, substituted with one or more R_{A}.

In some embodiments, Ring A is 4-membered heterocycloalkyl. In some embodiments, Ring A is 4-membered heterocycloalkyl, optionally substituted with one or more R_{A}. In some embodiments, Ring A is 4-membered heterocycloalkyl, substituted with one or more R_{A}.

In some embodiments, Ring A is 5-membered heterocycloalkyl. In some embodiments, Ring A is 5-membered heterocycloalkyl, optionally substituted with one or more R_{A}. In some embodiments, Ring A is 5-membered heterocycloalkyl, substituted with one or more R_{A}.

In some embodiments, Ring A is 6-membered heterocycloalkyl. In some embodiments, Ring A is 6-membered heterocycloalkyl, optionally substituted with one or more R_{A}. In some embodiments, Ring A is 6-membered heterocycloalkyl, substituted with one or more R_{A}.

In some embodiments, Ring A is 7-membered heterocycloalkyl. In some embodiments, Ring A is 7-membered heterocycloalkyl, optionally substituted with one or more R_{A}. In some embodiments, Ring A is 7-membered heterocycloalkyl, substituted with one or more R_{A}.

In some embodiments, Ring B is C₆-C₁₀ aryl, 5- to 10-membered heteroaryl, C₃-C₇ cycloalkyl, or 3- to 7-membered heterocycloalkyl.

In some embodiments, Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B}.

In some embodiments, Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{B}.

In some embodiments, Ring B is C₆-C₁₀ aryl or 5- to 10-membered heteroaryl.

In some embodiments, Ring B is C₆-C₁₀ aryl or 5- to 10-membered heteroaryl, wherein the aryl and heteroaryl are optionally substituted with one or more R_{B}.

In some embodiments, Ring B is C₆-C₁₀ aryl or 5- to 10-membered heteroaryl, wherein the aryl and heteroaryl are substituted with one or more R_{B}.

In some embodiments, Ring B is C₆-C₁₀ aryl. In some embodiments, Ring B is C₆-C₁₀ aryl, optionally substituted with one or more R_{B}. In some embodiments, Ring B is C₆-C₁₀ aryl, substituted with one or more R_{B}.

In some embodiments, Ring B is C₆ aryl (e.g., phenyl). In some embodiments, Ring B is C₆ aryl (e.g., phenyl), optionally substituted with one or more R_{B}. In some embodiments, Ring B is C₆ aryl (e.g., phenyl), substituted with one or more R_{B}.

In some embodiments, Ring B is C₅ aryl. In some embodiments, Ring B is C₅ aryl, optionally substituted with one or more R_{B}. In some embodiments, Ring B is C₅ aryl, substituted with one or more R_{B}.

In some embodiments, Ring B is C₁₀ aryl. In some embodiments, Ring B is C₁₀ aryl, optionally substituted with one or more R_{B}. In some embodiments, Ring B is C₁₀ aryl, substituted with one or more R_{B}.

In some embodiments, Ring B is 5- to 10-membered heteroaryl. In some embodiments, Ring B is 5- to 10-membered heteroaryl, optionally substituted with one or more R_{B}. In some embodiments, Ring B is 5- to 10-membered heteroaryl, substituted with one or more R_{B}.

In some embodiments, Ring B is 5-membered heteroaryl. In some embodiments, Ring B is 5-membered heteroaryl, optionally substituted with one or more R_{B}. In some embodiments, Ring B is 5-membered heteroaryl, substituted with one or more R_{B}.

In some embodiments, Ring B is 6-membered heteroaryl. In some embodiments, Ring B is 6-membered heteroaryl, optionally substituted with one or more R_{B}. In some embodiments, Ring B is 6-membered heteroaryl, substituted with one or more R_{B}.

In some embodiments, Ring B is 7-membered heteroaryl. In some embodiments, Ring B is 7-membered heteroaryl, optionally substituted with one or more R_{B}. In some embodiments, Ring B is 7-membered heteroaryl, substituted with one or more R_{B}.

In some embodiments, Ring B is 8-membered heteroaryl. In some embodiments, Ring B is 8-membered heteroaryl, optionally substituted with one or more R_{B}. In some embodiments, Ring B is 8-membered heteroaryl, substituted with one or more R_{B}.

In some embodiments, Ring B is 9-membered heteroaryl. In some embodiments, Ring B is 9-membered heteroaryl, optionally substituted with one or more R_{B}. In some embodiments, Ring B is 9-membered heteroaryl, substituted with one or more R_{B}.

In some embodiments, Ring B is 10-membered heteroaryl. In some embodiments, Ring B is 10-membered heteroaryl, optionally substituted with one or more R_{B}. In some embodiments, Ring B is 10-membered heteroaryl, substituted with one or more R_{B}.

In some embodiments, Ring B is C₃-C₇ cycloalkyl or 3- to 7-membered heterocycloalkyl. In some embodiments, Ring B is C₃-C₇ cycloalkyl or 3- to 7-membered heterocycloalkyl, wherein the cycloalkyl and heterocycloalkyl are optionally substituted with one or more R_{B}. In some embodiments, Ring B is C₃-C₇ cycloalkyl or 3- to 7-membered heterocycloalkyl, wherein the cycloalkyl and heterocycloalkyl are substituted with one or more R_{B}.

In some embodiments, Ring B is C₃-C₇ cycloalkyl. In some embodiments, Ring B is C₁-C₇ cycloalkyl, optionally substituted with one or more Rn. In some embodiments, Ring B is C₃-C₇ cycloalkyl, substituted with one or more R_{B}.

In some embodiments, Ring B is C₃ cycloalkyl. In some embodiments, Ring B is C₃ cycloalkyl, optionally substituted with one or more R_{B}. In some embodiments, Ring B is C₃ cycloalkyl, substituted with one or more R_{B}.

In some embodiments, Ring B is C₄ cycloalkyl. In some embodiments, Ring B is C₄ cycloalkyl, optionally substituted with one or more R_{B}. In some embodiments, Ring B is C₄ cycloalkyl, substituted with one or more Rn.

In some embodiments, Ring B is C₅ cycloalkyl. In some embodiments, Ring B is C₅ cycloalkyl, optionally substituted with one or more R_{B}. In some embodiments, Ring B is C₅ cycloalkyl, substituted with one or more Rn.

In some embodiments, Ring B is C₆ cycloalkyl. In some embodiments, Ring B is C₆ cycloalkyl, optionally substituted with one or more R_{B}. In some embodiments, Ring B is C₆ cycloalkyl, substituted with one or more R_{B}.

In some embodiments, Ring B is C₇ cycloalkyl. In some embodiments, Ring B is C₇ cycloalkyl, optionally substituted with one or more R_{B}. In some embodiments, Ring B is C₇ cycloalkyl, substituted with one or more R_{B}.

In some embodiments, Ring B is 3- to 7-membered heterocycloalkyl. In some embodiments, Ring B is 3- to 7-membered heterocycloalkyl, optionally substituted with one or more R_{B}. In some embodiments, Ring B is 3- to 7-membered heterocycloalkyl, substituted with one or more R_{B}.

In some embodiments, Ring B is 3-membered heterocycloalkyl. In some embodiments, Ring B is 3-membered heterocycloalkyl, optionally substituted with one or more R_{B}. In some embodiments, Ring B is 3-membered heterocycloalkyl, substituted with one or more R_{B}.

In some embodiments, Ring B is 4-membered heterocycloalkyl. In some embodiments, Ring B is 4-membered heterocycloalkyl, optionally substituted with one or more R_{B}. In some embodiments, Ring B is 4-membered heterocycloalkyl, substituted with one or more R_{B}.

In some embodiments, Ring B is 5-membered heterocycloalkyl. In some embodiments, Ring B is 5-membered heterocycloalkyl, optionally substituted with one or more R_{B}. In some embodiments, Ring B is 5-membered heterocycloalkyl, substituted with one or more R_{B}.

In some embodiments, Ring B is 6-membered heterocycloalkyl. In some embodiments, Ring B is 6-membered heterocycloalkyl, optionally substituted with one or more R_{B}. In some embodiments, Ring B is 6-membered heterocycloalkyl, substituted with one or more R_{B}.

In some embodiments, Ring B is 7-membered heterocycloalkyl. In some embodiments, Ring B is 7-membered heterocycloalkyl, optionally substituted with one or more R_{B}. In some embodiments, Ring B is 7-membered heterocycloalkyl, substituted with one or more R_{B}.

In some embodiments, R₁ is H, C₁-C₆ alkyl, C₂-C₆ alkenyl, C₂-C₆ alkynyl, or C₁-C₆ haloalkyl.

In some embodiments, R₁ is H.

In some embodiments, R₁ is C₁-C₆ alkyl, C₂-C₆ alkenyl, C₂-C₆ alkynyl, or C₁-C₆ haloalkyl.

In some embodiments, R₁ is C₁-C₆ alkyl, C₂-C₆ alkenyl, or C₂-C₆ alkynyl.

In some embodiments, R₁ is C₁-C₆ alkyl. In some embodiments, R₁ is methyl. In some embodiments, R₁ is ethyl. In some embodiments, R₁ is propyl. In some embodiments, R₁ is butyl. In some embodiments, R₁ is pentyl. In some embodiments, R₁ is hexyl. In some embodiments, R₁ is isopropyl. In some embodiments, R₁ is isobutyl. In some embodiments, R₁ is isopentyl. In some embodiments, R₁ is isohexyl. In some embodiments, R₁ is secbutyl. In some embodiments, R₁ is secpentyl. In some embodiments, R₁ is sechexyl. In some embodiments, R₁ is tertbutyl.

In some embodiments, R₁ is C₂-C₆ alkenyl. In some embodiments, R₁ is C₂ alkenyl. In some embodiments, R₁ is C₃ alkenyl. In some embodiments, R₁ is C₄ alkenyl. In some embodiments, R₁ is C₅ alkenyl. In some embodiments, R₁ is C₆ alkenyl.

In some embodiments, R₁ is C₂-C₆ alkynyl. In some embodiments, R₁ is C₂ alkynyl. In some embodiments, R₁ is C₃ alkynyl. In some embodiments, R₁ is C₄ alkynyl. In some embodiments, R₁ is C₅ alkynyl. In some embodiments, R₁ is C₆ alkynyl.

In some embodiments, R₁ is C₁-C₆ haloalkyl. In some embodiments, R₁ is halomethyl. In some embodiments, R₁ is haloethyl. In some embodiments, R₁ is halopropyl. In some embodiments, R₁ is halobutyl. In some embodiments, R₁ is halopentyl. In some embodiments, R₁ is halohexyl.

In some embodiments, each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, each R_{A} independently is halogen, —CN, —OH, —NH₂, —NH(C₁-C₆ alkyl), or —N(C₁-C₆ alkyl)₂.

In some embodiments, each R_{A} independently is halogen, —CN, or —OH.

In some embodiments, each R_{A} independently is halogen.

In some embodiments, each R_{A} independently is F, Cl, Br, or I. In some embodiments, each R_{A} independently is F, Cl, or Br. In some embodiments, each R_{A} independently is F or Cl. In some embodiments, each R_{A} independently is F. In some embodiments, each R_{A} independently is C₁. In some embodiments, each R_{A} independently is Br. In some embodiments, each R_{A} independently is I.

In some embodiments, each R_{A} independently is —CN. In some embodiments, each R_{A} independently is —OH.

In some embodiments, each R_{A} independently is —NH₂, —NH(C₁-C₆ alkyl), or —N(C₁-C₆ alkyl)₂.

In some embodiments, each R_{A} independently is —NH₂. In some embodiments, each R_{A} independently is —NH(C₁-C₆ alkyl). In some embodiments, each R_{A} independently is —N(C₁-C₆ alkyl)₂.

In some embodiments, each R_{A} independently is C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, each R_{A} independently is C₁-C₆ alkyl, C₂-C₆ alkenyl, C₂-C₆ alkynyl, C₁-C₆ haloalkyl, or C₁-C₆ alkoxy.

In some embodiments, each R_{A} independently is C₁-C₆ alkyl, C₂-C₆ alkenyl, or C₂-C₆ alkynyl.

In some embodiments, each R_{A} independently is C₁-C₆ alkyl. In some embodiments, each R_{A} independently is methyl. In some embodiments, each R_{A} independently is ethyl. In some embodiments, each R_{A} independently is propyl. In some embodiments, each R_{A} independently is butyl. In some embodiments, each R_{A} independently is pentyl. In some embodiments, each R_{A} independently is hexyl. In some embodiments, each R_{A} independently is isopropyl. In some embodiments, each R_{A} independently is isobutyl. In some embodiments, each R_{A} independently is isopentyl. In some embodiments, each R_{A} independently is isohexyl. In some embodiments, each R_{A} independently is secbutyl. In some embodiments, each R_{A} independently is secpentyl. In some embodiments, each R_{A} independently is sechexyl. In some embodiments, each R_{A} independently is tertbutyl.

In some embodiments, each R_{A} independently is C₂-C₆ alkenyl. In some embodiments, each R_{A} independently is C₂ alkenyl. In some embodiments, each R_{A} independently is C₃ alkenyl. In some embodiments, each R_{A} independently is C₄ alkenyl. In some embodiments, each R_{A} independently is C₅ alkenyl. In some embodiments, each R_{A} independently is C₆ alkenyl.

In some embodiments, each R_{A} independently is C₂-C₆ alkynyl. In some embodiments, each R_{A} independently is C₂ alkynyl. In some embodiments, each R_{A} independently is C₃ alkynyl. In some embodiments, each R_{A} independently is CA alkynyl. In some embodiments, each R_{A} independently is C₅ alkynyl. In some embodiments, each R_{A} independently is C₆ alkynyl.

In some embodiments, each R_{A} independently is C₁-C₆ haloalkyl or C₁-C₆ alkoxy.

In some embodiments, each R_{A} independently is C₁-C₆ haloalkyl. In some embodiments, each R_{A} independently is halomethyl. In some embodiments, each R_{A} independently is haloethyl. In some embodiments, each R_{A} independently is halopropyl. In some embodiments, each R_{A} independently is halobutyl. In some embodiments, each R_{A} independently is halopentyl. In some embodiments, each R_{A} independently is halohexyl.

In some embodiments, each R_{A} independently is C₁-C₆ alkoxy. In some embodiments, each R_{A} independently is methoxy. In some embodiments, each R_{A} independently is ethoxy. In some embodiments, each R_{A} independently is propoxy. In some embodiments, each R_{A} independently is butoxy. In some embodiments, each R_{A} independently is pentoxy. In some embodiments, each R_{A} independently is hexoxy.

In some embodiments, each R_{A} independently is 3- to 7-membered heterocycloalkyl or C₃-C₇ cycloalkyl.

In some embodiments, each R_{A} independently is 3- to 7-membered heterocycloalkyl.

In some embodiments, each R_{A} independently is 3-membered heterocycloalkyl. In some embodiments, each R_{A} independently is 4-membered heterocycloalkyl. In some embodiments, each R_{A} independently is 5-membered heterocycloalkyl. In some embodiments, each R_{A} independently is 6-membered heterocycloalkyl. In some embodiments, each R_{A} independently is 7-membered heterocycloalkyl.

In some embodiments, each R_{A} independently is C₃-C₇ cycloalkyl.

In some embodiments, each R_{A} independently is C₃ cycloalkyl. In some embodiments, each R_{A} independently is C₄ cycloalkyl. In some embodiments, each R_{A} independently is C₅ cycloalkyl. In some embodiments, each R_{A} independently is C₆ cycloalkyl. In some embodiments, each R_{A} independently is C₇ cycloalkyl.

In some embodiments, each R_{B} independently is halogen, —CN, —(CH₂)ₙ—OR_{B1}, —(CH₂)ₙ—N(R_{B1})(R_{B2}), —(CH₂)ₙ—S(R_{B1}), C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}).

In some embodiments, each R_{B} independently is halogen, —CN, —(CH₂)ₙ—OR_{B1}, —(CH₂)ₙ—N(R_{B1})(R_{B2}), —(CH₂)ₙ—S(R_{B1}), C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₁-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4}.

In some embodiments, each R_{B} independently is halogen, —CN, —(CH₂)ₙ—OR_{B1}, —(CH₂)ₙ N(R_{B1})(R_{B2}), —(CH₂)ₙ—S(R_{B1}), C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₃-C₇ cycloalkyl), —(CH₂)ₙ-(3- to 7-membered heterocycloalkyl), —C(O)R_{B}, —C(O)OR_{B1}, or —C(O)N(R_{B1})(R_{B2}), wherein the alkyl, alkenyl, alkynyl, aryl, heteroaryl, cycloalkyl, and heterocycloalkyl are substituted with one or more R_{B4}.

In some embodiments, each R_{B} independently is halogen, —CN, —(CH₂)ₙ—OR_{B1}, —(CH₂)ₙ N(R_{B1})(R_{B2}), —(CH₂)ₙ—S(R_{B1}), —C(O)R_{B1}, —C(O)OR_{B1}, or —C(O)N(R_{B1})(R_{B2}).

In some embodiments, each R_{B} independently is halogen or —CN.

In some embodiments, each Rn independently is halogen.

In some embodiments, each R_{B} independently is F, Cl, Br, or I. In some embodiments, each R_{B} independently is F, Cl, or Br. In some embodiments, each R_{B} independently is F or Cl. In some embodiments, each R_{B} independently is F. In some embodiments, each R_{B} independently is C₁. In some embodiments, each R_{B} independently is Br. In some embodiments, each R_{B} independently is I.

In some embodiments, each R_{B} independently is —CN.

In some embodiments, each R_{B} independently is —(CH₂)ₙ—OR_{B1}, —(CH₂)ₙ—N(R_{B1})(R_{B2}), or —(CH₂)ₙ—S(Rai).

In some embodiments, each R_{B} independently is —(CH₂)ₙ—OR_{B1}. In some embodiments, each R_{B} independently is —OR_{B1}. In some embodiments, each R_{B} independently is —(CH₂)—OR_{B1}. In some embodiments, each R_{B} independently is —(CH₂)₂—OR_{B1}. In some embodiments, each R_{B} independently is —(CH₂)₃—OR_{B1}.

In some embodiments, each R_{B} independently is —(CH₂)ₙ—N(R_{B1})(R_{B2}). In some embodiments, each R_{B} independently is —N(R_{B1})(R_{B2}). In some embodiments, each R_{B} independently is —(Cl₂)—N(R_{B1})(R_{B2}). In some embodiments, each R_{B} independently is —(CH₂)₂—N(R_{B1})(R_{B2}). In some embodiments, each R_{B} independently is —(CH₂)₃—N(R_{B1})(R_{B2}).

In some embodiments, each R_{B} independently is —(CH₂)—S(R_{B1}). In some embodiments, each R_{B} independently is —S(R_{B1}). In some embodiments, each R_{B} independently is —(CH₂)—S(R_{B1}). In some embodiments, each R_{B} independently is —(CH₂)₂—S(R_{B1}). In some embodiments, each R_{B} independently is —(CH₂)₃—S(R_{B1}).

In some embodiments, each R_{B} independently is —C(O)R_{B1}, —C(O)OR_{B1}, or —C(O)N(R_{B1})(R_{B2}).

In some embodiments, each R_{B} independently is —C(O)R_{B1}. In some embodiments, each R_{B} independently is —C(O)OR_{B1}. In some embodiments, each R_{B} independently is —C(O)N(R_{B1})(R_{B2}).

In some embodiments, each R_{B} independently is C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₃-C₇ cycloalkyl), or —(CH₂)ₙ-(3- to 7-membered heterocycloalkyl).

In some embodiments, each R_{B} independently is C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₃-C₇ cycloalkyl), or —(CH₂)ₙ-(3- to 7-membered heterocycloalkyl), wherein the alkyl, alkenyl, alkynyl, aryl, heteroaryl, cycloalkyl, and heterocycloalkyl are optionally substituted with one or more R_{B4}.

In some embodiments, each R_{B} independently is C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₃-C₇ cycloalkyl), or —(CH₂)ₙ-(3- to 7-membered heterocycloalkyl), wherein the alkyl, alkenyl, alkynyl, aryl, heteroaryl, cycloalkyl, and heterocycloalkyl are substituted with one or more R_{B4}.

In some embodiments, each R_{B} independently is C₁-C₆ alkyl, C₂-C₆ alkenyl, C₂-C₆ alkynyl, C₁-C₆ haloalkyl, or C₁-C₆ alkoxy.

In some embodiments, each R_{B} independently is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more R_{B4}.

In some embodiments, each R_{B} independently is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are substituted with one or more R_{B4}.

In some embodiments, each R_{B} independently is C₁-C₆ alkyl, C₂-C₆ alkenyl, or C₂-C₆ alkynyl.

In some embodiments, each R_{B} independently is C₁-C₆ alkyl, C₂-C₆ alkenyl, or C₂-C₆ alkynyl, wherein the alkyl, alkenyl, or alkynyl are optionally substituted with one or more R_{B4}.

In some embodiments, each R_{B} independently is C₁-C₆ alkyl, C₂-C₆ alkenyl, or C₂-C₆ alkynyl, wherein the alkyl, alkenyl, or alkynyl are substituted with one or more R_{B4}.

In some embodiments, each R_{B} independently is C₁-C₆ alkyl. In some embodiments, each R_{B} independently is C₁-C₆ alkyl optionally substituted with one or more R_{B4}. In some embodiments, each R_{B} independently is C₁-C₆ alkyl substituted with one or more R_{B4}.

In some embodiments, each R_{B} independently is methyl. In some embodiments, each R_{B} independently is ethyl. In some embodiments, each R_{B} independently is propyl. In some embodiments, each R_{B} independently is butyl. In some embodiments, each R_{B} independently is pentyl. In some embodiments, each R_{B} independently is hexyl. In some embodiments, each R_{B} independently is isopropyl. In some embodiments, each R_{B} independently is isobutyl. In some embodiments, each R_{B} independently is isopentyl. In some embodiments, each R_{B} independently is isohexyl. In some embodiments, each R_{B} independently is secbutyl. In some embodiments, each R_{B} independently is secpentyl. In some embodiments, each R_{B} independently is sechexyl. In some embodiments, each R_{B} independently is tertbutyl.

In some embodiments, each R_{B} independently is methyl optionally substituted with one or more R_{B4}. In some embodiments, each R_{B} independently is ethyl optionally substituted with one or more R_{B4}. In some embodiments, each R_{B} independently is propyl optionally substituted with one or more R_{B4}. In some embodiments, each R_{B} independently is butyl optionally substituted with one or more R_{B4}. In some embodiments, each R_{B} independently is pentyl optionally substituted with one or more R_{B4}. In some embodiments, each R_{B} independently is hexyl optionally substituted with one or more R_{B4}. In some embodiments, each R_{B} independently is isopropyl optionally substituted with one or more R_{B4}. In some embodiments, each R_{B} independently is isobutyl optionally substituted with one or more R_{B4}. In some embodiments, each R_{B} independently is isopentyl optionally substituted with one or more R_{B4}. In some embodiments, each R_{B} independently is isohexyl optionally substituted with one or more R_{B4}. In some embodiments, each R_{B} independently is secbutyl optionally substituted with one or more R_{B4}. In some embodiments, each R_{B} independently is secpentyl optionally substituted with one or more R_{B4}. In some embodiments, each R_{B} independently is sechexyl optionally substituted with one or more R_{B4}. In some embodiments, each R_{B} independently is tertbutyl optionally substituted with one or more R_{B4}.

In some embodiments, each R_{B} independently is methyl substituted with one or more R_{B4}. In some embodiments, each R_{B} independently is ethyl substituted with one or more R_{B4}. In some embodiments, each R_{B} independently is propyl substituted with one or more R_{B4}. In some embodiments, each R_{B} independently is butyl substituted with one or more R_{B4}. In some embodiments, each R_{B} independently is pentyl substituted with one or more R_{B4}. In some embodiments, each R_{B} independently is hexyl substituted with one or more R_{B4}. In some embodiments, each R_{B} independently is isopropyl substituted with one or more R_{B}. In some embodiments, each R_{B} independently is isobutyl substituted with one or more R_{B4}. In some embodiments, each R_{B} independently is isopentyl substituted with one or more R_{B4}. In some embodiments, each R_{B} independently is isohexyl substituted with one or more R_{B4}. In some embodiments, each R_{B} independently is secbutyl substituted with one or more R_{B4}. In some embodiments, each R_{B} independently is secpentyl substituted with one or more R_{B4}. In some embodiments, each R_{B} independently is sechexyl substituted with one or more R_{B4}. In some embodiments, each R_{B} independently is tertbutyl substituted with one or more R_{B4}.

In some embodiments, each R_{B} independently is C₂-C₆ alkenyl. In some embodiments, each R_{B} independently is C₂-C₆ alkenyl optionally substituted with one or more R_{B4}. In some embodiments, each R_{B} independently is C₂-C₆ alkenyl substituted with one or more R_{B4}.

In some embodiments, each R_{B} independently is C₂ alkenyl. In some embodiments, each R_{B} independently is C₃ alkenyl. In some embodiments, each R_{B} independently is C₄ alkenyl. In some embodiments, each R_{B} independently is C₅ alkenyl. In some embodiments, each R_{B} independently is C₆ alkenyl.

In some embodiments, each R_{B} independently is C₂ alkenyl optionally substituted with one or more R_{B4}. In some embodiments, each R_{B} independently is C₃ alkenyl optionally substituted with one or more R_{B4}. In some embodiments, each R_{B} independently is C₄ alkenyl optionally substituted with one or more R_{B4}. In some embodiments, each R_{B} independently is C₅ alkenyl optionally substituted with one or more R_{B4}. In some embodiments, each R_{B} independently is C₆ alkenyl optionally substituted with one or more R_{B4}.

In some embodiments, each R_{B} independently is C₂ alkenyl substituted with one or more R_{B4}. In some embodiments, each R_{B} independently is C alkenyl substituted with one or more R_{B4}. In some embodiments, each R_{B} independently is C₄ alkenyl substituted with one or more R_{B4}. In some embodiments, each R_{B} independently is C₅ alkenyl substituted with one or more R_{B4}. In some embodiments, each R_{B} independently is C₆ alkenyl substituted with one or more R_{B4}.

In some embodiments, each R_{B} independently is C₂-C₆ alkynyl. In some embodiments, each R_{B} independently is C₂-C₆ alkynyl optionally substituted with one or more R_{B4}. In some embodiments, each R_{B} independently is C₂-C₆ alkynyl substituted with one or more R_{B4}.

In some embodiments, each R_{B} independently is C₅ alkynyl. In some embodiments, each R_{B} independently is C₅ alkynyl. In some embodiments, each R_{B} independently is C₄ alkynyl. In some embodiments, each R_{B} independently is C₅ alkynyl. In some embodiments, each R_{B} independently is C₆ alkynyl.

In some embodiments, each R_{B} independently is C₂ alkynyl optionally substituted with one or more R_{B4}. In some embodiments, each R_{B} independently is C alkynyl optionally substituted with one or more R_{B4}. In some embodiments, each R_{B} independently is C₄ alkynyl optionally substituted with one or more R_{B4}. In some embodiments, each R_{B} independently is C₅ alkynyl optionally substituted with one or more R_{B4}. In some embodiments, each R_{B} independently is C₆ alkynyl optionally substituted with one or more R_{B4}.

In some embodiments, each R_{B} independently is C₂ alkynyl substituted with one or more R_{B4}. In some embodiments, each R_{B} independently is C₃ alkynyl substituted with one or more R_{B4}. In some embodiments, each R_{B} independently is C₄ alkynyl substituted with one or more R_{B4}. In some embodiments, each R_{B} independently is C₅ alkynyl substituted with one or more R_{B4}. In some embodiments, each R_{B} independently is C₆ alkynyl substituted with one or more R_{B4}.

In some embodiments, each R_{B} independently is C₁-C₆ haloalkyl or C₁-C₆ alkoxy.

In some embodiments, each R_{B} independently is C₁-C₆ haloalkyl or C₁-C₆ alkoxy, wherein the alkyl is optionally substituted with one or more R_{B4}.

In some embodiments, each R_{B} independently is C₁-C₆ haloalkyl or C₁-C₆ alkoxy, wherein the alkyl is substituted with one or more R_{B4}.

In some embodiments, each R_{B} independently is C₁-C₆ haloalkyl. In some embodiments, each R_{B} independently is C₁-C₆ haloalkyl optionally substituted with one or more R_{B4}. In some embodiments, each R_{B} independently is C₁-C₆ haloalkyl substituted with one or more R_{B4}.

In some embodiments, each R_{B} independently is halomethyl. In some embodiments, each R_{B} independently is haloethyl. In some embodiments, each R_{B} independently is halopropyl. In some embodiments, each R_{B} independently is halobutyl. In some embodiments, each R_{B} independently is halopentyl. In some embodiments, each R_{B} independently is halohexyl.

In some embodiments, each R_{B} independently is halomethyl optionally substituted with one or more R_{B}. In some embodiments, each R_{B} independently is haloethyl optionally substituted with one or more R_{B4}. In some embodiments, each R_{B} independently is halopropyl optionally substituted with one or more R_{B4}. In some embodiments, each R_{B} independently is halobutyl optionally substituted with one or more R_{B4}. In some embodiments, each R_{B} independently is halopentyl optionally substituted with one or more R_{B4}. In some embodiments, each R_{B} independently is halohexyl optionally substituted with one or more R_{B4}.

In some embodiments, each R_{B} independently is halomethyl substituted with one or more R_{B4}. In some embodiments, each R_{B} independently is haloethyl substituted with one or more R_{B4}. In some embodiments, each R_{B} independently is halopropyl substituted with one or more R_{B4}. In some embodiments, each R_{B} independently is halobutyl substituted with one or more R_{B4}. In some embodiments, each R_{B} independently is halopentyl substituted with one or more R_{B4}. In some embodiments, each R_{B} independently is halohexyl substituted with one or more R_{B4}.

In some embodiments, each R_{B} independently is C₁-C₆ alkoxy. In some embodiments, each R_{B} independently is C₁-C₆ alkoxy optionally substituted with one or more R_{B4}. In some embodiments, each R_{B} independently is C₁-C₆ alkoxy substituted with one or more R_{B4}.

In some embodiments, each R_{B} independently is methoxy. In some embodiments, each R_{B} independently is ethoxy. In some embodiments, each R_{B} independently is propoxy. In some embodiments, each R_{B} independently is butoxy. In some embodiments, each R_{B} independently is pentoxy. In some embodiments, each R_{B} independently is hexoxy.

In some embodiments, each R_{B} independently is methoxy optionally substituted with one or more R_{B4}. In some embodiments, each R_{B} independently is ethoxy optionally substituted with one or more R_{B4}. In some embodiments, each R_{B} independently is propoxy optionally substituted with one or more R_{B4}. In some embodiments, each R_{B} independently is butoxy optionally substituted with one or more R_{B4}. In some embodiments, each R_{B} independently is pentoxy optionally substituted with one or more R_{B4}. In some embodiments, each R_{B} independently is hexoxy optionally substituted with one or more R_{B4}.

In some embodiments, each R_{B} independently is optionally substituted with one or more R_{B4}. In some embodiments, each R_{B} independently is ethoxy substituted with one or more R_{B4}. In some embodiments, each R_{B} independently is propoxy substituted with one or more R_{B4}. In some embodiments, each R_{B} independently is butoxy substituted with one or more R_{B4}. In some embodiments, each R_{B} independently is pentoxy substituted with one or more R_{B4}. In some embodiments, each R_{B} independently is hexoxy substituted with one or more R_{B4}.

In some embodiments, each R_{B} independently is —(CH₂)ₙ—(C₆-C₁₀ aryl), —(CH₂)ₙ-(5- to 10-membered heteroaryl), —(CH₂)ₙ—(C₃-C₇ cycloalkyl), or —(CH₂)ₙ-(3- to 7-membered heterocycloalkyl).

In some embodiments, each R_{B} independently is —(CH₂)ₙ—(C₆-C₁₀ aryl), (5- to 10-membered heteroaryl), —(CH₂)ₙ—(C₁-C₇ cycloalkyl), or —(CH₂)ₙ-(3- to 7-membered heterocycloalkyl), wherein the aryl, heteroaryl, cycloalkyl, and heterocycloalkyl are optionally substituted with one or more R_{B4}.

In some embodiments, each R_{B} independently is —(CH₂)ₙ—(C₆-C₁₀ aryl), —(CH₂)ₙ-(5- to 10-membered heteroaryl), —(CH₂)ₙ—(C₃-C₇ cycloalkyl), or —(CH₂)ₙ-(3- to 7-membered heterocycloalkyl), wherein the aryl, heteroaryl, cycloalkyl, and heterocycloalkyl are substituted with one or more R_{B4}.

In some embodiments, each R_{B} independently is —(CH₂)ₙ—(C₆-C₁₀ aryl) or —(CH₂)ₙ-(5- to 10-membered heteroaryl).

In some embodiments, each R_{B} independently is —(CH₂)ₙ—(C₆-C₁₀ aryl) or —(CH₂)ₙ-(5- to 10-membered heteroaryl), wherein the aryl or heteroaryl are optionally substituted with one or more R_{B4}.

In some embodiments, each R_{B} independently is —(CH₂)ₙ—(C₆-C₁₀ aryl) or —(CH₂)ₙ-(5- to 10-membered heteroaryl), wherein the aryl or heteroaryl are substituted with one or more R_{B4}.

In some embodiments, each R_{B} independently is —(CH₂)ₙ—(C₆-C₁₀ aryl). In some embodiments, each R_{B} independently is —(CH₂)ₙ—(C₆-C₁₀ aryl), wherein the aryl is optionally substituted with one or more R_{B4}. In some embodiments, each R_{B} independently is —(CH₂)ₙ—(C₆-C₁₀ aryl), wherein the aryl is substituted with one or more R_{B4}.

In some embodiments, each R_{B} independently is —(CH₂)ₙ—(C₆ aryl) (e.g., phenyl). In some embodiments, each R_{B} independently is —(CH₂)ₙ—(C₆ aryl) (e.g., phenyl), wherein the aryl is optionally substituted with one or more R_{B4}. In some embodiments, each R_{B} independently is —(CH₂)ₙ—(C₆ aryl) (e.g., phenyl), wherein the aryl is substituted with one or more R_{B4}.

In some embodiments, each R_{B} independently is phenyl. In some embodiments, each R_{B} independently is phenyl optionally substituted with one or more R_{B4}. In some embodiments, each R_{B} independently is phenyl substituted with one or more R_{B4}.

In some embodiments, each R_{B} independently is —(CH₂)-(phenyl). In some embodiments, each R_{B} independently is —(CH₂)-(phenyl), wherein the phenyl is optionally substituted with one or more R_{B4}. In some embodiments, each R_{B} independently is —(CH₂)-(phenyl), wherein the phenyl is substituted with one or more R_{B4}.

In some embodiments, each R_{B} independently is —(CH₂)₂-(phenyl) (e.g., phenyl). In some embodiments, each R_{B} independently is —(CH₂)₂-(phenyl) (e.g., phenyl), wherein the aryl is optionally substituted with one or more R_{B4}. In some embodiments, each R_{B} independently is —(CH₂)₂-(phenyl), wherein the phenyl is substituted with one or more R_{B4}.

In some embodiments, each R_{B} independently is —(CH₂)₃-(phenyl) (e.g., phenyl). In some embodiments, each R_{B} independently is —(CH₂)₃-(phenyl) (e.g., phenyl), wherein the aryl is optionally substituted with one or more R_{B4}. In some embodiments, each R_{B} independently is —(CH₂)₃-(phenyl), wherein the phenyl is substituted with one or more R_{B4}.

In some embodiments, each R_{B} independently is —(CH₂)ₙ—(C₅ aryl). In some embodiments, each R_{B} independently is —(CH₂)ₙ—(C₅ aryl), wherein the aryl is optionally substituted with one or more R_{B4}. In some embodiments, each R_{B} independently is —(CH₂)ₙ—(C₅ aryl), wherein the aryl is substituted with one or more R_{B4}.

In some embodiments, each R_{B} independently is —(CH₂)ₙ—(C₁₀ aryl). In some embodiments, each R_{B} independently is —(CH₂)ₙ—(C₁₀ aryl), wherein the aryl is optionally substituted with one or more R_{B4}. In some embodiments, each R_{B} independently is —(CH₂)ₙ—(C₁₀ aryl), wherein the aryl is substituted with one or more R_{B4}.

In some embodiments, each R_{B} independently is —(CH₂)ₙ-(5- to 10-membered heteroaryl). In some embodiments, each R_{B} independently is —(CH₂)ₙ-(5- to 10-membered heteroaryl), wherein the heteroaryl is optionally substituted with one or more R_{B4}. In some embodiments, each R_{B} independently is —(CH₂)-(5- to 10-membered heteroaryl), wherein the heteroaryl is substituted with one or more R_{B4}.

In some embodiments, each R_{B} independently is —(CH₂)ₙ-(5-membered heteroaryl). In some embodiments, each R_{B} independently is —(CH₂)ₙ-(5-membered heteroaryl), wherein the heteroaryl is optionally substituted with one or more R_{B4}. In some embodiments, each R_{B} independently is —(CH₂)ₙ-(5-membered heteroaryl), wherein the heteroaryl is substituted with one or more R_{B4}.

In some embodiments, each R_{B} independently is —(CH₂)ₙ-(6-membered heteroaryl). In some embodiments, each R_{B} independently is —(CH₂)ₙ(6-membered heteroaryl), wherein the heteroaryl is optionally substituted with one or more R_{B4}. In some embodiments, each R_{B} independently is —(CH₂)ₙ-(6-membered heteroaryl), wherein the heteroaryl is substituted with one or more R_{B4}.

In some embodiments, each R_{B} independently is —(CH₂)ₙ-(7-membered heteroaryl). In some embodiments, each R_{B} independently is —(CH₂)ₙ-(7-membered heteroaryl), wherein the heteroaryl is optionally substituted with one or more R_{B4}. In some embodiments, each R_{B} independently is —(CH₂)-(7-membered heteroaryl), wherein the heteroaryl is substituted with one or more R_{B4}.

In some embodiments, each R_{B} independently is —(CH₂)ₙ-(8-membered heteroaryl). In some embodiments, each R_{B} independently is —(CH₂)ₙ-(8-membered heteroaryl), wherein the heteroaryl is optionally substituted with one or more R_{B4}. In some embodiments, each R_{B} independently is —(CH₂)ₙ-(8-membered heteroaryl), wherein the heteroaryl is substituted with one or more R_{B4}.

In some embodiments, each R_{B} independently is —(CH₂)ₙ-(9-membered heteroaryl). In some embodiments, each R_{B} independently is —(CH₂)ₙ-(9-membered heteroaryl), wherein the heteroaryl is optionally substituted with one or more R_{B4}. In some embodiments, each R_{B} independently is —(CH₂)ₙ-(9-membered heteroaryl), wherein the heteroaryl is substituted with one or more R_{B4}.

In some embodiments, each R_{B} independently is —(CH₂)ₙ-(10-membered heteroaryl). In some embodiments, each R_{B} independently is —(CH₂)ₙ-(10-membered heteroaryl), wherein the heteroaryl is optionally substituted with one or more R_{B4}. In some embodiments, each R_{B} independently is —(CH₂)ₙ-(10-membered heteroaryl), wherein the heteroaryl is substituted with one or more R_{B4}.

In some embodiments, each R_{B} independently is —(CH₂)ₙ—(C₃-C₇ cycloalkyl) or —(CH₂)ₙ-(3- to 7-membered heterocycloalkyl).

In some embodiments, each R_{B} independently is —(CH₂)ₙ—(C₃-C₇ cycloalkyl) or —(CH₂)ₙ-(3- to 7-membered heterocycloalkyl), wherein the cycloalkyl and heterocycloalkyl are optionally substituted with one or more R_{B4}.

In some embodiments, each R_{B} independently is —(CH₂)ₙ—(C₃-C₇ cycloalkyl) or —(CH₂)ₙ-(3- to 7-membered heterocycloalkyl), wherein the cycloalkyl and heterocycloalkyl are substituted with one or more R_{B4}.

In some embodiments, each R_{B} independently is —(CH₂)ₙ—(C₃-C₇ cycloalkyl). In some embodiments, each R_{B} independently is —(CH₂)ₙ—(C₃-C₇ cycloalkyl), wherein the cycloalkyl is optionally substituted with one or more R_{B4}. In some embodiments, each R_{B} independently is —(CH₂)ₙ(C₃-C₇ cycloalkyl), wherein the cycloalkyl is substituted with one or more R_{B4}.

In some embodiments, each R_{B} independently is —(CH₂)ₙ—(C₃ cycloalkyl). In some embodiments, each R_{B} independently is —(CH₂)ₙ—(C₃ cycloalkyl), wherein the cycloalkyl is optionally substituted with one or more R_{B4}. In some embodiments, each R_{B} independently is —(CH₂)ₙ—(C₃ cycloalkyl), wherein the cycloalkyl is substituted with one or more R_{B4}.

In some embodiments, each R_{B} independently is —(CH₂)ₙ—(C₄ cycloalkyl). In some embodiments, each R_{B} independently is —(CH₂)ₙ—(C₄ cycloalkyl), wherein the cycloalkyl is optionally substituted with one or more R_{B4}. In some embodiments, each R_{B} independently is —(CH₂)ₙ—(C₄ cycloalkyl), wherein the cycloalkyl is substituted with one or more R_{B4}.

In some embodiments, each R_{B} independently is —(CH₂)ₙ—(C₅ cycloalkyl). In some embodiments, each R_{B} independently is —(CH₂)ₙ—(C₅ cycloalkyl), wherein the cycloalkyl is optionally substituted with one or more R_{B4}. In some embodiments, each R_{B} independently is —(CH₂)ₙ(C₅ cycloalkyl), wherein the cycloalkyl is substituted with one or more R_{B4}.

In some embodiments, each R_{B} independently is —(CH₂)ₙ—(C₆ cycloalkyl). In some embodiments, each R_{B} independently is —(CH₂)ₙ—(C₆ cycloalkyl), wherein the cycloalkyl is optionally substituted with one or more R_{B4}. In some embodiments, each R_{B} independently is —(CH₂)ₙ—(C₆ cycloalkyl), wherein the cycloalkyl is substituted with one or more R_{B4}.

In some embodiments, each R_{B} independently is —(CH₂)ₙ—(C₇ cycloalkyl). In some embodiments, each R_{B} independently is —(CH₂)ₙ—(C₇ cycloalkyl), wherein the cycloalkyl is optionally substituted with one or more R_{B4}. In some embodiments, each R_{B} independently is —(CH₂)ₙ—(C₂ cycloalkyl), wherein the cycloalkyl is substituted with one or more R_{B4}.

In some embodiments, each R_{B} independently is —(CH₂)ₙ-(3- to 7-membered heterocycloalkyl). In some embodiments, each R_{B} independently is —(CH₂)ₙ-(3- to 7-membered heterocycloalkyl), wherein the heterocycloalkyl is optionally substituted with one or more R_{B4}. In some embodiments, each R_{B} independently is —(CH₂)ₙ-(3- to 7-membered heterocycloalkyl), wherein the heterocycloalkyl is substituted with one or more R_{B4}.

In some embodiments, each R_{B} independently is —(CH₂)ₙ-(3-membered heterocycloalkyl). In some embodiments, each R_{B} independently is —(CH₂)-(3-membered heterocycloalkyl), wherein the heterocycloalkyl is optionally substituted with one or more R_{B4}. In some embodiments, each R_{B} independently is —(CH₂)ₙ-(3-membered heterocycloalkyl), wherein the heterocycloalkyl is substituted with one or more R_{B4}.

In some embodiments, each R_{B} independently is —(CH₂)ₙ-(4-membered heterocycloalkyl). In some embodiments, each R_{B} independently is —(CH₂)-(4-membered heterocycloalkyl), wherein the heterocycloalkyl is optionally substituted with one or more R_{B4}. In some embodiments, each R_{B} independently is —(CH₂)ₙ-(4-membered heterocycloalkyl), wherein the heterocycloalkyl is substituted with one or more R_{B4}.

In some embodiments, each R_{B} independently is —(CH₂)ₙ-(5-membered heterocycloalkyl). In some embodiments, each R_{B} independently is —(CH₂)ₙ-(5-membered heterocycloalkyl), wherein the heterocycloalkyl is optionally substituted with one or more R_{B4}. In some embodiments, each R_{B} independently is —(CH₂)ₙ-(5-membered heterocycloalkyl), wherein the heterocycloalkyl is substituted with one or more R_{B4}.

In some embodiments, each R_{B} independently is —(CH₂)ₙ-(6-membered heterocycloalkyl). In some embodiments, each R_{B} independently is —(CH₂)ₙ-(6-membered heterocycloalkyl), wherein the heterocycloalkyl is optionally substituted with one or more R_{B4}. In some embodiments, each R_{B} independently is —(CH₂)ₙ-(6-membered heterocycloalkyl), wherein the heterocycloalkyl is substituted with one or more R_{B4}.

In some embodiments, each R_{B} independently is —(CH₂)ₙ-(7-membered heterocycloalkyl). In some embodiments, each R_{B} independently is —(CH₂)ₙ-(7-membered heterocycloalkyl), wherein the heterocycloalkyl is optionally substituted with one or more R_{B4}. In some embodiments, each R_{B} independently is —(CH₂)ₙ-(7-membered heterocycloalkyl), wherein the heterocycloalkyl is substituted with one or more R_{B4}.

In some embodiments, each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₁-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B1} is H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, R_{B1} is H, halogen, —CN, —OH, —NH₂, —NH(C₁-C₆alkyl), —NH(C₁-C₆ alkyl)-OH, —N(C₁-C₆, alkyl)₂, C₁-C₆, alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, R_{B1} is H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B1} is H.

In some embodiments, R_{B1} is halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆, alkynyl, C₁-C₆ haloalkyl, C₁-C₆ alkoxy, C₁-C₁₀ aryl, 5- to 10-membered heteroaryl, C₃-C₇ cycloalkyl, or 3- to 7-membered heterocycloalkyl.

In some embodiments, R_{B1} is halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, R_{B1} is halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B1} is halogen, —CN, —OH, —NH₂, —NH(C₁-C₆ alkyl), —NH(C₁-C₆ alkyl)-OH, or —N(C₁-C₆ alkyl)₂.

In some embodiments, R_{B1} is halogen, —CN, —OH, —NH₂, —NH(C₁-C₆ alkyl), —NH(C₁-C₆ alkyl)-OH, or —N(C₁-C₆ alkyl)₂, wherein the alkyl is optionally substituted with one or more R_{B3}.

In some embodiments, R_{B1} is halogen, —CN, —OH, —NH₂, —NH(C₁-C₆ alkyl), —NH(C₁-C₆ alkyl)-OH, or —N(C₁-C₆ alkyl)₂, wherein the alkyl is substituted with one or more R_{B3}.

In some embodiments, R_{B1} is halogen or —CN.

In some embodiments, R_{B1} is halogen.

In some embodiments, R_{B1} is F, Cl, Br, or I. In some embodiments, R_{B1} is F, Cl, or Br. In some embodiments, R_{B1} is F or Cl. In some embodiments, R_{B1} is F. In some embodiments, R_{B1} is C₁. In some embodiments, R_{B1} is Br. In some embodiments, R_{B1} is I.

In some embodiments, R_{B1} is —CN.

In some embodiments, R_{B1} is —OH, —NH₂, —NH(C₁-C₆ alkyl), —NH(C₁-C₆ alkyl)-OH, or —N(C₁-C₆ alkyl)₂.

In some embodiments, R_{B1} is —OH, —NH₂, —NH(C₁-C₆ alkyl), —NH(C₁-C₆ alkyl)-OH, or —N(C₁-C₆ alkyl)₂, wherein the alkyl is optionally substituted with one or more R_{B3}.

In some embodiments, R_{B1} is —OH, —NH₂, —NH(C₁-C₆ alkyl), —NH(C₁-C₆ alkyl)-OH, or —N(C₁-C₆ alkyl)₂, wherein the alkyl is substituted with one or more R_{B3}.

In some embodiments, R_{B1} is —OH. In some embodiments, R_{B1} is —NH₂.

In some embodiments, R_{B1} is —NH(C₁-C₆ alkyl), —NH(C₁-C₆ alkyl)-OH, or —N(C₁-C₆ alkyl)₂.

In some embodiments, R_{B1} is —NH(C₁-C₆ alkyl), —NH(C₁-C₆ alkyl)-OH, or —N(C₁-C₆ alkyl)₂, wherein the alkyl is optionally substituted with one or more R_{B}.

In some embodiments, R_{B1} is —NH(C₁-C₆ alkyl), —NH(C₁-C₆ alkyl)-OH, or —N(C₁-C₆ alkyl)₂, wherein the alkyl is substituted with one or more R_{B3}.

In some embodiments, R_{B1} is —NH(C₁-C₆ alkyl). In some embodiments, R_{B1} is —NH(C₁-C₆ alkyl), wherein the alkyl is optionally substituted with one or more R_{B3}. In some embodiments, R_{B1} is —NH(C₁-C₆ alkyl), wherein the alkyl is substituted with one or more R_{B3}.

In some embodiments, R_{B1} is —NH(C₁-C₆ alkyl)-OH. In some embodiments, R_{B1} is —NH(C₁-C₆ alkyl)-OH, wherein the alkyl is optionally substituted with one or more R_{B3}. In some embodiments, R_{B1} is —NH(C₁-C₆ alkyl)-OH, wherein the alkyl is substituted with one or more R_{B3}.

In some embodiments, R_{B1} is —N(C₁-C₆ alkyl)₂. In some embodiments, R_{B1} is —N(C₁-C₆ alkyl)₂, wherein the alkyl is optionally substituted with one or more R_{B3}. In some embodiments, R_{B1} is —N(C₁-C₆ alkyl)₂, wherein the alkyl is substituted with one or more R_{B3}.

In some embodiments, R_{B1} is C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, R_{B1} is C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, R_{B1} is C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B1} is C₁-C₆ alkyl, C₂-C₆ alkenyl, C₂-C₆ alkynyl, C₁-C₆ haloalkyl, or C₁-C₆ alkoxy.

In some embodiments, R_{B1} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more R_{B3}.

In some embodiments, R_{B1} is C₁-C₆ alkyl, C₂-C₆ alkenyl, C₂-C₆ alkynyl, C₁-C₆, haloalkyl, or C₁-C₆ alkoxy, wherein the alkyl, alkenyl, or alkynyl are substituted with one or more R_{B3}.

In some embodiments, R_{B1} is C₁-C₆ alkyl, C₂-C₆ alkenyl, or C₂-C₆ alkynyl.

In some embodiments, R_{B1} is C₁-C₆ alkyl, C₂-C₆ alkenyl, or C₂-C₆ alkynyl, wherein the alkyl, alkenyl, or alkynyl are optionally substituted with one or more R_{B3}.

In some embodiments, R_{B1} is C₁-C₆ alkyl, C₂-C₆ alkenyl, or C₂-C₆ alkynyl, wherein the alkyl, alkenyl, or alkynyl are substituted with one or more R_{B3}.

In some embodiments, R_{B1} is C₁-C₆ alkyl. In some embodiments, R_{B1} is C₁-C₆ alkyl optionally substituted with one or more R_{B3}. In some embodiments, R_{B1} is C₁-C₆ alkyl substituted with one or more R_{B3}.

In some embodiments, R_{B1} is methyl. In some embodiments, R_{B1} is ethyl. In some embodiments, R_{B1} is propyl. In some embodiments, R_{B1} is butyl. In some embodiments, R_{B1} is pentyl. In some embodiments, R_{B1} is hexyl. In some embodiments, R_{B1} is isopropyl. In some embodiments, R_{B1} is isobutyl. In some embodiments, R_{B1} is isopentyl. In some embodiments, R_{B1} is isohexyl. In some embodiments, R_{B1} is secbutyl. In some embodiments, R_{B1} is secpentyl. In some embodiments, R_{B1} is sechexyl. In some embodiments, R_{B1} is tertbutyl.

In some embodiments, R_{B1} is methyl optionally substituted with one or more R_{B3}. In some embodiments, R_{B1} is ethyl optionally substituted with one or more R_{B}. In some embodiments, R_{B1} is propyl optionally substituted with one or more R_{B}. In some embodiments, R_{B1} is butyl optionally substituted with one or more R_{B3}. In some embodiments, R_{B1} is pentyl optionally substituted with one or more R_{B3}. In some embodiments, R_{B1} is hexyl optionally substituted with one or more R_{B3}. In some embodiments, R_{B1} is isopropyl optionally substituted with one or more R_{B3}. In some embodiments, R_{B1} is isobutyl optionally substituted with one or more R_{B3}. In some embodiments, R_{B1} is isopentyl optionally substituted with one or more R_{B3}. In some embodiments, R_{B1} is isohexyl optionally substituted with one or more R_{B3}. In some embodiments, R_{B1} is secbutyl optionally substituted with one or more R_{B3}. In some embodiments, R_{B1} is secpentyl optionally substituted with one or more R_{B3}. In some embodiments, R_{B1} is sechexyl optionally substituted with one or more R_{B3}. In some embodiments, R_{B1} is tertbutyl optionally substituted with one or more R_{B}.

In some embodiments, R_{B1} is methyl substituted with one or more R_{B3}. In some embodiments, R_{B1} is ethyl substituted with one or more R_{B3}. In some embodiments, R_{B1} is propyl substituted with one or more R_{B3}. In some embodiments, R_{B1} is butyl substituted with one or more R_{B3}. In some embodiments, R_{B1} is pentyl substituted with one or more R_{B}. In some embodiments, R_{B1} is hexyl substituted with one or more R_{B3}. In some embodiments, R_{B1} is isopropyl substituted with one or more R_{B3}. In some embodiments, R_{B1} is isobutyl substituted with one or more R_{B3}. In some embodiments, R_{B1} is isopentyl substituted with one or more R_{B3}. In some embodiments, R_{B1} is isohexyl substituted with one or more R_{B3}. In some embodiments, R_{B1} is secbutyl substituted with one or more R_{B3}. In some embodiments, R_{B1} is secpentyl substituted with one or more R_{B3}. In some embodiments, R_{B1} is sechexyl substituted with one or more R_{B3}. In some embodiments, R_{B1} is tertbutyl substituted with one or more R_{B3}.

In some embodiments, R_{B1} is C₂-C₆ alkenyl. In some embodiments, R_{B1} is C₂-C₆ alkenyl optionally substituted with one or more R_{B3}. In some embodiments, R_{B1} is C₂-C₆ alkenyl substituted with one or more R_{B3}.

In some embodiments, R_{B1} is C₂ alkenyl. In some embodiments, R_{B1} is C₃ alkenyl. In some embodiments, R_{B1} is C₄ alkenyl. In some embodiments, R_{B1} is C₅ alkenyl. In some embodiments, R_{B1} is C₆ alkenyl.

In some embodiments, R_{B1} is C₂ alkenyl optionally substituted with one or more R_{B3}. In some embodiments, R_{B1} is C₃ alkenyl optionally substituted with one or more R_{B3}. In some embodiments, R_{B1} is C₄ alkenyl optionally substituted with one or more R_{B3}. In some embodiments, R_{B1} is C₅ alkenyl optionally substituted with one or more R_{B3}. In some embodiments, R_{B1} is C₆ alkenyl optionally substituted with one or more R_{B}.

In some embodiments, R_{B1} is C₂ alkenyl substituted with one or more R_{B3}. In some embodiments, R_{B1} is C₃ alkenyl substituted with one or more R_{B3}. In some embodiments, R_{B1} is CA alkenyl substituted with one or more R_{B3}. In some embodiments, R_{B1} is C₅ alkenyl substituted with one or more R_{B3}. In some embodiments, R_{B1} is C₆ alkenyl substituted with one or more R_{B3}.

In some embodiments, R_{B1} is C₂-C₆ alkynyl. In some embodiments, R_{B1} is C₂-C₆ alkynyl optionally substituted with one or more R_{B3}. In some embodiments, R_{B1} is C₂-C₆ alkynyl substituted with one or more R_{B3}.

In some embodiments, R_{B1} is C₂ alkynyl. In some embodiments, R_{B1} is C₃ alkynyl. In some embodiments, R_{B1} is C₄ alkynyl. In some embodiments, R_{B1} is C₅ alkynyl. In some embodiments, R_{B1} is C₆ alkynyl.

In some embodiments, R_{B1} is C₂ alkynyl optionally substituted with one or more R_{B3}. In some embodiments, R_{B1} is C₃ alkynyl optionally substituted with one or more R_{B3}. In some embodiments, R_{B1} is C₄ alkynyl optionally substituted with one or more R_{B3}. In some embodiments, R_{B1} is C₅ alkynyl optionally substituted with one or more R_{B3}. In some embodiments, R_{B1} is C₆ alkynyl optionally substituted with one or more R_{B3}.

In some embodiments, R_{B1} is C₂ alkynyl substituted with one or more R_{B3}. In some embodiments, R_{B1} is C₃ alkynyl substituted with one or more R_{B3}. In some embodiments, R_{B1} is C₄ alkynyl substituted with one or more R_{B3}. In some embodiments, R_{B1} is C₅ alkynyl substituted with one or more R_{B3}. In some embodiments, R_{B1} is C₆ alkynyl substituted with one or more R_{B3}.

In some embodiments, R_{B1} is C₁-C₆ haloalkyl or C₁-C₆ alkoxy.

In some embodiments, R_{B1} is C₁-C₆ haloalkyl or C₁-C₆ alkoxy, wherein the alkyl is optionally substituted with one or more R_{B3}.

In some embodiments, R_{B1} is C₁-C₆ haloalkyl or C₁-C₆ alkoxy, wherein the alkyl is substituted with one or more R_{B3}.

In some embodiments, R_{B1} is C₁-C₆ haloalkyl. In some embodiments, R_{B1} is C₁-C₆ haloalkyl, wherein the alkyl is optionally substituted with one or more R_{B3}. In some embodiments, R_{B1} is C₁-C₆ haloalkyl, wherein the alkyl is substituted with one or more R_{B3}.

In some embodiments, R_{B1} is halomethyl. In some embodiments, R_{B1} is haloethyl. In some embodiments, R_{B1} is halopropyl. In some embodiments, R_{B1} is halobutyl. In some embodiments, R_{B1} is halopentyl. In some embodiments, R_{B1} is halohexyl.

In some embodiments, R_{B1} is halomethyl, wherein the alkyl is optionally substituted with one or more R_{B3}. In some embodiments, R_{B1} is haloethyl, wherein the alkyl is optionally substituted with one or more R_{B3}. In some embodiments, R_{B1} is halopropyl, wherein the alkyl is optionally substituted with one or more R_{B3}. In some embodiments, R_{B1} is halobutyl, wherein the alkyl is optionally substituted with one or more R_{B3}. In some embodiments, R_{B1} is halopentyl, wherein the alkyl is optionally substituted with one or more R_{B3}. In some embodiments, R_{B1} is halohexyl, wherein the alkyl is optionally substituted with one or more R_{B}.

In some embodiments, R_{B1} is halomethyl, wherein the alkyl is substituted with one or more R_{B3}. In some embodiments, R_{B1} is haloethyl, wherein the alkyl is substituted with one or more R_{B3}. In some embodiments, R_{B1} is halopropyl, wherein the alkyl is substituted with one or more R_{B3}. In some embodiments, R_{B1} is halobutyl, wherein the alkyl is substituted with one or more R_{B3}. In some embodiments, R_{B1} is halopentyl, wherein the alkyl is substituted with one or more R_{B3}. In some embodiments, R_{B1} is halohexyl, wherein the alkyl is substituted with one or more R_{B3}.

In some embodiments, R_{B1} is C₁-C₆ alkoxy. In some embodiments, R_{B1} is C₁-C₆ alkoxy, wherein the alkyl is optionally substituted with one or more R_{B3}. In some embodiments, R_{B1} is C₁-C₆ alkoxy, wherein the alkyl is substituted with one or more R_{B3}.

In some embodiments, R_{B1} is methoxy. In some embodiments, R_{B1} is ethoxy. In some embodiments, R_{B1} is propoxy. In some embodiments, R_{B1} is butoxy. In some embodiments, R_{B1} is pentoxy. In some embodiments, R_{B1} is hexoxy.

In some embodiments, R_{B1} is methoxy, wherein the alkyl is optionally substituted with one or more R_{B3}. In some embodiments, R_{B1} is ethoxy, wherein the alkyl is optionally substituted with one or more R_{B3}. In some embodiments, R_{B1} is propoxy, wherein the alkyl is optionally substituted with one or more R_{B3}. In some embodiments, R_{B1} is butoxy, wherein the alkyl is optionally substituted with one or more R_{B3}. In some embodiments, R_{B1} is pentoxy, wherein the alkyl is optionally substituted with one or more R_{B3}. In some embodiments, R_{B1} is hexoxy, wherein the alkyl is optionally substituted with one or more R_{B3}.

In some embodiments, R_{B1} is methoxy, wherein the alkyl is substituted with one or more R_{B3}. In some embodiments, R_{B1} is ethoxy, wherein the alkyl is substituted with one or more R_{B}. In some embodiments, R_{B1} is propoxy, wherein the alkyl is substituted with one or more R_{B3}. In some embodiments, R_{B1} is butoxy, wherein the alkyl is substituted with one or more R_{B3}. In some embodiments, R_{B1} is pentoxy, wherein the alkyl is substituted with one or more R_{B3}. In some embodiments, R_{B1} is hexoxy, wherein the alkyl is substituted with one or more R_{B3}.

In some embodiments, R_{B1} is C₆-C₁₀ aryl, 5- to 10-membered heteroaryl, C₃-C₇ cycloalkyl, or 3- to 7-membered heterocycloalkyl.

In some embodiments, R_{B1} is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B3}.

In some embodiments, R_{B1} is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{B3}.

In some embodiments, R_{B1} is C₆-C₁₀ aryl or 5- to 10-membered heteroaryl. In some embodiments, R_{B1} is C₁-C₁₀ aryl or 5- to 10-membered heteroaryl, wherein the aryl or heteroaryl, are optionally substituted with one or more R_{B3}. In some embodiments, R_{B1} is C₆-C₁₀ aryl or 5- to 10-membered heteroaryl, wherein the aryl or heteroaryl are substituted with one or more R_{B3}.

In some embodiments, R_{B1} is C₆-C₁₀ aryl. In some embodiments, R_{B1} is C₆-C₁₀ aryl optionally substituted with one or more R_{B3}. In some embodiments, R_{B1} is C₆-C₁₀ aryl substituted with one or more R_{B3}.

In some embodiments, R_{B1} is C₆ aryl (e.g., phenyl). In some embodiments, R_{B1} is C₆ aryl (e.g., phenyl) optionally substituted with one or more R_{B3}. In some embodiments, R_{B1} is C₆ aryl (e.g., phenyl) substituted with one or more R_{B3}.

In some embodiments, R_{B1} is C₅ aryl. In some embodiments, R_{B1} is C₅ aryl optionally substituted with one or more R_{B3}. In some embodiments, R_{B1} is C₅ aryl substituted with one or more R_{B3}.

In some embodiments, R_{B1} is C₁₀ aryl. In some embodiments, R_{B1} is C₁₀ aryl optionally substituted with one or more R_{B3}. In some embodiments, R_{B1} is C₁₀ aryl substituted with one or more R_{B3}.

In some embodiments, R_{B1} is 5- to 10-membered heteroaryl. In some embodiments, R_{B1} is 5- to 10-membered heteroaryl optionally substituted with one or more R_{B3}. In some embodiments, R_{B1} is 5- to 10-membered heteroaryl substituted with one or more R_{B3}.

In some embodiments, R_{B1} is 5-membered heteroaryl. In some embodiments, R_{B1} is 5-membered heteroaryl optionally substituted with one or more R₃. In some embodiments, R_{B1} is 5-membered heteroaryl substituted with one or more R_{B3}.

In some embodiments, R_{B1} is 6-membered heteroaryl. In some embodiments, R_{B1} is 6-membered heteroaryl optionally substituted with one or more R_{B3}. In some embodiments, R_{B1} is 6-membered heteroaryl substituted with one or more R_{B3}.

In some embodiments, R_{B1} is 7-membered heteroaryl. In some embodiments, R_{B1} is 7-membered heteroaryl optionally substituted with one or more R_{B3}. In some embodiments, R_{B1} is 7-membered heteroaryl substituted with one or more R_{B3}.

In some embodiments, R_{B1} is 8-membered heteroaryl. In some embodiments, R_{B1} is 8-membered heteroaryl optionally substituted with one or more R_{B3}. In some embodiments, R_{B1} is 8-membered heteroaryl substituted with one or more R_{B3}.

In some embodiments, R_{B1} is 9-membered heteroaryl. In some embodiments, R_{B1} is 9-membered heteroaryl optionally substituted with one or more R_{B3}. In some embodiments, R_{B1} is 9-membered heteroaryl substituted with one or more R_{B3}.

In some embodiments, R_{B1} is 10-membered heteroaryl. In some embodiments, R_{B1} is 10-membered heteroaryl optionally substituted with one or more R_{B3}. In some embodiments, R_{B1} is 10-membered heteroaryl substituted with one or more R_{B3}.

In some embodiments, R_{B1} is C₃-C₇ cycloalkyl or 3- to 7-membered heterocycloalkyl. In some embodiments, R_{B1} is C₃-C₇ cycloalkyl or 3- to 7-membered heterocycloalkyl, wherein the cycloalkyl and heterocycloalkyl are optionally substituted with one or more R_{B3}. In some embodiments, R_{B1} is C₃-C₇ cycloalkyl or 3- to 7-membered heterocycloalkyl, wherein the cycloalkyl and heterocycloalkyl are substituted with one or more R_{B3}.

In some embodiments, R_{B1} is C₁-C₇ cycloalkyl. In some embodiments, R_{B1} is C₃-C₇ cycloalkyl are optionally substituted with one or more R_{B3}. In some embodiments, R_{B1} is C₃-C₇ cycloalkyl substituted with one or more R_{B3}.

In some embodiments, R_{B1} is C₃ cycloalkyl. In some embodiments, R_{B1} is C₃ cycloalkyl are optionally substituted with one or more R_{B3}. In some embodiments, R_{B1} is C₃ cycloalkyl substituted with one or more R_{B3}.

In some embodiments, R_{B1} is C₄ cycloalkyl. In some embodiments, R_{B1} is C₄ cycloalkyl are optionally substituted with one or more R_{B3}. In some embodiments, R_{B1} is C₄ cycloalkyl substituted with one or more R_{B3}.

In some embodiments, R_{B1} is C₅ cycloalkyl. In some embodiments, R_{B1} is C₅ cycloalkyl are optionally substituted with one or more R_{B3}. In some embodiments, R_{B1} is C₅ cycloalkyl substituted with one or more R_{B3}.

In some embodiments, R_{B1} is C₆ cycloalkyl. In some embodiments, R_{B1} is C₆ cycloalkyl are optionally substituted with one or more R_{B3}. In some embodiments, R_{B1} is C₆ cycloalkyl substituted with one or more R_{B3}.

In some embodiments, R_{B1} is C₇ cycloalkyl. In some embodiments, R_{B1} is C₇ cycloalkyl are optionally substituted with one or more R_{B3}. In some embodiments, R_{B1} is C₇ cycloalkyl substituted with one or more R_{B3}.

In some embodiments, R_{B1} is 3- to 7-membered heterocycloalkyl. In some embodiments, R_{B1} is 3- to 7-membered heterocycloalkyl optionally substituted with one or more R_{A}. In some embodiments, R_{B1} is 3- to 7-membered heterocycloalkyl substituted with one or more R_{B3}.

In some embodiments, R_{B1} is 3-membered heterocycloalkyl. In some embodiments, R_{B1} is 3-membered heterocycloalkyl optionally substituted with one or more R_{B3}. In some embodiments, R_{B1} is 3-membered heterocycloalkyl substituted with one or more R_{B3}.

In some embodiments, R_{B1} is 4-membered heterocycloalkyl. In some embodiments, R_{B1} is 4-membered heterocycloalkyl optionally substituted with one or more R_{B3}. In some embodiments, R_{B1} is 4-membered heterocycloalkyl substituted with one or more R_{B3}.

In some embodiments, R_{B1} is 5-membered heterocycloalkyl. In some embodiments, R_{B1} is 5-membered heterocycloalkyl optionally substituted with one or more R_{B3}. In some embodiments, R_{B1} is 5-membered heterocycloalkyl substituted with one or more R_{B3}.

In some embodiments, R_{B1} is 6-membered heterocycloalkyl. In some embodiments, R_{B1} is 6-membered heterocycloalkyl optionally substituted with one or more R_{B3}. In some embodiments, R_{B1} is 6-membered heterocycloalkyl substituted with one or more R_{B3}.

In some embodiments, R_{B1} is 7-membered heterocycloalkyl. In some embodiments, R_{B1} is 7-membered heterocycloalkyl optionally substituted with one or more R_{B3}. In some embodiments, R_{B1} is 7-membered heterocycloalkyl substituted with one or more R_{B3}.

In some embodiments, R_{B2} is H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, R_{B2} is H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, R_{B2} is H, halogen, —CN, —OH, —NH₂, —NH(C₁-C₆, alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₁-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B2} is H.

In some embodiments, R_{B2} is halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, R_{B2} is halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, R_{B2} is halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B2} is halogen, —CN, —OH, —NH₂, —NH(C₁-C₆ alkyl), —NH(C₁-C₆ alkyl)-OH, or —N(C₁-C₆ alkyl)₂.

In some embodiments, R_{B2} is halogen, —CN, —OH, —NH₂, —NH(C₁-C₆ alkyl), —NH(C₁-C₆ alkyl)-OH, or —N(C₁-C₆ alkyl)₂, wherein the alkyl is optionally substituted with one or more R_{B3}.

In some embodiments, R_{B2} is halogen, —CN, —OH, —NH₂, —NH(C₁-C₆ alkyl), —NH(C₁-C₆ alkyl)-OH, or —N(C₁-C₆ alkyl)₂, wherein the alkyl is substituted with one or more R_{B3}.

In some embodiments, R_{B2} is halogen or —CN.

In some embodiments, R_{B2} is halogen.

In some embodiments, R_{B2} is F, Cl, Br, or I. In some embodiments, R_{B2} is F, Cl, or Br. In some embodiments, R_{B2} is F or Cl. In some embodiments, R_{B2} is F. In some embodiments, R_{B2} is C₁. In some embodiments, R_{B2} is Br. In some embodiments, R_{B2} is I.

In some embodiments, R_{B2} is —CN.

In some embodiments, R_{B2} is —OH, —NH₂, —NH(C₁-C₆ alkyl), —NH(C₁-C₆ alkyl)-OH, or —N(C₁-C₆ alkyl)₂.

In some embodiments, R_{B2} is —OH, —NH₂, —NH(C₁-C₆ alkyl), —NH(C₁-C₆ alkyl)-OH, or —N(C₁-C₆ alkyl)₂, wherein the alkyl is optionally substituted with one or more R_{B3}.

In some embodiments, R_{B2} is —OH, —NH₂, —NH(C₁-C₆ alkyl), —NH(C₁-C₆ alkyl)-OH, or —N(C₁-C₆ alkyl)₂, wherein the alkyl is substituted with one or more R_{B3}.

In some embodiments, R_{B2} is —OH. In some embodiments, R_{B2} is —NH₂.

In some embodiments, R_{B2} is —NH(C₁-C₆ alkyl), —NH(C₁-C₆ alkyl)-OH, or —N(C₁-C₆ alkyl)₂.

In some embodiments, R_{B2} is —NH(C₁-C₆ alkyl), —NH(C₁-C₆ alkyl)-OH, or —N(C₁-C₆ alkyl)₂, wherein the alkyl is optionally substituted with one or more R_{B3}.

In some embodiments, R_{B2} is —NH(C₁-C₆ alkyl), —NH(C₁-C₆ alkyl)-OH, or —N(C₁-C₆ alkyl)₂, wherein the alkyl is substituted with one or more R_{B3}.

In some embodiments, R_{B2} is —NH(C₁-C₆ alkyl). In some embodiments, R_{B2} is —NH(C₁-C₆ alkyl), wherein the alkyl is optionally substituted with one or more R_{B2}. In some embodiments, R_{B2} is —NH(C₁-C₆ alkyl), wherein the alkyl is substituted with one or more R_{B3}.

In some embodiments, R_{B2} is —NH(C₁-C₆ alkyl)-OH. In some embodiments, R_{B2} is —NH(C₁-C₆ alkyl)-OH, wherein the alkyl is optionally substituted with one or more R_{B3}. In some embodiments, R_{B2} is —NH(C₁-C₆ alkyl)-OH, wherein the alkyl is substituted with one or more R_{B3}.

In some embodiments, R_{B2} is —N(C₁-C₆ alkyl)₂. In some embodiments, R_{B2} is —N(C₁-C₆ alkyl)₂, wherein the alkyl is optionally substituted with one or more R_{B3}. In some embodiments, R_{B2} is —N(C₁-C₆ alkyl)₂, wherein the alkyl is substituted with one or more R_{B3}.

In some embodiments, R_{B2} is C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, R_{B2} is C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

In some embodiments, R_{B2} is C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3}.

In some embodiments, R_{B2} is C₁-C₆ alkyl, C₂-C₆ alkenyl, C₂-C₆ alkynyl, C₁-C₆ haloalkyl, or C₁-C₆ alkoxy.

In some embodiments, R_{B2} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more R_{B3}.

In some embodiments, R_{B2} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are substituted with one or more R_{B3}.

In some embodiments, R_{B2} is C₁-C₆ alkyl, C₂-C₆ alkenyl, or C₂-C₆ alkynyl.

In some embodiments, R_{B2} is C₁-C₆ alkyl, C₂-C₆ alkenyl, or C₂-C₆ alkynyl, wherein the alkyl, alkenyl, or alkynyl are optionally substituted with one or more R_{B3}.

In some embodiments, R_{B2} is C₁-C₆ alkyl, C₂-C₆ alkenyl, or C₂-C₆ alkynyl, wherein the alkyl, alkenyl, or alkynyl are substituted with one or more R_{B3}.

In some embodiments, R_{B2} is C₁-C₆ alkyl. In some embodiments, R_{B2} is C₁-C₆ alkyl optionally substituted with one or more R_{B3}. In some embodiments, R_{B2} is C₁-C₆ alkyl substituted with one or more R_{B3}.

In some embodiments, R_{B2} is methyl. In some embodiments, R_{B2} is ethyl. In some embodiments, R_{B2} is propyl. In some embodiments, R_{B2} is butyl. In some embodiments, R_{B2} is pentyl. In some embodiments, R_{B2} is hexyl. In some embodiments, R_{B2} is isopropyl. In some embodiments, R_{B2} is isobutyl. In some embodiments, R_{B2} is isopentyl. In some embodiments, R_{B2} is isohexyl. In some embodiments, R_{B2} is secbutyl. In some embodiments, R_{B2} is secpentyl. In some embodiments, R_{B2} is sechexyl. In some embodiments, R_{B2} is tertbutyl.

In some embodiments, R_{B2} is methyl optionally substituted with one or more R_{B3}. In some embodiments, R_{B2} is ethyl optionally substituted with one or more R_{B3}. In some embodiments, R_{B2} is propyl optionally substituted with one or more R_{B3}. In some embodiments, R_{B2} is butyl optionally substituted with one or more R_{B3}. In some embodiments, R_{B2} is pentyl optionally substituted with one or more R_{B3}. In some embodiments, R_{B2} is hexyl optionally substituted with one or more R_{B3}. In some embodiments, R_{B2} is isopropyl optionally substituted with one or more R_{B3}. In some embodiments, R_{B2} is isobutyl optionally substituted with one or more R_{B3}. In some embodiments, R_{B} is isopentyl optionally substituted with one or more R_{B3}. In some embodiments, R_{B2} is isohexyl optionally substituted with one or more R_{B3}. In some embodiments, R_{B2} is secbutyl optionally substituted with one or more R_{B3}. In some embodiments, R_{B2} is secpentyl optionally substituted with one or more R_{B3}. In some embodiments, R_{B2} is sechexyl optionally substituted with one or more R_{B3}. In some embodiments, R_{B2} is tertbutyl optionally substituted with one or more R_{B3}.

In some embodiments, R_{B2} is methyl substituted with one or more R_{B3}. In some embodiments, R_{B2} is ethyl substituted with one or more R_{B3}. In some embodiments, R_{B2} is propyl substituted with one or more R_{B3}. In some embodiments, R_{B2} is butyl substituted with one or more R_{B3}. In some embodiments, R_{B2} is pentyl substituted with one or more R_{B3}. In some embodiments, R_{B2} is hexyl substituted with one or more R_{B3}. In some embodiments, R_{B2} is isopropyl substituted with one or more R_{B3}. In some embodiments, R_{B2} is isobutyl substituted with one or more R_{B3}. In some embodiments, R_{B2} is isopentyl substituted with one or more R_{B3}. In some embodiments, R_{B2} is isohexyl substituted with one or more R_{B3}. In some embodiments, R_{B2} is secbutyl substituted with one or more R_{B3}. In some embodiments, R_{B2} is secpentyl substituted with one or more R_{B3}. In some embodiments, R_{B2} is sechexyl substituted with one or more R_{B3}. In some embodiments, R_{B2} is tertbutyl substituted with one or more R_{B3}.

In some embodiments, R_{B2} is C₂-C₆ alkenyl. In some embodiments, R_{B2} is C₂-C₆ alkenyl optionally substituted with one or more R_{B3}. In some embodiments, R_{B2} is C₂-C₆ alkenyl substituted with one or more R_{B3}.

In some embodiments, R_{B2} is C₂ alkenyl. In some embodiments, R_{B2} is C₃ alkenyl. In some embodiments, R_{B2} is C₄ alkenyl. In some embodiments, R_{B2} is C₅ alkenyl. In some embodiments, R_{B2} is C₆ alkenyl.

In some embodiments, R_{B2} is C₂ alkenyl optionally substituted with one or more R_{B3}. In some embodiments, R_{B2} is C₃ alkenyl optionally substituted with one or more R_{B3}. In some embodiments, R_{B2} is C₄ alkenyl optionally substituted with one or more R_{B3}. In some embodiments, R_{B2} is C₅ alkenyl optionally substituted with one or more R_{B3}. In some embodiments, R_{B2} is C₆ alkenyl optionally substituted with one or more R_{B3}.

In some embodiments, R_{B2} is C₂ alkenyl substituted with one or more R_{B3}. In some embodiments, R_{B2} is C₃ alkenyl substituted with one or more R_{B3}. In some embodiments, R_{B2} is C₄ alkenyl substituted with one or more R_{B3}. In some embodiments, R_{B2} is C₅ alkenyl substituted with one or more R_{B3}. In some embodiments, R_{B2} is C₆ alkenyl substituted with one or more R_{B3}.

In some embodiments, R_{B2} is C₂-C₆ alkynyl. In some embodiments, R_{B2} is C₂-C₆ alkynyl optionally substituted with one or more R_{B3}. In some embodiments, R_{B2} is C₂-C₆ alkynyl substituted with one or more R_{B3}.

In some embodiments, R_{B2} is C₂ alkynyl. In some embodiments, R_{B2} is C₃ alkynyl. In some embodiments, R_{B2} is C₄ alkynyl. In some embodiments, R_{B2} is C₅ alkynyl. In some embodiments, R_{B2} is C₆ alkynyl.

In some embodiments, R_{B2} is C₂ alkynyl optionally substituted with one or more R_{B3}. In some embodiments, R_{B2} is C₃ alkynyl optionally substituted with one or more R_{B3}. In some embodiments, R_{B2} is C₄ alkynyl optionally substituted with one or more R_{B3}. In some embodiments, R_{B2} is C₅ alkynyl optionally substituted with one or more R_{B3}. In some embodiments, R_{B2} is C₆ alkynyl optionally substituted with one or more R_{B3}.

In some embodiments, R_{B2} is C₂ alkynyl substituted with one or more R_{B3}. In some embodiments, R_{B2} is C₃ alkynyl substituted with one or more R_{B3}. In some embodiments, R_{B2} is CA alkynyl substituted with one or more R_{B3}. In some embodiments, R_{B2} is C₅ alkynyl substituted with one or more R_{B3}. In some embodiments, R_{B2} is C₆ alkynyl substituted with one or more R_{B3}.

In some embodiments, R_{B2} is C₁-C₆ haloalkyl or C₁-C₆ alkoxy.

In some embodiments, R_{B2} is C₁-C₆ haloalkyl or C₁-C₆ alkoxy, wherein the alkyl is optionally substituted with one or more R_{B3}.

In some embodiments, R_{B2} is C₁-C₆ haloalkyl or C₁-C₆ alkoxy, wherein the alkyl is substituted with one or more R_{B3}.

In some embodiments, R_{B2} is C₁-C₆ haloalkyl. In some embodiments, R_{B2} is C₁-C₆ haloalkyl, wherein the alkyl is optionally substituted with one or more R_{B3}. In some embodiments, R_{B2} is C₁-C₆ haloalkyl, wherein the alkyl is substituted with one or more R_{B3}.

In some embodiments, R_{B2} is halomethyl. In some embodiments, R_{B2} is haloethyl. In some embodiments, R_{B2} is halopropyl. In some embodiments, R_{B2} is halobutyl. In some embodiments, R_{B2} is halopentyl. In some embodiments, R_{B2} is halohexyl.

In some embodiments, R_{B2} is halomethyl, wherein the alkyl is optionally substituted with one or more R_{B3}. In some embodiments, R_{B2} is haloethyl, wherein the alkyl is optionally substituted with one or more R_{B3}. In some embodiments, R_{B2} is halopropyl, wherein the alkyl is optionally substituted with one or more R_{B}. In some embodiments, R_{B2} is halobutyl, wherein the alkyl is optionally substituted with one or more R_{B3}. In some embodiments, R_{B2} is halopentyl, wherein the alkyl is optionally substituted with one or more R_{B3}. In some embodiments, R_{B2} is halohexyl, wherein the alkyl is optionally substituted with one or more R_{B3}.

In some embodiments, R_{B2} is halomethyl, wherein the alkyl is substituted with one or more R_{B3}. In some embodiments, R_{B2} is haloethyl, wherein the alkyl is substituted with one or more R_{B3}. In some embodiments, R_{B2} is halopropyl, wherein the alkyl is substituted with one or more R_{B3}. In some embodiments, R_{B2} is halobutyl, wherein the alkyl is substituted with one or more R_{B3}. In some embodiments, R_{B2} is halopentyl, wherein the alkyl is substituted with one or more R_{B3}. In some embodiments, R_{B2} is halohexyl, wherein the alkyl is substituted with one or more R_{B3}.

In some embodiments, R_{B2} is C₁-C₆ alkoxy. In some embodiments, R_{B2} is C₁-C₆ alkoxy, wherein the alkyl is optionally substituted with one or more R_{B3}. In some embodiments, R_{B2} is C₁-C₆ alkoxy, wherein the alkyl is substituted with one or more R_{B3}.

In some embodiments, R_{B2} is methoxy. In some embodiments, R_{B2} is ethoxy. In some embodiments, R_{B2} is propoxy. In some embodiments, R_{B2} is butoxy. In some embodiments, R_{B2} is pentoxy. In some embodiments, R_{B2} is hexoxy.

In some embodiments, R_{B2} is methoxy, wherein the alkyl is optionally substituted with one or more R_{B3}. In some embodiments, R_{B2} is ethoxy, wherein the alkyl is optionally substituted with one or more R_{B3}. In some embodiments, R_{B2} is propoxy, wherein the alkyl is optionally substituted with one or more R_{B3}. In some embodiments, R_{B2} is butoxy, wherein the alkyl is optionally substituted with one or more R_{B3}. In some embodiments, R_{B2} is pentoxy, wherein the alkyl is optionally substituted with one or more R_{B}. In some embodiments, R_{B2} is hexoxy, wherein the alkyl is optionally substituted with one or more R_{B3}.

In some embodiments, R_{B2} is methoxy, wherein the alkyl is substituted with one or more R_{B3}. In some embodiments, R_{B2} is ethoxy, wherein the alkyl is substituted with one or more R_{B}. In some embodiments, R_{B2} is propoxy, wherein the alkyl is substituted with one or more R_{B3}. In some embodiments, R_{B2} is butoxy, wherein the alkyl is substituted with one or more R_{B3}. In some embodiments, R_{B2} is pentoxy, wherein the alkyl is substituted with one or more R_{B3}. In some embodiments, R_{B2} is hexoxy, wherein the alkyl is substituted with one or more R_{B3}.

In some embodiments, R_{B2} is C₁-C₁₀ aryl, 5- to 10-membered heteroaryl, C₃-C₇ cycloalkyl, or 3- to 7-membered heterocycloalkyl.

In some embodiments, R_{B2} is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B3}.

In some embodiments, R_{B32} is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{B3}.

In some embodiments, R_{B2} is C₆-C₁₀ aryl or 5- to 10-membered heteroaryl. In some embodiments, R_{B2} is C₆-C₁₀ aryl or 5- to 10-membered heteroaryl, wherein the aryl or heteroaryl, are optionally substituted with one or more R_{B3}. In some embodiments, R_{B2} is C₆-C₁₀ aryl or 5- to 10-membered heteroaryl, wherein the aryl or heteroaryl are substituted with one or more R_{B3}.

In some embodiments, R_{B2} is C₆-C₁₀ aryl. In some embodiments, R_{B2} is C₆-C₁₀ aryl optionally substituted with one or more R_{B3}. In some embodiments, R_{B2} is C₆-C₁₀ aryl substituted with one or more R_{B3}.

In some embodiments, R_{B2} is C₆ aryl (e.g., phenyl). In some embodiments, R_{B2} is C₆ aryl (e.g., phenyl) optionally substituted with one or more R_{B3}. In some embodiments, R_{B2} is C₆ aryl (e.g., phenyl) substituted with one or more R_{B3}.

In some embodiments, R_{B2} is C₅ aryl. In some embodiments, R_{B2} is C₅ aryl optionally substituted with one or more R_{B3}. In some embodiments, R_{B2} is C₅ aryl substituted with one or more R_{B3}.

In some embodiments, R_{B2} is C₁₀ aryl. In some embodiments, R_{B2} is C₁₀ aryl optionally substituted with one or more R_{B3}. In some embodiments, R_{B2} is C₁₀ aryl substituted with one or more R_{B3}.

In some embodiments, R_{B2} is 5- to 10-membered heteroaryl. In some embodiments, R_{B2} is 5- to 10-membered heteroaryl optionally substituted with one or more R_{B}. In some embodiments, R_{B2} is 5- to 10-membered heteroaryl substituted with one or more R_{B3}.

In some embodiments, R_{B2} is 5-membered heteroaryl. In some embodiments, R_{B2} is 5-membered heteroaryl optionally substituted with one or more R_{B}. In some embodiments, R_{B2} is 5-membered heteroaryl substituted with one or more R_{B3}.

In some embodiments, R_{B} is 6-membered heteroaryl. In some embodiments, R_{B2} is 6-membered heteroaryl optionally substituted with one or more R_{B3}. In some embodiments, R_{B2} is 6-membered heteroaryl substituted with one or more R_{B3}.

In some embodiments, R_{B2} is 7-membered heteroaryl. In some embodiments, R_{B2} is 7-membered heteroaryl optionally substituted with one or more R_{B3}. In some embodiments, R_{B2} is 7-membered heteroaryl substituted with one or more R_{B3}.

In some embodiments, R_{B2} is 8-membered heteroaryl. In some embodiments, R_{B2} is 8-membered heteroaryl optionally substituted with one or more R_{B3}. In some embodiments, R_{B2} is 8-membered heteroaryl substituted with one or more R_{B3}.

In some embodiments, R_{B2} is 9-membered heteroaryl. In some embodiments, R_{B2} is 9-membered heteroaryl optionally substituted with one or more R_{B3}. In some embodiments, R_{B2} is 9-membered heteroaryl substituted with one or more R_{B3}.

In some embodiments, R_{B2} is 10-membered heteroaryl. In some embodiments, R_{B2} is 10-membered heteroaryl optionally substituted with one or more R_{B2}. In some embodiments, R_{B2} is 10-membered heteroaryl substituted with one or more R_{B3}.

In some embodiments, R_{B2} is 3-C₇ cycloalkyl or 3- to 7-membered heterocycloalkyl.

In some embodiments, R_{B2} is C₁-C₇ cycloalkyl or 3- to 7-membered heterocycloalkyl, wherein the cycloalkyl and heterocycloalkyl are optionally substituted with one or more R_{B3}.

In some embodiments, R_{B2} is C₁-C₇ cycloalkyl or 3- to 7-membered heterocycloalkyl, wherein the cycloalkyl and heterocycloalkyl are substituted with one or more R_{B3}.

In some embodiments, R_{B2} is C₃-C₇ cycloalkyl. In some embodiments, R_{B2} is C₃-C₇ cycloalkyl are optionally substituted with one or more R_{B3}. In some embodiments, R_{B2} is C₃-C₇ cycloalkyl substituted with one or more R_{B3}.

In some embodiments, R_{B2} is C₃ cycloalkyl. In some embodiments, R_{B2} is C₃ cycloalkyl are optionally substituted with one or more R_{B3}. In some embodiments, R_{B2} is C₃ cycloalkyl substituted with one or more R_{B3}.

In some embodiments, R_{B2} is C₄ cycloalkyl. In some embodiments, R_{B2} is C₄ cycloalkyl are optionally substituted with one or more R_{B3}. In some embodiments, R_{B2} is C₄ cycloalkyl substituted with one or more R_{B3}.

In some embodiments, R_{B2} is C₅ cycloalkyl. In some embodiments, R_{B2} is C₅ cycloalkyl are optionally substituted with one or more R_{B3}. In some embodiments, R_{B2} is C₅ cycloalkyl substituted with one or more R_{B3}.

In some embodiments, R_{B2} is C₆ cycloalkyl. In some embodiments, R_{B2} is C₆ cycloalkyl are optionally substituted with one or more R_{B3}. In some embodiments, R_{B2} is C₆ cycloalkyl substituted with one or more R_{B3}.

In some embodiments, R_{B2} is C₇ cycloalkyl. In some embodiments, R_{B2} is C₇ cycloalkyl are optionally substituted with one or more R_{B3}. In some embodiments, R_{B2} is C₇ cycloalkyl substituted with one or more R_{B3}.

In some embodiments, R_{B2} is 3- to 7-membered heterocycloalkyl. In some embodiments, R_{B2} is 3- to 7-membered heterocycloalkyl optionally substituted with one or more R₃. In some embodiments, R_{B2} is 3- to 7-membered heterocycloalkyl substituted with one or more R_{B3}.

In some embodiments, R_{B2} is 3-membered heterocycloalkyl. In some embodiments, R_{B2} is 3-membered heterocycloalkyl optionally substituted with one or more R_{B3}. In some embodiments, R_{B2} is 3-membered heterocycloalkyl substituted with one or more R_{B3}.

In some embodiments, R_{B2} is 4-membered heterocycloalkyl. In some embodiments, R_{B2} is 4-membered heterocycloalkyl optionally substituted with one or more R_{B3}. In some embodiments, R_{B2} is 4-membered heterocycloalkyl substituted with one or more R_{B3}.

In some embodiments, R_{B2} is 5-membered heterocycloalkyl. In some embodiments, R_{B2} is 5-membered heterocycloalkyl optionally substituted with one or more R_{B3}. In some embodiments, R_{B2} is 5-membered heterocycloalkyl substituted with one or more R_{B3}.

In some embodiments, R_{B2} is 6-membered heterocycloalkyl. In some embodiments, R_{B2} is 6-membered heterocycloalkyl optionally substituted with one or more R_{B3}. In some embodiments, R_{B2} is 6-membered heterocycloalkyl substituted with one or more R_{B3}.

In some embodiments, R_{B2} is 7-membered heterocycloalkyl. In some embodiments, R_{B2} is 7-membered heterocycloalkyl optionally substituted with one or more R_{B3}. In some embodiments, R_{B2} is 7-membered heterocycloalkyl substituted with one or more R_{B}.

In some embodiments, R_{B1} and R_{B2}, together with the atom to which they are attached, form a 3- to 7-membered heterocycloalkyl.

In some embodiments, R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 3- to 7-membered heterocycloalkyl, substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 3-membered heterocycloalkyl.

In some embodiments, R_{B1} and R_{B2}, together with the atom to which they are attached, form a 3-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 3-membered heterocycloalkyl, substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 4-membered heterocycloalkyl.

In some embodiments, R_{B1} and R_{B2}, together with the atom to which they are attached, form a 4-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 4-membered heterocycloalkyl, substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 5-membered heterocycloalkyl.

In some embodiments, R_{B1} and R_{B2}, together with the atom to which they are attached, form a 5-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 5-membered heterocycloalkyl, substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 6-membered heterocycloalkyl.

In some embodiments, R_{B1} and R_{B2}, together with the atom to which they are attached, form a 6-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 6-membered heterocycloalkyl, substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 7-membered heterocycloalkyl.

In some embodiments, R_{B1} and R_{B2}, together with the atom to which they are attached, form a 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, R_{B1} and R_{B2}, together with the atom to which they are attached, form a 7-membered heterocycloalkyl, substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl.

In some embodiments, each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′}.

In some embodiments, each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are substituted with one or more R_{B3′}.

In some embodiments, each R_{B3} is independently C₁-C₆ alkyl, C₂-C₆ alkenyl, C₂-C₆ alkynyl, C₁-C₆ haloalkyl, or C₁-C₆ alkoxy.

In some embodiments, each R_{B3} is independently C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more R_{B3′}.

In some embodiments, each R_{B3} is independently C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are substituted with one or more R_{B3′}.

In some embodiments, each R_{B3} is independently C₁-C₆ alkyl, C₂-C₆ alkenyl, or C₂-C₆ alkynyl.

In some embodiments, each R_{B3} is independently C₁-C₆ alkyl, C₂-C₆ alkenyl, or C₂-C₆ alkynyl, wherein the alkyl, alkenyl, or alkynyl are optionally substituted with one or more R_{B3′}.

In some embodiments, each R_{B3} is independently C₁-C₆ alkyl, C₂-C₆ alkenyl, or C₂-C₆ alkynyl, wherein the alkyl, alkenyl, or alkynyl are substituted with one or more R_{B3′}.

In some embodiments, each R_{B3} is independently C₁-C₆ alkyl. In some embodiments, each R_{B3} is independently C₁-C₆ alkyl optionally substituted with one or more R_{B3′}. In some embodiments, each R_{B3} is independently C₁-C₆ alkyl substituted with one or more R_{B3′}.

In some embodiments, each R_{B3} is independently methyl. In some embodiments, each R_{B3} is independently ethyl. In some embodiments, each R_{B3} is independently propyl. In some embodiments, each R_{B3} is independently butyl. In some embodiments, each R_{B3} is independently pentyl. In some embodiments, each R_{B3} is independently hexyl. In some embodiments, each R_{B3} is independently isopropyl. In some embodiments, each R_{B3} is independently isobutyl. In some embodiments, each R_{B3} is independently isopentyl. In some embodiments, each R_{B3} is independently isohexyl. In some embodiments, each R_{B3} is independently secbutyl. In some embodiments, each R_{B3} is independently secpentyl. In some embodiments, each R_{B3} is independently sechexyl. In some embodiments, each R_{B3} is independently tertbutyl.

In some embodiments, each R_{B3} is independently methyl optionally substituted with one or more R_{B3′}. In some embodiments, each R_{B3} is independently ethyl optionally substituted with one or more Ray. In some embodiments, each R_{B3} is independently propyl optionally substituted with one or more R_{B3′}. In some embodiments, each R_{B3} is independently butyl optionally substituted with one or more R_{B3}. In some embodiments, each R_{B3} is independently pentyl optionally substituted with one or more R_{B3′}. In some embodiments, each R_{B3} is independently hexyl optionally substituted with one or more R_{B3′}. In some embodiments, each R_{B3} is independently isopropyl optionally substituted with one or more R_{B3}. In some embodiments, each R_{B3} is independently isobutyl optionally substituted with one or more R_{B3′}. In some embodiments, each R_{B3} is independently isopentyl optionally substituted with one or more R_{B3}. In some embodiments, each R_{B3} is independently isohexyl optionally substituted with one or more R_{B}r. In some embodiments, each R_{B3} is independently secbutyl optionally substituted with one or more R_{B3′}. In some embodiments, each R_{B3} is independently secpentyl optionally substituted with one or more R_{B}. In some embodiments, each R_{B3} is independently sechexyl optionally substituted with one or more R_{B3′}. In some embodiments, each R_{B3} is independently tertbutyl optionally substituted with one or more R_{B3′}.

In some embodiments, each R_{B3} is independently methyl substituted with one or more R_{B3′}. In some embodiments, each R_{B3} is independently ethyl substituted with one or more R_{B3′}. In some embodiments, each R_{B3} is independently propyl substituted with one or more R_{B3′}. In some embodiments, each R_{B3} is independently butyl substituted with one or more R_{B3}. In some embodiments, each R_{B3} is independently pentyl substituted with one or more R_{B3′}. In some embodiments, each R_{B3} is independently hexyl substituted with one or more R_{B3′}. In some embodiments, each R_{B3} is independently isopropyl substituted with one or more R_{B3′}. In some embodiments, each R_{B3} is independently isobutyl substituted with one or more R_{B3′}. In some embodiments, each R_{B3} is independently isopentyl substituted with one or more R_{B3′}. In some embodiments, each R_{B3} is independently isohexyl substituted with one or more R_{B3′}. In some embodiments, each R_{B3} is independently secbutyl substituted with one or more R_{B3′}. In some embodiments, each R_{B3} is independently secpentyl substituted with one or more R_{B3′}. In some embodiments, each R_{B3} is independently sechexyl substituted with one or more R_{B3′}. In some embodiments, each R_{B3} is independently tertbutyl substituted with one or more R_{B3′.}

In some embodiments, each R_{B3} is independently C₂-C₆ alkenyl. In some embodiments, each R_{B3} is independently C₂-C₆ alkenyl optionally substituted with one or more R_{B3′}. In some embodiments, each R_{B3} is independently C₂-C₆ alkenyl substituted with one or more R_{B3′}.

In some embodiments, each R_{B3} is independently C₂ alkenyl. In some embodiments, each R_{B3} is independently C₃ alkenyl. In some embodiments, each R_{B3} is independently C₄ alkenyl. In some embodiments, each R_{B3} is independently C₅ alkenyl. In some embodiments, each R_{B3} is independently C₆ alkenyl.

In some embodiments, each R_{B3} is independently C₂ alkenyl optionally substituted with one or more R_{B3′}. In some embodiments, each R_{B3} is independently C₃ alkenyl optionally substituted with one or more R_{B3′}. In some embodiments, each R_{B3} is independently C₄ alkenyl optionally substituted with one or more R_{B3′}. In some embodiments, each R_{B3} is independently C₅ alkenyl optionally substituted with one or more R_{B3′}. In some embodiments, each R_{B3} is independently C₆ alkenyl optionally substituted with one or more R_{B3′}.

In some embodiments, each R_{B3} is independently C₂ alkenyl substituted with one or more R_{B3′}. In some embodiments, each R_{B3} is independently C₃ alkenyl substituted with one or more R_{B3′}. In some embodiments, each R_{B3} is independently C₄ alkenyl substituted with one or more R_{B3′}. In some embodiments, each R_{B3} is independently C₅ alkenyl substituted with one or more R_{B3′}. In some embodiments, each R_{B3} is independently C₆ alkenyl substituted with one or more R_{B3′}.

In some embodiments, each R_{B3} is independently C₂-C₆ alkynyl. In some embodiments, each R_{B3} is independently C₂-C₆ alkynyl optionally substituted with one or more R_{B3′}. In some embodiments, each R_{B3} is independently C₂-C₆ alkynyl substituted with one or more R_{B3}.

In some embodiments, each R_{B3} is independently C₂ alkynyl. In some embodiments, each R_{B3} is independently C₃ alkynyl. In some embodiments, each R_{B3} is independently C₄ alkynyl. In some embodiments, each R_{B3} is independently C₅ alkynyl. In some embodiments, each R_{B3} is independently C₆ alkynyl.

In some embodiments, each R_{B3} is independently C₂ alkynyl optionally substituted with one or more R_{B3′}. In some embodiments, each R_{B3} is independently C₃ alkynyl optionally substituted with one or more R_{B3}. In some embodiments, each R_{B3} is independently C₄ alkynyl optionally substituted with one or more R_{B3′}. In some embodiments, each R_{B3} is independently C₅ alkynyl optionally substituted with one or more R_{B3′}. In some embodiments, each R_{B3} is independently C₆ alkynyl optionally substituted with one or more R_{B3′}.

In some embodiments, each R_{B3} is independently C₂ alkynyl substituted with one or more R_{B3′}. In some embodiments, each R_{B3} is independently C₃ alkynyl substituted with one or more R_{B3′}. In some embodiments, each R_{B3} is independently C₄ alkynyl substituted with one or more R_{B3′}. In some embodiments, each R_{B3} is independently C₅ alkynyl substituted with one or more R_{B3′}. In some embodiments, each R_{B3} is independently C₆ alkynyl substituted with one or more R_{B3′}.

In some embodiments, each R_{B3} is independently C₁-C₆ haloalkyl or C₁-C₆ alkoxy.

In some embodiments, each R_{B3} is independently C₁-C₆ haloalkyl or C₁-C₆ alkoxy, wherein the alkyl is optionally substituted with one or more R_{B3}.

In some embodiments, each R_{B3} is independently C₁-C₆ haloalkyl or C₁-C₆ alkoxy, wherein the alkyl is substituted with one or more R_{B3′}.

In some embodiments, each R_{B3} is independently C₁-C₆ haloalkyl. In some embodiments, each R_{B3} is independently C₁-C₆ haloalkyl, wherein the alkyl is optionally substituted with one or more R_{B3}. In some embodiments, each R_{B3} is independently C₁-C₆ haloalkyl, wherein the alkyl is substituted with one or more R_{B}.

In some embodiments, each R_{B3} is independently halomethyl. In some embodiments, each R_{B3} is independently haloethyl. In some embodiments, each R_{B1} is independently halopropyl. In some embodiments, each R_{B3} is independently halobutyl. In some embodiments, each R_{B3} is independently halopentyl. In some embodiments, each R_{B3} is independently halohexyl.

In some embodiments, each R_{B3} is independently halomethyl, wherein the alkyl is optionally substituted with one or more R_{B3′}. In some embodiments, each R_{B3} is independently haloethyl, wherein the alkyl is optionally substituted with one or more R_{B3′}. In some embodiments, each R_{B3} is independently halopropyl, wherein the alkyl is optionally substituted with one or more Ray. In some embodiments, each R_{B} is independently halobutyl, wherein the alkyl is optionally substituted with one or more R_{B3′}. In some embodiments, each R_{B3} is independently halopentyl, wherein the alkyl is optionally substituted with one or more R_{B3′}. In some embodiments, each R_{B3} is independently halohexyl, wherein the alkyl is optionally substituted with one or more R_{B3′}.

In some embodiments, each R_{B3} is independently halomethyl, wherein the alkyl is substituted with one or more R_{B3′}. In some embodiments, each R_{B3} is independently haloethyl, wherein the alkyl is substituted with one or more R_{B3′}. In some embodiments, each R_{B3} is independently halopropyl, wherein the alkyl is substituted with one or more R_{B3′}. In some embodiments, each R_{B} is independently halobutyl, wherein the alkyl is substituted with one or more R_{B3′}. In some embodiments, each R_{B3} is independently halopentyl, wherein the alkyl is substituted with one or more R_{B3′}. In some embodiments, each R_{B} is independently halohexyl, wherein the alkyl is substituted with one or more R_{B3′}.

In some embodiments, each R_{B3} is independently C₁-C₆ alkoxy. In some embodiments, each R_{B3} is independently C₁-C₆ alkoxy, wherein the alkyl is optionally substituted with one or more R_{B3′}. In some embodiments, each R_{B3} is independently C₁-C₆ alkoxy, wherein the alkyl is substituted with one or more R_{B3′}.

In some embodiments, each R_{B3} is independently methoxy. In some embodiments, each R_{B3} is independently ethoxy. In some embodiments, each R_{B3} is independently propoxy. In some embodiments, each R_{B3} is independently butoxy. In some embodiments, each R_{B3} is independently pentoxy. In some embodiments, each R_{B3} is independently hexoxy.

In some embodiments, each R_{B3} is independently methoxy, wherein the alkyl is optionally substituted with one or more R_{B3′}. In some embodiments, each R_{B3} is independently ethoxy, wherein the alkyl is optionally substituted with one or more R_{B3′}. In some embodiments, each R_{B3} is independently propoxy, wherein the alkyl is optionally substituted with one or more R_{B3′}. In some embodiments, each Raw is independently butoxy, wherein the alkyl is optionally substituted with one or more R_{B3}. In some embodiments, each R_{B3} is independently pentoxy, wherein the alkyl is optionally substituted with one or more R_{B3′}. In some embodiments, each R_{B3} is independently hexoxy, wherein the alkyl is optionally substituted with one or more R_{B3′}.

In some embodiments, each R_{B3} is independently methoxy, wherein the alkyl is substituted with one or more R_{B3′}. In some embodiments, each R_{B3} is independently ethoxy, wherein the alkyl is substituted with one or more R_{B3′}. In some embodiments, each R_{B3} is independently propoxy, wherein the alkyl is substituted with one or more R_{B3′}. In some embodiments, each R_{B3} is independently butoxy, wherein the alkyl is substituted with one or more R_{B3′}. In some embodiments, each R_{B3}3 is independently pentoxy, wherein the alkyl is substituted with one or more R_{B3′}. In some embodiments, each R_{B3} is independently hexoxy, wherein the alkyl is substituted with one or more R_{B3′}.

In some embodiments, each R_{B3} is independently C₆-C₁₀ aryl, 5- to 10-membered heteroaryl, C₃-C₇ cycloalkyl, or 3- to 7-membered heterocycloalkyl.

In some embodiments, each R_{B3} is independently C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B3′}.

In some embodiments, each R_{B3} is independently C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{B3′}.

In some embodiments, each R_{B3} is independently C₆-C₁₀ aryl or 5- to 10-membered heteroaryl. In some embodiments, each R_{B3} is independently C₆-C₁₀ aryl or 5- to 10-membered heteroaryl, wherein the aryl or heteroaryl are optionally substituted with one or more R_{B3′}. In some embodiments, each R_{B3} is independently C₆-C₁₀ aryl or 5- to 10-membered heteroaryl, wherein the aryl or heteroaryl are substituted with one or more R_{B3′}.

In some embodiments, each R_{B3} is independently C₆-C₁₀ aryl. In some embodiments, each R_{B3} is independently C₆-C₁₀ aryl optionally substituted with one or more Ray. In some embodiments, each R_{B3} is independently C₆-C₁₀ aryl substituted with one or more Ray.

In some embodiments, each R_{B3} is independently C₆ aryl (e.g., phenyl). In some embodiments, each R_{B3} is independently C₆ aryl (e.g., phenyl) optionally substituted with one or more R_{B3′}. In some embodiments, each R_{B3} is independently C₆ aryl (e.g., phenyl) substituted with one or more R_{B3}.

In some embodiments, each R_{B} is independently C₅ aryl. In some embodiments, each R_{B3} is independently C₅ aryl optionally substituted with one or more R_{B}. In some embodiments, each R_{B3} is independently C₅ aryl substituted with one or more R_{B3}.

In some embodiments, each R_{B3} is independently C₁₀ aryl. In some embodiments, each R_{B3} is independently C₁₀ aryl optionally substituted with one or more R_{B3′}. In some embodiments, each R_{B3} is independently C₁₀ aryl substituted with one or more R_{B3′}.

In some embodiments, each R_{B3} is independently 5- to 10-membered heteroaryl. In some embodiments, each R_{B3} is independently 5- to 10-membered heteroaryl optionally substituted with one or more R_{B3′}. In some embodiments, each R_{B3} is independently 5- to 10-membered heteroaryl substituted with one or more R_{B3′}.

In some embodiments, each R_{B3} is independently 5-membered heteroaryl. In some embodiments, each R_{B3} is independently 5-membered heteroaryl optionally substituted with one or more R_{B3}. In some embodiments, each R_{B3} is independently 5-membered heteroaryl substituted with one or more R_{B3′}.

In some embodiments, each R_{B3} is independently 6-membered heteroaryl. In some embodiments, each R_{B3} is independently 6-membered heteroaryl optionally substituted with one or more R_{B}. In some embodiments, each R_{B3} is independently 6-membered heteroaryl substituted with one or more Ray.

In some embodiments, each R_{B3} is independently 7-membered heteroaryl. In some embodiments, each R_{B3} is independently 7-membered heteroaryl optionally substituted with one or more R_{B3}. In some embodiments, each R_{B3} is independently 7-membered heteroaryl substituted with one or more Ray.

In some embodiments, each R_{B3} is independently 8-membered heteroaryl. In some embodiments, each R_{B3} is independently 8-membered heteroaryl optionally substituted with one or more R_{B3′}. In some embodiments, each R_{B3} is independently 8-membered heteroaryl substituted with one or more R_{B3′}.

In some embodiments, each R_{B3} is independently 9-membered heteroaryl. In some embodiments, each R_{B3} is independently 9-membered heteroaryl optionally substituted with one or more R_{B3′}. In some embodiments, each R_{B3} is independently 9-membered heteroaryl substituted with one or more R_{B3′}.

In some embodiments, each R_{B3} is independently 10-membered heteroaryl. In some embodiments, each R_{B3}, is independently 10-membered heteroaryl optionally substituted with one or more R_{B3}. In some embodiments, each R_{B3} is independently 10-membered heteroaryl substituted with one or more R_{B3′}.

In some embodiments, each R_{B3} is independently C₃-C₇ cycloalkyl or 3- to 7-membered heterocycloalkyl.

In some embodiments, each R_{B3} is independently C₃-C₇ cycloalkyl or 3- to 7-membered heterocycloalkyl, wherein the cycloalkyl and heterocycloalkyl are optionally substituted with one or more R_{B3′}.

In some embodiments, each R_{B3}3 is independently C₃-C₇ cycloalkyl or 3- to 7-membered heterocycloalkyl, wherein the cycloalkyl and heterocycloalkyl are substituted with one or more R_{B3′}.

In some embodiments, each R_{B3}3 is independently C₃-C₇ cycloalkyl. In some embodiments, each R_{B3} is independently C₃-C₇ cycloalkyl optionally substituted with one or more R_{B3′}. In some embodiments, each R_{B3} is independently C₃-C₇ cycloalkyl substituted with one or more R_{B3′}.

In some embodiments, each R_{B3} is independently C₃ cycloalkyl. In some embodiments, each R_{B3} is independently C₃ cycloalkyl optionally substituted with one or more R_{B3′}. In some embodiments, each R_{B3} is independently C₃ cycloalkyl substituted with one or more R_{B3′}.

In some embodiments, each R_{B3}3 is independently C₄ cycloalkyl. In some embodiments, each R_{B3} is independently C cycloalkyl optionally substituted with one or more R_{B3′}. In some embodiments, each R_{B3} is independently C₄ cycloalkyl substituted with one or more R_{B3′}.

In some embodiments, each R_{B3} is independently C₅ cycloalkyl. In some embodiments, each R_{B3} is independently C₅ cycloalkyl optionally substituted with one or more R_{B3′}. In some embodiments, each R_{B3} is independently C₅ cycloalkyl substituted with one or more R_{B3′}.

In some embodiments, each R_{B3} is independently C₆ cycloalkyl. In some embodiments, each R_{B3} is independently C₆ cycloalkyl optionally substituted with one or more R_{B3′}. In some embodiments, each R_{B3} is independently C₆ cycloalkyl substituted with one or more R_{B3′}.

In some embodiments, each R_{B3} is independently C₇ cycloalkyl. In some embodiments, each R_{B3} is independently C₇ cycloalkyl optionally substituted with one or more R_{B3′}. In some embodiments, each R_{B3} is independently C₇ cycloalkyl substituted with one or more R_{B3′}.

In some embodiments, each R_{B3} is independently 3- to 7-membered heterocycloalkyl. In some embodiments, each Raw is independently 3- to 7-membered heterocycloalkyl optionally substituted with one or more R_{B3′}. In some embodiments, each R_{B3} is independently 3- to 7-membered heterocycloalkyl substituted with one or more R_{B3′}.

In some embodiments, each R_{B3} is independently 3-membered heterocycloalkyl. In some embodiments, each R_{B3} is independently 3-membered heterocycloalkyl optionally substituted with one or more R_{B3′}. In some embodiments, each R_{B3} is independently 3-membered heterocycloalkyl substituted with one or more R_{B3′}.

In some embodiments, each R_{B3} is independently 4-membered heterocycloalkyl. In some embodiments, each R_{B3} is independently 4-membered heterocycloalkyl optionally substituted with one or more R_{B3′}. In some embodiments, each R_{B3} is independently 4-membered heterocycloalkyl substituted with one or more R_{B3′}.

In some embodiments, each Ru is independently 5-membered heterocycloalkyl. In some embodiments, each R_{B3} is independently 5-membered heterocycloalkyl optionally substituted with one or more R_{B3′}. In some embodiments, each R_{B3} is independently 5-membered heterocycloalkyl substituted with one or more R_{B3}.

In some embodiments, each R_{B3} is independently 6-membered heterocycloalkyl. In some embodiments, each R_{B3} is independently 6-membered heterocycloalkyl optionally substituted with one or more R_{B3′}. In some embodiments, each R_{B3} is independently 6-membered heterocycloalkyl substituted with one or more R_{B3}.

In some embodiments, each R_{B3} is independently 7-membered heterocycloalkyl. In some embodiments, each R_{B3} is independently 7-membered heterocycloalkyl optionally substituted with one or more R_{B3}. In some embodiments, each R_{B3} is independently 7-membered heterocycloalkyl substituted with one or more R_{B3′}.

In some embodiments, each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy.

In some embodiments, each R_{B3} is independently halogen, —CN, —OH, —NH₂, —NH(C₁-C₆ alkyl), —NH(C₁-C₆ alkyl)-OH, or —N(C₁-C₆ alkyl)₂.

In some embodiments, each R_{B3}, is independently halogen or —CN.

In some embodiments, each R_{B3′} is independently halogen.

In some embodiments, each R_{B3′} is independently F, Cl, Br, or I. In some embodiments, each R_{B3′} is independently F, Cl, or Br. In some embodiments, each R_{B3′} is independently F or Cl. In some embodiments, each R_{B3′} is independently F. In some embodiments, each R_{3B′} is independently C₁. In some embodiments, each R_{B3′} is independently Br. In some embodiments, each R_{B3′} is independently I.

In some embodiments, each R_{B3′} is independently —CN.

In some embodiments, each R_{B3′} is independently —OH, —NH₂, —NH(C₁-C₆ alkyl), —NH(C₁-C₆ alkyl)-OH, or —N(C₁-C₆ alkyl)₂.

In some embodiments, each R_{B3′} is independently —OH. In some embodiments, each R_{B3′}, is independently —NH₂.

In some embodiments, each R_{B3}, is independently —NH(C₁-C₆ alkyl), —NH(C₁-C₆ alkyl)-OH, or —N(C₁-C₆ alkyl)₂.

In some embodiments, each R_{B3′} is independently —NH(C₁-C₆ alkyl). In some embodiments, each R_{B3″} is independently —NH(C₁-C₆ alkyl)-OH. In some embodiments, each R_{B3″} is independently —N(C₁-C₆ alkyl)₂.

In some embodiments, each R_{B3} is independently C₁-C₆ alkyl, C₂-C₆ alkenyl, C₂-C₆ alkynyl, C₁-C₆ haloalkyl, or C₁-C₆ alkoxy.

In some embodiments, each R_{B3′} is independently C₁-C₆ alkyl, C₂-C₆ alkenyl, or C₂-C₆ alkynyl.

In some embodiments, each R_{B3′} is independently C₁-C₆ alkyl.

In some embodiments, each R_{B3′} is independently methyl. In some embodiments, each R_{B3} is independently ethyl. In some embodiments, each R_{B3′} is independently propyl. In some embodiments, each R_{B3′} is independently butyl. In some embodiments, each R_{B3′} is independently pentyl. In some embodiments, each R_{3B′} is independently hexyl. In some embodiments, each R_{B3′} is independently isopropyl. In some embodiments, each R_{B3′} is independently isobutyl. In some embodiments, each R_{B3′} is independently isopentyl. In some embodiments, each R_{B3′} is independently isohexyl. In some embodiments, each R_{B3′} is independently secbutyl. In some embodiments, each R_{B3} is independently secpentyl. In some embodiments, each R_{B3′} is independently sechexyl. In some embodiments, each R_{B3′} is independently tertbutyl.

In some embodiments, each R_{B3′} is independently C₂-C₆ alkenyl.

In some embodiments, each R_{B3}, is independently C₂ alkenyl. In some embodiments, each R_{B1} is independently C₃ alkenyl. In some embodiments, each R_{B3′} is independently C₄ alkenyl. In some embodiments, each R_{B3′} is independently C₅ alkenyl. In some embodiments, each R_{B3′} is independently C₆ alkenyl.

In some embodiments, each R_{B3′} is independently C₂-C₆ alkynyl.

In some embodiments, each R_{B3′} is independently C₂ alkynyl. In some embodiments, each R_{B3′} is independently C₃ alkynyl. In some embodiments, each R_{B3} is independently C₄ alkynyl. In some embodiments, each R_{B3} is independently C₅ alkynyl. In some embodiments, each R_{B3} is independently C₆ alkynyl.

In some embodiments, each R_{B3′} is independently C₁-C₆ haloalkyl or C₁-C₆ alkoxy.

In some embodiments, each R_{B3′} is independently C₁-C₆ haloalkyl.

In some embodiments, each R_{B3′} is independently halomethyl. In some embodiments, each R_{B3′} is independently haloethyl. In some embodiments, each R_{B3′} is independently halopropyl. In some embodiments, each R_{B3′} is independently halobutyl. In some embodiments, each R_{B3}, is independently halopentyl. In some embodiments, each R_{B3′} is independently halohexyl.

In some embodiments, each R_{B3′} is independently C₁-C₆ alkoxy.

In some embodiments, each R_{B3}, is independently methoxy. In some embodiments, each R_{B3′} is independently ethoxy. In some embodiments, each R_{B3′} is independently propoxy. In some embodiments, each R_{B3′} is independently butoxy. In some embodiments, each R_{B3′} is independently pentoxy. In some embodiments, each R_{B3′} is independently hexoxy.

In some embodiments, each R_{B4} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₘ—C(O)R_{B4}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4″}).

In some embodiments, each R_{B4} is independently oxo, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₆ cycloalkyl, or 3- to 7-membered heterocycloalkyl.

In some embodiments, each R_{B4} is independently oxo, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4″}).

In some embodiments, each R_{B4} is independently oxo, halogen, —CN, —OH, —NH₂, —NH(C₁-C₆, alkyl), —NH(C₁-C₆ alkyl)-OH, —N(C₁-C₆ alkyl)₂, C₁-C₆ alkyl, C₂-C₆ alkenyl, C₂-C₆, alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is substituted with one or more halogen, —CN, —OR_{B4′}, or —N(R_{B4′})(R_{B4″}).

In some embodiments, each R_{B4} is independently oxo, halogen, —CN, —OH, —NH₂, —NH(C₁-C₆ alkyl), —NH(C₁-C₆ alkyl)-OH, or —N(C₁-C₆ alkyl)₂.

In some embodiments, each R_{B4} is independently oxo, halogen, —CN, —OH, —NH₂, —NH(C₁-C₆ alkyl), —NH(C₁-C₆ alkyl)-OH, or —N(C₁-C₆ alkyl)₂, wherein the alkyl is optionally substituted with one or more halogen, —CN, —OR_{B4′}, or —N(R_{B4′})(R_{B4″}).

In some embodiments, each R_{B4} is independently oxo, halogen, —CN, —OH, —NH₂, —NH(C₁-C₆ alkyl), —NH(C₁-C₆ alkyl)-OH, or —N(C₁-C₆ alkyl)₂, wherein the is substituted with one or more halogen, —CN, —OR_{B4′}, or —N(R_{B4′})(R_{B″}).

In some embodiments, each R_{B4} is independently oxo.

In some embodiments, each R_{B4} is independently halogen or —CN.

In some embodiments, each R_{B4} is independently halogen.

In some embodiments, each R_{B4} is independently F, Cl, Br, or I. In some embodiments, each R_{B4} is independently F, Cl, or Br. In some embodiments, each R_{B4} is independently F or Cl. In some embodiments, each R_{B4} is independently F. In some embodiments, each R_{B4} is independently C₁. In some embodiments, each R_{B4} is independently Br. In some embodiments, each R_{B4} is independently I.

In some embodiments, each R_{B4} is independently —CN.

In some embodiments, each R_{B4} is independently —OH, —NH₂, —NH(C₁-C₆ alkyl), —NH(C₁-C₆ alkyl)-OH, or —N(C₁-C₆ alkyl)₂.

In some embodiments, each R_{B4} is independently —OH, —NH₂, —NH(C₁-C₆ alkyl), —NH(C₁-C₆ alkyl)-OH, or —N(C₁-C₆ alkyl)₂, wherein the alkyl is optionally substituted with one or more halogen, —CN, —OR_{B4′}, or —N(R_{B4′})(R_{B4″}).

In some embodiments, each R_{B4} is independently —OH, —NH₂, —NH(C₁-C₆ alkyl), —NH(C₁-C₆ alkyl)-OH, or —N(C₁-C₆ alkyl)₂, wherein the alkyl is substituted with one or more halogen, —CN, —OR_{B4′}, or —N(R_{B4′})(R_{B4″}).

In some embodiments, each R_{B4} is independently —OH. In some embodiments, each R_{B4} is independently —NH₂.

In some embodiments, each R₄ is independently —NH(C₁-C₆ alkyl), —NH(C₁-C₆ alkyl)-OH, or —N(C₁-C₆ alkyl)₂.

In some embodiments, each R_{B4} is independently —NH(C₁-C₆ alkyl), —NH(C₁-C₆ alkyl)-OH, or —N(C₁-C₆ alkyl)₂, wherein the alkyl is optionally substituted with one or more halogen, —CN, —OR_{B4′}, or —N(R_{B4′})(R_{B4″}).

In some embodiments, each R_{B4} is independently —NH(C₁-C₆ alkyl), —NH(C₁-C₆ alkyl)-OH, or —N(C₁-C₆ alkyl)₂, wherein the alkyl is substituted with one or more halogen, —CN, —OR_{B3}, or —N(R_{B4′})(R_{B4″}).

In some embodiments, each R_{B4} is independently —NH(C₁-C₆ alkyl). In some embodiments, each R_{B3} is independently —NH(C₁-C₆ alkyl), wherein the alkyl is optionally substituted with one or more halogen, —CN, —OR_{B4′}, or —N(R_{B4′})(R_{B4″}). In some embodiments, each R_{B4} is independently —NH(C₁-C₆ alkyl), wherein the alkyl is substituted with one or more halogen, —CN, —OR_{B4′}, or —N(R_{B4′})(R_{B4″}).

In some embodiments, each R_{B4} is independently —NH(C₁-C₆ alkyl)-OH. In some embodiments, each R_{B4} is independently —NH(C₁-C₆ alkyl)-OH, wherein the alkyl is optionally substituted with one or more halogen, —CN, —OR_{B4′}, or —N(R_{B4′})(R_{B4″}). In some embodiments, each R_{B3} is independently —NH(C₁-C₆ alkyl)-OH, wherein the alkyl is substituted with one or more halogen, —CN, —OR_{B4′}, or —N(R_{B4′})(R_{B4″}).

In some embodiments, each R_{B4} is independently —N(C₁-C₆ alkyl)₂. In some embodiments, each R_{B4} is independently —N(C₁-C₆ alkyl)₂, wherein the alkyl is optionally substituted with one or more halogen, —CN, —OR_{B4′}, or —N(R_{B4′})(R_{B4″}). In some embodiments, each R_{B4} is independently —N(C₁-C₆ alkyl)₂, wherein the alkyl is substituted with one or more halogen, —CN, —OR_{B}c, or —N(R_{B4′})(R_{B3″}).

In some embodiments, each R_{B4} is independently —(CH₂)ₘ—C(O)R_{B4}.

In some embodiments, each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl.

In some embodiments, each R_{B4} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4″}).

In some embodiments, each R_{B4} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₁-C₇ cycloalkyl, or 3- to 7-membered heterocycloalkyl, wherein the alkyl, alkenyl, alkynyl, aryl, heteroaryl, cycloalkyl, or heterocycloalkyl is substituted with one or more halogen, —CN, —OR_{B4′}, or —N(R_{B4′})(R_{B3″}).

In some embodiments, each R_{B4} is independently C₁-C₆ alkyl, C₂-C₆ alkenyl, C₂-C₆ alkynyl, C₁-C₆ haloalkyl, or C₁-C₆ alkoxy.

In some embodiments, each R_{B4} is independently C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is optionally substituted with one or more halogen, —CN, —OR_{B4′}, or —N(R_{B4′})(R_{B3″}).

In some embodiments, each R_{B4} is independently C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl is substituted with one or more halogen, —CN, —OR_{B4′}, or —N(R_{B4′})(R_{B4″}).

In some embodiments, each R_{B4} is independently C₁-C₆ alkyl, C₂-C₆ alkenyl, or C₂-C₆ alkynyl.

In some embodiments, each R_{B4} is independently C₁-C₆ alkyl, C₂-C₆ alkenyl, or C₂-C₆ alkynyl, wherein the alkyl, alkenyl, or alkynyl is optionally substituted with one or more halogen, —CN, —OR_{B4′}, or —N(R_{B4′})(R_{B4″}).

In some embodiments, each R_{B4} is independently C₁-C₆ alkyl, C₂-C₆ alkenyl, or C₂-C₆ alkynyl, wherein the alkyl, alkenyl, or alkynyl is substituted with one or more halogen, —CN, —OR_{B4′}, or —N(R_{B4′})(R_{B4″}).

In some embodiments, each R_{B4} is independently C₁-C₆ alkyl. In some embodiments, each R_{B} is independently C₁-C₆ alkyl optionally substituted with one or more halogen, —CN, —OR_{B4′}, or —N(R_{B4′})(R_{B4″}). In some embodiments, each R_{B4} is independently C₁-C₆ alkyl substituted with one or more halogen, —CN, —OR_{B4′}, or —N(R_{B4′})(R_{B4″}).

In some embodiments, each R_{B4} is independently methyl. In some embodiments, each R_{B4} is independently ethyl. In some embodiments, each R_{B4} is independently propyl. In some embodiments, each R_{B4} is independently butyl. In some embodiments, each R_{B4} is independently pentyl. In some embodiments, each R_{B4} is independently hexyl. In some embodiments, each R_{B4} is independently isopropyl. In some embodiments, each R_{B4} is independently isobutyl. In some embodiments, each R_{B4} is independently isopentyl. In some embodiments, each R_{B4} is independently isohexyl. In some embodiments, each R_{B4} is independently secbutyl. In some embodiments, each R_{B4} is independently secpentyl. In some embodiments, each R_{B4} is independently sechexyl. In some embodiments, each R_{B4} is independently tertbutyl.

In some embodiments, each R_{B4} is independently methyl optionally substituted with one or more halogen, —CN, —OR_{B4′}, or —N(R_{B4′})(R_{B4″}). In some embodiments, each R_{B4} is independently ethyl optionally substituted with one or more halogen, —CN, —OR_{B4′}, or —N(R_{B4′})(R_{B4″}). In some embodiments, each R_{B4} is independently propyl optionally substituted with one or more halogen, —CN, —OR_{B4′}, or —N(R_{B4′})(R_{B4″}). In some embodiments, each R_{B4} is independently butyl optionally substituted with one or more halogen, —CN, —OR_{B4′}, or —N(R_{B4′})(R_{B4″}). In some embodiments, each R_{B4} is independently pentyl optionally substituted with one or more halogen, —CN, —OR_{B4′}, or —N(R_{B4′})(R_{B4″}). In some embodiments, each R_{B4} is independently hexyl optionally substituted with one or more halogen, —CN, —OR_{B4′}, or —N(R_{B4′})(R_{B4″}). In some embodiments, each R_{B4} is independently isopropyl optionally substituted with one or more halogen, —CN, —OR_{B4′}, or —N(R_{B4′})(R_{B4″}). In some embodiments, each R_{B4} is independently isobutyl optionally substituted with one or more halogen, —CN, —OR_{B4′}, or —N(R_{B4′})(R_{B4″}). In some embodiments, each R_{B4} is independently isopentyl optionally substituted with one or more halogen, —CN, —OR_{B4′}, or —N(R_{B4′})(R_{B4″}). In some embodiments, each R_{B4} is independently isohexyl optionally substituted with one or more halogen, —CN, —OR_{B4′}, or —N(R_{B4′})(R_{B4″}). In some embodiments, each R_{B4} is independently secbutyl optionally substituted with one or more halogen, —CN, —OR_{B4′}, or —N(R_{B4′})(R_{B4″}). In some embodiments, each R_{B4} is independently secpentyl optionally substituted with one or more halogen, —CN, —OR_{B4′}, or —N(R_{B4′})(R_{B4″}). In some embodiments, each R_{B4} is independently sechexyl optionally substituted with one or more halogen, —CN, —OR_{B4′}, or —N(R_{B4′})(R_{B4″}). In some embodiments, each R_{B4} is independently tertbutyl optionally substituted with one or more halogen, —CN, —OR_{B4′}, or —N(R_{B4′})(R_{B4″}).

In some embodiments, each R_{B4} is independently methyl substituted with one or more halogen, —CN, —OR_{B4′}, or —N(R_{B4′})(R_{B4″}). In some embodiments, each R_{B4} is independently ethyl substituted with one or more halogen, —CN, —OR_{B4′}, or —N(R_{B4′})(R_{B4″}). In some embodiments, each R_{B4} is independently propyl substituted with one or more halogen, —CN, —OR_{B4′}, or —N(R_{B4′})(R_{B4″}). In some embodiments, each R_{B4} is independently butyl substituted with one or more halogen, —CN, —OR_{B4′}, or —N(R_{B4′})(R_{B4″}). In some embodiments, each R_{B4} is independently pentyl substituted with one or more halogen, —CN, —OR_{B4′}, or —N(R_{B4′})(R_{B4″}). In some embodiments, each R_{B4} is independently hexyl substituted with one or more halogen, —CN, —OR_{B4′}, or —N(R_{B4′})(R_{B4″}). In some embodiments, each R_{B4} is independently isopropyl substituted with one or more halogen, —CN, —OR_{B4′}, or —N(R_{B4′})(R_{B4″}). In some embodiments, each R_{B4} is independently isobutyl substituted with one or more halogen, —CN, —OR_{B4′}, or —N(R_{B4′})(R_{B4″}). In some embodiments, each R_{B4} is independently isopentyl substituted with one or more halogen, —CN, —OR_{B4′}, or —N(R_{B4′})(R_{B4″}). In some embodiments, each R_{B4} is independently isohexyl substituted with one or more halogen, —CN, —OR_{B4′}, or —N(R_{B4′})(R_{B4″}). In some embodiments, each R_{B4} is independently secbutyl substituted with one or more halogen, —CN, —OR_{B4′}, or —N(R_{B4′})(R_{B4″}). In some embodiments, each R_{B4} is independently secpentyl substituted with one or more halogen, —CN, —OR_{B4′}, or —N(R_{B4′})(R_{B4″}). In some embodiments, each R_{B4} is independently sechexyl substituted with one or more halogen, —CN, —OR_{B4′}, or —N(R_{B4′})(R_{B4″}). In some embodiments, each R_{B4} is independently tertbutyl substituted with one or more halogen, —CN, —OR_{B4′}, or —N(R_{B4′})(R_{B4}″).

In some embodiments, each R_{B4} is independently C₂-C₆ alkenyl. In some embodiments, each R_{B4} is independently C₂-C₆ alkenyl optionally substituted with one or more halogen, —CN, —OR_{B4′}, or —N(R_{B4′})(R_{B4″}). In some embodiments, each R_{B4} is independently C₂-C₆ alkenyl substituted with one or more halogen, —CN, —OR_{B4′}, or —N(R_{B4′})(R_{B4″}).

In some embodiments, each R_{B4} is independently C₂ alkenyl. In some embodiments, each R_{B4} is independently C₃ alkenyl. In some embodiments, each R_{B4} is independently C₄ alkenyl. In some embodiments, each R_{B4} is independently C₅ alkenyl. In some embodiments, each R_{B4} is independently C₆ alkenyl.

In some embodiments, each R_{B4} is independently C₂ alkenyl optionally substituted with one or more halogen, —CN, —OR_{B4′}, or —N(R_{B4′})(R_{B4″}). In some embodiments, each R_{B4} is independently C₃ alkenyl optionally substituted with one or more halogen, —CN, —OR_{B4′}, or —N(R_{B4′})(R_{B4″}). In some embodiments, each R_{B4} is independently C₄ alkenyl optionally substituted with one or more halogen, —CN, —OR_{B4′}, or —N(R_{B4′})(R_{B4″}). In some embodiments, each R_{B4} is independently C₅ alkenyl optionally substituted with one or more halogen, —CN, —OR_{B4′}, or —N(R_{B4′})(R_{B4″}). In some embodiments, each R_{B4} is independently C₆ alkenyl optionally substituted with one or more halogen, —CN, —OR_{B4′}, or —N(R_{B4′})(R_{B4″}).

In some embodiments, each R_{B4} is independently C₂ alkenyl substituted with one or more halogen, —CN, —OR_{B4′}, or —N(R_{B4′})(R_{B4″}). In some embodiments, each R_{B4} is independently C₃ alkenyl substituted with one or more halogen, —CN, —OR_{B4′}, or —N(R_{B4′})(R_{B4″}). In some embodiments, each R_{B4} is independently C₄ alkenyl substituted with one or more halogen, —CN, —OR_{B4′}, or —N(R_{B4′})(R_{B4″}). In some embodiments, each R_{B4} is independently C₅ alkenyl substituted with one or more halogen, —CN, —OR_{B4′}, or —N(R_{B4′})(R_{B4″}). In some embodiments, each R_{B4} is independently C₆ alkenyl substituted with one or more halogen, —CN, —OR_{B4′}, or —N(R_{B4′})(R_{B4″}).

In some embodiments, each R_{B4} is independently C₂-C₆ alkynyl. In some embodiments, each R_{B4} is independently C₂-C₆ alkynyl optionally substituted with one or more halogen, —CN, —OR_{B4′}, or —N(R_{B4′})(R_{B4″}). In some embodiments, each R_{B4} is independently C₂-C₆ alkynyl substituted with one or more halogen, —CN, —OR_{B4′}, or —N(R_{B4′})(R_{B4″}).

In some embodiments, each R_{B4} is independently C₂ alkynyl. In some embodiments, each R_{B4} is independently C₃ alkynyl. In some embodiments, each R_{B4} is independently C₄ alkynyl. In some embodiments, each R_{B4} is independently C₅ alkynyl. In some embodiments, each R_{B4} is independently C₆ alkynyl.

In some embodiments, each R_{B4} is independently C₂ alkynyl optionally substituted with one or more halogen, —CN, —OR_{B4′}, or —N(R_{B4′})(R_{B4″}). In some embodiments, each R_{B4} is independently C₃ alkynyl optionally substituted with one or more halogen, —CN, —OR_{B4′}, or —N(R_{B4′})(R_{B4″}). In some embodiments, each R_{B4} is independently C₄ alkynyl optionally substituted with one or more halogen, —CN, —OR_{B4′}, or —N(R_{B4′})(R_{B4″}). In some embodiments, each R_{B4} is independently C₅ alkynyl optionally substituted with one or more halogen, —CN, —OR_{B4′}, or —N(R_{B4′})(R_{B4″}). In some embodiments, each R_{B4} is independently C₆ alkynyl optionally substituted with one or more halogen, —CN, —OR_{B4′}, or —N(R_{B4′})(R_{B4″}).

In some embodiments, each R_{B4} is independently C₂ alkynyl substituted with one or more halogen, —CN, —OR_{B4′}, or —N(R_{B4′})(R_{B4″}). In some embodiments, each R_{B4} is independently C alkynyl substituted with one or more halogen, —CN, —OR_{B4′}, or —N(R_{B4′})(R_{B4″}). In some embodiments, each R_{B4} is independently C₄ alkynyl substituted with one or more halogen, —CN, —OR_{B4′}, or —N(R_{B4′})(R_{B4″}). In some embodiments, each R_{B4} is independently C₅ alkynyl substituted with one or more halogen, —CN, —OR_{B4′}, or —N(R_{B4′})(R_{B4″}). In some embodiments, each R_{B4} is independently C₆ alkynyl substituted with one or more halogen, —CN, —OR_{B4′}, or —N(R_{B4′})(R_{B4″}).

In some embodiments, each R_{B4} is independently C₁-C₆ haloalkyl or C₁-C₆ alkoxy.

In some embodiments, each R_{B4} is independently C₁-C₅ haloalkyl or C₁-C₆ alkoxy, wherein the alkyl is optionally substituted with one or more halogen, —CN, —OR_{B4′}, or —N(R_{B4′})(R_{B4″}).

In some embodiments, each R_{B4} is independently C₁-C₆ haloalkyl or C₁-C₆ alkoxy, wherein the alkyl is substituted with one or more halogen, —CN, —OR_{B4′}, or —N(R_{B4′})(R_{B4″}).

In some embodiments, each R_{B4} is independently C₁-C₆ haloalkyl. In some embodiments, each R_{B4} is independently C₁-C₆ haloalkyl, wherein the alkyl is optionally substituted with one or more halogen, —CN, —OR_{B4′}, or —N(R_{B4′})(R_{B4″}). In some embodiments, each R_{B4} is independently C₁-C₆ haloalkyl, wherein the alkyl is substituted with one or more halogen, —CN, —OR_{B4′}, or —N(R_{B4′})(R_{B4″}).

In some embodiments, each R_{B4} is independently halomethyl. In some embodiments, each R_{B4} is independently haloethyl. In some embodiments, each R_{B4} is independently halopropyl. In some embodiments, each R_{B4} is independently halobutyl. In some embodiments, each R_{B4} is independently halopentyl. In some embodiments, each R_{B4} is independently halohexyl.

In some embodiments, each R_{B4} is independently halomethyl, wherein the alkyl is optionally substituted with one or more halogen, —CN, —OR_{B4′}, or —N(R_{B4″})(R_{B4″}). In some embodiments, each R_{B4} is independently haloethyl, wherein the alkyl is optionally substituted with one or more halogen, —CN, —OR_{B4′}, or —N(R_{B4′})(R_{B4″}). In some embodiments, each R_{B4} is independently halopropyl, wherein the alkyl is optionally substituted with one or more halogen, —CN, —OR_{B4′}, or —N(R_{B4′})(R_{B4″}). In some embodiments, each R_{B4} is independently halobutyl, wherein the alkyl is optionally substituted with one or more halogen, —CN, —OR_{B4′}, or —N(R_{B4′})(R_{B4″}). In some embodiments, each R_{B4} is independently halopentyl, wherein the alkyl is optionally substituted with one or more halogen, —CN, —OR_{B4′}, or —N(R_{B4′})(R_{B4″}). In some embodiments, each R_{B4} is independently halohexyl, wherein the alkyl is optionally substituted with one or more halogen, —CN, —OR_{B4′}, or —N(R_{B4′})(R_{B4″}).

In some embodiments, each R_{B4} is independently halomethyl, wherein the alkyl is substituted with one or more halogen, —CN, —OR_{B4′}, or —N(R_{B4′})(R_{B4″}). In some embodiments, each R_{B4} is independently haloethyl, wherein the alkyl is substituted with one or more halogen, —CN, —OR_{B4′}, or —N(R_{B4′})(R_{B4″}). In some embodiments, each R_{B4} is independently halopropyl, wherein the alkyl is substituted with one or more halogen, —CN, —OR_{B4′} or —N(R_{B4′})(R_{B4″}). In some embodiments, each R_{B4} is independently halobutyl, wherein the alkyl is substituted with one or more halogen, —CN, —OR_{B4′}, or —N(R_{B4′})(R_{B44″}). In some embodiments, each R_{B4} is independently halopentyl, wherein the alkyl is substituted with one or more halogen, —CN, —OR_{B4′}, or —N(R_{B4′})(R_{B4″}). In some embodiments, each R_{B4} is independently halohexyl, wherein the alkyl is substituted with one or more halogen, —CN, —OR_{B4′}, or —N(R_{B4′})(R_{B4″}).

In some embodiments, each R_{B4} is independently C₁-C₆ alkoxy. In some embodiments, each R_{B4} is independently C₁-C₆ alkoxy, wherein the alkyl is optionally substituted with one or more halogen, —CN, —OR_{B4′}, or —N(R_{B4′})(R_{B4″}). In some embodiments, each R_{B4} is independently C₁-C₆ alkoxy, wherein the alkyl is substituted with one or more halogen, —CN, —OR_{B4′}, or —N(R_{B4′})(R_{B4″}).

In some embodiments, each R_{B4} is independently methoxy. In some embodiments, each R_{B4} is independently ethoxy. In some embodiments, each R_{B4} is independently propoxy. In some embodiments, each R_{B4} is independently butoxy. In some embodiments, each R_{B4} is independently pentoxy. In some embodiments, each R_{B4} is independently hexoxy.

In some embodiments, each R_{B4} is independently methoxy, wherein the alkyl is optionally substituted with one or more halogen, —CN, —OR_{B4′}, or —N(R_{B4′})(R_{B4″}). In some embodiments, each R_{B4} is independently ethoxy, wherein the alkyl is optionally substituted with one or more halogen, —CN, —OR_{B4′}, or —N(R_{B4′})(R_{B4″}). In some embodiments, each R_{B4} is independently propoxy, wherein the alkyl is optionally substituted with one or more halogen, —CN, —OR_{B4′}, or —N(R_{B4′})(R_{B4″}). In some embodiments, each R_{B4} is independently butoxy, wherein the alkyl is optionally substituted with one or more halogen, —CN, —OR_{B4′}, or —N(R_{B4′})(R_{B4″}). In some embodiments, each R_{B4} is independently pentoxy, wherein the alkyl is optionally substituted with one or more halogen, —CN, —OR_{B4′}, or —N(R_{B4′})(R_{B4″}). In some embodiments, each R_{B4} is independently hexoxy, wherein the alkyl is optionally substituted with one or more halogen, —CN, —OR_{B4′}, or —N(R_{B4′})(R_{B4″}).

In some embodiments, each R_{B4} is independently methoxy, wherein the alkyl is substituted with one or more halogen, —CN, —OR_{B4′}, or —N(R_{B4′})(R_{B4″}). In some embodiments, each R_{B4} is independently ethoxy, wherein the alkyl is substituted with one or more halogen, —CN, —OR_{B3′}, or —N(R_{B4′})(R_{B4″}). In some embodiments, each R_{B4} is independently propoxy, wherein the alkyl is substituted with one or more halogen, —CN, —OR_{B4′}, or —N(R_{B4′})(R_{B4″}). In some embodiments, each R_{B4} is independently butoxy, wherein the alkyl is substituted with one or more halogen, —CN, —OR_{B4′}, or —N(R_{B4′})(R_{B4″}). In some embodiments, each R_{B4} is independently pentoxy, wherein the alkyl is substituted with one or more halogen, —CN, —OR_{B4′}, or —N(R_{B4′})(R_{B4″}). In some embodiments, each R_{B4} is independently hexoxy, wherein the alkyl is substituted with one or more halogen, —CN, —OR_{B4′}, or —N(R_{B4′})(R_{B4″}).

In some embodiments, each R_{B4} is independently C₆-C₁₀ aryl, 5- to 10-membered heteroaryl, C₃-C₇ cycloalkyl, or 3- to 7-membered heterocycloalkyl.

In some embodiments, each R_{B4} is independently C₆-C₁₀ aryl, 5- to 10-membered heteroaryl, C₃-C₇ cycloalkyl, or 3- to 7-membered heterocycloalkyl, wherein the aryl, heteroaryl, cycloalkyl, or heterocycloalkyl is optionally substituted with one or more halogen, —CN, —OR_{B4′}, or —N(R_{B4′})(R_{B4″}).

In some embodiments, each R_{B4} is independently C₆-C₁₀ aryl, 5- to 10-membered heteroaryl, C₃-C₇ cycloalkyl, or 3- to 7-membered heterocycloalkyl, wherein the aryl, heteroaryl, cycloalkyl, or heterocycloalkyl is substituted with one or more halogen, —CN, —OR_{B4′}, or —N(R_{B4′})(R_{B4″}).

In some embodiments, each R_{B4} is independently C₆-C₁₀ aryl or 5- to 10-membered heteroaryl. In some embodiments, each R_{B4} is independently C₆-C₁₀ aryl or 5- to 10-membered heteroaryl, wherein the aryl or heteroaryl is optionally substituted with one or more halogen, —CN, —OR_{B4′}, or —N(R_{B4′})(R_{B4″}). In some embodiments, each R_{B4} is independently C₆-C₁₀ aryl or 5- to 10-membered heteroaryl, wherein the aryl or heteroaryl is substituted with one or more halogen, —CN, —OR_{B4′}, or —N(R_{B4′})(R_{B4″}).

In some embodiments, each R_{B4} is independently C₆-C₁₀ aryl. In some embodiments, each R_{B} is independently C₆-C₁₀ aryl optionally substituted with one or more halogen, —CN, —OR_{B4′}, or —N(R_{B4′})(R_{4B″}). In some embodiments, each R_{B4} is independently C₆-C₁₀ aryl substituted with one or more halogen, —CN, —OR_{B4′}, or —N(R_{B4′})(R_{B4″}).

In some embodiments, each R_{B4} is independently C₆ aryl (e.g., phenyl). In some embodiments, each R_{B4} is independently C₆ aryl (e.g., phenyl) optionally substituted with one or more halogen, —CN, —OR_{B4′}, or —N(R_{B4′})(R_{B4″}). In some embodiments, each R_{B} is independently C₆, aryl (e.g., phenyl) substituted with one or more halogen, —CN, —OR_{B4′}, or —N(R_{B4′})(R_{B4″}).

In some embodiments, each R_{B4} is independently C₅ aryl. In some embodiments, each R_{B2} is independently Ca aryl optionally substituted with one or more halogen, —CN, —OR_{B4′}, or —N(R_{B4′})(R_{B4″}). In some embodiments, each R_{B} is independently C₅ aryl substituted with one or more halogen, —CN, —OR_{B4′}, or —N(R_{B4′})(R_{B4″}).

In some embodiments, each R_{B} is independently C₁₀ aryl. In some embodiments, each R_{B} is independently C₁₀ aryl optionally substituted with one or more halogen, —CN, —OR_{B4′}, or —N(R_{B4′})(R_{B4″}). In some embodiments, each R_{B4} is independently C₁₀ aryl substituted with one or more halogen, —CN, —OR_{B4′}, or —N(R_{B4′})(R_{B4″}).

In some embodiments, each R_{B4} is independently 5- to 10-membered heteroaryl. In some embodiments, each R_{B4} is independently 5- to 10-membered heteroaryl optionally substituted with one or more halogen, —CN, —OR_{B4′}, or —N(R_{B4′})(R_{B4″}). In some embodiments, each R_{B} is independently 5- to 10-membered heteroaryl substituted with one or more halogen, —CN, —OR_{B4′}, or —N(R_{B4′})(R_{B4″}).

In some embodiments, each R_{B4} is independently 5-membered heteroaryl. In some embodiments, each R_{B} is independently 5-membered heteroaryl optionally substituted with one or more halogen, —CN, —OR_{B4′}, or —N(R_{B4′})(R_{B4″}). In some embodiments, each R_{B4} is independently 5-membered heteroaryl substituted with one or more halogen, —CN, —OR_{B4′}, or —N(R_{B4′})(R_{B4″}).

In some embodiments, each R_{B4} is independently 6-membered heteroaryl. In some embodiments, each R_{B4} is independently 6-membered heteroaryl optionally substituted with one or more halogen, —CN, —OR_{B4′}, or —N(R_{B4′})(R_{B4″}). In some embodiments, each R_{B4} is independently 6-membered heteroaryl substituted with one or more halogen, —CN, —OR_{B4′}, or —N(R_{B4′})(R_{B4″}).

In some embodiments, each R_{B4} is independently 7-membered heteroaryl. In some embodiments, each R_{B4} is independently 7-membered heteroaryl optionally substituted with one or more halogen, —CN, —OR_{B4′} or —N(R_{B4′})(R_{B4″}). In some embodiments, each R_{B4} is independently 7-membered heteroaryl substituted with one or more halogen, —CN, —OR_{B4′}, or —N(R_{B4′})(R_{B4″}).

In some embodiments, each R_{B4} is independently 8-membered heteroaryl. In some embodiments, each R_{B4} is independently 8-membered heteroaryl optionally substituted with one or more halogen, —CN, —OR_{B4′}, or —N(R_{B4′})(R_{B4}″). In some embodiments, each R_{B4} is independently 8-membered heteroaryl substituted with one or more halogen, —CN, —OR_{B4′}, or —N(R_{B4′})(R_{B4″}).

In some embodiments, each R_{B4} is independently 9-membered heteroaryl. In some embodiments, each R_{B4} is independently 9-membered heteroaryl optionally substituted with one or more halogen, —CN, —OR_{B4′}, or —N(R_{B4′})(R_{B4″}). In some embodiments, each R_{B4} is independently 9-membered heteroaryl substituted with one or more halogen, —CN, —OR_{B4′}, or —N(R_{B4′})(R_{B4″}).

In some embodiments, each R_{B4} is independently 10-membered heteroaryl. In some embodiments, each R_{B4} is independently 10-membered heteroaryl optionally substituted with one or more halogen, —CN, —OR_{B4′}, or —N(R_{B4′})(R_{B4″}). In some embodiments, each R_{B4} is independently 10-membered heteroaryl substituted with one or more halogen, —CN, —OR_{B4′}, or —N(R_{B4′})(R_{B4″}).

In some embodiments, each R_{B4} is independently C₃-C₇ cycloalkyl or 3- to 7-membered heterocycloalkyl. In some embodiments, each R_{B4} is independently C₃-C₇ cycloalkyl or 3- to 7-membered heterocycloalkyl, wherein the cycloalkyl or heterocycloalkyl is optionally substituted with one or more halogen, —CN, —OR_{B4′}, or —N(R_{B4′})(R_{B4″}). In some embodiments, each R_{B4} is independently C₃-C₇ cycloalkyl or 3- to 7-membered heterocycloalkyl, wherein the cycloalkyl or heterocycloalkyl is substituted with one or more halogen, —CN, —OR_{B4′}, or —N(R_{B4′})(R_{B4″}).

In some embodiments, each R_{B} is independently C₃-C₇ cycloalkyl. In some embodiments, each R_{B4} is independently C₃-C₇ cycloalkyl optionally substituted with one or more halogen, —CN, —OR_{B4′}, or —N(R_{B4′})(R_{B4″}). In some embodiments, each R_{B4} is independently C₃-C₇ cycloalkyl substituted with one or more halogen, —CN, —OR_{B4′}, or —N(R_{B4″})(R_{B4″}).

In some embodiments, each R_{B4} is independently C₃ cycloalkyl. In some embodiments, each R_{B4} is independently C₃ cycloalkyl optionally substituted with one or more halogen, —CN, —OR_{B4′}, or —N(R_{B4′})(R_{B4″}). In some embodiments, each R_{B4} is independently C cycloalkyl substituted with one or more halogen, —CN, —OR_{B4′}, or —N(R_{B4′})(R_{B4″}).

In some embodiments, each R_{B4} is independently C₄ cycloalkyl. In some embodiments, each R_{B4} is independently CA cycloalkyl optionally substituted with one or more halogen, —CN, —OR_{B4′}, or —N(R_{B4′})(R_{B4″}). In some embodiments, each R_{B4} is independently C₄ cycloalkyl substituted with one or more halogen, —CN, —OR_{B4′}, or —N(R_{B4′})(R_{B4″}).

In some embodiments, each R_{B4} is independently C₅ cycloalkyl. In some embodiments, each R_{B4} is independently C₅ cycloalkyl optionally substituted with one or more halogen, —CN, —OR_{B4′}, or —N(R_{B4′})(R_{B4″}). In some embodiments, each R_{B4} is independently C₅ cycloalkyl substituted with one or more halogen, —CN, —OR_{B4′}, or —N(R_{B4′})(R_{B4″}).

In some embodiments, each R_{B4} is independently C₆ cycloalkyl. In some embodiments, each R_{B4} is independently C₆ cycloalkyl optionally substituted with one or more halogen, —CN, —OR_{B4′}, or —N(R_{B4′})(R_{B4″}). In some embodiments, each R_{B4} is independently C₆ cycloalkyl substituted with one or more halogen, —CN, —OR_{B4′}, or —N(R_{B4′})(R_{B4″}).

In some embodiments, each R_{B4} is independently C₇ cycloalkyl. In some embodiments, each R_{B4} is independently C₇ cycloalkyl optionally substituted with one or more halogen, —CN, —OR_{B4′}, or —N(R_{B4′})(R_{B4″}). In some embodiments, each R_{B4} is independently C₇ cycloalkyl substituted with one or more halogen, —CN, —OR_{B4′}, or —N(R_{B4′})(R_{B4″}).

In some embodiments, each R_{B4} is independently 3- to 7-membered heterocycloalkyl. In some embodiments, each R_{B4} is independently 3- to 7-membered heterocycloalkyl optionally substituted with one or more halogen, —CN, —OR_{B4′}, or —N(R_{B4′})(R_{B4″}). In some embodiments, each R_{B4} is independently 3- to 7-membered heterocycloalkyl substituted with one or more halogen, —CN, —OR_{B4′}, or —N(R_{B4′})(R_{B4″}).

In some embodiments, each R_{B4} is independently 3-membered heterocycloalkyl. In some embodiments, each R_{B4} is independently 3-membered heterocycloalkyl optionally substituted with one or more halogen, —CN, —OR_{B4′}, or —N(R_{B4′})(R_{B4″}). In some embodiments, each R_{B4} is independently 3-membered heterocycloalkyl substituted with one or more halogen, —CN, —OR_{B4′}, or —N(R_{B4′})(R_{B4″}).

In some embodiments, each R_{B4} is independently 4-membered heterocycloalkyl. In some embodiments, each R_{B1} is independently 4-membered heterocycloalkyl optionally substituted with one or more halogen, —CN, —OR_{B4′}, or —N(R_{B4′})(R_{B4″}). In some embodiments, each Rum is independently 4-membered heterocycloalkyl substituted with one or more halogen, —CN, —OR_{B4′}, or —N(R_{B4′})(R_{B4″}).

In some embodiments, each R_{B4} is independently 5-membered heterocycloalkyl. In some embodiments, each R_{B4} is independently 5-membered heterocycloalkyl optionally substituted with one or more halogen, —CN, —OR_{B4′}, or —N(R_{B4′})(R_{B4″}). In some embodiments, each R_{B4} is independently 5-membered heterocycloalkyl substituted with one or more halogen, —CN, —OR_{B4′}, or —N(R_{B4′})(R_{B4″}).

In some embodiments, each R_{B4} is independently 6-membered heterocycloalkyl. In some embodiments, each R_{B4} is independently 6-membered heterocycloalkyl optionally substituted with one or more halogen, —CN, —OR_{B4′}, or —N(R_{B4′})(R_{B4″}). In some embodiments, each R_{B4} is independently 6-membered heterocycloalkyl substituted with one or more halogen, —CN, —OR_{B4′}, or —N(R_{B4′})(R_{B″}).

In some embodiments, each R_{B4} is independently 7-membered heterocycloalkyl. In some embodiments, each R_{B4} is independently 7-membered heterocycloalkyl optionally substituted with one or more halogen, —CN, —OR_{B4′}, or —N(R_{B4′})(R_{B4″}). In some embodiments, each R_{B4} is independently 7-membered heterocycloalkyl substituted with one or more halogen, —CN, —OR_{B4′}, or —N(R_{B4′})(R_{B4″}).

In some embodiments, each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

In some embodiments, each R_{B4′} and R_{B4″} is independently H, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl.

In some embodiments, each R_{B4′} and R_{B4″} is independently H, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

In some embodiments, each R_{B4′} and R_{B4″} is independently H, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are substituted with one or more oxo or —OH.

In some embodiments, R_{B4′} is H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl.

In some embodiments, R_{B4′} is H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

In some embodiments, R_{B4′} is H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are substituted with one or more oxo or —OH.

In some embodiments, R_{B4′} is H.

In some embodiments, R_{B4′} is —OH.

In some embodiments, R_{B4′} is —NH₂.

In some embodiments, R_{B4′} is C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl.

In some embodiments, R_{B4′} is C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

In some embodiments, R_{B4′} is C₁-C₆ alkyl, C₂-C₆ alkenyl, C₂-C₆ alkynyl, C₁-C₆ haloalkyl, C₁-C₆ alkoxy, C₃-C₆ cycloalkyl, or 3- to 7-membered heterocycloalkyl, wherein the alkyl, alkenyl, alkynyl, cycloalkyl, and heterocycloalkyl are substituted with one or more oxo or —OH.

In some embodiments, R_{B4′} is C₁-C₆ alkyl, C₂-C₆ alkenyl, C₂-C₆ alkynyl, C₁-C₆ haloalkyl, or C₁-C₆ alkoxy.

In some embodiments, R_{B4′} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more oxo or —OH.

In some embodiments, R_{B4′} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are substituted with one or more oxo or —OH.

In some embodiments, R_{B4′} is C₁-C₆ alkyl, C₂-C₆ alkenyl, or C₂-C₆ alkynyl.

In some embodiments, R_{B4′} is C₁-C₆ alkyl, C₂-C₆ alkenyl, or C₂-C₆ alkynyl, wherein the alkyl, alkenyl, or alkynyl are optionally substituted with one or more oxo or —OH.

In some embodiments, R_{B4′} is C₁-C₆ alkyl, C₂-C₆ alkenyl, or C₂-C₆ alkynyl, wherein the alkyl, alkenyl, or alkynyl are substituted with one or more oxo or —OH.

In some embodiments, R_{B4′} is C₁-C₆ alkyl. In some embodiments, R_{B4′} is C₁-C₆ alkyl optionally substituted with one or more oxo. In some embodiments, R_{B4} is C₁-C₆ alkyl substituted with one or more oxo or —OH.

In some embodiments, R_{B4′} is methyl. In some embodiments, R_{B4′} is ethyl. In some embodiments, R_{B4′} is propyl. In some embodiments, R_{B4′} is butyl. In some embodiments, R_{B4′} is pentyl. In some embodiments, R_{B4′} is hexyl. In some embodiments, R_{B4′} is isopropyl. In some embodiments, R_{B4′} is isobutyl. In some embodiments, R_{B4′} is isopentyl. In some embodiments, R_{B4′} is isohexyl. In some embodiments, R_{B4′} is secbutyl. In some embodiments, R_{B4′} is secpentyl. In some embodiments, R_{B′} is sechexyl. In some embodiments, R_{B4′} is tertbutyl.

In some embodiments, R_{B4′} is methyl optionally substituted with one or more oxo or —OH. In some embodiments, R_{B4′} is ethyl optionally substituted with one or more oxo or —OH. In some embodiments, R_{B4′} is propyl optionally substituted with one or more oxo or —OH. In some embodiments, R_{B4′} is butyl optionally substituted with one or more oxo or —OH. In some embodiments, R_{B4′} is pentyl optionally substituted with one or more oxo or —OH. In some embodiments, R_{B4′} is hexyl optionally substituted with one or more oxo or —OH. In some embodiments, R_{B4′} is isopropyl optionally substituted with one or more oxo or —OH. In some embodiments, R_{B4′} is isobutyl optionally substituted with one or more oxo or —OH. In some embodiments, R_{B4′} is isopentyl optionally substituted with one or more oxo or —OH or —OH. In some embodiments, R_{B4′} is isohexyl optionally substituted with one or more oxo. In some embodiments, R_{B4′} is secbutyl optionally substituted with one or more oxo or —OH. In some embodiments, R_{B4′} is secpentyl optionally substituted with one or more oxo or —OH. In some embodiments, R_{B4′} is sechexyl optionally substituted with one or more oxo or —OH. In some embodiments, R_{B4′} is tertbutyl optionally substituted with one or more oxo or —OH.

In some embodiments, R_{B4′} is methyl substituted with one or more oxo or —OH. In some embodiments, R_{B4′} is ethyl substituted with one or more oxo or —OH. In some embodiments, R_{B}e is propyl substituted with one or more oxo or —OH. In some embodiments, R_{B4′} is butyl substituted with one or more oxo or —OH. In some embodiments, R_{B4′} is pentyl substituted with one or more oxo or —OH. In some embodiments, R_{B4′} is hexyl substituted with one or more oxo or —OH. In some embodiments, R_{B4′} is isopropyl substituted with one or more oxo or —OH. In some embodiments, R_{B4′} is isobutyl substituted with one or more oxo or —OH. In some embodiments, R_{B4′} is isopentyl substituted with one or more oxo or —OH. In some embodiments, R_{B4′} is isohexyl substituted with one or more oxo or —OH. In some embodiments, R_{B4′} is secbutyl substituted with one or more oxo or —OH. In some embodiments, R_{B4′} is secpentyl substituted with one or more oxo or —OH. In some embodiments, R_{B4′} is sechexyl substituted with one or more oxo or —OH. In some embodiments, Rim, is tertbutyl substituted with one or more oxo or —OH.

In some embodiments, R_{B4′} is C₂-C₆ alkenyl. In some embodiments, R_{B4′} is C₂-C₆ alkenyl optionally substituted with one or more oxo or —OH. In some embodiments, R_{B4′} is C₂-C₆ alkenyl substituted with one or more oxo or —OH.

In some embodiments, R_{B4′} is C₂ alkenyl. In some embodiments, R_{B4′} is C₃ alkenyl. In some embodiments, R_{B4′} is C₄ alkenyl. In some embodiments, R_{B4′} is C₅ alkenyl. In some embodiments, R_{B4′} is C₆ alkenyl.

In some embodiments, R_{4B′} is C₂ alkenyl optionally substituted with one or more oxo or —OH. In some embodiments, R_{B4′} is C₃ alkenyl optionally substituted with one or more oxo or —OH. In some embodiments, R_{B4′} is C₄ alkenyl optionally substituted with one or more oxo or —OH. In some embodiments, R_{B4′} is C₅ alkenyl optionally substituted with one or more oxo or —OH. In some embodiments, R_{B4′} is C₆ alkenyl optionally substituted with one or more oxo or —OH.

In some embodiments, R_{B4′} is C₂ alkenyl substituted with one or more oxo or —OH. In some embodiments, R_{B4′} is C₃ alkenyl substituted with one or more oxo or —OH. In some embodiments, R_{B4′} is C₄ alkenyl substituted with one or more oxo or —OH. In some embodiments, R_{B4′} is C₅ alkenyl substituted with one or more oxo or —OH. In some embodiments, R_{B4′} is C₆ alkenyl substituted with one or more oxo or —OH.

In some embodiments, R_{B4} is C₂-C₆ alkynyl. In some embodiments, R_{B4} is C₂-C₆ alkynyl optionally substituted with one or more oxo or —OH. In some embodiments. R_{B4′} is C₂-C₆ alkynyl substituted with one or more oxo or —OH.

In some embodiments, R_{B4′} is C₂ alkynyl. In some embodiments, R_{B4′} is C₃ alkynyl. In some embodiments, R_{B} is C₄ alkynyl. In some embodiments, R_{B4′} is C₅ alkynyl. In some embodiments, R_{B4′} is C₆ alkynyl.

In some embodiments, R_{B4′} is C₂ alkynyl optionally substituted with one or more oxo or —OH. In some embodiments, R_{B4′} is C₃ alkynyl optionally substituted with one or more oxo or —OH. In some embodiments, R_{B4′} is C₄ alkynyl optionally substituted with one or more oxo or —OH. In some embodiments, R_{B4′} is C₅ alkynyl optionally substituted with one or more oxo or —OH. In some embodiments, R_{B4′} is C₆ alkynyl optionally substituted with one or more oxo or —OH.

In some embodiments, R_{B4′} is C₂ alkynyl substituted with one or more oxo or —OH. In some embodiments, R_{B4′} is C₃ alkynyl substituted with one or more oxo or —OH. In some embodiments, R_{B4′} is C₄ alkynyl substituted with one or more oxo or —OH. In some embodiments, R_{B4′} is C₅ alkynyl substituted with one or more oxo or —OH. In some embodiments, R_{B4′} is C₆ alkynyl substituted with one or more oxo or —OH.

In some embodiments, R_{B4′} is C₁-C₆ haloalkyl or C₁-C₆ alkoxy. In some embodiments, R_{B4′} is C₁-C₆ haloalkyl or C₁-C₆ alkoxy, wherein the alkyl is optionally substituted with one or more oxo or —OH. In some embodiments, R_{B4′} is C₁-C₆ haloalkyl or C₁-C₆ alkoxy, wherein the alkyl is substituted with one or more oxo or —OH.

In some embodiments, R_{B4′} is C₁-C₆ haloalkyl. In some embodiments, R_{B4′} is C₁-C₆ haloalkyl, wherein the alkyl is optionally substituted with one or more oxo or —OH. In some embodiments, R_{B4′} is C₁-C₆ haloalkyl, wherein the alkyl is substituted with one or more oxo or —OH.

In some embodiments, R_{B4′} is halomethyl. In some embodiments, R_{B4′} is haloethyl. In some embodiments, R_{B4′} is halopropyl. In some embodiments, R_{B4′} is halobutyl. In some embodiments, R_{B4′} is halopentyl. In some embodiments, R_{B4′} is halohexyl.

In some embodiments, R_{B4′} is halomethyl, wherein the alkyl is optionally substituted with one or more oxo or —OH. In some embodiments, R_{B4′} is haloethyl, wherein the alkyl is optionally substituted with one or more oxo or —OH. In some embodiments, R_{B4′} is halopropyl, wherein the alkyl is optionally substituted with one or more oxo or —OH. In some embodiments, R_{B4′} is halobutyl, wherein the alkyl is optionally substituted with one or more oxo or —OH. In some embodiments, R_{B4′} is halopentyl, wherein the alkyl is optionally substituted with one or more oxo or —OH. In some embodiments, R_{B4′} is halohexyl, wherein the alkyl is optionally substituted with one or more oxo or —OH.

In some embodiments, R_{B4′} is halomethyl, wherein the alkyl is substituted with one or more oxo or —OH. In some embodiments, R_{B4′} is haloethyl, wherein the alkyl is substituted with one or more oxo or —OH. In some embodiments, R_{B4′} is halopropyl, wherein the alkyl is substituted with one or more oxo or —OH. In some embodiments, R_{B4′} is halobutyl, wherein the alkyl is substituted with one or more oxo or —OH. In some embodiments, R_{B4′} is halopentyl, wherein the alkyl is substituted with one or more oxo or —OH. In some embodiments, R_{B4′} is halohexyl, wherein the alkyl is substituted with one or more oxo or —OH.

In some embodiments, R_{B4′} is C₁-C₆ alkoxy. In some embodiments, R_{B4′} is C₁-C₆ alkoxy, wherein the alkyl is optionally substituted with one or more oxo or —OH. In some embodiments, R_{B4′} is C₁-C₆ alkoxy, wherein the alkyl is substituted with one or more oxo or —OH.

In some embodiments, R_{B4′} is methoxy. In some embodiments, R_{B4′} is ethoxy. In some embodiments, R_{B4′} is propoxy. In some embodiments, R_{B4′} is butoxy. In some embodiments, R_{B4′} is pentoxy. In some embodiments, R_{B4′} is hexoxy.

In some embodiments, R_{B4′} is methoxy, wherein the alkyl is optionally substituted with one or more oxo or —OH. In some embodiments, R_{B4′} is ethoxy, wherein the alkyl is optionally substituted with one or more oxo or —OH. In some embodiments, R_{B4′} is propoxy, wherein the alkyl is optionally substituted with one or more oxo or —OH. In some embodiments, R_{B4′} is butoxy, wherein the alkyl is optionally substituted with one or more oxo or —OH. In some embodiments, R_{B4′} is pentoxy, wherein the alkyl is optionally substituted with one or more oxo or —OH. In some embodiments, R_{B4′} is hexoxy, wherein the alkyl is optionally substituted with one or more oxo or —OH.

In some embodiments, R_{B4′} is methoxy, wherein the alkyl is substituted with one or more oxo or —OH. In some embodiments, R_{B4′} is ethoxy, wherein the alkyl is substituted with one or more oxo or —OH. In some embodiments, R_{B4′} is propoxy, wherein the alkyl is substituted with one or more oxo or —OH. In some embodiments, R_{B4′} is butoxy, wherein the alkyl is substituted with one or more oxo or —OH. In some embodiments, R_{B4′} is pentoxy, wherein the alkyl is substituted with one or more oxo or —OH. In some embodiments, R_{B4′} is hexoxy, wherein the alkyl is substituted with one or more oxo or —OH.

In some embodiments, R_{B4′} is C₃-C₇ cycloalkyl or 3- to 7-membered heterocycloalkyl.

In some embodiments, R_{B4′} is C₃-C₇ cycloalkyl or 3- to 7-membered heterocycloalkyl, wherein the cycloalkyl and heterocycloalkyl are optionally substituted with one or more oxo or —OH.

In some embodiments, R_{B4′} is C₃-C₇ cycloalkyl or 3- to 7-membered heterocycloalkyl, wherein the cycloalkyl and heterocycloalkyl are substituted with one or more oxo or —OH.

In some embodiments, R_{B4′} is C₃-C₇ cycloalkyl. In some embodiments, R_{B4′} is C₃-C₇ cycloalkyl optionally substituted with one or more oxo or —OH. In some embodiments, R_{B} is C₁-C₇ cycloalkyl substituted with one or more oxo or —OH.

In some embodiments, R_{B4′} is C cycloalkyl. In some embodiments, R_{B4′} is C cycloalkyl optionally substituted with one or more oxo or —OH. In some embodiments, R_{B4′} is C₃ cycloalkyl substituted with one or more oxo or —OH.

In some embodiments, R_{B4′} is C₄ cycloalkyl. In some embodiments, R_{B4′} is C₄ cycloalkyl optionally substituted with one or more oxo or —OH. In some embodiments, R_{B4′} is C₄ cycloalkyl substituted with one or more oxo or —OH.

In some embodiments, R_{B4′} is C₅ cycloalkyl. In some embodiments, R_{B4′} is C₅ cycloalkyl optionally substituted with one or more oxo or —OH. In some embodiments, R_{B4′} is C₅ cycloalkyl substituted with one or more oxo or —OH.

In some embodiments, R_{B4′} is C₆ cycloalkyl. In some embodiments, R_{B4′} is C₆ cycloalkyl optionally substituted with one or more oxo or —OH. In some embodiments, R_{B4′} is C₆ cycloalkyl substituted with one or more oxo or —OH.

In some embodiments, R_{B4′} is C₇ cycloalkyl. In some embodiments, R_{B4′} is C₇ cycloalkyl optionally substituted with one or more oxo or —OH. In some embodiments, R_{B4′} is C₇ cycloalkyl substituted with one or more oxo or —OH.

In some embodiments, R_{B4′} is 3- to 7-membered heterocycloalkyl. In some embodiments, R_{B4′} is 3- to 7-membered heterocycloalkyl optionally substituted with one or more oxo or —OH. In some embodiments, R_{B4′} is 3- to 7-membered heterocycloalkyl substituted with one or more oxo or —OH.

In some embodiments, R_{B4′} is 3-membered heterocycloalkyl. In some embodiments, R_{B4′} is 3-membered heterocycloalkyl optionally substituted with one or more oxo or —OH. In some embodiments, R_{B4′} is 3-membered heterocycloalkyl substituted with one or more oxo or —OH.

In some embodiments, R_{B4′} is 4-membered heterocycloalkyl. In some embodiments, R_{B4′} is 4-membered heterocycloalkyl optionally substituted with one or more oxo or —OH. In some embodiments, R_{B4′} is 4-membered heterocycloalkyl substituted with one or more oxo or —OH.

In some embodiments, R_{B4′} is 5-membered heterocycloalkyl. In some embodiments, R_{B4′} is 5-membered heterocycloalkyl optionally substituted with one or more oxo or —OH. In some embodiments, R_{B4′} is 5-membered heterocycloalkyl substituted with one or more oxo or —OH.

In some embodiments, R_{B4′} is 6-membered heterocycloalkyl. In some embodiments, R_{B4′} is 6-membered heterocycloalkyl optionally substituted with one or more oxo or —OH. In some embodiments, R_{B4′} is 6-membered heterocycloalkyl substituted with one or more oxo or —OH.

In some embodiments, R_{B4′} is 7-membered heterocycloalkyl. In some embodiments, R_{B4′} is 7-membered heterocycloalkyl optionally substituted with one or more oxo or —OH. In some embodiments, R_{B4′} is 7-membered heterocycloalkyl substituted with one or more oxo or —OH.

In some embodiments, R_{B4″} is H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl.

In some embodiments, R_{B4″} is H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

In some embodiments, R_{B4″} is H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are substituted with one or more oxo or —OH.

In some embodiments, R_{B4″} is H.

In some embodiments, R_{B4″} is —OH.

In some embodiments, R_{B4″} is —NH₂.

In some embodiments, R_{B4″} is C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl.

In some embodiments, R_{B4″} is C₁-C₆ alkyl, C₂-C₆ alkenyl, C₂-C₆ alkynyl, C₁-C₆ haloalkyl, C₁-C₆ alkoxy, C₁-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

In some embodiments, R_{B4″} is C₁-C₆ alkyl, C₂-C₆ alkenyl, C₂-C₆ alkynyl, C₁-C₆ haloalkyl, C₁-C₆ alkoxy, C₃-C₂ cycloalkyl, or 3- to 7-membered heterocycloalkyl, wherein the alkyl, alkenyl, alkynyl, cycloalkyl, and heterocycloalkyl are substituted with one or more oxo or —OH.

In some embodiments, R_{B4″} is C₁-C₆ alkyl, C₂-C₆ alkenyl, C₂-C₆ alkynyl, C₁-C₆ haloalkyl, or C₁-C₆ alkoxy.

In some embodiments, R_{B4″} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more oxo or —OH.

In some embodiments, R_{B4″} is C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are substituted with one or more oxo or —OH.

In some embodiments, R_{B4″} is C₁-C₆ alkyl, C₂-C₆ alkenyl, or C₂-C₆ alkynyl.

In some embodiments, R_{B4″} is C₁-C₆ alkyl, C₂-C₆ alkenyl, or C₂-C₆ alkynyl, wherein the alkyl, alkenyl, or alkynyl are optionally substituted with one or more oxo or —OH.

In some embodiments, R_{B4″} is C₁-C₆ alkyl, C₂-C₆ alkenyl, or C₂-C₆ alkynyl, wherein the alkyl, alkenyl, or alkynyl are substituted with one or more oxo or —OH.

In some embodiments, R_{B4″} is C₁-C₆ alkyl. In some embodiments, R_{B4″} is C₁-C₆ alkyl optionally substituted with one or more oxo or —OH. In some embodiments, Rime is C₁-C₆ alkyl substituted with one or more oxo or —OH.

In some embodiments, R_{B4″} is methyl. In some embodiments, R_{B4″} is ethyl. In some embodiments, R_{B4″} is propyl. In some embodiments, R_{B4″} is butyl. In some embodiments, R_{B4″} is pentyl. In some embodiments, R_{B4″} is hexyl. In some embodiments, R_{B4″} is isopropyl. In some embodiments, R_{B4″} is isobutyl. In some embodiments, R_{B4″} is isopentyl. In some embodiments, R_{B4″} is isohexyl. In some embodiments, R_{B4″} is secbutyl. In some embodiments, R_{B4″} is secpentyl. In some embodiments, R_{B4″} is sechexyl. In some embodiments, R_{B4″} is tertbutyl.

In some embodiments, R_{B4″} is methyl optionally substituted with one or more oxo or —OH. In some embodiments, R_{B4″} is ethyl optionally substituted with one or more oxo or —OH. In some embodiments, R_{B4″} is propyl optionally substituted with one or more oxo or —OH. In some embodiments, R_{B4″} is butyl optionally substituted with one or more oxo or —OH. In some embodiments, R_{B4″} is pentyl optionally substituted with one or more oxo or —OH. In some embodiments, R_{B4″} is hexyl optionally substituted with one or more oxo or —OH. In some embodiments, R_{B4″} is isopropyl optionally substituted with one or more oxo or —OH. In some embodiments, R_{B4″} is isobutyl optionally substituted with one or more oxo or —OH. In some embodiments, R_{B4″} is isopentyl optionally substituted with one or more oxo or —OH. In some embodiments, R_{B4″} is isohexyl optionally substituted with one or more oxo or —OH. In some embodiments, R_{B4″} is secbutyl optionally substituted with one or more oxo or —OH. In some embodiments, R_{B4″} is secpentyl optionally substituted with one or more oxo or —OH. In some embodiments, R_{B4″} is sechexyl optionally substituted with one or more oxo or —OH. In some embodiments, R_{B4″} is tertbutyl optionally substituted with one or more oxo or —OH.

In some embodiments, R_{B4″} is methyl substituted with one or more oxo or —OH. In some embodiments, R_{B4″} is ethyl substituted with one or more oxo or —OH. In some embodiments, R_{B4″} is propyl substituted with one or more oxo or —OH. In some embodiments, R_{B4″} is butyl substituted with one or more oxo or —OH. In some embodiments, R_{B4″} is pentyl substituted with one or more oxo or —OH. In some embodiments, R_{B4″} is hexyl substituted with one or more oxo or —OH. In some embodiments, R_{B4″} is isopropyl substituted with one or more oxo or —OH. In some embodiments, R_{B4″} is isobutyl substituted with one or more oxo or —OH. In some embodiments, R_{B4″} is isopentyl substituted with one or more oxo or —OH. In some embodiments, R_{B4″} is isohexyl substituted with one or more oxo or —OH. In some embodiments, R_{B4″} is secbutyl substituted with one or more oxo or —OH. In some embodiments, R_{B4″} is secpentyl substituted with one or more oxo or —OH. In some embodiments, R_{B4″} is sechexyl substituted with one or more oxo or —OH. In some embodiments, R_{B1″} is tertbutyl substituted with one or more oxo or —OH.

In some embodiments, R_{B4″} is C₂-C₆ alkenyl. In some embodiments, R_{B4″} is C₂-C₆ alkenyl optionally substituted with one or more oxo or —OH. In some embodiments, R_{B4″} is C₂-C₆ alkenyl substituted with one or more oxo or —OH.

In some embodiments, R_{B4″} is C₂ alkenyl. In some embodiments, R_{B4″} is C₃ alkenyl. In some embodiments, R_{B4″} is C₄ alkenyl. In some embodiments, R_{B4″} is C₅ alkenyl. In some embodiments, R_{B4″} is C₆ alkenyl.

In some embodiments, R_{B4″} is C₂ alkenyl optionally substituted with one or more oxo or —OH. In some embodiments, R_{B4″} is C₃ alkenyl optionally substituted with one or more oxo or —OH. In some embodiments, R_{B4″} is C₄ alkenyl optionally substituted with one or more oxo or —OH. In some embodiments, R_{B4″} is C₅ alkenyl optionally substituted with one or more oxo or —OH. In some embodiments, R_{B4″} is C₆ alkenyl optionally substituted with one or more oxo or —OH.

In some embodiments, R_{B4″} is C₂ alkenyl substituted with one or more oxo or —OH. In some embodiments, R_{B4″} is C₃ alkenyl substituted with one or more oxo or —OH. In some embodiments, R_{B4″} is C₄ alkenyl substituted with one or more oxo or —OH. In some embodiments, R_{B4″} is C₅ alkenyl substituted with one or more oxo or —OH. In some embodiments, R_{B4″} is C₆ alkenyl substituted with one or more oxo or —OH.

In some embodiments, R_{B4″} is C₂-C₆ alkynyl. In some embodiments, R_{B4″} is C₂-C₆ alkynyl optionally substituted with one or more oxo or —OH. In some embodiments, R_{B4″}, is C₂-C₆ alkynyl substituted with one or more oxo or —OH.

In some embodiments, R_{B4″} is C₂ alkynyl. In some embodiments, R_{B4″} is C₃ alkynyl. In some embodiments, R_{B4″} is C₄ alkynyl. In some embodiments, R_{B4″} is C₅ alkynyl. In some embodiments, R_{B4″} is C₆ alkynyl.

In some embodiments, R_{B4″} is C₂ alkynyl optionally substituted with one or more oxo or —OH. In some embodiments, R_{B4″} is C₃ alkynyl optionally substituted with one or more oxo or —OH. In some embodiments, R_{B4″} is C₄ alkynyl optionally substituted with one or more oxo or —OH. In some embodiments, R_{B4″} is C₅ alkynyl optionally substituted with one or more oxo or —OH. In some embodiments, R_{B4″} is C₆ alkynyl optionally substituted with one or more oxo or —OH.

In some embodiments, R_{B4″} is C₂ alkynyl substituted with one or more oxo or —OH. In some embodiments, R_{B4″} is C₃ alkynyl substituted with one or more oxo or —OH. In some embodiments, R_{B4″} is C₄ alkynyl substituted with one or more oxo or —OH. In some embodiments, R_{B4″} is C₅ alkynyl substituted with one or more oxo or —OH. In some embodiments, R_{B4″} is C₆ alkynyl substituted with one or more oxo or —OH.

In some embodiments, R_{B4″} is C₁-C₆ haloalkyl or C₁-C₆ alkoxy. In some embodiments, R_{B4″} is C₁-C₆ haloalkyl or C₁-C₆ alkoxy, wherein the alkyl is optionally substituted with one or more oxo or —OH. In some embodiments, R_{B4″} is C₁-C₆ haloalkyl or C₁-C₆ alkoxy, wherein the alkyl is substituted with one or more oxo or —OH.

In some embodiments, R_{B4″} is C₁-C₆ haloalkyl. In some embodiments, R_{B4″} is C₁-C₆ haloalkyl, wherein the alkyl is optionally substituted with one or more oxo or —OH. In some embodiments, R_{B4″} is C₁-C₆ haloalkyl, wherein the alkyl is substituted with one or more oxo or —OH.

In some embodiments, R_{B4″} is halomethyl. In some embodiments, R_{B4″} is haloethyl. In some embodiments, R_{B4″} is halopropyl. In some embodiments, R_{B4″} is halobutyl. In some embodiments, R_{B4″} is halopentyl. In some embodiments, R_{B4″} is halohexyl.

In some embodiments, R_{B4″} is halomethyl, wherein the alkyl is optionally substituted with one or more oxo or —OH. In some embodiments, R_{B4″} is haloethyl, wherein the alkyl is optionally substituted with one or more oxo or —OH. In some embodiments, R_{B4″} is halopropyl, wherein the alkyl is optionally substituted with one or more oxo or —OH. In some embodiments, R_{B4″} is halobutyl, wherein the alkyl is optionally substituted with one or more oxo or —OH. In some embodiments, R_{B4″} is halopentyl, wherein the alkyl is optionally substituted with one or more oxo or —OH. In some embodiments, R_{B4″} is halohexyl, wherein the alkyl is optionally substituted with one or more oxo or —OH.

In some embodiments, R_{B4″} is halomethyl, wherein the alkyl is substituted with one or more oxo or —OH. In some embodiments, R_{B4″} is haloethyl, wherein the alkyl is substituted with one or more oxo or —OH. In some embodiments, R_{B4″} is halopropyl, wherein the alkyl is substituted with one or more oxo or —OH. In some embodiments, R_{B4″} is halobutyl, wherein the alkyl is substituted with one or more oxo or —OH. In some embodiments, R_{B4″} is halopentyl, wherein the alkyl is substituted with one or more oxo or —OH. In some embodiments, R_{B4″} is halohexyl, wherein the alkyl is substituted with one or more oxo or —OH.

In some embodiments, R_{B4″} is C₁-C₆ alkoxy. In some embodiments, R_{B4″} is C₁-C₆ alkoxy, wherein the alkyl is optionally substituted with one or more oxo or —OH. In some embodiments, R_{B4″} is C₁-C₆ alkoxy, wherein the alkyl is substituted with one or more oxo or —OH.

In some embodiments, R_{B4″} is methoxy. In some embodiments, R_{B4″} is ethoxy. In some embodiments, R_{B4″} is propoxy. In some embodiments, R_{B4″} is butoxy. In some embodiments, R_{B4″} is pentoxy. In some embodiments, R_{B4″} is hexoxy.

In some embodiments, R_{B4″} is methoxy, wherein the alkyl is optionally substituted with one or more oxo or —OH. In some embodiments, R_{B4″} is ethoxy, wherein the alkyl is optionally substituted with one or more oxo or —OH. In some embodiments, R_{B4″} is propoxy, wherein the alkyl is optionally substituted with one or more oxo or —OH. In some embodiments, R_{B4″} is butoxy, wherein the alkyl is optionally substituted with one or more oxo or —OH. In some embodiments, R_{B4″} is pentoxy, wherein the alkyl is optionally substituted with one or more oxo or —OH. In some embodiments, R_{B4″} is hexoxy, wherein the alkyl is optionally substituted with one or more oxo or —OH.

In some embodiments, R_{B4″} is methoxy, wherein the alkyl is substituted with one or more oxo or —OH. In some embodiments, R_{B4″} is ethoxy, wherein the alkyl is substituted with one or more oxo or —OH. In some embodiments, R_{B4″} is propoxy, wherein the alkyl is substituted with one or more oxo or —OH. In some embodiments, R_{B4″} is butoxy, wherein the alkyl is substituted with one or more oxo or —OH. In some embodiments, R_{B4″} is pentoxy, wherein the alkyl is substituted with one or more oxo or —OH. In some embodiments, R_{B4″} is hexoxy, wherein the alkyl is substituted with one or more oxo or —OH.

In some embodiments, R_{B4″} is C₃-C₇ cycloalkyl or 3- to 7-membered heterocycloalkyl. In some embodiments, R_{B4″} is C₃-C₇ cycloalkyl or 3- to 7-membered heterocycloalkyl, wherein the cycloalkyl and heterocycloalkyl are optionally substituted with one or more oxo or —OH. In some embodiments, R_{B4″} is C₃-C₇ cycloalkyl or 3- to 7-membered heterocycloalkyl, wherein the cycloalkyl and heterocycloalkyl are substituted with one or more oxo or —OH.

In some embodiments, R_{B4″} is C₃-C₇ cycloalkyl. In some embodiments, R_{B4″} is C₃-C₇ cycloalkyl optionally substituted with one or more oxo or —OH. In some embodiments, R_{B4″} is C₃-C₇ cycloalkyl substituted with one or more oxo or —OH.

In some embodiments, R_{B4″} is C₃ cycloalkyl. In some embodiments, R_{B4″} is C₃ cycloalkyl optionally substituted with one or more oxo or —OH. In some embodiments, R_{B4″} is C₃ cycloalkyl substituted with one or more oxo or —OH.

In some embodiments, R_{B4″} is C₄ cycloalkyl. In some embodiments, R_{B4″} is C₄ cycloalkyl optionally substituted with one or more oxo or —OH. In some embodiments, R_{B4″} is C₄ cycloalkyl substituted with one or more oxo or —OH.

In some embodiments, R_{B4″} is C₅ cycloalkyl. In some embodiments, R_{B4″} is C₅ cycloalkyl optionally substituted with one or more oxo or —OH. In some embodiments, R_{B4″} is C₅ cycloalkyl substituted with one or more oxo or —OH.

In some embodiments, R_{B4″} is C₆ cycloalkyl. In some embodiments, R_{B4″} is C₆ cycloalkyl optionally substituted with one or more oxo or —OH. In some embodiments, R_{B4″} is C₆ cycloalkyl substituted with one or more oxo or —OH.

In some embodiments, R_{B4″} is C₇ cycloalkyl. In some embodiments, R_{B4″} is C₇ cycloalkyl optionally substituted with one or more oxo or —OH. In some embodiments, R_{B4″} is C₇ cycloalkyl substituted with one or more oxo or —OH.

In some embodiments, R_{B4″} is 3- to 7-membered heterocycloalkyl. In some embodiments, R_{B4″} is 3- to 7-membered heterocycloalkyl optionally substituted with one or more oxo or —OH. In some embodiments, R_{B4″} is 3- to 7-membered heterocycloalkyl substituted with one or more oxo or —OH.

In some embodiments, R_{B4″} is 3-membered heterocycloalkyl. In some embodiments, R_{B4″} is 3-membered heterocycloalkyl optionally substituted with one or more oxo or —OH. In some embodiments, R_{B4″} is 3-membered heterocycloalkyl substituted with one or more oxo or —OH.

In some embodiments, R_{B4″} is 4-membered heterocycloalkyl. In some embodiments, R_{B4″} is 4-membered heterocycloalkyl optionally substituted with one or more oxo or —OH. In some embodiments, R_{B4″} is 4-membered heterocycloalkyl substituted with one or more oxo or —OH.

In some embodiments, R_{B4″} is 5-membered heterocycloalkyl. In some embodiments, R_{B4″} is 5-membered heterocycloalkyl optionally substituted with one or more oxo or —OH. In some embodiments, R_{B4″} is 5-membered heterocycloalkyl substituted with one or more oxo or —OH.

In some embodiments, R_{B4″} is 6-membered heterocycloalkyl. In some embodiments, R_{B4″} is 6-membered heterocycloalkyl optionally substituted with one or more oxo or —OH. In some embodiments, R_{B4″} is 6-membered heterocycloalkyl substituted with one or more oxo or —OH.

In some embodiments, R_{B4″} is 7-membered heterocycloalkyl. In some embodiments, R_{B4″} is 7-membered heterocycloalkyl optionally substituted with one or more oxo or —OH. In some embodiments, Rim is 7-membered heterocycloalkyl substituted with one or more oxo or —OH.

In some embodiments, n is 0, 1, 2, 3, 4, or 5.

In some embodiments, n is 0. In some embodiments, n is 1. In some embodiments, n is 2. In some embodiments, n is 3. In some embodiments, n is 4. In some embodiments, n is 5.

In some embodiments, m is 0, 1, 2, 3, 4, or 5.

In some embodiments, m is 0. In some embodiments, m is 1. In some embodiments, m is 2. In some embodiments, m is 3. In some embodiments, m is 4. In some embodiments, m is 5.

In some embodiments, when R_{B} is a substituted or unsubstituted alkyl, Ring A is substituted by at least one R_{A}.

In some embodiments, when R_{B} is a substituted or unsubstituted alkyl, Ring A is substituted by one R_{A}. In some embodiments, when R_{B} is a substituted or unsubstituted alkyl, Ring A is substituted by two R_{A}. In some embodiments, when R_{B} is a substituted or unsubstituted alkyl, Ring A is substituted by three R_{A}. In some embodiments, when R_{B} is a substituted or unsubstituted alkyl, Ring A is substituted by four R_{A}.

In some embodiments, Ring A is substituted by at least one R_{A}.

In some embodiments, the compound is of Formula (I′-a) or (I′-b):

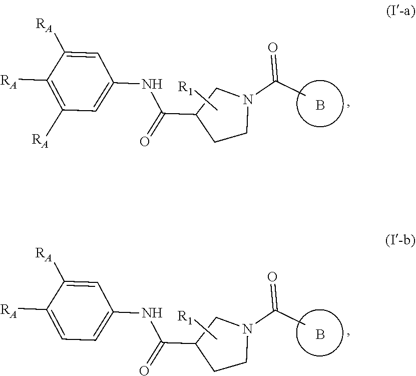

or a prodrug, solvate, or pharmaceutically acceptable salt thereof.

In some embodiments, the compound is of Formula (I′-a) or a prodrug, solvate, or pharmaceutically acceptable salt thereof.

In some embodiments, the compound is of Formula (I′-b) or a prodrug, solvate, or pharmaceutically acceptable salt thereof.

In some embodiments, the compound is of Formula (I-a) or (I-b):

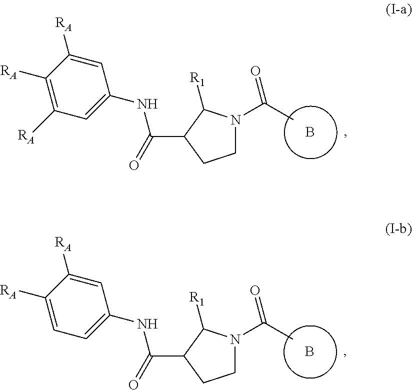

or a prodrug, solvate, or pharmaceutically acceptable salt thereof.

In some embodiments, the compound is of Formula (I-a) or a prodrug, solvate, or pharmaceutically acceptable salt thereof.

In some embodiments, the compound is of Formula (I-b) or a prodrug, solvate, or pharmaceutically acceptable salt thereof.

In some embodiments, the compound is of Formula (I′-c) or (I′-d):

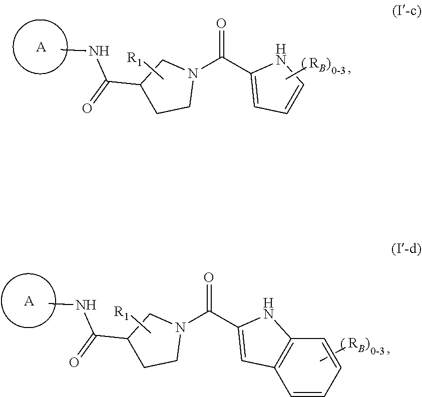

or a prodrug, solvate, or pharmaceutically acceptable salt thereof.

In some embodiments, the compound is of Formula (I′-c) or a prodrug, solvate, or pharmaceutically acceptable salt thereof.

In some embodiments, the compound is of Formula (I′-d) or a prodrug, solvate, or pharmaceutically acceptable salt thereof.

In some embodiments, the compound is of Formula (I-c) or (I-d):

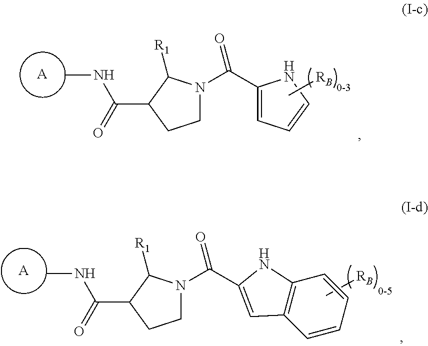

or a prodrug, solvate, or pharmaceutically acceptable salt thereof.

In some embodiments, the compound is of Formula (I-c) or a prodrug, solvate, or pharmaceutically acceptable salt thereof.

In some embodiments, the compound is of Formula (I-d) or a prodrug, solvate, or pharmaceutically acceptable salt thereof.

In some embodiments, the compound is of Formula (I′-c1):

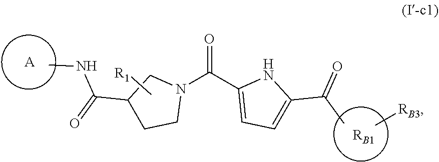

or a prodrug, solvate, or pharmaceutically acceptable salt thereof.

In some embodiments, the compound is of Formula (I′-c1) or a prodrug, solvate, or pharmaceutically acceptable salt thereof.

In some embodiments, the compound is of Formula (I-c1):

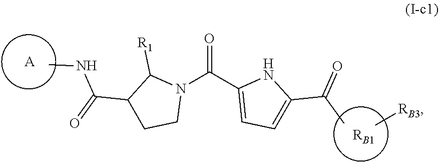

or a prodrug, solvate, or pharmaceutically acceptable salt thereof.

In some embodiments, the compound is of Formula (I-c1) or a prodrug, solvate, or pharmaceutically acceptable salt thereof.

In some embodiments, the compound is of Formula (I-a′) or (I-b′):

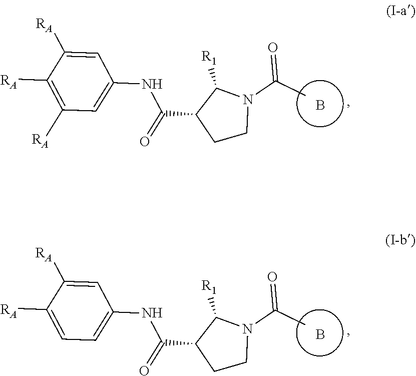

or a prodrug, solvate, or pharmaceutically acceptable salt thereof.

In some embodiments, the compound is of Formula (I-a′) or a prodrug, solvate, or pharmaceutically acceptable salt thereof.

In some embodiments, the compound is of Formula (I-b′) or a prodrug, solvate, or pharmaceutically acceptable salt thereof.

In some embodiments the compound is of Formula I-c′ or (I-d′):

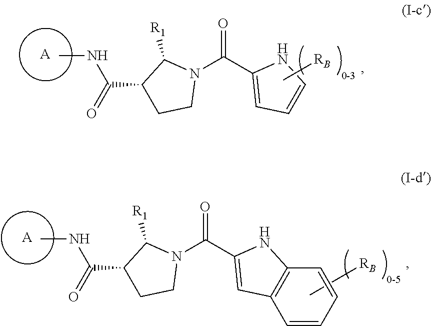

or a prodrug, solvate, or pharmaceutically acceptable salt thereof.

In some embodiments, the compound is of Formula (I-c′) or a prodrug, solvate, or pharmaceutically acceptable salt thereof.

In some embodiments, the compound is of Formula (I-d′) or a prodrug, solvate, or pharmaceutically acceptable salt thereof.

In some embodiments, the compound is of Formula (I-c1′):

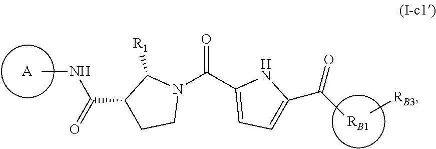

or a prodrug, solvate, or pharmaceutically acceptable salt thereof.

In some embodiments, the compound is of Formula (I-c1′) or a prodrug, solvate, or pharmaceutically acceptable salt thereof.

It is understood that, for a compound of Formula (I) or (I′), X, Y, R_{X}, R_{Y}, Ring A, Ring B, R₁, R_{A}, R_{B}, R_{B1}, R_{B2}, R_{B3}, R_{B3′}, R_{B4}, R_{B4′}, R_{B4″}, n, and m can each be, where applicable, selected from the groups described herein, and any group described herein for any of X, Y, R_{X}, R_{Y}, Ring A, Ring B, R₁, R_{A}, R_{B}, R_{B1}, R_{B2}, R_{B3}, R_{B3′}, R_{B4}, R_{4′}, R_{B4″}, n, and m can be combined, where applicable, with any group described herein for one or more of the remainder of X, Y, R_{X}, R_{Y}, Ring A, Ring B, R₁, R_{A}, R_{B}, R_{B1}, R_{B2}, R_{B2′}, R_{B3′}, R_{B4}, R_{B4′}, R_{B4″}, n, and m.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of compounds described in Table 1 and pharmaceutically acceptable salts thereof.

In some embodiments, the compound is selected from the compounds described in Table 1.

TABLE 1

| Compound | |
|---|---|
| No | Structure |
| 1 | 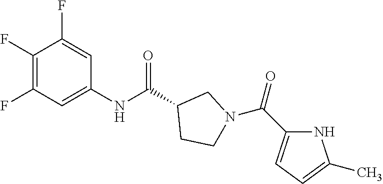 [SMILES: Cc1ccc(C(=O)N2CC[C@H](C(=O)Nc3cc(F)c(F)c(F)c3)C2)[nH]1] |
| | (S)-1-(5-methyl-1H-pyrrole-2-carbonyl)-N-(3,4,5- |
| | trifluorophenyl)pyrrolidine-3-carboxamide |
| 2 | 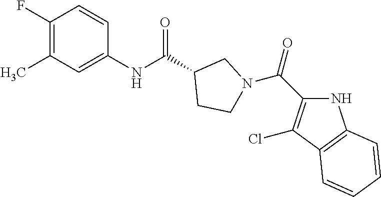 [SMILES: Cc1cc(NC(=O)[C@H]2CCN(C(=O)c3[nH]c4ccccc4c3Cl)C2)ccc1F] |
| | (S)-1-(3-chloro-1H-indole-2-carbonyl)-N-(4-fluoro-3- |
| | methylphenyl)pyrrolidine-3-carboxamide |
| 3 | 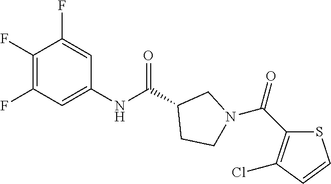 [SMILES: O=C(Nc1cc(F)c(F)c(F)c1)[C@H]1CCN(C(=O)c2sccc2Cl)C1] |
| | (S)-1-(3-chlorothiophene-2-carbonyl)-N-(3,4,5- |
| | trifluorophenyl)pyrrolidine-3-carboxamide |
| 4 | 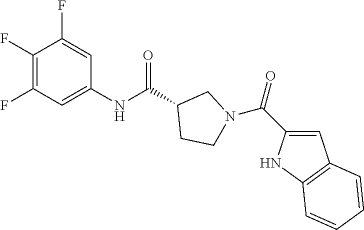 [SMILES: O=C(Nc1cc(F)c(F)c(F)c1)[C@H]1CCN(C(=O)c2cc3ccccc3[nH]2)C1] |
| | (S)-1-(1H-indole-2-carbonyl)-N-(3,4,5-trifluorophenyl)pyrrolidine-3- |
| | carboxamide |
| 5 | 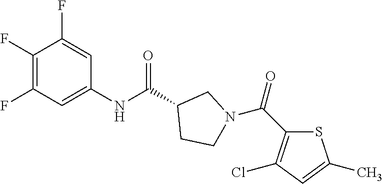 [SMILES: Cc1cc(Cl)c(C(=O)N2CC[C@H](C(=O)Nc3cc(F)c(F)c(F)c3)C2)s1] |
| | (S)-1-(3-chloro-5-methylthiophene-2-carbonyl)-N-(3,4,5- |
| | trifluorophenyl)pyrrolidine-3-carboxamide |
| 6 | 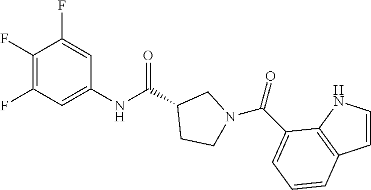 [SMILES: O=C(Nc1cc(F)c(F)c(F)c1)[C@H]1CCN(C(=O)c2cccc3cc[nH]c23)C1] |
| | (S)-1-(1H-indole-7-carbonyl)-N-(3,4,5-trifluorophenyl)pyrrolidine-3- |
| | carboxamide |
| 7 | 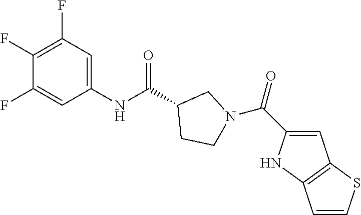 [SMILES: O=C(Nc1cc(F)c(F)c(F)c1)[C@H]1CCN(C(=O)c2cc3sccc3[nH]2)C1] |
| | (S)-1-(4H-thieno[3,2-b]pyrrole-5-carbonyl)-N-(3,4,5-trifluoro- |
| | phenyl)pyrrolidine-3-carboxamide |
| 8 | 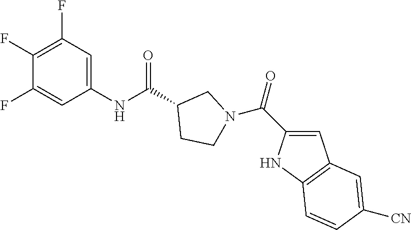 [SMILES: N#Cc1ccc2[nH]c(C(=O)N3CC[C@H](C(=O)Nc4cc(F)c(F)c(F)c4)C3)cc2c1] |
| | (S)-1-(5-cyano-1H-indole-2-carbonyl)-N-(3,4,5- |
| | trifluorophenyl)pyrrolidine-3-carboxamide |
| 9 | 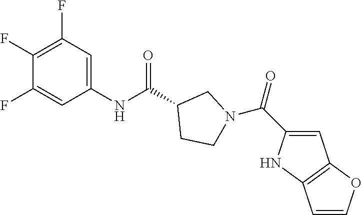 [SMILES: O=C(Nc1cc(F)c(F)c(F)c1)[C@H]1CCN(C(=O)c2cc3occc3[nH]2)C1] |
| | (S)-1-(4H-furo[3,2-b]pyrrole-5-carbonyl)-N-(3,4,5- |
| | trifluorophenyl)pyrrolidine-3-carboxamide |
| 10 | 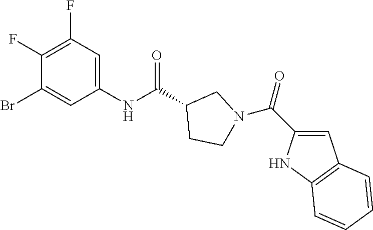 [SMILES: O=C(Nc1cc(F)c(F)c(Br)c1)[C@H]1CCN(C(=O)c2cc3ccccc3[nH]2)C1] |
| | (S)-N-(3-bromo-4,5-difluorophenyl)-1-(1H-indole-2- |
| | carbonyl)pyrrolidine-3-carboxamide |
| 11 | 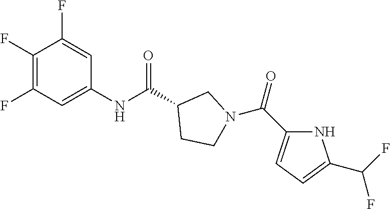 [SMILES: O=C(Nc1cc(F)c(F)c(F)c1)[C@H]1CCN(C(=O)c2ccc(C(F)F)[nH]2)C1] |
| | (S)-1-(5-(difluoromethyl)-1H-pyrrole-2-carbonyl)-N-(3,4,5- |
| | trifluorophenyl)pyrrolidine-3-carboxamide |
| 12 | 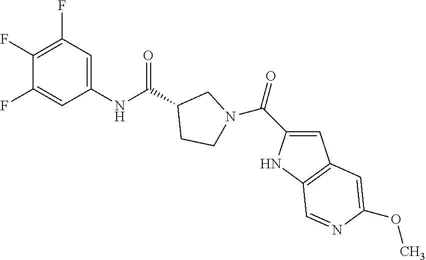 [SMILES: COc1cc2cc(C(=O)N3CC[C@H](C(=O)Nc4cc(F)c(F)c(F)c4)C3)[nH]c2cn1] |
| | (S)-1-(5-methoxy-1H-pyrrolo[2,3-c]pyridine-2-carbonyl)-N-(3,4,5- |
| | trifluorophenyl)pyrrolidine-3-carboxamide |
| 13 | 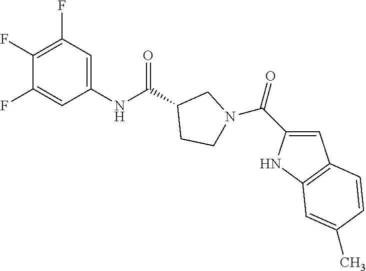 [SMILES: Cc1ccc2cc(C(=O)N3CC[C@H](C(=O)Nc4cc(F)c(F)c(F)c4)C3)[nH]c2c1] |
| | (S)-1-(6-methyl-1H-indole-2-carbonyl)-N-(3,4,5- |
| | trifluorophenyl)pyrrolidine-3-carboxamide |
| 14 | 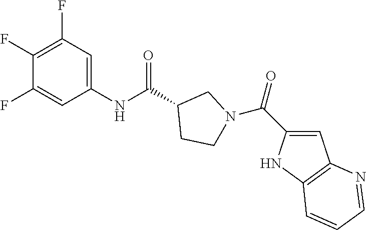 [SMILES: O=C(Nc1cc(F)c(F)c(F)c1)[C@H]1CCN(C(=O)c2cc3ncccc3[nH]2)C1] |
| | (S)-1-(1H-pyrrolo[3,2-b]pyridine-2-carbonyl)-N-(3,4,5- |
| | trifluorophenyl)pyrrolidine-3-carboxamide |
| 15 | 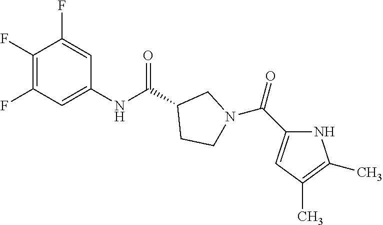 [SMILES: Cc1cc(C(=O)N2CC[C@H](C(=O)Nc3cc(F)c(F)c(F)c3)C2)[nH]c1C] |
| | (S)-1-(4,5-dimethyl-1H-pyrrole-2-carbonyl)-N-(3,4,5- |
| | trifluorophenyl)pyrrolidine-3-carboxamide |
| 16 | 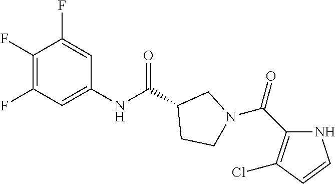 [SMILES: O=C(Nc1cc(F)c(F)c(F)c1)[C@H]1CCN(C(=O)c2[nH]ccc2Cl)C1] |
| | (S)-1-(3-chloro-1H-pyrrole-2-carbonyl)-N-(3,4,5- |
| | trifluorophenyl)pyrrolidine-3-carboxamide |
| 17 | 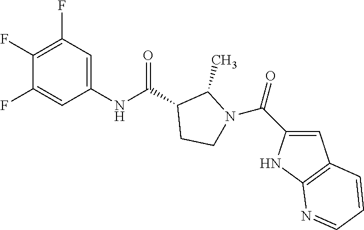 [SMILES: C[C@H]1[C@@H](C(=O)Nc2cc(F)c(F)c(F)c2)CCN1C(=O)c1cc2cccnc2[nH]1] |
| | (2S,3S)-2-methyl-1-(1H-pyrrolo[2,3-b]pyridine-2-carbonyl)-N-(3,4,5- |
| | trifluorophenyl)pyrrolidine-3-carboxamide |
| 18 | 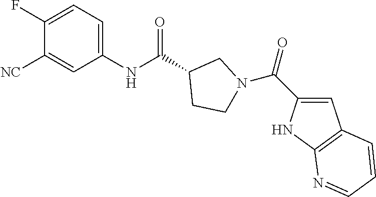 [SMILES: N#Cc1cc(NC(=O)[C@H]2CCN(C(=O)c3cc4cccnc4[nH]3)C2)ccc1F] |
| | (S)-N-(3-cyano-4-fluorophenyl)-1-(1H-pyrrolo[2,3-b]pyridine-2- |
| | carbonyl)pyrrolidine-3-carboxamide |
| 19 | 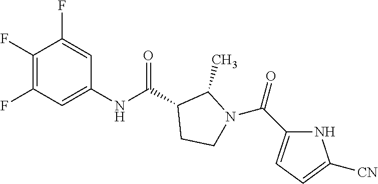 [SMILES: C[C@H]1[C@@H](C(=O)Nc2cc(F)c(F)c(F)c2)CCN1C(=O)c1ccc(C#N)[nH]1] |
| | (2S,3S)-1-(5-cyano-1H-pyrrole-2-carbonyl)-2-methyl-N-(3,4,5- |
| | trifluorophenyl)pyrrolidine-3-carboxamide |
| 20 | 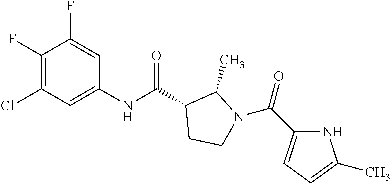 [SMILES: Cc1ccc(C(=O)N2CC[C@H](C(=O)Nc3cc(F)c(F)c(Cl)c3)[C@@H]2C)[nH]1] |
| | (2S,3S)-N-(3-chloro-4,5-difluorophenyl)-2-methyl-1-(5-methyl-1H- |
| | pyrrole-2-carbonyl)pyrrolidine-3-carboxamide |
| 21 | 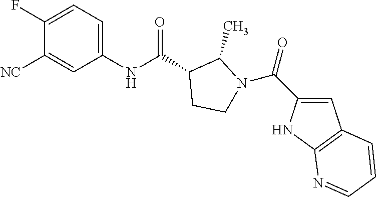 [SMILES: C[C@H]1[C@@H](C(=O)Nc2ccc(F)c(C#N)c2)CCN1C(=O)c1cc2cccnc2[nH]1] |
| | (2S,3S)-N-(3-cyano-4-fluorophenyl)-2-methyl-1-(1H-pyrrolo[2,3- |
| | b]pyridine-2-carbonyl)pyrrolidine-3-carboxamide |
| 22 | 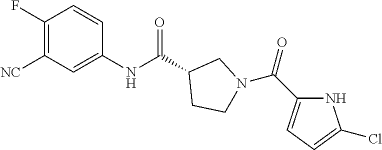 [SMILES: N#Cc1cc(NC(=O)[C@H]2CCN(C(=O)c3ccc(Cl)[nH]3)C2)ccc1F] |
| | (S)-1-(5-chloro-1H-pyrrole-2-carbonyl)-N-(3-cyano-4- |
| | fluorophenyl)pyrrolidine-3-carboxamide |
| 23 | 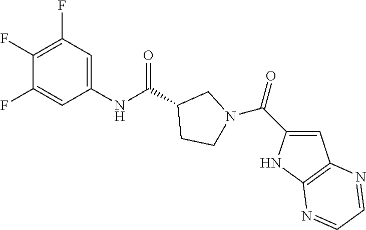 [SMILES: O=C(Nc1cc(F)c(F)c(F)c1)[C@H]1CCN(C(=O)c2cc3nccnc3[nH]2)C1] |
| | (S)-1-(5H-pyrrolo[2,3-b]pyrazine-6-carbonyl)-N-(3,4,5- |
| | trifluorophenyl)pyrrolidine-3-carboxamide |
| 24 | 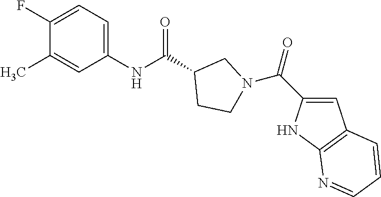 [SMILES: Cc1cc(NC(=O)[C@H]2CCN(C(=O)c3cc4cccnc4[nH]3)C2)ccc1F] |
| | (S)-N-(4-fluoro-3-methylphenyl)-1-(1H-pyrrolo[2,3-b]pyridine-2- |
| | carbonyl)pyrrolidine-3-carboxamide |
| 25 | 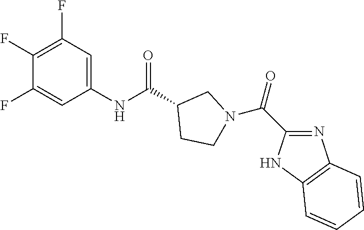 [SMILES: O=C(Nc1cc(F)c(F)c(F)c1)[C@H]1CCN(C(=O)c2nc3ccccc3[nH]2)C1] |
| | (S)-1-(1H-benzo[d]imidazole-2-carbonyl)-N-(3,4,5- |
| | trifluorophenyl)pyrrolidine-3-carboxamide |
| 26 | 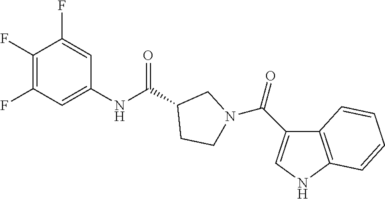 [SMILES: O=C(Nc1cc(F)c(F)c(F)c1)[C@H]1CCN(C(=O)c2c[nH]c3ccccc23)C1] |
| | (S)-1-(1H-indole-3-carbonyl)-N-(3,4,5-trifluorophenyl)pyrrolidine-3- |
| | carboxamide |
| 27 | 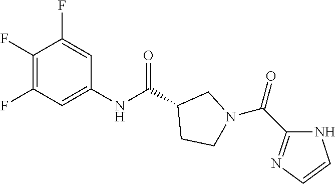 [SMILES: O=C(Nc1cc(F)c(F)c(F)c1)[C@H]1CCN(C(=O)c2ncc[nH]2)C1] |
| | (S)-1-(1H-imidazole-2-carbonyl)-N-(3,4,5-trifluorophenyl)pyrrolidine-3- |
| | carboxamide |
| 28 | 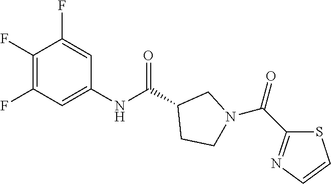 [SMILES: O=C(Nc1cc(F)c(F)c(F)c1)[C@H]1CCN(C(=O)c2nccs2)C1] |
| | (S)-1-(thiazole-2-carbonyl)-N-(3,4,5-trifluorophenyl)pyrrolidine-3- |
| | carboxamide |
| 29 | 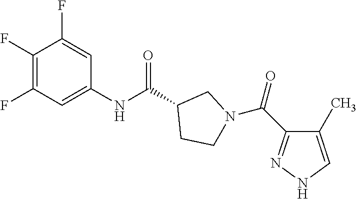 [SMILES: Cc1c[nH]nc1C(=O)N1CC[C@H](C(=O)Nc2cc(F)c(F)c(F)c2)C1] |
| | (S)-1-(4-methyl-1H-pyrazole-3-carbonyl)-N-(3,4,5- |
| | trifluorophenyl)pyrrolidine-3-carboxamide |
| 30 | 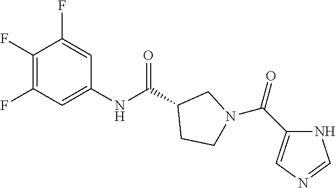 [SMILES: O=C(Nc1cc(F)c(F)c(F)c1)[C@H]1CCN(C(=O)c2cnc[nH]2)C1] |
| | (S)-1-(1H-imidazole-5-carbonyl)-N-(3,4,5-trifluorophenyl)pyrrolidine-3- |
| | carboxamide |
| 31 | 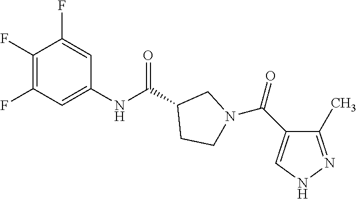 [SMILES: Cc1n[nH]cc1C(=O)N1CC[C@H](C(=O)Nc2cc(F)c(F)c(F)c2)C1] |
| | (S)-1-(3-methyl-1H-pyrazole-4-carbonyl)-N-(3,4,5- |
| | trifluorophenyl)pyrrolidine-3-carboxamide |
| 32 | 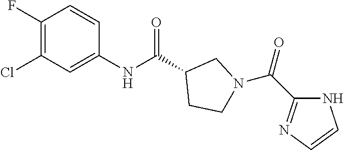 [SMILES: O=C(Nc1ccc(F)c(Cl)c1)[C@H]1CCN(C(=O)c2ncc[nH]2)C1] |
| | (S)-N-(3-chloro-4-fluorophenyl)-1-(1H-imidazole-2-carbonyl)pyrrolidine- |
| | 3-carboxamide |
| 33 | 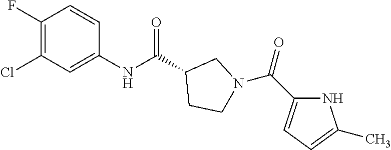 [SMILES: Cc1ccc(C(=O)N2CC[C@H](C(=O)Nc3ccc(F)c(Cl)c3)C2)[nH]1] |
| | (S)-N-(3-chloro-4-fluorophenyl)-1-(5-methyl-1H-pyrrole-2- |
| | carbonyl)pyrrolidine-3-carboxamide |
| 34 | 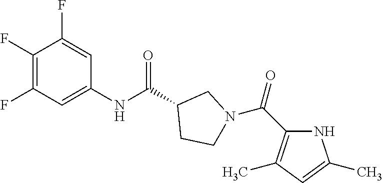 [SMILES: Cc1cc(C)c(C(=O)N2CC[C@H](C(=O)Nc3cc(F)c(F)c(F)c3)C2)[nH]1] |
| | (S)-1-(3,5-dimethyl-1H-pyrrole-2-carbonyl)-N-(3,4,5- |
| | trifluorophenyl)pyrrolidine-3-carboxamide |
| 35 | 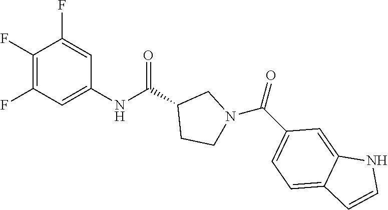 [SMILES: O=C(Nc1cc(F)c(F)c(F)c1)[C@H]1CCN(C(=O)c2ccc3cc[nH]c3c2)C1] |
| | (S)-1-(1H-indole-6-carbonyl)-N-(3,4,5-trifluorophenyl)pyrrolidine-3- |
| | carboxamide |
| 36 | 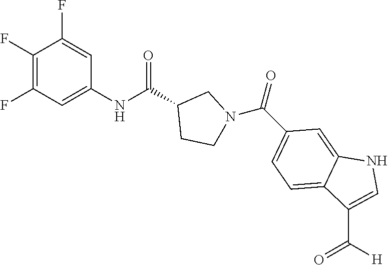 [SMILES: O=Cc1c[nH]c2cc(C(=O)N3CC[C@H](C(=O)Nc4cc(F)c(F)c(F)c4)C3)ccc12] |
| | (S)-1-(3-formyl-1H-indole-6-carbonyl)-N-(3,4,5- |
| | trifluorophenyl)pyrrolidine-3-carboxamide |
| 37 | 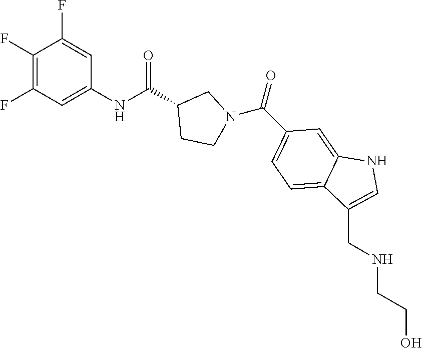 [SMILES: O=C(Nc1cc(F)c(F)c(F)c1)[C@H]1CCN(C(=O)c2ccc3c(CNCCO)c[nH]c3c2)C1] |
| | (S)-1-(3-(((2-hydroxyethyl)amino)methyl)-1H-indole-6-carbonyl)-N- |
| | (3,4,5-trifluorophenyl)pyrrolidine-3-carboxamide |
| 38 | 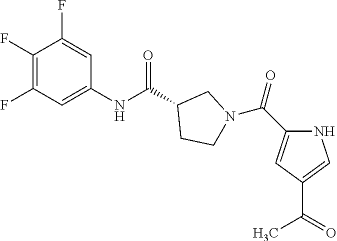 [SMILES: CC(=O)c1c[nH]c(C(=O)N2CC[C@H](C(=O)Nc3cc(F)c(F)c(F)c3)C2)c1] |
| | (S)-1-(4-acetyl-1H-pyrrole-2-carbonyl)-N-(3,4,5- |
| | trifluorophenyl)pyrrolidine-3-carboxamide |
| 39 | 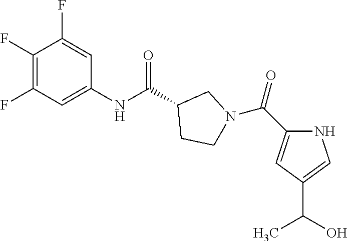 [SMILES: CC(O)c1c[nH]c(C(=O)N2CC[C@H](C(=O)Nc3cc(F)c(F)c(F)c3)C2)c1] |
| | (3S)-1-(4-(1-hydroxyethyl)-1H-pyrrole-2-carbo-nyl)-N-(3,4,5- |
| | trifluorophenyl)pyrrolidine-3-carboxamide |
| 40 | 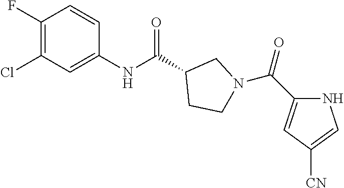 [SMILES: N#Cc1c[nH]c(C(=O)N2CC[C@H](C(=O)Nc3ccc(F)c(Cl)c3)C2)c1] |
| | (S)-N-(3-chloro-4-fluorophenyl)-1-(4-cyano-1H-pyrrole-2- |
| | carbonyl)pyrrolidine-3-carboxamide |
| 41 | 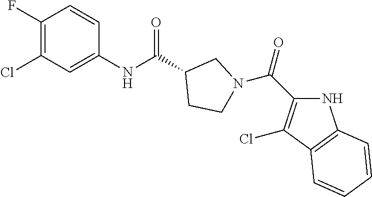 [SMILES: O=C(Nc1ccc(F)c(Cl)c1)[C@H]1CCN(C(=O)c2[nH]c3ccccc3c2Cl)C1] |
| | (S)-1-(3-chloro-1H-indole-2-carbonyl)-N-(3-chloro-4-fluoro- |
| | phenyl)pyrrolidine-3-carboxamide |
| 42 | 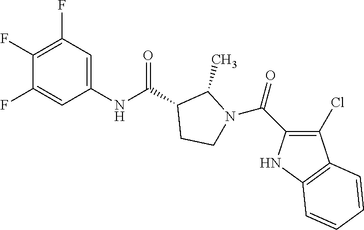 [SMILES: C[C@H]1[C@@H](C(=O)Nc2cc(F)c(F)c(F)c2)CCN1C(=O)c1[nH]c2ccccc2c1Cl] |
| | (2S,3S)-1-(3-chloro-1H-indole-2-carbonyl)-2-methyl-N-(3,4,5- |
| | trifluorophenyl)pyrrolidine-3-carboxamide |
| 43 | 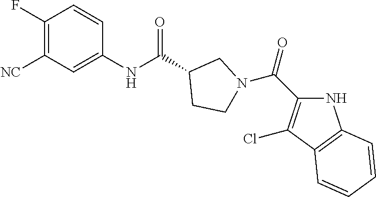 [SMILES: N#Cc1cc(NC(=O)[C@H]2CCN(C(=O)c3[nH]c4ccccc4c3Cl)C2)ccc1F] |
| | (S)-1-(3-chloro-1H-indole-2-carbonyl)-N-(3-cyano-4- |
| | fluorophenyl)pyrrolidine-3-carboxamide |
| 44 | 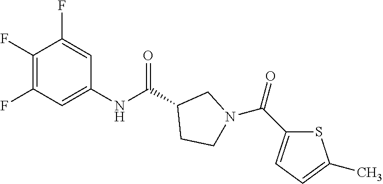 [SMILES: Cc1ccc(C(=O)N2CC[C@H](C(=O)Nc3cc(F)c(F)c(F)c3)C2)s1] |
| | (S)-1-(5-methylthiophene-2-carbonyl)-N-(3,4,5- |
| | trifluorophenyl)pyrrolidine-3-carboxamide |
| 45 | 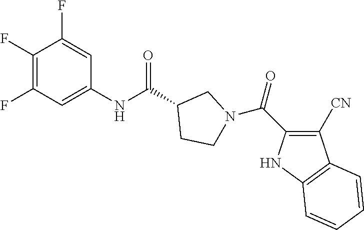 [SMILES: N#Cc1c(C(=O)N2CC[C@H](C(=O)Nc3cc(F)c(F)c(F)c3)C2)[nH]c2ccccc12] |
| | (S)-1-(3-cyano-1H-indole-2-carbonyl)-N-(3,4,5- |
| | trifluorophenyl)pyrrolidine-3-carboxamide |
| 46 | 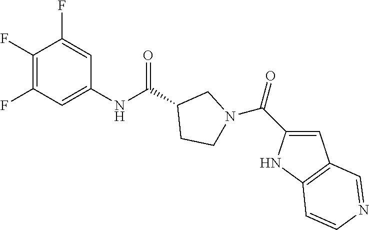 [SMILES: O=C(Nc1cc(F)c(F)c(F)c1)[C@H]1CCN(C(=O)c2cc3cnccc3[nH]2)C1] |
| | (S)-1-(1H-pyrrolo[3,2-c]pyridine-2-carbonyl)-N-(3,4,5- |
| | trifluorophenyl)pyrrolidine-3-carboxamide |
| 47 | 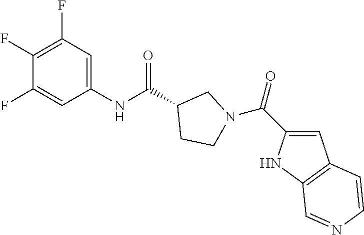 [SMILES: O=C(Nc1cc(F)c(F)c(F)c1)[C@H]1CCN(C(=O)c2cc3ccncc3[nH]2)C1] |
| | (S)-1-(1H-pyrrolo[2,3-c]pyridine-2-carbonyl)-N-(3,4,5- |
| | trifluorophenyl)pyrrolidine-3-carboxamide |
| 48 | 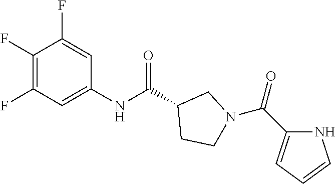 [SMILES: O=C(Nc1cc(F)c(F)c(F)c1)[C@H]1CCN(C(=O)c2ccc[nH]2)C1] |
| | (S)-1-(1H-pyrrole-2-carbonyl)-N-(3,4,5-trifluorophenyl)pyrrolidine-3- |
| | carboxamide |
| 49 | 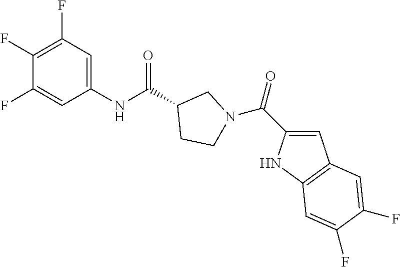 [SMILES: O=C(Nc1cc(F)c(F)c(F)c1)[C@H]1CCN(C(=O)c2cc3cc(F)c(F)cc3[nH]2)C1] |
| | (S)-1-(5,6-difluoro-1H-indole-2-carbonyl)-N-(3,4,5- |
| | trifluorophenyl)pyrrolidine-3-carboxamide |
| 50 | 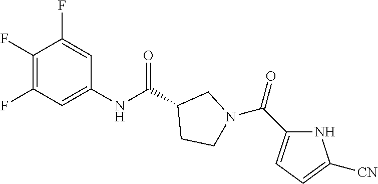 [SMILES: N#Cc1ccc(C(=O)N2CC[C@H](C(=O)Nc3cc(F)c(F)c(F)c3)C2)[nH]1] |
| | (S)-1-(5-cyano-1H-pyrrole-2-carbonyl)-N-(3,4,5- |
| | trifluorophenyl)pyrrolidine-3-carboxamide |
| 51 | 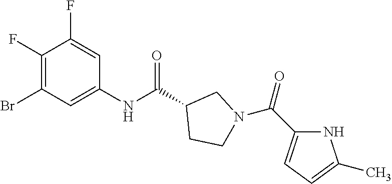 [SMILES: Cc1ccc(C(=O)N2CC[C@H](C(=O)Nc3cc(F)c(F)c(Br)c3)C2)[nH]1] |
| | (S)-N-(3-bromo-4,5-difluorophenyl)-1-(5-methyl-1H-pyrrole-2- |
| | carbonyl)pyrrolidine-3-carboxamide |
| 52 | 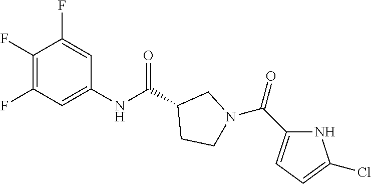 [SMILES: O=C(Nc1cc(F)c(F)c(F)c1)[C@H]1CCN(C(=O)c2ccc(Cl)[nH]2)C1] |
| | (S)-1-(5-chloro-1H-pyrrole-2-carbonyl)-N-(3,4,5- |
| | trifluorophenyl)pyrrolidine-3-carboxamide |
| 53 | 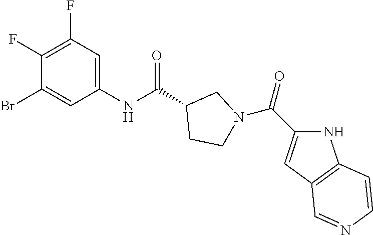 [SMILES: O=C(Nc1cc(F)c(F)c(Br)c1)[C@H]1CCN(C(=O)c2cc3cnccc3[nH]2)C1] |
| | (S)-N-(3-bromo-4,5-difluorophenyl)-1-(1H-pyrrolo[3,2-c]pyridine-2- |
| | carbonyl)pyrrolidine-3-carboxamide |
| 54 | 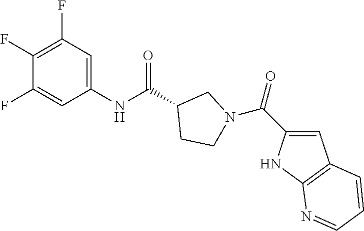 [SMILES: O=C(Nc1cc(F)c(F)c(F)c1)[C@H]1CCN(C(=O)c2cc3cccnc3[nH]2)C1] |
| | (S)-1-(1H-pyrrolo[2,3-b]pyridine-2-carbonyl)-N-(3,4,5- |
| | trifluorophenyl)pyrrolidine-3-carboxamide |
| 55 | 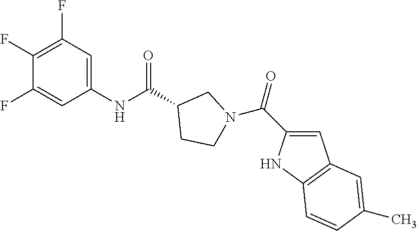 [SMILES: Cc1ccc2[nH]c(C(=O)N3CC[C@H](C(=O)Nc4cc(F)c(F)c(F)c4)C3)cc2c1] |
| | (S)-1-(5-methyl-1H-indole-2-carbonyl)-N-(3,4,5- |
| | trifluorphenyl)pyrrolidine-3-carboxamide |
| 56 | 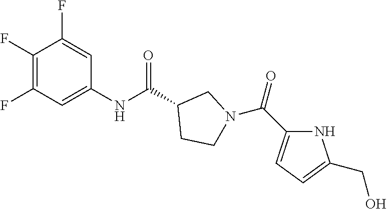 [SMILES: O=C(Nc1cc(F)c(F)c(F)c1)[C@H]1CCN(C(=O)c2ccc(CO)[nH]2)C1] |
| | (S)-1-(5-(hydroxymethyl)-1H-pyrrole-2-carbonyl)-N-(3,4,5- |
| | trifluorophenyl)pyrrolidine-3-carboxamide |
| 57 | 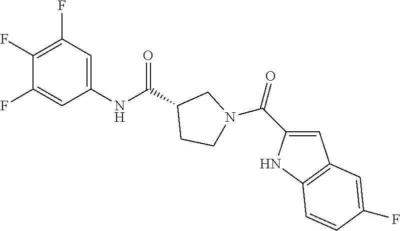 [SMILES: O=C(Nc1cc(F)c(F)c(F)c1)[C@H]1CCN(C(=O)c2cc3cc(F)ccc3[nH]2)C1] |
| | (S)-1-(5-fluoro-1H-indole-2-carbonyl)-N-(3,4,5- |
| | trifluorophenyl)pyrrolidine-3-carboxamide |
| 58 | 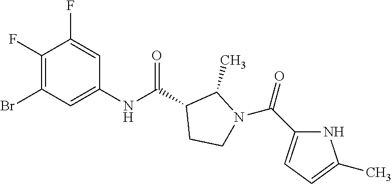 [SMILES: Cc1ccc(C(=O)N2CC[C@H](C(=O)Nc3cc(F)c(F)c(Br)c3)[C@@H]2C)[nH]1] |
| | (2S,3S)-N-(3-bromo-4,5-difluorophenyl)-2-methyl-1-(5-methyl-1H- |
| | pyrrole-2-carbonyl)pyrrolidine-3-carboxamide |
| 59 | 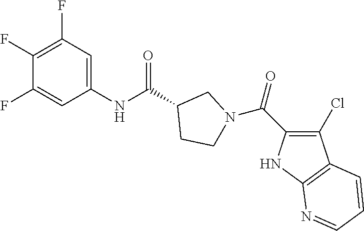 [SMILES: O=C(Nc1cc(F)c(F)c(F)c1)[C@H]1CCN(C(=O)c2[nH]c3ncccc3c2Cl)C1] |
| | (S)-1-(3-chloro-1H-pyrrolo[2,3-b]pyridine-2-carbonyl)-N-(3,4,5- |
| | trifluorophenyl)pyrrolidine-3-carboxamide |
| 60 | 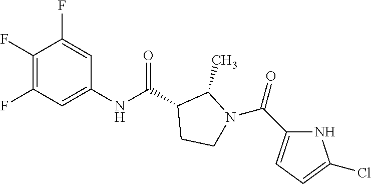 [SMILES: C[C@H]1[C@@H](C(=O)Nc2cc(F)c(F)c(F)c2)CCN1C(=O)c1ccc(Cl)[nH]1] |
| | (2S,3S)-1-(5-chloro-1H-pyrrole-2-carbonyl)-2-methyl-N-(3,4,5- |
| | trifluorophenyl)pyrrolidine-3-carboxamide |
| 61 | 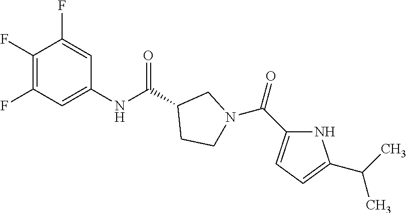 [SMILES: CC(C)c1ccc(C(=O)N2CC[C@H](C(=O)Nc3cc(F)c(F)c(F)c3)C2)[nH]1] |
| | (S)-1-(5-isopropyl-1H-pyrrole-2-carbonyl)-N-(3,4,5- |
| | trifluorophenyl)pyrrolidine-3-carboxamide |
| 62 | 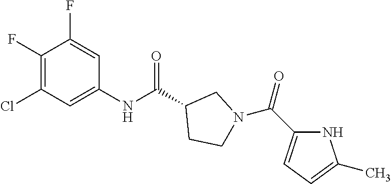 [SMILES: Cc1ccc(C(=O)N2CC[C@H](C(=O)Nc3cc(F)c(F)c(Cl)c3)C2)[nH]1] |
| | (S)-N-(3-chloro-4,5-difluorophenyl)-1-(5-methyl-1H-pyrrole-2- |
| | carbonyl)pyrrolidine-3-carboxamide |
| 63 | 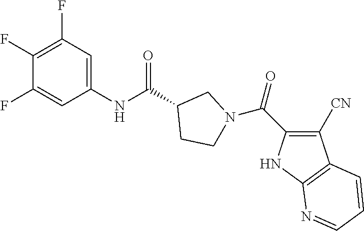 [SMILES: N#Cc1c(C(=O)N2CC[C@H](C(=O)Nc3cc(F)c(F)c(F)c3)C2)[nH]c2ncccc12] |
| | (S)-1-(3-cyano-1H-pyrrolo[2,3-b]pyridine-2-carbonyl)-N-(3,4,5- |
| | trifluorophenyl)pyrrolidine-3-carboxamide |
| 64 | 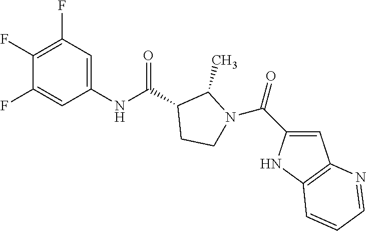 [SMILES: C[C@H]1[C@@H](C(=O)Nc2cc(F)c(F)c(F)c2)CCN1C(=O)c1cc2ncccc2[nH]1] |
| | (2S,3S)-2-methyl-1-(1H-pyrrolo[3,2-b]pyridine-2-carbonyl)-N-(3,4,5- |
| | trifluorophenyl)pyrrolidine-3-carboxamide |
| 65 | 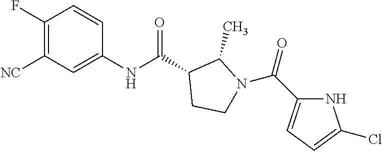 [SMILES: C[C@H]1[C@@H](C(=O)Nc2ccc(F)c(C#N)c2)CCN1C(=O)c1ccc(Cl)[nH]1] |
| | (2S,3S)-1-(5-chloro-1H-pyrrole-2-carbonyl)-N-(3-cyano-4-fluorophenyl)- |
| | 2-methylpyrrolidine-3-carboxamide |
| 66 | 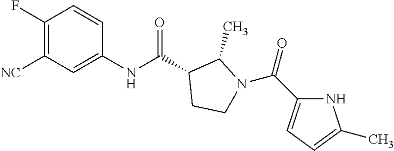 [SMILES: Cc1ccc(C(=O)N2CC[C@H](C(=O)Nc3ccc(F)c(C#N)c3)[C@@H]2C)[nH]1] |
| | (2S,3S)-N-(3-cyano-4-fluorophenyl)-2-methyl-1-(5-methyl-1H-pyrrole-2- |
| | carbonyl)pyrrolidine-3-carboxamide |
| 67 | 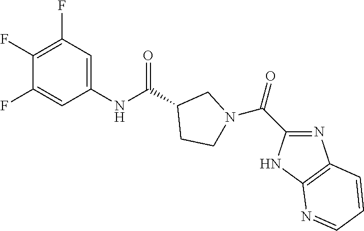 [SMILES: O=C(Nc1cc(F)c(F)c(F)c1)[C@H]1CCN(C(=O)c2nc3cccnc3[nH]2)C1] |
| | (S)-1-(3H-imidazo[4,5-b]pyridine-2-carbonyl)-N-(3,4,5- |
| | trifluorophenyl)pyrrolidine-3-carboxamide |
| 68 | 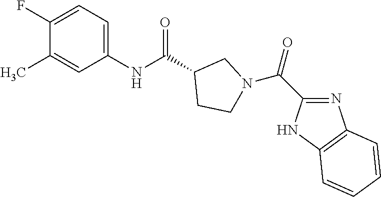 [SMILES: Cc1cc(NC(=O)[C@H]2CCN(C(=O)c3nc4ccccc4[nH]3)C2)ccc1F] |
| | (S)-1-(1H-benzo[d]imidazole-2-carbonyl)-N-(4-fluoro-3- |
| | methylphenyl)pyrrolidine-3-carboxamide |
| 69 | 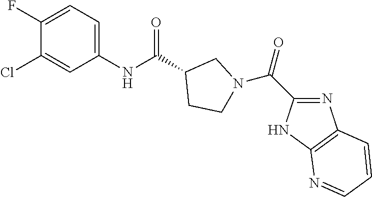 [SMILES: O=C(Nc1ccc(F)c(Cl)c1)[C@H]1CCN(C(=O)c2nc3cccnc3[nH]2)C1] |
| | (S)-N-(3-chloro-4-fluorophenyl)-1-(3H-imidazo[4,5-b]pyridine-2- |
| | carbonyl)pyrrolidine-3-carboxamide |
| 70 | 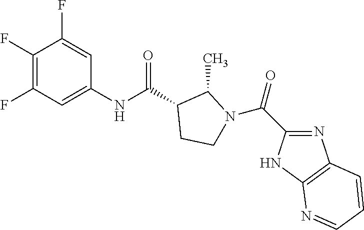 [SMILES: C[C@H]1[C@@H](C(=O)Nc2cc(F)c(F)c(F)c2)CCN1C(=O)c1nc2cccnc2[nH]1] |
| | (2S,3S)-1-(3H-imidazo[4,5-b]pyridine-2-carbonyl)-2-methyl-N-(3,4,5- |
| | trifluorophenyl)pyrrolidine-3-carboxamide |
| 71 | 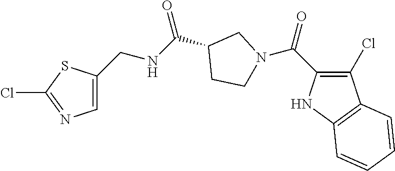 [SMILES: O=C(NCc1cnc(Cl)s1)[C@H]1CCN(C(=O)c2[nH]c3ccccc3c2Cl)C1] |
| | (S)-1-(3-chloro-1H-indole-2-carbonyl)-N-((2-chlorothiazol-5- |
| | yl)methyl)pyrrolidine-3-carboxamide |
| 72 | 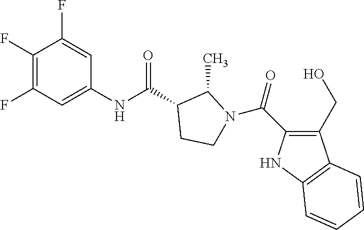 [SMILES: C[C@H]1[C@@H](C(=O)Nc2cc(F)c(F)c(F)c2)CCN1C(=O)c1[nH]c2ccccc2c1CO] |
| | (2S,3S)-1-(3-(hydroxymethyl)-1H-indole-2-carbonyl)-2-methyl-N-(3,4,5- |
| | trifluorophenyl)pyrrolidine-3-carboxamide |
| 73 | 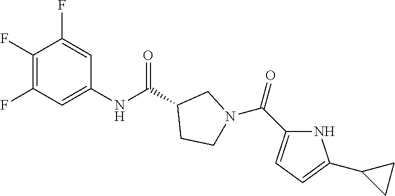 [SMILES: O=C(Nc1cc(F)c(F)c(F)c1)[C@H]1CCN(C(=O)c2ccc(C3CC3)[nH]2)C1] |
| | (S)-1-(5-cyclopropyl-1H-pyrrole-2-carbonyl)-N-(3,4,5- |
| | trifluorophenyl)pyrrolidine-3-carboxamide |
| 74 | 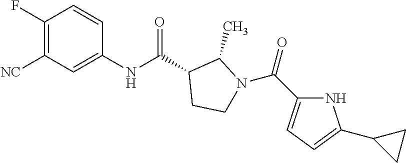 [SMILES: C[C@H]1[C@@H](C(=O)Nc2ccc(F)c(C#N)c2)CCN1C(=O)c1ccc(C2CC2)[nH]1] |
| | (2S,3S)-N-(3-cyano-4-fluorophenyl)-1-(5-cyclopropyl-1H-pyrrole-2- |
| | carbonyl)-2-methylpyrrolidine-3-carboxamide |
| 75 | 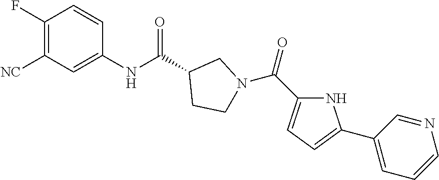 [SMILES: N#Cc1cc(NC(=O)[C@H]2CCN(C(=O)c3ccc(-c4cccnc4)[nH]3)C2)ccc1F] |
| | (S)-N-(3-cyano-4-fluorophenyl)-1-(5-(pyridin-3-yl)-1H-pyrrole-2- |
| | carbonyl)pyrrolidine-3-carboxamide |
| 76 | 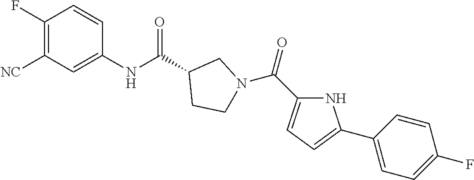 [SMILES: N#Cc1cc(NC(=O)[C@H]2CCN(C(=O)c3ccc(-c4ccc(F)cc4)[nH]3)C2)ccc1F] |
| | (S)-N-(3-cyano-4-fluorophenyl)-1-(5-(4-fluorophenyl)-1H-pyrrole-2- |
| | carbonyl)pyrrolidine-3-carboxamide |
| 77 | 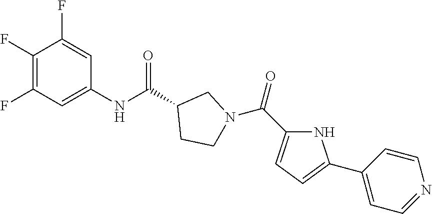 [SMILES: O=C(Nc1cc(F)c(F)c(F)c1)[C@H]1CCN(C(=O)c2ccc(-c3ccncc3)[nH]2)C1] |
| | (S)-1-(5-(pyridin-4-yl)-1H-pyrrole-2-carbonyl)-N-(3,4,5- |
| | trifluorophenyl)pyrrolidine-3-carboxamide |
| 78 | 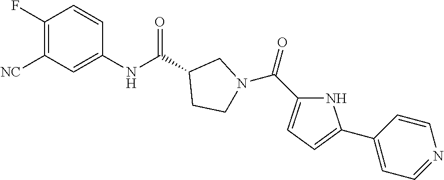 [SMILES: N#Cc1cc(NC(=O)[C@H]2CCN(C(=O)c3ccc(-c4ccncc4)[nH]3)C2)ccc1F] |
| | (S)-N-(3-cyano-4-fluorophenyl)-1-(5-(pyridin-4-yl)-1H-pyrrole-2- |
| | carbonyl)pyrrolidine-3-carboxamide |
| 79 | 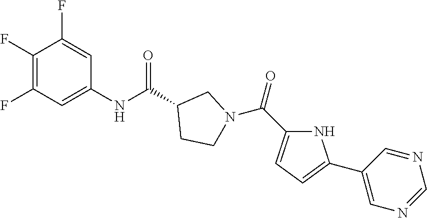 [SMILES: O=C(Nc1cc(F)c(F)c(F)c1)[C@H]1CCN(C(=O)c2ccc(-c3cncnc3)[nH]2)C1] |
| | (S)-1-(5-(pyrimidin-5-yl)-1H-pyrrole-2-carbonyl)-N-(3,4,5- |
| | trifluorophenyl)pyrrolidine-3-carboxamide |
| 80 | 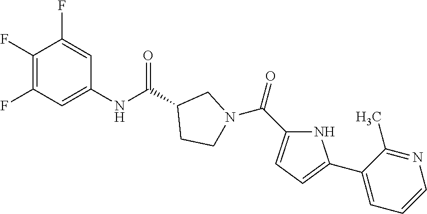 [SMILES: Cc1ncccc1-c1ccc(C(=O)N2CC[C@H](C(=O)Nc3cc(F)c(F)c(F)c3)C2)[nH]1] |
| | (S)-1-(5-(2-methylpyridin-3-yl)-1H-pyrrole-2-carbonyl)-N-(3,4,5- |
| | trifluorophenyl)pyrrolidine-3-carboxamide |
| 81 | 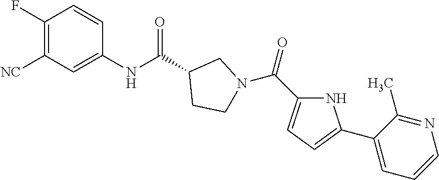 [SMILES: Cc1ncccc1-c1ccc(C(=O)N2CC[C@H](C(=O)Nc3ccc(F)c(C#N)c3)C2)[nH]1] |
| | (S)-N-(3-cyano-4-fluorophenyl)-1-(5-(2-methylpyridin-3-yl)-1H-pyrrole- |
| | 2-carbonyl)pyrrolidine-3-carboxamide |
| 82 | 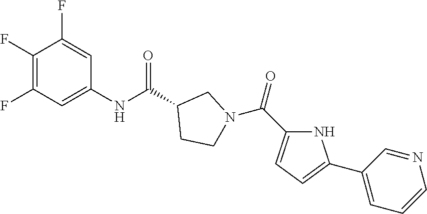 [SMILES: O=C(Nc1cc(F)c(F)c(F)c1)[C@H]1CCN(C(=O)c2ccc(-c3cccnc3)[nH]2)C1] |
| | (S)-1-(5-(pyridin-3-yl)-1H-pyrrole-2-carbonyl)-N-(3,4,5- |
| | trifluorophenyl)pyrrolidine-3-carboxamide |
| 83 | 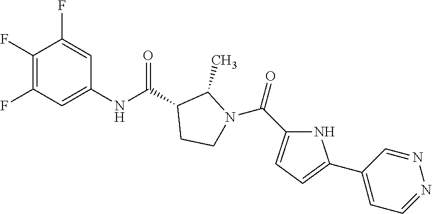 [SMILES: C[C@H]1[C@@H](C(=O)Nc2cc(F)c(F)c(F)c2)CCN1C(=O)c1ccc(-c2ccnnc2)[nH]1] |
| | (2S,3S)-2-methyl-1-(5-(pyridazin-4-yl)-1H-pyrrole-2-carbonyl)-N-(3,4,5- |
| | trifluorophenyl)pyrrolidine-3-carboxamide |
| 84 | 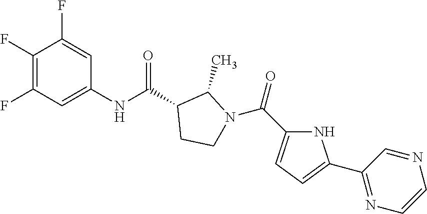 [SMILES: C[C@H]1[C@@H](C(=O)Nc2cc(F)c(F)c(F)c2)CCN1C(=O)c1ccc(-c2cnccn2)[nH]1] |
| | (2S,3S)-2-methyl-1-(5-(pyrazin-2-yl)-1H-pyrrole-2-carbonyl)-N-(3,4,5- |
| | trifluorophenyl)pyrrolidine-3-carboxamide |
| 85 | 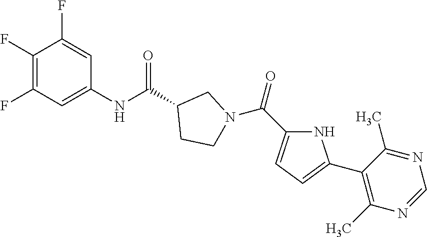 [SMILES: Cc1ncnc(C)c1-c1ccc(C(=O)N2CC[C@H](C(=O)Nc3cc(F)c(F)c(F)c3)C2)[nH]1] |
| | (S)-1-(5-(4,6-dimethylpyrimidin-5-yl)-1H-pyrrole-2-carbonyl)-N-(3,4,5- |
| | trifluorophenyl)pyrrolidine-3-carboxamide |
| 86 | 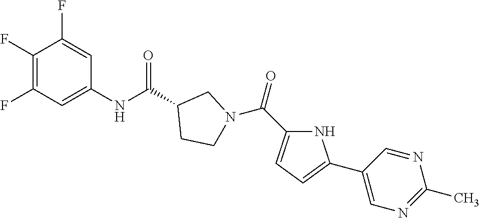 [SMILES: Cc1ncc(-c2ccc(C(=O)N3CC[C@H](C(=O)Nc4cc(F)c(F)c(F)c4)C3)[nH]2)cn1] |
| | (S)-1-(5-(2-methylpyrimidin-5-yl)-1H-pyrrole-2-carbonyl)-N-(3,4,5- |
| | trifluorophenyl)pyrrolidine-3-carboxamide |
| 87 | 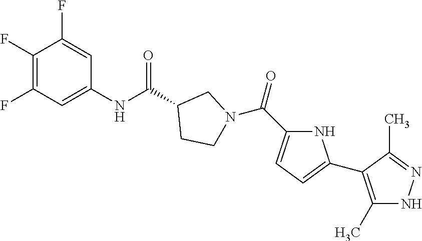 [SMILES: Cc1n[nH]c(C)c1-c1ccc(C(=O)N2CC[C@H](C(=O)Nc3cc(F)c(F)c(F)c3)C2)[nH]1] |
| | (S)-1-(5-(3,5-dimethyl-1H-pyrazol-4-yl)-1H-pyrrole-2-carbonyl)-N- |
| | (3,4,5-trifluorophenyl)pyrrolidine-3-carboxamide |
| 88 | 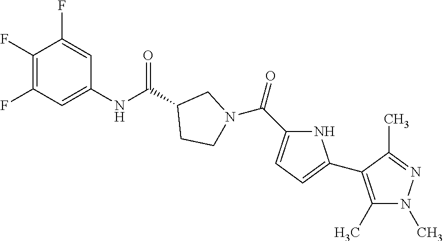 [SMILES: Cc1nn(C)c(C)c1-c1ccc(C(=O)N2CC[C@H](C(=O)Nc3cc(F)c(F)c(F)c3)C2)[nH]1] |
| | (S)-N-(3,4,5-trifluorophenyl)-1-(5-(1,3,5-trimethyl-1H-pyrazol-4-yl)-1H- |
| | pyrrole-2-carbonyl)pyrrolidine-3-carboxamide |
| 89 | 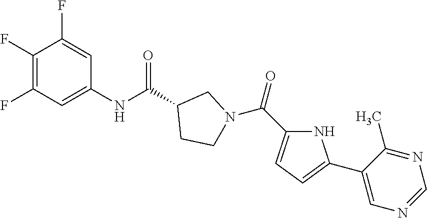 [SMILES: Cc1ncncc1-c1ccc(C(=O)N2CC[C@H](C(=O)Nc3cc(F)c(F)c(F)c3)C2)[nH]1] |
| | (S)-1-(5-(4-methylpyrimidin-5-yl)-1H-pyrrole-2-carbonyl)-N-(3,4,5- |
| | trifluorophenyl)pyrrolidine-3-carboxamide |
| 90 | 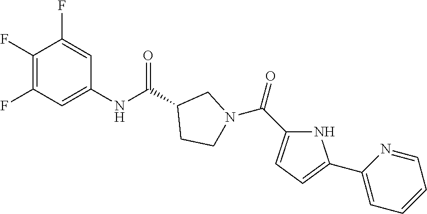 [SMILES: O=C(Nc1cc(F)c(F)c(F)c1)[C@H]1CCN(C(=O)c2ccc(-c3ccccn3)[nH]2)C1] |
| | (S)-1-(5-(pyridin-2-yl)-1H-pyrrole-2-carbonyl)-N-(3,4,5- |
| | trifluorophenyl)pyrrolidine-3-carboxamide |
| 91 | 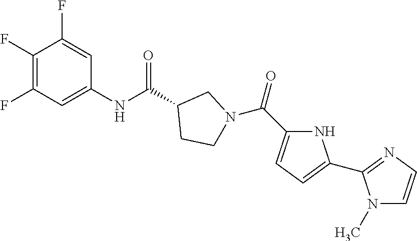 [SMILES: Cn1ccnc1-c1ccc(C(=O)N2CC[C@H](C(=O)Nc3cc(F)c(F)c(F)c3)C2)[nH]1] |
| | (S)-1-(5-(1-methyl-1H-imidazol-2-yl)-1H-pyrrole-2-carbonyl)-N-(3,4,5- |
| | trifluorophenyl)pyrrolidine-3-carboxamide |
| 92 | 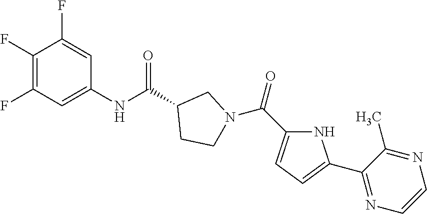 [SMILES: Cc1nccnc1-c1ccc(C(=O)N2CC[C@H](C(=O)Nc3cc(F)c(F)c(F)c3)C2)[nH]1] |
| | (S)-1-(5-(3-methylpyrazin-2-yl)-1H-pyrrole-2-carbonyl)-N-(3,4,5- |
| | trifluorophenyl)pyrrolidine-3-carboxamide |
| 93 | 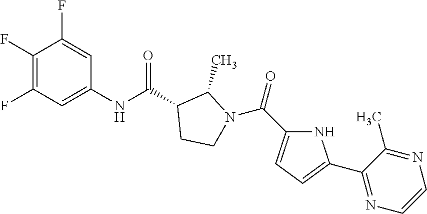 [SMILES: Cc1nccnc1-c1ccc(C(=O)N2CC[C@H](C(=O)Nc3cc(F)c(F)c(F)c3)[C@@H]2C)[nH]1] |
| | (2S,3S)-2-methyl-1-(5-(3-methylpyrazin-2-yl)-1H-pyrrole-2-carbonyl)-N- |
| | (3,4,5-trifluorophenyl)pyrrolidine-3-carboxamide |
| 94 | 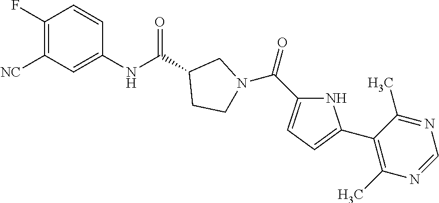 [SMILES: Cc1ncnc(C)c1-c1ccc(C(=O)N2CC[C@H](C(=O)Nc3ccc(F)c(C#N)c3)C2)[nH]1] |
| | (S)-N-(3-cyano-4-fluorophenyl)-1-(5-(4,6-dimethylpyrimidin-5-yl)-1H- |
| | pyrrole-2-carbonyl)pyrrolidine-3-carboxamide |
| 95 | 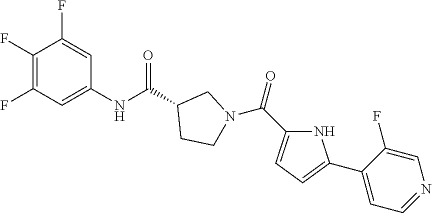 [SMILES: O=C(Nc1cc(F)c(F)c(F)c1)[C@H]1CCN(C(=O)c2ccc(-c3ccncc3F)[nH]2)C1] |
| | (S)-1-(5-(3-fluoropyridin-4-yl)-1H-pyrrole-2-carbonyl)-N-(3,4,5- |
| | trifluorophenyl)pyrrolidine-3-carboxamide |
| 96 | 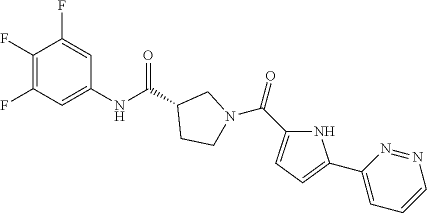 [SMILES: O=C(Nc1cc(F)c(F)c(F)c1)[C@H]1CCN(C(=O)c2ccc(-c3cccnn3)[nH]2)C1] |
| | (S)-1-(5-(pyridazin-3-yl)-1H-pyrrole-2-carbonyl)-N-(3,4,5- |
| | trifluorophenyl)pyrrolidine-3-carboxamide |
| 97 | 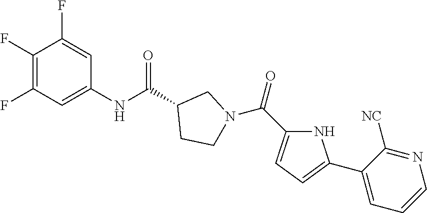 [SMILES: N#Cc1ncccc1-c1ccc(C(=O)N2CC[C@H](C(=O)Nc3cc(F)c(F)c(F)c3)C2)[nH]1] |
| | (S)-1-(5-(2-cyanopyridin-3-yl)-1H-pyrrole-2-carbonyl)-N-(3,4,5- |
| | trifluorophenyl)pyrrolidine-3-carboxamide |
| 98 | 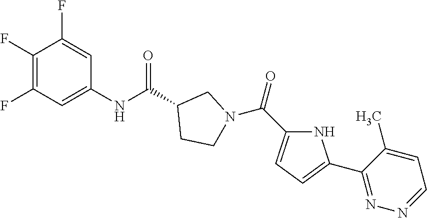 [SMILES: Cc1ccnnc1-c1ccc(C(=O)N2CC[C@H](C(=O)Nc3cc(F)c(F)c(F)c3)C2)[nH]1] |
| | (S)-1-(5-(4-methylpyridazin-3-yl)-1H-pyrrole-2-carbonyl)-N-(3,4,5- |
| | trifluorophenyl)pyrrolidine-3-carboxamide |
| 99 | 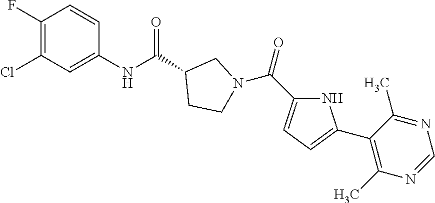 [SMILES: Cc1ncnc(C)c1-c1ccc(C(=O)N2CC[C@H](C(=O)Nc3ccc(F)c(Cl)c3)C2)[nH]1] |
| | (S)-N-(3-chloro-4-fluorophenyl)-1-(5-(4,6-dimethylpyrimidin-5-yl)-1H- |
| | pyrrole-2-carbonyl)pyrrolidine-3-carboxamide |
| 100 | 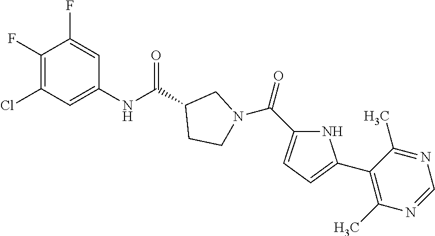 [SMILES: Cc1ncnc(C)c1-c1ccc(C(=O)N2CC[C@H](C(=O)Nc3cc(F)c(F)c(Cl)c3)C2)[nH]1] |
| | (S)-N-(3-chloro-4,5-difluorophenyl)-1-(5-(4,6-dimethylpyrimidin-5-yl)- |
| | 1H-pyrrole-2-carbonyl)pyrrolidine-3-carboxamide |
| 101 | 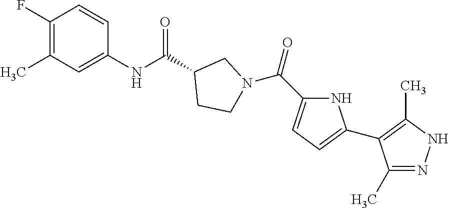 [SMILES: Cc1cc(NC(=O)[C@H]2CCN(C(=O)c3ccc(-c4c(C)n[nH]c4C)[nH]3)C2)ccc1F] |
| | (S)-1-(5-(3,5-dimethyl-1H-pyrazol-4-yl)-1H-pyrrole-2-carbonyl)-N-(4- |
| | fluoro-3-methylphenyl)pyrrolidine-3-carboxamide |
| 102 | 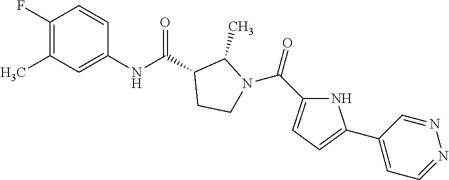 [SMILES: Cc1cc(NC(=O)[C@H]2CCN(C(=O)c3ccc(-c4ccnnc4)[nH]3)[C@H]2C)ccc1F] |
| | (2S,3S)-N-(4-fluoro-3-methylphenyl)-2-methyl-1-(5-(pyridazin-4-yl)-1H- |
| | pyrrole-2-carbonyl)pyrrolidine-3-carboxamide |
| 103 | 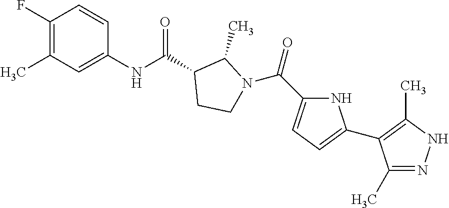 [SMILES: Cc1cc(NC(=O)[C@H]2CCN(C(=O)c3ccc(-c4c(C)n[nH]c4C)[nH]3)[C@H]2C)ccc1F] |
| | (2S,3S)-1-(5-(3,5-dimethyl-1H-pyrazol-4-yl)-1H-pyrrole-2-carbonyl)-N- |
| | (4-fluoro-3-methylphenyl)-2-methylpyrrolidine-3-carboxamide |
| 104 | 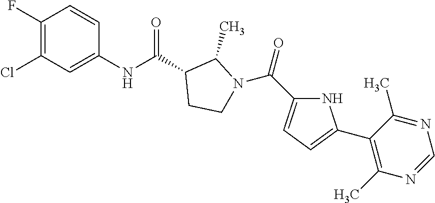 [SMILES: Cc1ncnc(C)c1-c1ccc(C(=O)N2CC[C@H](C(=O)Nc3ccc(F)c(Cl)c3)[C@@H]2C)[nH]1] |
| | (2S,3S)-N-(3-chloro-4-fluorophenyl)-1-(5-(4,6-dimethylpyrimidin-5-yl)- |
| | 1H-pyrrole-2-carbonyl)-2-methylpyrrolidine-3-carboxamide |
| 105 | 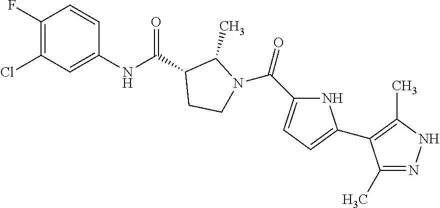 [SMILES: Cc1n[nH]c(C)c1-c1ccc(C(=O)N2CC[C@H](C(=O)Nc3ccc(F)c(Cl)c3)[C@@H]2C)[nH]1] |
| | (2S,3S)-N-(3-chloro-4-fluorophenyl)-1-(5-(3,5-dimethyl-1H-pyrazol-4- |
| | yl)-1H-pyrrole-2-carbonyl)-2-methylpyrrolidine-3-carboxamide |
| 106 | 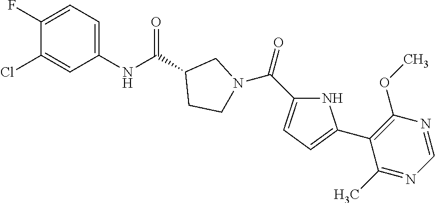 [SMILES: COc1ncnc(C)c1-c1ccc(C(=O)N2CC[C@H](C(=O)Nc3ccc(F)c(Cl)c3)C2)[nH]1] |
| | (S)-N-(3-chloro-4-fluorophenyl)-1-(5-(4-methoxy-6-methylpyrimidin-5- |
| | yl)-1H-pyrrole-2-carbonyl)pyrrolidine-3-carboxamide |
| 107 | 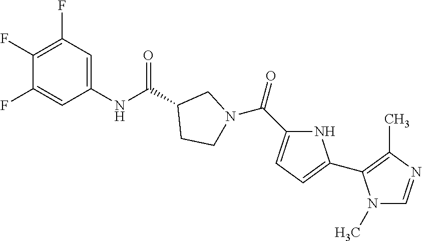 [SMILES: Cc1ncn(C)c1-c1ccc(C(=O)N2CC[C@H](C(=O)Nc3cc(F)c(F)c(F)c3)C2)[nH]1] |
| | (S)-1-(5-(1,4-dimethyl-1H-imidazol-5-yl)-1H-pyrrole-2-carbonyl)-N- |
| | (3,4,5-trifluorophenyl)pyrrolidine-3-carboxamide |
| 108 | 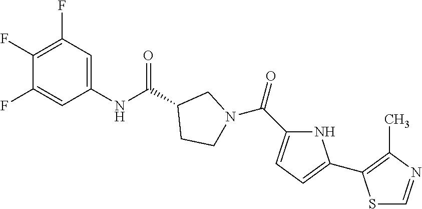 [SMILES: Cc1ncsc1-c1ccc(C(=O)N2CC[C@H](C(=O)Nc3cc(F)c(F)c(F)c3)C2)[nH]1] |
| | (S)-1-(5-(4-methylthiazol-5-yl)-1H-pyrrole-2-carbonyl)-N-(3,4,5- |
| | trifluorophenyl)pyrrolidine-3-carboxamide |
| 109 | 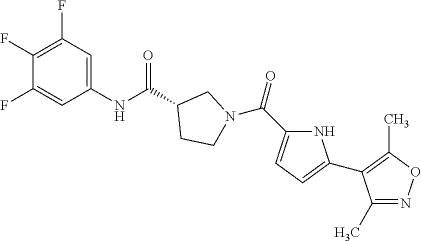 [SMILES: Cc1noc(C)c1-c1ccc(C(=O)N2CC[C@H](C(=O)Nc3cc(F)c(F)c(F)c3)C2)[nH]1] |
| | (S)-1-(5-(3,5-dimethylisoxazol-4-yl)-1H-pyrrole-2-carbonyl)-N-(3,4,5- |
| | trifluorophenyl)pyrrolidine-3-carboxamide |
| 110 | 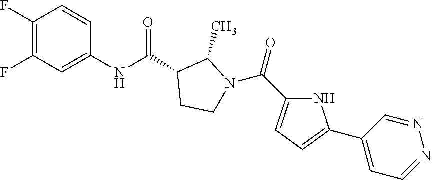 [SMILES: C[C@H]1[C@@H](C(=O)Nc2ccc(F)c(F)c2)CCN1C(=O)c1ccc(-c2ccnnc2)[nH]1] |
| | (2S,3S)-N-(3,4-difluorophenyl)-2-methyl-1-(5-(pyridazin-4-yl)-1H- |
| | pyrrole-2-carbonyl)pyrrolidine-3-carboxamide |
| 111 | 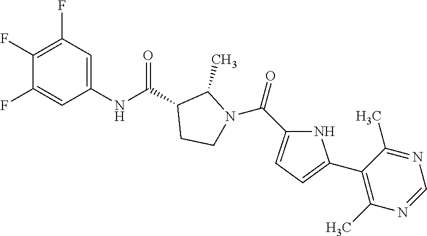 [SMILES: Cc1ncnc(C)c1-c1ccc(C(=O)N2CC[C@H](C(=O)Nc3cc(F)c(F)c(F)c3)[C@@H]2C)[nH]1] |
| | (2S,3S)-1-(5-(4,6-dimethylpyrimidin-5-yl)-1H-pyrrole-2-carbonyl)-2- |
| | methyl-N-(3,4,5-trifluorophenyl)pyrrolidine-3-carboxamide |
| 112 | 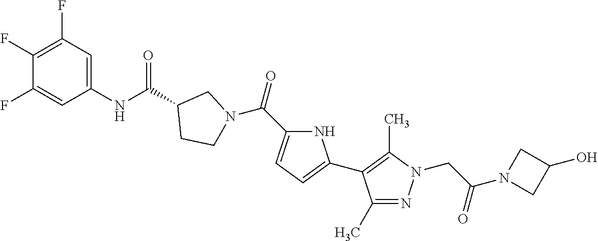 [SMILES: Cc1nn(CC(=O)N2CC(O)C2)c(C)c1-c1ccc(C(=O)N2CC[C@H](C(=O)Nc3cc(F)c(F)c(F)c3)C2)[nH]1] |
| | (S)-1-(5-(1-(2-(3-hydroxyazetidin-1-yl)-2-oxoethyl)-3,5-dimethyl-1H- |
| | pyrazol-4-yl)-1H-pyrrole-2-carbonyl)-N-(3,4,5- |
| | trifluorophenyl)pyrrolidine-3-carboxamide |
| 113 | 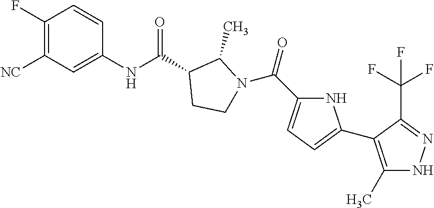 [SMILES: Cc1[nH]nc(C(F)(F)F)c1-c1ccc(C(=O)N2CC[C@H](C(=O)Nc3ccc(F)c(C#N)c3)[C@@H]2C)[nH]1] |
| | (2S,3S)-N-(3-cyano-4-fluorophenyl)-2-methyl-1-(5-(5-methyl-3- |
| | (trifluoromethyl)-1H-pyrazol-4-yl)-1H-pyrrole-2-carbonyl)pyrrolidine-3- |
| | carboxamide |
| 114 | 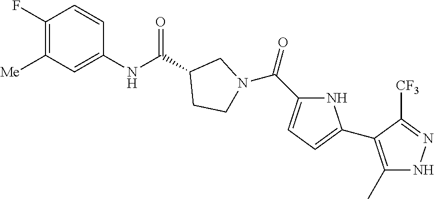 [SMILES: Cc1cc(NC(=O)[C@H]2CCN(C(=O)c3ccc(-c4c(C(F)(F)F)n[nH]c4C)[nH]3)C2)ccc1F] |
| | (S)-N-(4-fluoro-3-methylphenyl)-1-(5-(5-methyl-3-(trifluoromethyl)-1H- |
| | pyrazole-4-carbonyl)-1H-pyrrole-2-carbonyl)pyrrolidine-3-carboxamide |
| 115 | 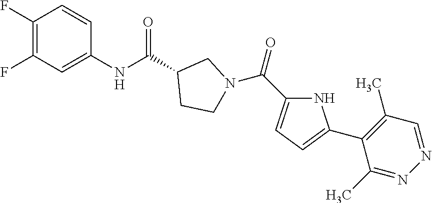 [SMILES: Cc1cnnc(C)c1-c1ccc(C(=O)N2CC[C@H](C(=O)Nc3ccc(F)c(F)c3)C2)[nH]1] |
| | (S)-N-(3,4-difluorophenyl)-1-(5-(3,5-dimethylpyridazin-4-yl)-1H-pyrrole- |
| | 2-carbonyl)pyrrolidine-3-carboxamide |
| 116 | 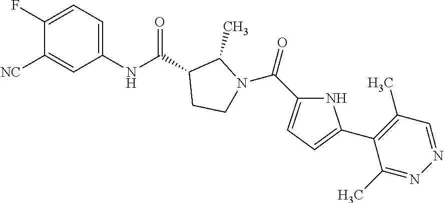 [SMILES: Cc1cnnc(C)c1-c1ccc(C(=O)N2CC[C@H](C(=O)Nc3ccc(F)c(C#N)c3)[C@@H]2C)[nH]1] |
| | (2S,3S)-N-(3-cyano-4-fluorophenyl)-1-(5-(3,5-dimethylpyridazin-4-yl)- |
| | 1H-pyrrole-2-carbonyl)-2-methylpyrrolidine-3-carboxamide |
| 117 | 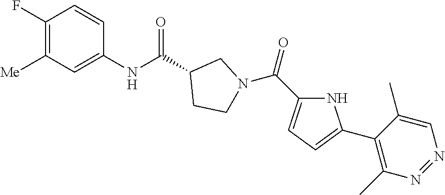 [SMILES: Cc1cc(NC(=O)[C@H]2CCN(C(=O)c3ccc(-c4c(C)cnnc4C)[nH]3)C2)ccc1F] |
| | (S)-1-(5-(3,5-dimethylpyridazin-4-yl)-1H-pyrrole-2-carbonyl)-N-(4- |
| | fluoro-3-methylphenyl)pyrrolidine-3-carboxamide |
| 118 | 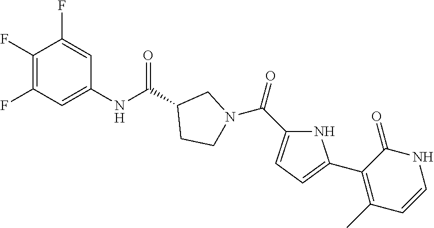 [SMILES: Cc1cc[nH]c(=O)c1-c1ccc(C(=O)N2CC[C@H](C(=O)Nc3cc(F)c(F)c(F)c3)C2)[nH]1] |
| | (S)-1-(5-(4-methyl-2-oxo-1,2-dihydropyridin-3-yl)-1H-pyrrole-2- |
| | carbonyl)-N-(3,4,5-trifluorophenyl)pyrrolidine-3-carboxamide |
| 119 | 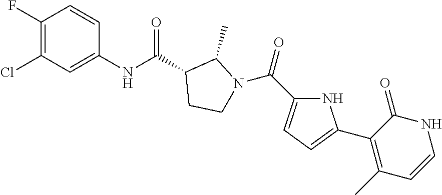 [SMILES: Cc1cc[nH]c(=O)c1-c1ccc(C(=O)N2CC[C@H](C(=O)Nc3ccc(F)c(Cl)c3)[C@@H]2C)[nH]1] |
| | (2S,3S)-N-(3-chloro-4-fluorophenyl)-2-methyl-1-(5-(4-methyl-2-oxo-1,2- |
| | dihydropyridin-3-yl)-1H-pyrrole-2-carbonyl)pyrrolidine-3-carboxamide |
| 120 | 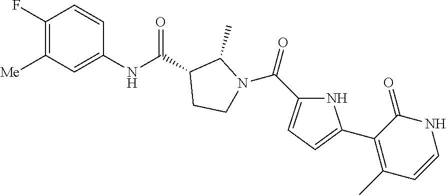 [SMILES: Cc1cc(NC(=O)[C@H]2CCN(C(=O)c3ccc(-c4c(C)cc[nH]c4=O)[nH]3)[C@H]2C)ccc1F] |
| | (2S,3S)-N-(4-fluoro-3-methylphenyl)-2-methyl-1-(5-(4-methyl-2-oxo-1,2- |
| | dihydropyridin-3-yl)-1H-pyrrole-2-carbonyl)pyrrolidine-3-carboxamide |
| 121 | 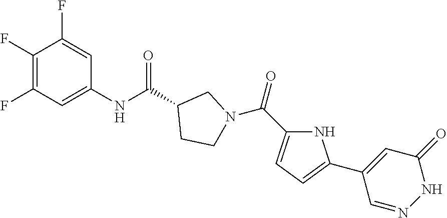 [SMILES: O=C(Nc1cc(F)c(F)c(F)c1)[C@H]1CCN(C(=O)c2ccc(-c3cn[nH]c(=O)c3)[nH]2)C1] |
| | (S)-1-(5-(6-oxo-1,6-dihydropyridazin-4-yl)-1H-pyrrole-2-carbonyl)-N- |
| | (3,4,5-trifluorophenyl)pyrrolidine-3-carboxamide |
| 122 | 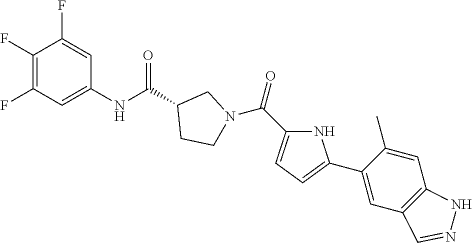 [SMILES: Cc1cc2[nH]ncc2cc1-c1ccc(C(=O)N2CC[C@H](C(=O)Nc3cc(F)c(F)c(F)c3)C2)[nH]1] |
| | (S)-1-(5-(6-methyl-1H-indazol-5-yl)-1H-pyrrole-2-carbonyl)-N-(3,4,5- |
| | trifluorophenyl)pyrrolidine-3-carboxamide |
| 123 | 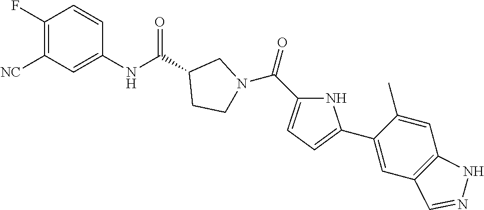 [SMILES: Cc1cc2[nH]ncc2cc1-c1ccc(C(=O)N2CC[C@H](C(=O)Nc3ccc(F)c(C#N)c3)C2)[nH]1] |
| | (S)-N-(3-cyano-4-fluorophenyl)-1-(5-(6-methyl-1H-indazol-5-yl)-1H- |
| | pyrrole-2-carbonyl)pyrrolidine-3-carboxamide |
| 124 | 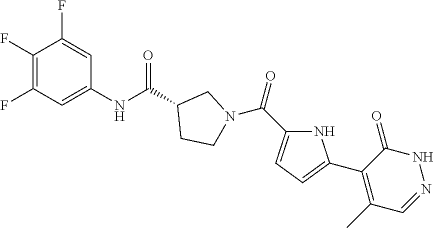 [SMILES: Cc1cn[nH]c(=O)c1-c1ccc(C(=O)N2CC[C@H](C(=O)Nc3cc(F)c(F)c(F)c3)C2)[nH]1] |
| | (S)-1-(5-(5-methyl-3-oxo-2,3-dihydropyridazin-4-yl)-1H-pyrrole-2- |
| | carbonyl)-N-(3,4,5-trifluorophenyl)pyrrolidine-3-carboxamide |
| 125 | 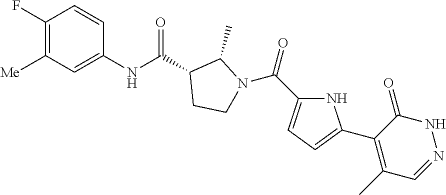 [SMILES: Cc1cc(NC(=O)[C@H]2CCN(C(=O)c3ccc(-c4c(C)cn[nH]c4=O)[nH]3)[C@H]2C)ccc1F] |
| | (2S,3S)-N-(4-fluoro-3-methylphenyl)-2-methyl-1-(5-(5-methyl-3-oxo-2,3- |
| | dihydropyridazin-4-yl)-1H-pyrrole-2-carbonyl)pyrrolidine-3-carboxamide |
| 126 | 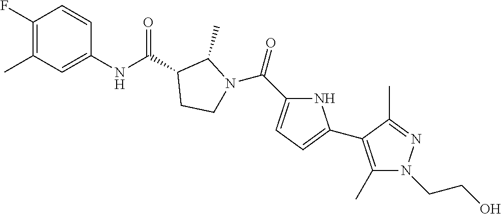 [SMILES: Cc1cc(NC(=O)[C@H]2CCN(C(=O)c3ccc(-c4c(C)nn(CCO)c4C)[nH]3)[C@H]2C)ccc1F] |
| | (2S,3S)-N-(4-fluoro-3-methylphenyl)-1-(5-(1-(2-hydroxyethyl)-3,5- |
| | dimethyl-1H-pyrazol-4-yl)-1H-pyrrole-2-carbonyl)-2-methylpyrrolidine- |
| | 3-carboxamide |
| 127 | 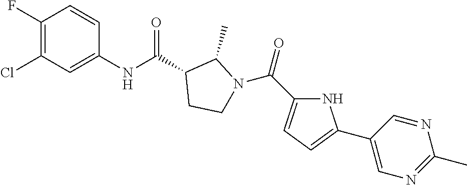 [SMILES: Cc1ncc(-c2ccc(C(=O)N3CC[C@H](C(=O)Nc4ccc(F)c(Cl)c4)[C@@H]3C)[nH]2)cn1] |
| | (2S,3S)-N-(3-chloro-4-fluorophenyl)-2-methyl-1-(5-(2-methylpyrimidin- |
| | 5-yl)-1H-pyrrole-2-carbonyl)pyrrolidine-3-carboxamide |
| 128 | 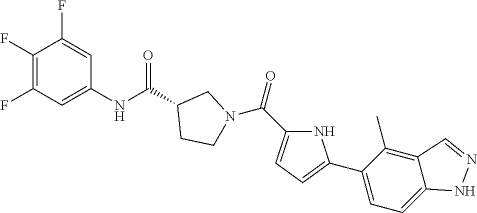 [SMILES: Cc1c(-c2ccc(C(=O)N3CC[C@H](C(=O)Nc4cc(F)c(F)c(F)c4)C3)[nH]2)ccc2[nH]ncc12] |
| | (S)-1-(5-(4-methyl-1H-indazol-5-yl)-1H-pyrrole-2-carbonyl)-N-(3,4,5- |
| | trifluorophenyl)pyrrolidine-3-carboxamide |
| 129 | 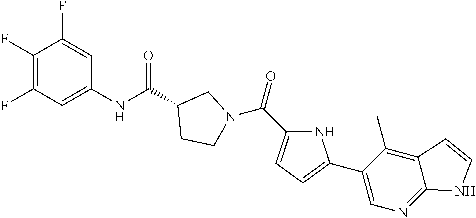 [SMILES: Cc1c(-c2ccc(C(=O)N3CC[C@H](C(=O)Nc4cc(F)c(F)c(F)c4)C3)[nH]2)cnc2[nH]ccc12] |
| | (S)-1-(5-(4-methyl-1H-pyrrolo[2,3-b]pyridin-5-yl)-1H-pyrrole-2- |
| | carbonyl)-N-(3,4,5-trifluorophenyl)pyrrolidine-3-carboxamide |
| 130 | 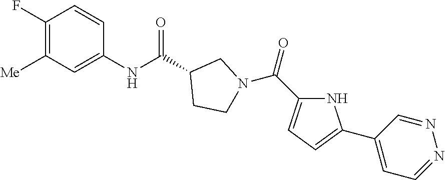 [SMILES: Cc1cc(NC(=O)[C@H]2CCN(C(=O)c3ccc(-c4ccnnc4)[nH]3)C2)ccc1F] |
| | (S)-N-(4-fluoro-3-methylphenyl)-1-(5-(pyridazin-4-yl)-1H-pyrrole-2- |
| | carbonyl)pyrrolidine-3-carboxamide |
| 131 | 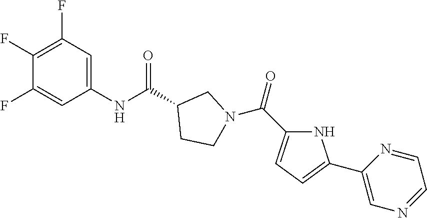 [SMILES: O=C(Nc1cc(F)c(F)c(F)c1)[C@H]1CCN(C(=O)c2ccc(-c3cnccn3)[nH]2)C1] |
| | (S)-1-(5-(pyrazin-2-yl)-1H-pyrrole-2-carbonyl)-N-(3,4,5- |
| | trifluorophenyl)pyrrolidine-3-carboxamide |
| 132 | 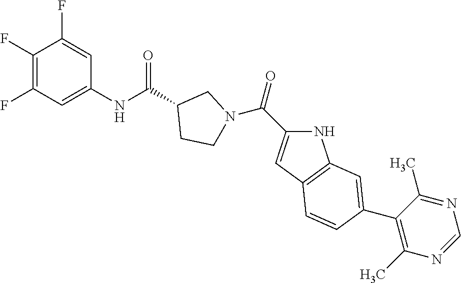 [SMILES: Cc1ncnc(C)c1-c1ccc2cc(C(=O)N3CC[C@H](C(=O)Nc4cc(F)c(F)c(F)c4)C3)[nH]c2c1] |
| | (S)-1-(6-(4,6-dimethylpyrimidin-5-yl)-1H-indole-2-carbonyl)-N-(3,4,5- |
| | trifluorophenyl)pyrrolidine-3-carboxamide |
| 133 | 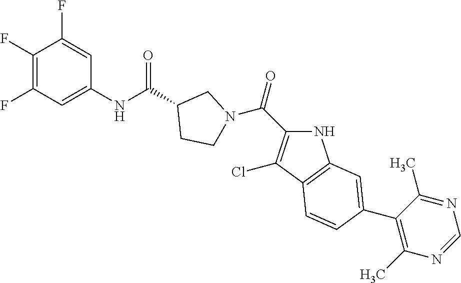 [SMILES: Cc1ncnc(C)c1-c1ccc2c(Cl)c(C(=O)N3CC[C@H](C(=O)Nc4cc(F)c(F)c(F)c4)C3)[nH]c2c1] |
| | (S)-1-(3-chloro-6-(4,6-dimethylpyrimidin-5-yl)-1H-indole-2-carbonyl)-N- |
| | (3,4,5-trifluorophenyl)pyrrolidine-3-carboxamide |
| 134 | 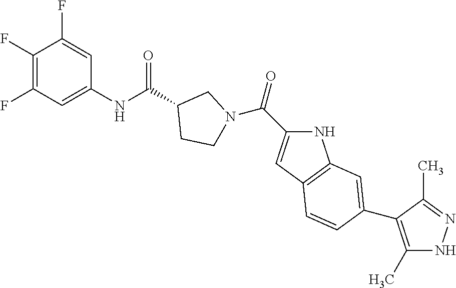 [SMILES: Cc1n[nH]c(C)c1-c1ccc2cc(C(=O)N3CC[C@H](C(=O)Nc4cc(F)c(F)c(F)c4)C3)[nH]c2c1] |
| | (S)-1-(6-(3,5-dimethyl-1H-pyrazol-4-yl)-1H-indole-2-carbonyl)-N-(3,4,5- |
| | trifluorophenyl)pyrrolidine-3-carboxamide |
| 135 | 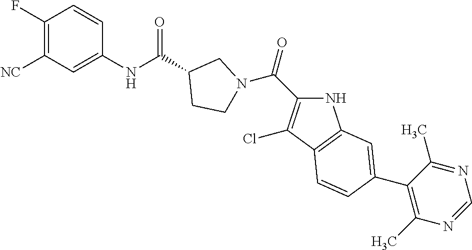 [SMILES: Cc1ncnc(C)c1-c1ccc2c(Cl)c(C(=O)N3CC[C@H](C(=O)Nc4ccc(F)c(C#N)c4)C3)[nH]c2c1] |
| | (S)-1-(3-chloro-6-(4,6-dimethylpyrimidin-5-yl)-1H-indole-2-carbonyl)-N- |
| | (3-cyano-4-fluorophenyl)pyrrolidine-3-carboxamide |
| 136 | 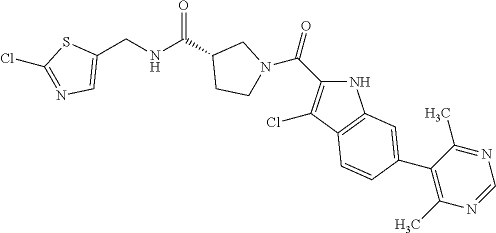 [SMILES: Cc1ncnc(C)c1-c1ccc2c(Cl)c(C(=O)N3CC[C@H](C(=O)NCc4cnc(Cl)s4)C3)[nH]c2c1] |
| | (S)-1-(3-chloro-6-(4,6-dimethylpyrimidin-5-yl)-1H-indole-2-carbonyl)-N- |
| | ((2-chlorothiazol-5-yl)methyl)pyrrolidine-3-carboxamide |
| 137 | 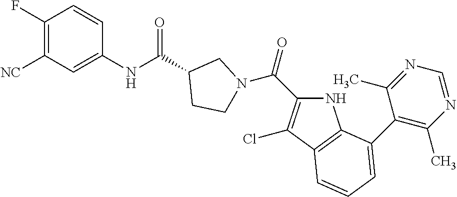 [SMILES: Cc1ncnc(C)c1-c1cccc2c(Cl)c(C(=O)N3CC[C@H](C(=O)Nc4ccc(F)c(C#N)c4)C3)[nH]c12] |
| | (S)-1-(3-chloro-7-(4,6-dimethylpyrimidin-5-yl)-1H-indole-2-carbonyl)-N- |
| | (3-cyano-4-fluorophenyl)pyrrolidine-3-carboxamide |
| 138 | 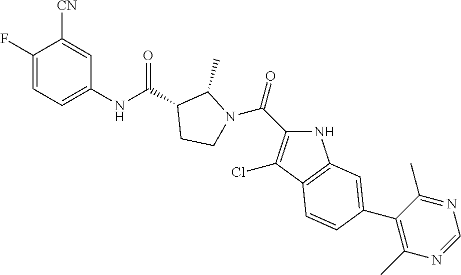 [SMILES: Cc1ncnc(C)c1-c1ccc2c(Cl)c(C(=O)N3CC[C@H](C(=O)Nc4ccc(F)c(C#N)c4)[C@@H]3C)[nH]c2c1] |
| | (2S,3S)-1-(3-chloro-6-(4,6-dimethylpyrimidin-5-yl)-1H-indole-2- |
| | carbonyl)-N-(3-cyano-4-fluorophenyl)-2-methylpyrrolidine-3- |
| | carboxamide |
| 139 | 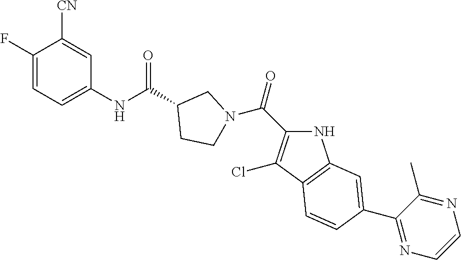 [SMILES: Cc1nccnc1-c1ccc2c(Cl)c(C(=O)N3CC[C@H](C(=O)Nc4ccc(F)c(C#N)c4)C3)[nH]c2c1] |
| | (S)-1-(3-chloro-6-(3-methylpyrazin-2-yl)-1H-indole-2-carbonyl)-N-(3- |
| | cyano-4-fluorophenyl)pyrrolidine-3-carboxamide |
| 140 | 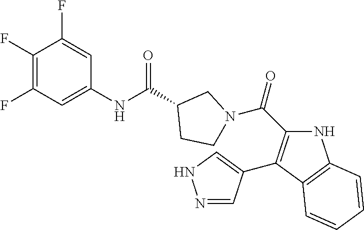 [SMILES: O=C(Nc1cc(F)c(F)c(F)c1)[C@H]1CCN(C(=O)c2[nH]c3ccccc3c2-c2cn[nH]c2)C1] |
| | (S)-1-(3-(1H-pyrazol-4-yl)-1H-indole-2-carbonyl)-N-(3,4,5- |
| | trifluorophenyl)pyrrolidine-3-carboxamide |
| 141 | 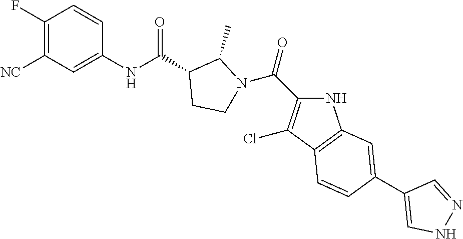 [SMILES: C[C@H]1[C@@H](C(=O)Nc2ccc(F)c(C#N)c2)CCN1C(=O)c1[nH]c2cc(-c3cn[nH]c3)ccc2c1Cl] |
| | (2S,3S)-1-(3-chloro-6-(1H-pyrazol-4-yl)-1H-indole-2-carbonyl)-N-(3- |
| | cyano-4-fluorophenyl)-2-methylpyrrolidine-3-carboxamide |
| 142 | 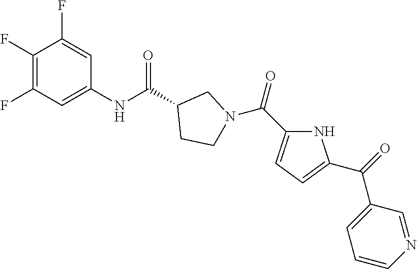 [SMILES: O=C(c1cccnc1)c1ccc(C(=O)N2CC[C@H](C(=O)Nc3cc(F)c(F)c(F)c3)C2)[nH]1] |
| | (S)-1-(5-nicotinoyl-1H-pyrrole-2-carbonyl)-N-(3,4,5- |
| | trifluorophenyl)pyrrolidine-3-carboxamide |
| 143 | 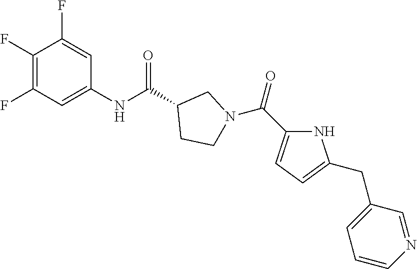 [SMILES: O=C(Nc1cc(F)c(F)c(F)c1)[C@H]1CCN(C(=O)c2ccc(Cc3cccnc3)[nH]2)C1] |
| | (S)-1-(5-(pyridine-3-ylmethyl)-1H-pyrrole-2-carbonyl)-N-(3,4,5- |
| | trifluorophenyl)pyrrolidine-3-carboxamide |
| 144 | 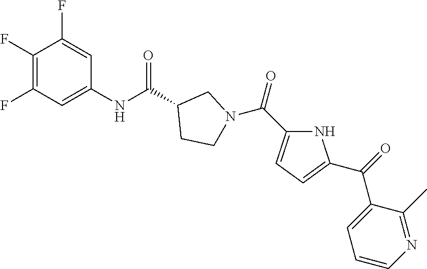 [SMILES: Cc1ncccc1C(=O)c1ccc(C(=O)N2CC[C@H](C(=O)Nc3cc(F)c(F)c(F)c3)C2)[nH]1] |
| | (S)-1-(5-(2-methylnicotinoyl)-1H-pyrrole-2-carbonyl)-N-(3,4,5- |
| | trifluorophenyl)pyrrolidine-3-carboxamide |
| 145 | 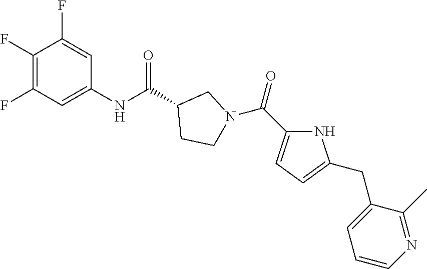 [SMILES: Cc1ncccc1Cc1ccc(C(=O)N2CC[C@H](C(=O)Nc3cc(F)c(F)c(F)c3)C2)[nH]1] |
| | (S)-1-(5-((2-methylpyridin-3-yl)methyl)-1H-pyrrole-2-carbonyl)-N- |
| | (3,4,5-trifluorophenyl)pyrrolidin-3-carboxamide |
| 146 | 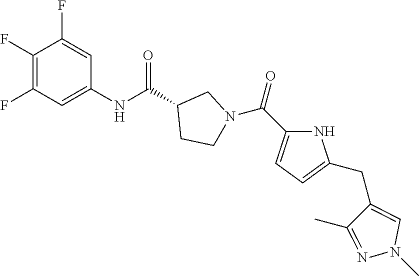 [SMILES: Cc1nn(C)cc1Cc1ccc(C(=O)N2CC[C@H](C(=O)Nc3cc(F)c(F)c(F)c3)C2)[nH]1] |
| | (S)-1-(5-((1,3-dimethyl-1H-pyrazol-4-yl)methyl)-1H-pyrrole-2-carbonyl)- |
| | N-(3,4,5-trifluorophenyl)pyrrolidine-3-carboxamide |
| 147 | 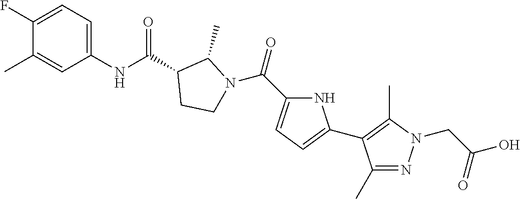 [SMILES: Cc1cc(NC(=O)[C@H]2CCN(C(=O)c3ccc(-c4c(C)nn(CC(=O)O)c4C)[nH]3)[C@H]2C)ccc1F] |
| | 2))-5)-4)-2S,3S)--4))-3fluoro--3methylphenyl)carbamoyl)--2 |
| | methylpyrrolidine--1carbonyl)-1H-pyrrol--2yl)-3,-5dimethyl-1H-pyrazol-- |
| | 1yl)acetic acid |
| 148 | 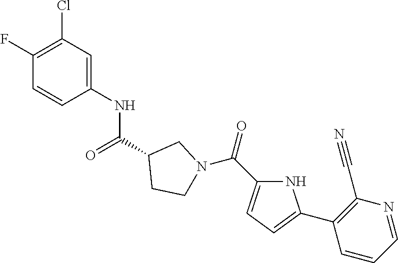 [SMILES: N#Cc1ncccc1-c1ccc(C(=O)N2CC[C@H](C(=O)Nc3ccc(F)c(Cl)c3)C2)[nH]1] |
| | (S)-N-(-3chloro--4fluorophenyl)--2)-5)-1cyanopyridin--3yl)-1H-pyrrole-- |
| | 2carbonyl)pyrrolidine--3carboxamide |
| 149 | 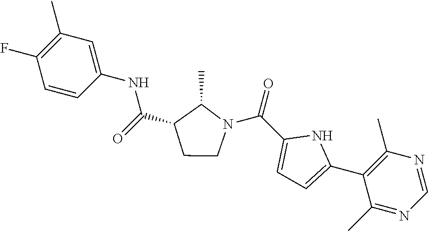 [SMILES: Cc1cc(NC(=O)[C@H]2CCN(C(=O)c3ccc(-c4c(C)ncnc4C)[nH]3)[C@H]2C)ccc1F] |
| | (2S,3S)-4)-5)-1,-6dimethylpyrimidin--5yl)-1H-pyrrole--2carbonyl)-N-(-4 |
| | fluoro--3methylphenyl)--2methylpyrrolidine--3carboxamide |
| 150 | 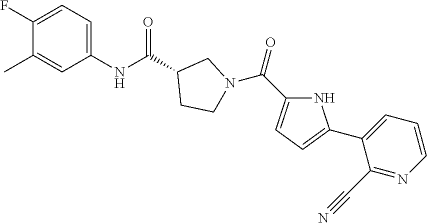 [SMILES: Cc1cc(NC(=O)[C@H]2CCN(C(=O)c3ccc(-c4cccnc4C#N)[nH]3)C2)ccc1F] |
| | (S)--2)-5)-1cyanopyridin--3yl)-1H-pyrrole--2carbonyl)-N-(-4fluoro--3 |
| | methylphenyl)pyrrolidine--3carboxamide |
| 151 | 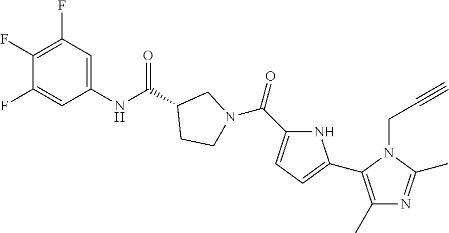 [SMILES: C#CCn1c(C)nc(C)c1-c1ccc(C(=O)N2CC[C@H](C(=O)Nc3cc(F)c(F)c(F)c3)C2)[nH]1] |
| | (S)-1-(5-(2,4-dimethyl-1-(prop-2-yn-1-yl)-1H-imidazol-5-yl)-1H-pyrrole- |
| | 2-carbonyl)-N-(3,4,5-trifluorophenyl)pyrrolidine-3-carboxamide |
| 152 | 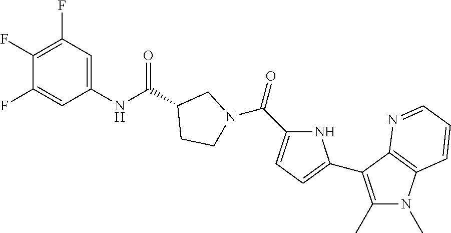 [SMILES: Cc1c(-c2ccc(C(=O)N3CC[C@H](C(=O)Nc4cc(F)c(F)c(F)c4)C3)[nH]2)c2ncccc2n1C] |
| | (S)-1-(5-(1,2-dimethyl-1H-pyrrolo[3,2-b]pyridin-3-yl)-1H-pyrrole-2- |
| | carbonyl)-N-(3,4,5-trifluorophenyl)pyrrolidine-3-carboxamide |
| 153 | 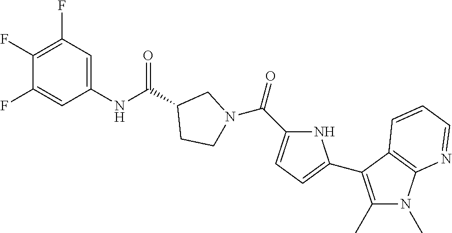 [SMILES: Cc1c(-c2ccc(C(=O)N3CC[C@H](C(=O)Nc4cc(F)c(F)c(F)c4)C3)[nH]2)c2cccnc2n1C] |
| | (S)-1-(5-(1,2-dimethyl-1H-pyrrolo[2,3-b]pyridin-3-yl)-1H-pyrrole-2- |
| | carbonyl)-N-(3,4,5-trifluorophenyl)pyrrolidine-3-carboxamide |
| 154 | 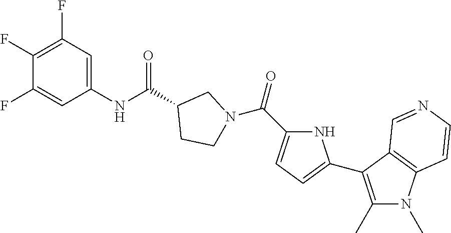 [SMILES: Cc1c(-c2ccc(C(=O)N3CC[C@H](C(=O)Nc4cc(F)c(F)c(F)c4)C3)[nH]2)c2cnccc2n1C] |
| | (S)-1-(5-(1,2-dimethyl-1H-pyrrolo[3,2-c]pyridin-3-yl)-1H-pyrrole-2- |
| | carbonyl)-N-(3,4,5-trifluorophenyl)pyrrolidine-3-carboxamide |
| 155 | 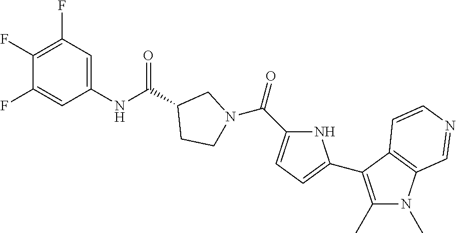 [SMILES: Cc1c(-c2ccc(C(=O)N3CC[C@H](C(=O)Nc4cc(F)c(F)c(F)c4)C3)[nH]2)c2ccncc2n1C] |
| | (S)-1-(5-(1,2-dimethyl-1H-pyrrolo[2,3-c]pyridin-3-yl)-1H-pyrrole-2- |
| | carbonyl)-N-(3,4,5-trifluorophenyl)pyrrolidine-3-carboxamide |
| 156 | 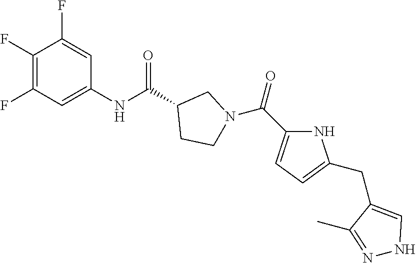 [SMILES: Cc1n[nH]cc1Cc1ccc(C(=O)N2CC[C@H](C(=O)Nc3cc(F)c(F)c(F)c3)C2)[nH]1] |
| | (S)-1-(5-((3-methyl-1H-pyrazol-4-yl)methyl)-1H-pyrrole-2-carbonyl)-N- |
| | (3,4,5-trifluorophenyl)pyrrolidine-3-carboxamide |
| 157 | 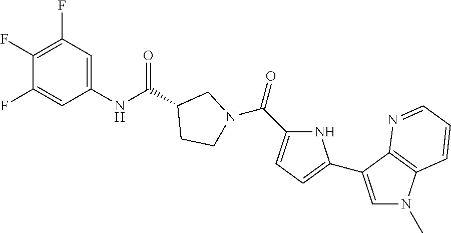 [SMILES: Cn1cc(-c2ccc(C(=O)N3CC[C@H](C(=O)Nc4cc(F)c(F)c(F)c4)C3)[nH]2)c2ncccc21] |
| | (S)-1-(5-(1-methyl-1H-pyrrolo[3,2-b]pyridin-3-yl)-1H-pyrrole-2- |
| | carbonyl)-N-(3,4,5-trifluorophenyl)pyrrolidine-3-carboxamide |
| 158 | 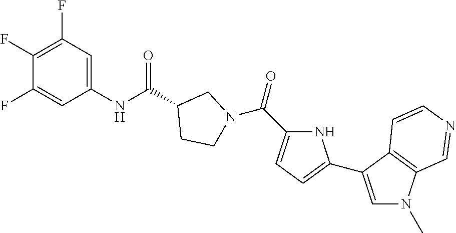 [SMILES: Cn1cc(-c2ccc(C(=O)N3CC[C@H](C(=O)Nc4cc(F)c(F)c(F)c4)C3)[nH]2)c2ccncc21] |
| | (S)-1-(5-(1-methyl-1H-pyrrolo[2,3-c]pyridin-3-yl)-1H-pyrrole-2- |
| | carbonyl)-N-(3,4,5-trifluorophenyl)pyrrolidine-3-carboxamide |
| 159 | 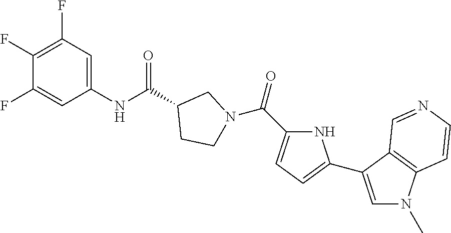 [SMILES: Cn1cc(-c2ccc(C(=O)N3CC[C@H](C(=O)Nc4cc(F)c(F)c(F)c4)C3)[nH]2)c2cnccc21] |
| | (S)-1-(5-(1-methyl-1H-pyrrolo[3,2-c]pyridin-3-yl)-1H-pyrrole-2- |
| | carbonyl)-N-(3,4,5-trifluorophenyl)pyrrolidine-3-carboxamide |
| 160 | 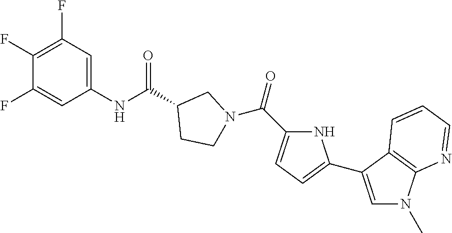 [SMILES: Cn1cc(-c2ccc(C(=O)N3CC[C@H](C(=O)Nc4cc(F)c(F)c(F)c4)C3)[nH]2)c2cccnc21] |
| | (S)-1-(5-(1-methyl-1H-pyrrolo[2,3-b]pyridin-3-yl)-1H-pyrrole-2- |
| | carbonyl)-N-(3,4,5-trifluorophenyl)pyrrolidine-3-carboxamide |
| 161 | 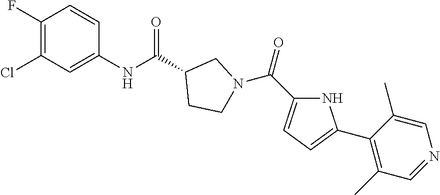 [SMILES: Cc1cncc(C)c1-c1ccc(C(=O)N2CC[C@H](C(=O)Nc3ccc(F)c(Cl)c3)C2)[nH]1] |
| | (S)-N-(3-chloro-4-fluorophenyl)-1-(5-(3,5-dimethylpyridin-4-yl)-1H- |
| | pyrrole-2-carbonyl)pyrrolidine-3-carboxamide |
| 162 | 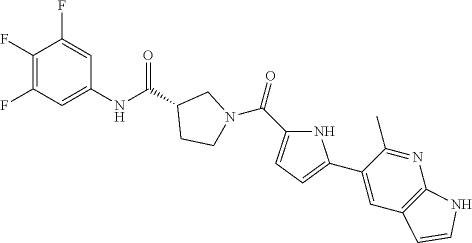 [SMILES: Cc1nc2[nH]ccc2cc1-c1ccc(C(=O)N2CC[C@H](C(=O)Nc3cc(F)c(F)c(F)c3)C2)[nH]1] |
| | (S)-1-(5-(6-methyl-1H-pyrrolo[2,3-b]pyridin-5-yl)-1H-pyrrole-2- |
| | carbonyl)-N-(3,4,5-trifluorophenyl)pyrrolidine-3-carboxamide |
| 163 | 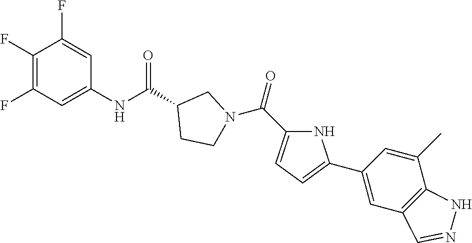 [SMILES: Cc1cc(-c2ccc(C(=O)N3CC[C@H](C(=O)Nc4cc(F)c(F)c(F)c4)C3)[nH]2)cc2cn[nH]c12] |
| | (S)-1-(5-(7-methyl-1H-indazol-5-yl)-1H-pyrrole-2-carbonyl)-N-(3,4,5- |
| | trifluorophenyl)pyrrolidine-3-carboxamide |
| 164 | 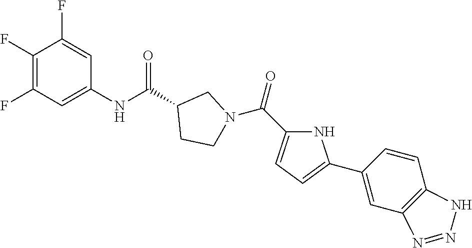 [SMILES: O=C(Nc1cc(F)c(F)c(F)c1)[C@H]1CCN(C(=O)c2ccc(-c3ccc4[nH]nnc4c3)[nH]2)C1] |
| | (S)-1-(5-(1H-benzo[d][1,2,3]triazol-5-yl)-1H-pyrrole-2-carbonyl)-N- |
| | (3,4,5-trifluorophenyl)pyrrolidine-3-carboxamide |
| 165 | 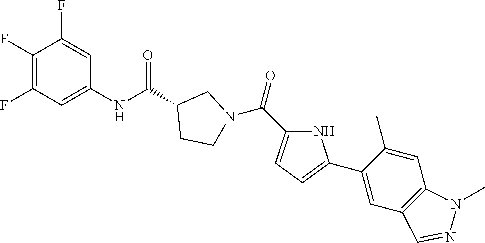 [SMILES: Cc1cc2c(cnn2C)cc1-c1ccc(C(=O)N2CC[C@H](C(=O)Nc3cc(F)c(F)c(F)c3)C2)[nH]1] |
| | (S)-1-(5-(1,6-dimethyl-1H-indazol-5-yl)-1H-pyrrole-2-carbonyl)-N- |
| | (3,4,5-trifluorophenyl)pyrrolidine-3-carboxamide |
| 166 | 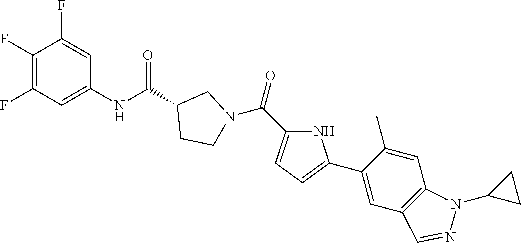 [SMILES: Cc1cc2c(cnn2C2CC2)cc1-c1ccc(C(=O)N2CC[C@H](C(=O)Nc3cc(F)c(F)c(F)c3)C2)[nH]1] |
| | (S)-1-(5-(1-cyclopropyl-6-methyl-1H-indazol-5-yl)-1H-pyrrole-2- |
| | carbonyl)-N-(3,4,5-trifluorophenyl)pyrrolidine-3-carboxamide |
| 167 | 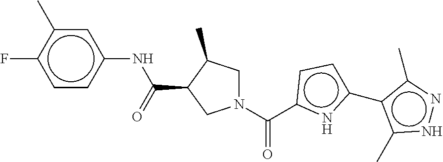 |
| | (3S,4R)-1-(5-(3,5-dimethyl-1H-pyrazol-4-yl)-1H-pyrrole-2-carbonyl)-N- |
| | (4-fluoro-3-methylphenyl)-4-methylpyrrolidine-3-carboxamide |
| 168 | 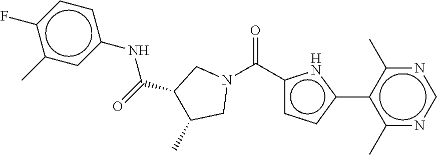 |
| | (3S,4R)-1-(5-(4,6-dimethylpyrimidin-5-yl)-1H-pyrrole-2-carbonyl)-N-(4- |
| | fluoro-3-methylphenyl)-4-methylpyrrolidine-3-carboxamide |
| 169 | 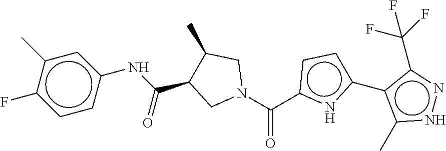 |
| | (3S,4R)-N-(4-fluoro-3-methylphenyl)-4-methyl-1-(5-(5-methyl-3- |
| | (trifluoromethyl)-1H-pyrazol-4-yl)-1H-pyrrole-2-carbonyl)pyrrolidine-3- |
| | carboxamide |
| 170 | 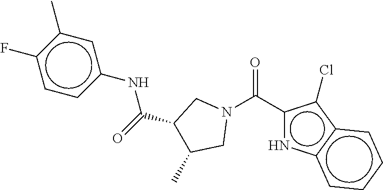 |
| | (3S,4R)-1-(3-chloro-1H-indole-2-carbonyl)-N-(4-fluoro-3-methylphenyl)- |
| | 4-methylpyrrolidine-3-carboxamide |
| 171 | 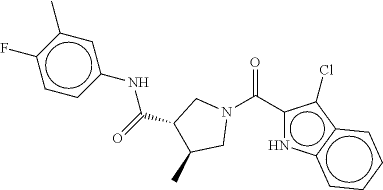 |
| | (3S,4S)-1-(3-chloro-1H-indole-2-carbonyl)-N-(4-fluoro-3-methylphenyl)- |
| | 4-methylpyrrolidine-3-carboxamide |
| 172 | 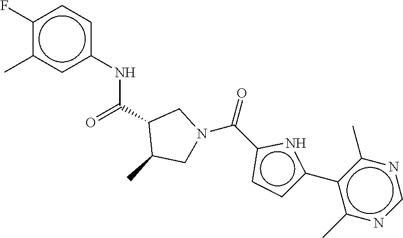 |
| | (3S,4S)-1-(5-(4,6-dimethylpyrimidin-5-yl)-1H-pyrrole-2-carbonyl)-N-(4- |
| | fluoro-3-methylphenyl)-4-methylpyrrolidine-3-carboxamide |
| 173 | 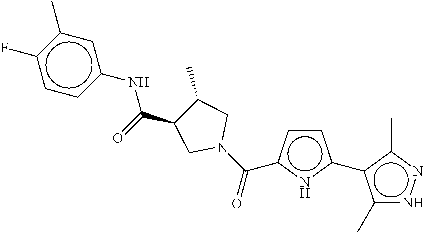 |
| | (3S,4S)-1-(5-(3,5-dimethyl-1H-pyrazol-4-yl)-1H-pyrrole-2-carbonyl)-N- |
| | (4-fluoro-3-methylphenyl)-4-methylpyrrolidine-3-carboxamide |
| 174 | 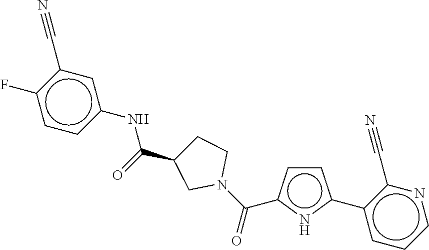 |
| | (S)-N-(3-cyano-4-fluorophenyl)-1-(5-(2-cyanopyridin-3-yl)-1H-pyrrole-2- |
| | carbonyl)pyrrolidine-3-carboxamide |
| 175 | 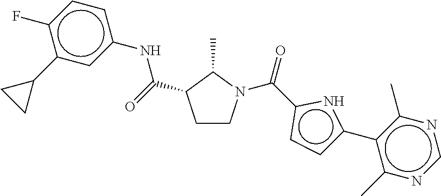 |
| | (2S,3S)-N-(3-cyclopropyl-4-fluorophenyl)-1-(5-(4,6-dimethylpyrimidin- |
| | 5-yl)-1H-pyrrole-2-carbonyl)-2-methylpyrrolidine-3-carboxamide |

In some embodiments, the compound is a pharmaceutically acceptable salt of any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) or Formula (I′) is isotopically enriched with regard to, or labelled with, one or more isotopes as compared to the corresponding compound of Formula (I) or Formula (I′). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with ²H with regard to one or more atoms thereof). In some embodiments, the compound is a ¹⁸F labeled compound. In some embodiments, the compound is a ¹²³I labeled compound, a ¹²⁴I labeled compound, a ¹²⁵I labeled compound, a ¹²⁹I labeled compound, a ¹³¹I labeled compound, a ³⁵¹I labeled compound, or any combination thereof. In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³²S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the invention or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³²S, ³⁴S, ³⁵S, and ³⁶S atom(s) is within the scope of the invention. Further, substitution with isotope (e.g., ¹⁸F, ¹²³I, ¹²⁴I, ¹²³I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

The various functional groups and substituents making up the compounds of the Formula (I) or Formula (I′) are typically chosen such that the molecular weight of the compound does not exceed 1000 daltons. More usually, the molecular weight of the compound will be less than 900, for example less than 800, or less than 750, or less than 700, or less than 650 daltons. More conveniently, the molecular weight is less than 600 and, for example, is 550 daltons or less.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterised by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess HBV replication cycle modulatory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

The compound of any one of the Formulae described herein may be protonated at a physiological pH. Thus, a compound may have a positive or partial positive charge at physiological pH. Such compounds may be referred to as cationic or ionizable compounds. The compound of any one of the Formulae described herein may also be zwitterionic, i.e., neutral molecules having both a positive and a negative charge.

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichloroetric or non-stoichloroetric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess HBV replication cycle modulatory activity.

It is also to be understood that certain compounds of any one of the Formulae disclosed herein may exhibit polymorphism, and that the disclosure encompasses all such forms, or mixtures thereof, which possess HBV replication cycle modulatory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) or Formula (I′) include all such forms. For the avoidance of doubt, where a compound can exist in one of several tautomeric forms, and only one is specifically described or shown, all others are nevertheless embraced by Formula (I) or Formula (I′). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

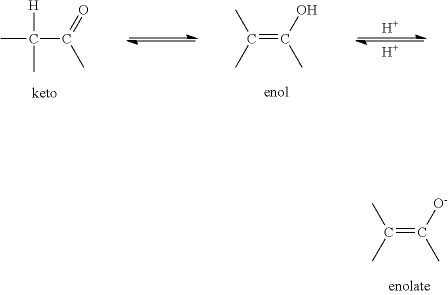

Compounds of any one of the Formulae disclosed herein containing an amine function may also form N-oxides. A reference herein to a compound of Formula (I) or Formula (I′) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidizing agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically-produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆, alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁-4 alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

Suitably, the present disclosure excludes any individual compounds not possessing the biological activity defined herein.

### Methods of Synthesis

In some aspects, the present disclosure provides a method of preparing a compound of the present disclosure.

In some aspects, the present disclosure provides a method of a compound, comprising one or more steps as described herein.

In some aspects, the present disclosure provides a compound obtainable by, or obtained by, or directly obtained by a method for preparing a compound as described herein.

In some aspects, the present disclosure provides an intermediate as described herein, being suitable for use in a method for preparing a compound as described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of Formula (I) or Formula (I′) has been synthesized by any one of the processes defined herein, the processes may then further comprise the additional steps of; (i) removing any protecting groups present; (ii) converting the compound Formula (I) or Formula (I′) into another compound of Formula (I) or Formula (I′); (iii) forming a pharmaceutically acceptable salt, hydrate or solvate thereof; and/or (iv) forming a prodrug thereof.

The resultant compounds of Formula (I) or Formula (I′) can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilising the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognise which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes 1-4 herein.

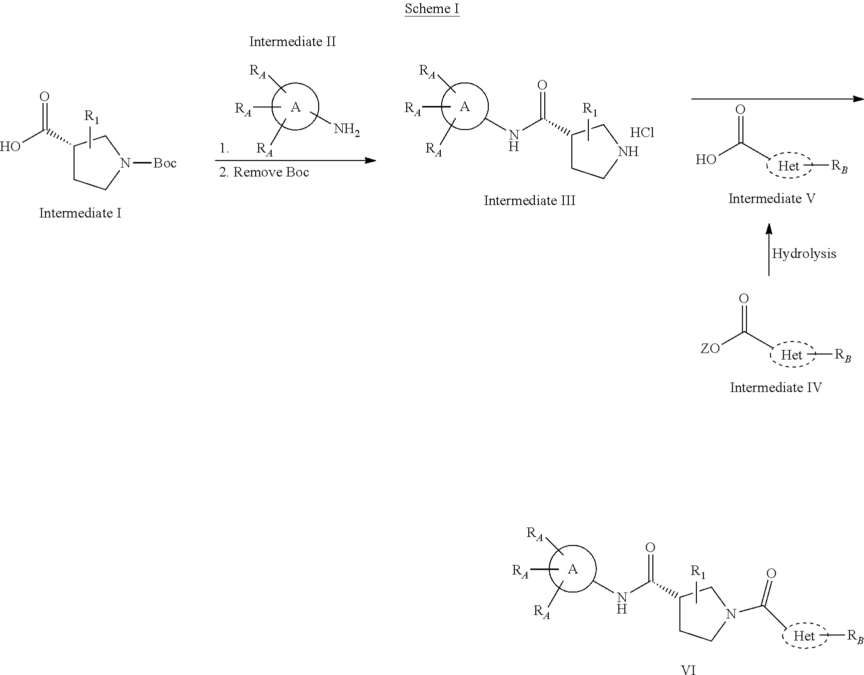

A synthesis of compounds of Formula VI is described in Scheme I. A N-Boc-protected aminocarboxylic acid of Intermediate I is coupled with an amine, Intermediate II, in the presence of an amide coupling reagent (e.g., HATU and an organic base like DIPEA) in an aprotic solvent (e.g. dichloromethane, DMF), resulting in an amide adduct, followed by deprotection of the N-Boc using 4M HCl/dioxane, resulting in a compound of Intermediate III. The heterocyclic carboxylic acid, Intermediate V, can be converted to an amide by use of a coupling agent (e.g. HATU) in an aprotic solvent (e.g. dichloromethane, DMF), along with an organic base (e.g. DIPEA), and Intermediate III resulting in a compound of formula VI. Intermediate IV may be hydrolyzed to carboxylic acid, Intermediate V, by known methods (e.g. addition of an aqueous base).

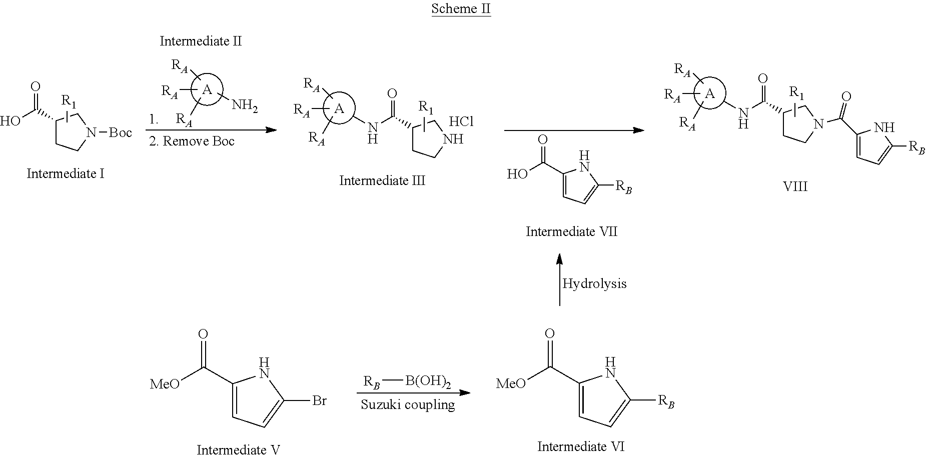

A synthesis of compounds of Formula VIII is described in Scheme II. To obtain Intermediate III, the synthesis was followed as described previously. Methyl 5-bromo-1H-pyrrole-2-carboxylate (IV) underwent Suzuki cross-coupling with a boronic acid derivative (R_{B}—B(OH)₂), palladium catalyst (e.g. Pd(PPh₃)₄), and inorganic base (e.g. Li₂CO₃, Na₂CO₃, K₂CO₃) in organic solvent (e.g. DMF, 1,4-dioxane) under inert gas and heat to yield a compound of Intermediate VI. Then, Intermediate VI was hydrolyzed in aqueous metal hydroxide solution (e.g. LiOH, NaOH). Substituted pyrrole of Intermediate VII was converted to an amide by use of a coupling agent (e.g. HATU) in an aprotic solvent (e.g. dichloromethane, DMF), along with an organic base (e.g. DIPEA), and Intermediate III resulting in a compound of Formula VIII.

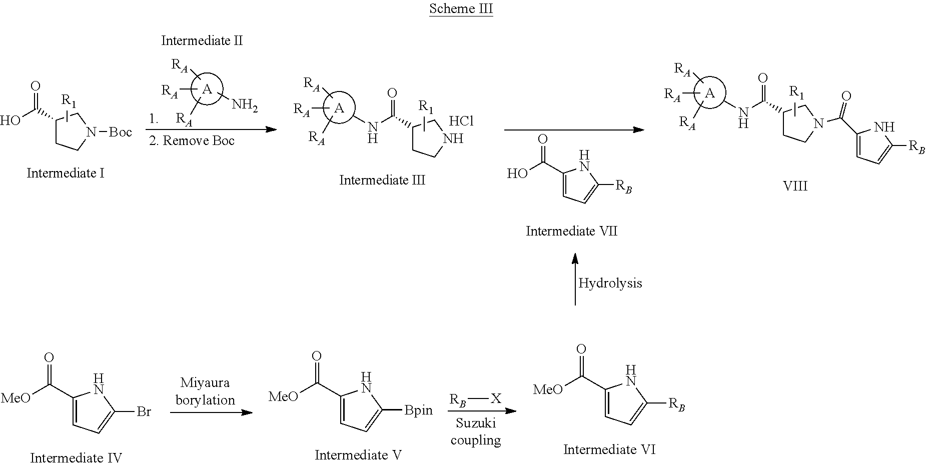

A synthesis of compounds of Formula VIII is described in Scheme III. To obtain compounds of Intermediate III, the synthesis was followed as described previously. Methyl 5-bromo-1H-pyrrole-2-carboxylate, Intermediate IV, was converted into Intermediate V by Miyaura reaction. Intermediate IV was heated with Bis(pinacolato)diboron, in the presence of palladium catalyst (eq. Pd(dppf)Cl₂), inorganic base (e.g. KOAc), and organic solvent (e.g. DMF, 1,4-dioxane). Then Intermediate V was heated with a halide derivatives (e.g., R_{B}X), palladium catalyst (e.g. Pd(PPh₃)₄), and inorganic base (e.g. Li₂CO₃, Na₂CO₃, K₂CO₃) in organic solvent (e.g. DMF, 1,4-dioxane) under inert gas to undergo Suzuki cross coupling and produce Intermediate VT. Intermediate VI was hydrolyzed in aqueous metal hydroxide solution (e.g. LiOH, NaOH) to form Intermediate VII. Intermediate VII was converted to an amide functional group by use of a coupling agent (e.g. HATU) in an aprotic solvent (e.g. dichloromethane, DMF), along with an organic base (e.g. DIPEA), and amines (III) resulting in a compounds of Formula VIII.

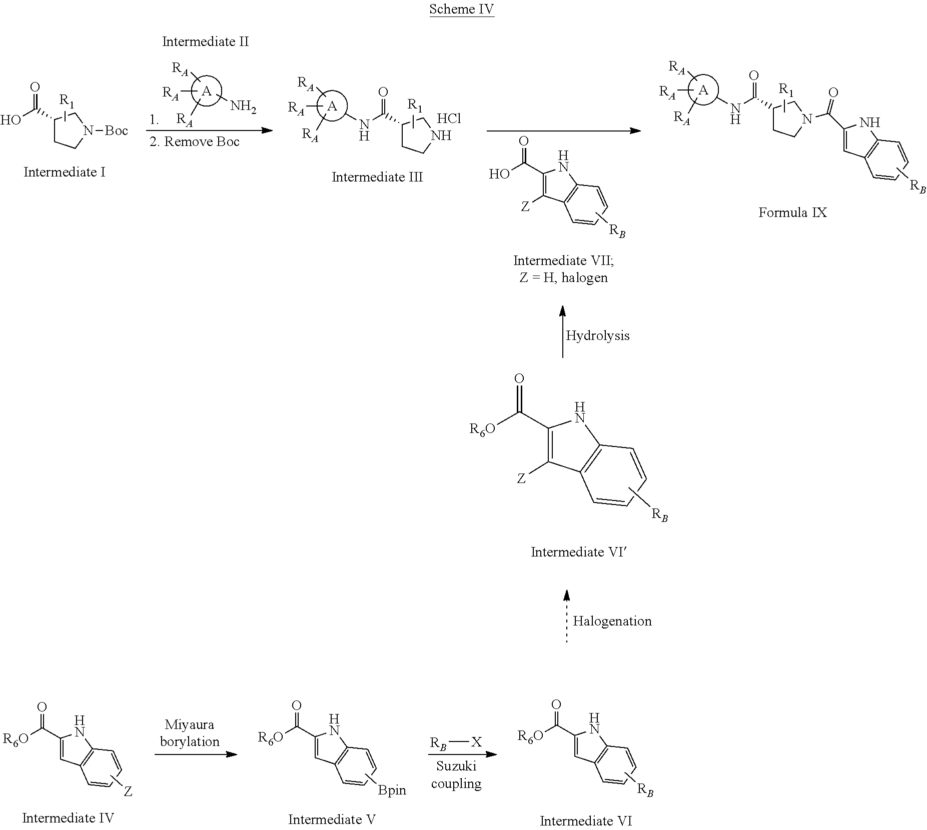

A synthesis of compounds of Formula IX is described in Scheme IV. To obtain compounds of Intermediate III, the synthesis was followed as described previously. Then, to afford Intermediate VII, Intermediate IV was converted into Intermediate V by Miyaura reaction. Intermediate IV was heated with Bis(pinacolato)diboron, in the presence of palladium catalyst (e.g. Pd(dppf)Cl₂), inorganic base (e.g. KOAc), and organic solvent (e.g. DMF, 1,4-dioxane). Then, Intermediate V was heated with a halide derivative (e.g., R_{B}X), palladium catalyst (e.g. Pd(PPh₃)₄), and inorganic base (e.g. Li₂CO₃, Na₂CO₃, K₂CO₃) in organic solvent (e.g. DMF, 1,4-dioxane) under inert gas to undergo Suzuki cross coupling [symbol] obtain Intermediate VI. Intermediate VI was halogenated (e.g., chlorinated) by treating N-chlorosuccinimide in DCM at room temperature for two days and hydrolyzed in aqueous metal hydroxide solution (e.g. LiOH, NaOH). Intermediate VII was converted Formula IX by using a coupling agent (e.g. HATU) in an aprotic solvent (e.g. dichloromethane, DMF), along with an organic base (e.g. DIPEA), and Intermediate III resulting in a compound of Formula IX.

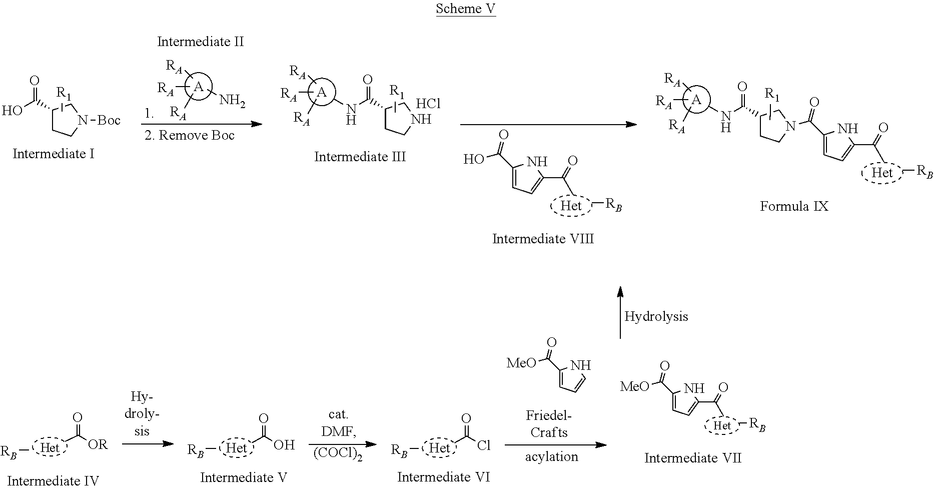

A synthesis of compounds of Formula IX is described in Scheme V. To obtain compounds of Intermediate III, the synthesis was followed as described previously. Then to achieve compounds of Intermediate VIII, Intermediate IV was hydrolyzed in aqueous metal hydroxide solution (e.g. LiOH, NaOH). Then, the Intermediate V was converted into Intermediate VI by using oxalyl chloride in the presence of DMF as a catalyst. Intermediate VI was reacted with methyl 1H-pyrrole-2-carboxylate via Friedel-Crafts acylation to obtain Intermediate VII. Intermediate VII was hydrolyzed to obtain Intermediate VIII by using aqueous metal hydroxide solution (e.g. LiOH, NaOH). Intermediate VIII was mixed to a coupling agent (e.g. HATU) in an aprotic solvent (e.g. dichloromethane, DMF), along with an organic base (e.g. DIPEA), and Intermediate III resulting in compounds of Formula IX.

### Biological Assays

The HBV is an enveloped, partially double-stranded DNA (dsDNA) virus of the hepadnavirus family (Hepadnaviridae). HBV capsid protein (HBV-CP) plays essential roles in HBV replication. The predominant biological function of HBV-CP is to act as a structural protein encapsidate pre-genomic RNA and form immature capsid particles, which spontaneously self-assemble from many copies of capsid protein dimers in the cytoplasm.

HBV-CP also regulates viral DNA synthesis through differential phosphorylation states of its C-terminal phosphorylation sites. Also, HBV-CP might facilitate the nuclear translocation of viral relaxed circular genome by means of the nuclear localization signals located in the arginine-rich domain of the C-terminal region of HBV-CP.

In the nucleus, as a component of the viral cccDNA mini-chromosome, HBV-CP could play a structural and regulatory role in the functionality of cccDNA mini-chromosomes. HBV-CP also interacts with viral large envelope protein in the endoplasmic reticulum (ER), and triggers the release of intact viral particles from hepatocytes.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays are may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

Stable transfected HBV cell line may be used for drug screening. To identify the anti-HBV activity, the cells are seeded with high glucose medium (DMEM) containing fetal bovine serum (FBS), G418, and non-essential amino acids (NEAA). The cells are allowed to stand overnight in an incubator. The cells may then be treated with the medium containing a compound for a length of time (e.g., three days) in the incubator. Next, the medium can be removed and fresh medium containing the compound was added to the cells for another length of time (e.g., three days). After treatment, intracellular HBV DNA may be extracted and determined by using quantitative real-time PCR (qPCR) technique with specific primers. Intrahepatic HBV DNA viral load may be reported as percentage compared to vehicle control (e.g., 0.5% DMSO).

To determine the effect on the levels of HBV DNA, HBV cells may be treated with compounds, at various concentrations in a medium. Following treatment for a period of time (e.g., three days), the medium may be removed and fresh medium containing the compound can be added to the cells for another period of times (e.g., three days). After treatment, intrahepatic HBV DNA from cell lysates may be isolated. Then qPCR reaction of DNA can be performed to measure total HBV DNA levels using specific primer set. The fifty-percent effective concentrations (EC₅₀) for HBV DNA inhibition, relative to no drug controls, can be determined using nonlinear fitting curve model.

To determine the cytotoxicity, HepG2.2.15 cells may be seeded onto a culture plate and allowed to adhere for a set amount of time (e.g., 24 hours). The cells may be treated with various concentrations of the compounds. After treatment, the culture media may be discarded and further incubated with serum-free media for a set amount of time (e.g., 2 hour). The resulting formazan crystals may be completely dissolved (e.g., in dimethyl sulfoxide), and then measured absorbance (e.g., at 570 nm) using a microplate reader. The concentrations of compounds producing 509% cell death (CC₅₀) may be determined from a fitting of concentration-response curve (% cell viability versus concentration) to a four-parameter equation.

Hepatitis B virus (HBV) capsid is a step in virus propagation and is mediated by the core protein. HBV core C-terminally truncated protein (HBV Cp149) may assemble into a capsid. Capsid formation may be analyzed for the instant compounds as compared to a class I or class U compound. A class I compound induces the formation of morphologically aberrant empty structures (e.g., BAY 41-4109) and a class U compound induces the formation of intact empty capsids (e.g., NVR 3-778).

HBV C149 protein may be prepared based on a published method [Zlotnick, A et al; Nat Protoc 2007, 2(3), 490-498] with slight modifications. A compound may be incubated with HBV core protein in buffer, followed by incubation. After incubation, the reaction mixture may be analyzed by SEC using a size exclusion column with a running buffer. The UV absorbance may be monitored (e.g., at 280 nm). Compound-induced capsid assembly can be determined from the ratio of the area under the curve of the Cp149 dimer to that of the capsid fraction.

For EM study, the reaction mixture may be negatively stained with 1% uranyl acetate and visualized on an electron microscope.

In some embodiments, a compound of the instant disclosure induces capsid assembly.

In some embodiments, the biological assay is described in the Examples herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-O-cyclodextrin, triacetyl-β-cyclodextrin, peracetylated-β-cyclodextrin, carboxymethyl-β-cyclodextrin, hydroxyethyl-β-cyclodextrin, 2-hydroxy-β-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an HBV replication cycle related condition referred to herein, slow its progression and/or reduce the symptoms associated with the condition.

An effective amount of a compound of the present disclosure for use in therapy is an amount sufficient to treat an HBV replication cycle related condition referred to herein, slow its progression and/or reduce the symptoms associated with the condition.

The size of the dose for therapeutic or prophylactic purposes of a compound of Formula (I) or Formula (I′) will naturally vary according to the nature and severity of the conditions, the age and sex of the animal or patient and the route of administration, according to well-known principles of medicine.

### Methods of Use

In some aspects, the present disclosure provides a method of modulating the HBV replication cycle (e.g., in vitro or in vivo), comprising contacting a cell with an effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of curing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a viral infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a viral infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of curing a viral infection in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing hepatitis B virus in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating hepatitis B virus in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of curing hepatitis B virus in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in modulating the HBV replication cycle (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in curing a disease or disorder disclosed herein.

In some aspects, the present disclosure provides a compound of the present disclosure or a pharmaceutically acceptable salt thereof for use in treating or preventing a viral infection in a subject in need thereof.

In some aspects, the present disclosure provides a compound of the present disclosure or a pharmaceutically acceptable salt thereof for use in treating a viral infection in a subject in need thereof.

In some aspects, the present disclosure provides a compound of the present disclosure or a pharmaceutically acceptable salt thereof for use in curing a viral infection in a subject in need thereof.

In some aspects, the present disclosure provides a compound of the present disclosure or a pharmaceutically acceptable salt thereof for use in treating or preventing hepatitis B virus in a subject in need thereof.

In some aspects, the present disclosure provides a compound of the present disclosure or a pharmaceutically acceptable salt thereof for use in treating hepatitis B virus in a subject in need thereof.

In some aspects, the present disclosure provides a compound of the present disclosure or a pharmaceutically acceptable salt thereof for use in curing hepatitis B virus in a subject in need thereof.

In some aspects, the present disclosure provides use of a compound of the present disclosure or a pharmaceutically acceptable salt thereof in the manufacture of a medicament for modulating the HBV replication cycle (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for curing a disease or disorder disclosed herein.

In some aspects, the present disclosure provides use of a compound of the present disclosure or a pharmaceutically acceptable salt thereof in the manufacture of a medicament for treating or preventing a viral infection in a subject in need thereof.

In some aspects, the present disclosure provides use of a compound of the present disclosure or a pharmaceutically acceptable salt thereof in the manufacture of a medicament for treating a viral infection in a subject in need thereof.

In some aspects, the present disclosure provides use of a compound of the present disclosure or a pharmaceutically acceptable salt thereof in the manufacture of a medicament for curing a viral infection in a subject in need thereof.

In some aspects, the present disclosure provides use of a compound of the present disclosure or a pharmaceutically acceptable salt thereof in the manufacture of a medicament for treating or preventing hepatitis B virus in a subject in need thereof.

In some aspects, the present disclosure provides use of a compound of the present disclosure or a pharmaceutically acceptable salt thereof in the manufacture of a medicament for treating hepatitis B virus in a subject in need thereof.

In some aspects, the present disclosure provides use of a compound of the present disclosure or a pharmaceutically acceptable salt thereof in the manufacture of a medicament for curing hepatitis B virus in a subject in need thereof.

The present disclosure provides compounds that function as modulators of the HBV replication cycle.

In some embodiments, modulation is inhibition.

Effectiveness of compounds of the disclosure can be determined by industry-accepted assays/disease models according to standard practices of elucidating the same as described in the art and are found in the current general knowledge.

In some embodiments, the disease or disorder is a viral infection. In some embodiments, the viral infection is hepatitis B virus. In some embodiments, the viral infection is a Flaviviridae virus (e.g., West Nile virus, hepatitis C virus, Dengue Fever, or Zika virus).

### Routes of Administration

Compounds of the present disclosure, or pharmaceutically acceptable salts thereof, may be administered alone as a sole therapy or can be administered in addition with one or more other substances and/or treatments. Such conjoint treatment may be achieved by way of the simultaneous, sequential or separate administration of the individual components of the treatment.

For example, therapeutic effectiveness may be enhanced by administration of an adjuvant (i.e. by itself the adjuvant may only have minimal therapeutic benefit, but in combination with another therapeutic agent, the overall therapeutic benefit to the individual is enhanced). Alternatively, by way of example only, the benefit experienced by an individual may be increased by administering the compound of Formula (I) or Formula (I′) with another therapeutic agent (which also includes a therapeutic regimen) that also has therapeutic benefit.

In the instances where the compound of the present disclosure is administered in combination with other therapeutic agents, the compound of the disclosure need not be administered via the same route as other therapeutic agents, and may, because of different physical and chemical characteristics, be administered by a different route. For example, the compound of the disclosure may be administered orally to generate and maintain good blood levels thereof, while the other therapeutic agent may be administered intravenously. The initial administration may be made according to established protocols known in the art, and then, based upon the observed effects, the dosage, modes of administration and times of administration can be modified by the skilled clinician.

The particular choice of other therapeutic agent will depend upon the diagnosis of the attending physicians and their judgment of the condition of the individual and the appropriate treatment protocol. According to this aspect of the disclosure there is provided a combination for use in the treatment of a disease in which the HBV replication cycle is implicated comprising a compound of the disclosure as defined hereinbefore, or a pharmaceutically acceptable salt thereof, and another suitable agent.

According to a further aspect of the disclosure there is provided a pharmaceutical composition which comprises a compound of the disclosure, or a pharmaceutically acceptable salt thereof, in combination with a suitable, in association with a pharmaceutically acceptable diluent or carrier.

In addition to its use in therapeutic medicine, compounds of Formula (I) or Formula (I′) and pharmaceutically acceptable salts thereof are also useful as pharmacological tools in the development and standardization of in vitro and in vivo test systems for the evaluation of the effects of the treatment of hepatitis B virus in laboratory animals such as dogs, rabbits, monkeys, rats and mice, as part of the search for new therapeutic agents.

In any of the above-mentioned pharmaceutical composition, process, method, use, medicament, and manufacturing features of the instant disclosure, any of the alternate embodiments of macromolecules of the present disclosure described herein also apply.

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapy

In some embodiments, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In some embodiments, kits comprising a compound of the present disclosure, or a pharmaceutically acceptable salt thereof, in combination with one or more (e.g., one, two, three, four, one or two, one to three, or one to four) additional therapeutic agent are provided.

In some aspects, the present disclosure provides a method of treating or preventing hepatitis B virus in a subject in need thereof, comprising administering to the subject a therapeutically effective amount of a compound of the present disclosure or a pharmaceutically acceptable salt thereof, or a pharmaceutical composition of the present disclosure, in combination with one or more (e.g., one, two, three, four, one or two, one to three, or one to four) additional therapeutic agent.

In some aspects, the present disclosure provides a method of treating hepatitis B virus in a subject in need thereof, comprising administering to the subject a therapeutically effective amount of a compound of the present disclosure or a pharmaceutically acceptable salt thereof, or a pharmaceutical composition of the present disclosure, in combination with one or more (e.g., one, two, three, four, one or two, one to three, or one to four) additional therapeutic agent.

In some aspects, the present disclosure provides a method of curing hepatitis B virus in a subject in need thereof, comprising administering to the subject a therapeutically effective amount of a compound of the present disclosure or a pharmaceutically acceptable salt thereof, or a pharmaceutical composition of the present disclosure, in combination with one or more (e.g., one, two, three, four, one or two, one to three, or one to four) additional therapeutic agent.

In some aspects, the present disclosure provides a compound of the present disclosure or a pharmaceutically acceptable salt thereof for use in treating or preventing hepatitis B virus in a subject in need thereof, in combination with one or more (e.g., one, two, three, four, one or two, one to three, or one to four) additional therapeutic agent.

In some aspects, the present disclosure provides a compound of the present disclosure or a pharmaceutically acceptable salt thereof for use in treating hepatitis B virus in a subject in need thereof, in combination with one or more (e.g., one, two, three, four, one or two, one to three, or one to four) additional therapeutic agent.

In some aspects, the present disclosure provides a compound of the present disclosure or a pharmaceutically acceptable salt thereof for use in curing hepatitis B virus in a subject in need thereof, in combination with one or more (e.g., one, two, three, four, one or two, one to three, or one to four) additional therapeutic agent.

In some aspects, the present disclosure provides use of a compound of the present disclosure or a pharmaceutically acceptable salt thereof in the manufacture of a medicament for treating or preventing hepatitis B virus in a subject in need thereof, in combination with one or more (e.g., one, two, three, four, one or two, one to three, or one to four) additional therapeutic agent.

In some aspects, the present disclosure provides use of a compound of the present disclosure or a pharmaceutically acceptable salt thereof in the manufacture of a medicament for treating hepatitis B virus in a subject in need thereof, in combination with one or more (e.g., one, two, three, four, one or two, one to three, or one to four) additional therapeutic agent.

In some aspects, the present disclosure provides use of a compound of the present disclosure or a pharmaceutically acceptable salt thereof in the manufacture of a medicament for curing hepatitis B virus in a subject in need thereof, in combination with one or more (e.g., one, two, three, four, one or two, one to three, or one to four) additional therapeutic agent.

In some embodiments, a compound disclosed herein, or a pharmaceutically acceptable salt thereof, is combined with one, two, three, four, or more additional therapeutic agent(s). In some embodiments, a compound disclosed herein, or a pharmaceutically acceptable salt thereof, is combined with one additional therapeutic agent. In some embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agent(s) can be different therapeutic agents selected from the same class of therapeutic agents, and/or they can be selected from different classes of therapeutic agents.

### Administration of HBV Combination Therapy

In some embodiments, when a compound disclosed herein is combined with one or more additional therapeutic agent as described above, the components of the composition are administered as a simultaneous or sequential regimen. When administered sequentially, the combination may be administered in two or more administrations.

Co-administration of a compound disclosed herein with one or more additional therapeutic agent generally refers to simultaneous or sequential administration of a compound disclosed herein and one or more additional therapeutic agent, such that therapeutically effective amounts of each agent are present in the body of the patient.

Co-administration includes administration of unit dosages of the compounds disclosed herein before or after administration of unit dosages of one or more additional therapeutic agent. The compound disclosed herein may be administered within seconds, minutes, or hours of the administration of one or more additional therapeutic agent. In some embodiments, a unit dose of a compound disclosed herein is administered first, followed within seconds or minutes by administration of a unit dose of one or more additional therapeutic agent. In some embodiments, a unit dose of one or more additional therapeutic agent is administered first, followed by administration of a unit dose of a compound disclosed herein within seconds or minutes. In some embodiments, a unit dose of a compound disclosed herein is administered first, followed, after a period of hours (e.g., 1-12 hours), by administration of a unit dose of one or more additional therapeutic agent. In some embodiments, a unit dose of one or more additional therapeutic agent is administered first, followed, after a period of hours (e.g., 1-12 hours), by administration of a unit dose of a compound disclosed herein.

In some embodiments, a compound disclosed herein is combined with one or more additional therapeutic agent in a unit dosage form for simultaneous administration to a patient, for example as a solid dosage form for oral administration.

### HBV Combination Therapy

In some embodiments, compounds of the present disclosure may be used or combined with one or more of a chemotherapeutic agent, an immunomodulator, an immunotherapeutic agent, a therapeutic antibody, a therapeutic vaccine, a bispecific antibody and “antibody-like” therapeutic protein (e.g., DARTs®, Duobodies®, Bites®, XmAbs®&, TandAbs®, Fab derivatives), an antibody-drug conjugate (ADC), gene modifiers or gene editors (such as CRISPR Cas9, zinc finger nucleases, homing endonucleases, synthetic nucleases, TALENs), cell therapies such as CART (chimeric antigen receptor T-cell), and TCR-T (an engineered T cell receptor) agent or any combination thereof.

In some embodiments, the additional therapeutic agent may be an anti-HBV agent. In some embodiments, the additional therapeutic agent may be selected from the group consisting of HBV combination therapeutics, additional therapeutics for treating HBV, 3-dioxygenase (IDO) inhibitors, antisense oligonucleotide targeting viral mRNA, Apolipoprotein A1 modulator, arginase inhibitors, B- and T-lymphocyte attenuator inhibitors, Bruton's tyrosine kinase (BTK) inhibitors, CCR2 chemokine antagonist, CD137 inhibitors, CD160 inhibitors, CD305 inhibitors, CD4 agonist and modulator, compounds targeting HBcAg, compounds targeting hepatitis B core antigen (HBcAg), covalently closed circular DNA (cccDNA) inhibitors, cyclophilin inhibitors, cytokines, cytotoxic T-lymphocyte-associated protein 4 (ipi4) inhibitors, DNA polymerase inhibitor, Endonuclease modulator, epigenetic modifiers, Farnesoid X receptor agonist, gene modifiers or editors, HBsAg inhibitors, HBsAg secretion or assembly inhibitors, HBV antibodies, HBV DNA polymerase inhibitors, HBV replication inhibitors, HBV RNAse inhibitors, HBV vaccines, HBV viral entry inhibitors, HBx inhibitors, Hepatitis B large envelope protein modulator, Hepatitis B large envelope protein stimulator, Hepatitis B structural protein modulator, hepatitis B surface antigen (HBsAg) inhibitors, hepatitis B surface antigen (HBsAg) secretion or assembly inhibitors, hepatitis B virus E antigen inhibitors, hepatitis B virus replication inhibitors, Hepatitis virus structural protein inhibitor, HIV-1 reverse transcriptase inhibitor, Hyaluronidase inhibitor, IAPs inhibitors, IL-2 agonist, IL-7 agonist, Immunoglobulin agonist, Immunoglobulin G modulator, immunomodulators, indoleamine-2, inhibitors of ribonucleotide reductase, Interferon agonist, Interferon alpha 1 ligand, Interferon alpha 2 ligand, Interferon alpha 5 ligand modulator, Interferon alpha ligand, Interferon alpha ligand modulator, interferon alpha receptor ligands, Interferon beta ligand, Interferon ligand, Interferon receptor modulator, Interleukin-2 ligand, ipi4 inhibitors, lysine demethylase inhibitors, histone demethylase inhibitors, KDM5 inhibitors, KDMI inhibitors, killer cell lectin-like receptor subfamily G member 1 inhibitors, lymphocyte-activation gene 3 inhibitors, lymphotoxin beta receptor activators, microRNA (miRNA) gene therapy agents, modulators of Ax1, modulators of B7-H3, modulators of B7-H4, modulators of CD160, modulators of CD161, modulators of CD27, modulators of CD47, modulators of CD70, modulators of GITR, modulators of HEVEM, modulators of ICOS, modulators of Mer, modulators of NKG2A, modulators of NKG2D, modulators of 0X40, modulators of SIRPalpha, modulators of TIGIT, modulators of Tim-4, modulators of Tyro, Na+-taurocholate cotransporting polypeptide (NTCP) inhibitors, natural killer cell receptor 2B4 inhibitors, NOD2 gene stimulator, Nucleoprotein inhibitor, nucleoprotein modulators, PD-1 inhibitors, PD-L1 inhibitors, PEG-Interferon Lambda, Peptidylprolyl isomerase inhibitor, phosphatidylinositol-3 kinase (PI3K) inhibitors, recombinant scavenger receptor A (SRA) proteins, recombinant thymosin alpha-1, Retinoic acid-inducible gene 1 stimulator, Reverse transcriptase inhibitor, Ribonuclease inhibitor, RNA DNA polymerase inhibitor, short interfering RNAs (siRNA), short synthetic hairpin RNAs (sshRNAs), SLC10A1 gene inhibitor, SMAC mimetics, Src tyrosine kinase inhibitor, stimulator of interferon gene (STING) agonists, stimulators of NOD1, T cell surface glycoprotein CD28 inhibitor, T-cell surface glycoprotein CDS modulator, Thymosin agonist, Thymosin alpha 1 ligand, Tim-3 inhibitors, TLR-3 agonist, TLR-7 agonist, TLR-9 agonist, TLR9 gene stimulator, toll-like receptor (TLR) modulators, Viral ribonucleotide reductase inhibitor, zinc finger nucleases or synthetic nucleases (TALENs), and combinations thereof.

### HBV Combination Therapeutics

In some embodiments, examples of combination drugs for the treatment of HBV include tenofovir disoproxil fumarate and emtricitabine; ABX-203, lamivudine, and PEG-IFN-alpha; ABX-203 adefovir, and PEG-IFNalpha; and INO-1800 (INO-9112 and RG7944).

### Additional HBV Therapeutics

In some embodiments, examples of other drugs for the treatment of HBV include alpha-hydroxytropolones, amdoxovir, beta-hydroxy cytosine nucleosides, AL-034, CCC-0975, elvucitabine, ezetimibe, cyclosporin A, gentiopicrin (gentiopicroside), JNJ-561 36379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-mRNA, cTP-5 (rTP-5), HSK-U-2, HEISCO-106-1, HEISCO-106, Flepbama, IBPB-006IA, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, UB-551, and ZH-2N.

### HBV DNA Polymerase Inhibitors

In some embodiments, examples of HBV DNA polymerase inhibitors include adefovir, emtricitabine, tenofovir disoproxil fumarate, tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, besifovir, entecavir, entecavir maleate, telbivudine, pradefovir, devudine, ribavirin, lamivudine, phosphazide, famciclovir, fusolin, metacavir, SNC-019754, FMCA, AGX-1009, AR-II-04-26, HIP-1302, tenofovir disoproxil aspartate, tenofovir disoproxil orotate, HS-10234, and filocilovir.

### Immunomodulators

In some embodiments, examples of immunomodulators include rintatolimod, imidol hydrochloride, ingaron, dermaVir, plaquenil (hydroxychloroquine), proleukin, hydroxyurea, mycophenol ate mofetil (MIPA) and its ester derivative mycophenolate mofetil (MMF), WF-10, ribavirin, IL-12, 1NO-9 112, polymer polyethyleneimine (PEI), Gepon, VGV-1, MOR-22, BMS-936559, RO-701 1785, RO-6871765, AIC-649, IR-103, JNJ-440, AB-452, CRV-431, JNJ-0535, TG-1050, ABI-H2158, GS-9688, RG-7854, and AB-506.

### Interferon Alpha Receptor Ligands

In some embodiments, examples of interferon alpha receptor ligands include interferon alpha-2b, pegylated interferon alpha-2a, PEGylated interferon alpha-1b, interferon alpha 1b, Veldona, Infradure, Roferon-A, YPEG-interferon alfa-2a (YPEGrhIFNalpha-2a), P-1 101, Algeron, Alfarona, Ingaron (interferon gamma), rSIFN-co (recombinant super compound interferon), Ypeginterferon alfa-2b (YPEG-rhIFNalpha-2b), MOR-22, peginterferon alfa-2b, Bioferon, Novaferon, Inmutag (Inferon), interferon alfa-n1, interferon beta-1a, ropeginterferon alfa-2b, rHSA-IFN alpha-2a (recombinant subject serum albumin intereferon alpha 2a fusion protein), rHSA-IFN alpha 2b, recombinant subject interferon alpha-(1b, 2a, 2b), Reaferon-EC, Proquiferon, Uniferon, Urifron, Anterferon, Shanferon, Layfferon, Shang Sheng Lei Tai, INTEFEN, SINOGEN, Fukangtai, Pegstat, rHSA-IFN alpha-2b, SFR-9216, and Interapo (Interapa).

### PD-1 Inhibitors

In some embodiments, examples of PD-1 inhibitors include nivolumab, pembrolizumab, pidilizumab, BGB-108, SHR-1210, PDR-001, PF-06801591, IBI-308, GB-226, STI-1 110, mDX-400, cemiplimab, STI-A1014, JNJ-63723283, CA-170, durvalumab, atezolizumab, JS-001, camrelizumab, sintilimab, sintilimab, tislelizumab, BCD-100, BGB-A333 JNJ-63723283, GLS-010 (WBP-3055), CX-072, AGEN-2034, GNS-1480 (epidermal growth factor receptor antagonist; programmed cell death ligand 1 inhibitor), CS-1001, M-7824 (PD-L1/TGF-b bifunctional fusion protein), Genolimzumab, and BMS-936559.

### PD-L1 Inhibitors

In some embodiments, examples of PD-L1 inhibitors include atezolizumab, avelumab, AMP-224, MEDI-0680, RG-7446, GX-P2, durvalumab, KY-1003, KD-033, MSB-00 0718C, TSR-042, ALNPDL, STI-A1014, CX-072, and BMS-936559. Additional examples of PD-L1 inhibitors include GS-4224, INCB086550, and INCB090244.

### Bruton's Tyrosine Kinase (BTK) Inhibitors

In some embodiments, examples of BTK inhibitors include ABBV-105, acalabrutinib (ACP-196), ARQ-531, BMS-986142, dasatinib, ibrutinib, GDC-0853, PRN-1008, SNS-062, ONO-4059, BGB-3111, ML-319, MSC-2364447, RDX-022, X-022, AC-058, RG-7845, spebrutinib, TAS-5315, TP-0158, TP-4207, HM-71224, KBP-7536, M-2951, TAK-020, AC-0025, and the compounds disclosed in US20140330015 (Ono Pharmaceutical), US20130079327 (Ono Pharmaceutical), and US20130217880 (Ono Pharmaceutical).

### Kits

In one aspect, the present disclosure provides a kit comprising a compound of the present disclosure or a pharmaceutically acceptable salt thereof. The kit may further comprise instructions for use, e.g., for use in treating a HBV infection. The instructions for use are generally written instructions, although electronic storage media (e.g, magnetic diskette or optical disk) containing instructions are also acceptable.

In some embodiments, the present disclosure also provides a pharmaceutical kit comprising one or more containers comprising a compound of the present disclosure or a pharmaceutically acceptable salt thereof. Optionally associated with such container(s) can be a notice in the form prescribed by a governmental agency regulating the manufacture, use or sale of pharmaceuticals, which notice reflects approval by the agency for the manufacture, use or sale for subject administration. Each component (if there is more than one component) can be packaged in separate containers or some components can be combined in one container where cross-reactivity and shelf life permit. The kits may be in unit dosage forms, bulk packages (e.g., multi-dose packages) or subunit doses. Kits may also include multiple unit doses of the compounds and instructions for use and be packaged in quantities sufficient for storage and use in pharmacies (e.g., hospital pharmacies and compounding pharmacies).

In some embodiments, the kit includes articles of manufacture comprising a unit dosage of a compound of the present disclosure or a pharmaceutically acceptable salt thereof, in suitable packaging for use in the methods described herein. Suitable packaging is known in the art and includes, for example, vials, vessels, ampules, bottles, jars, flexible packaging and the like. An article of manufacture may further be sterilized and/or sealed.

### Exemplary Embodiments

Exemplary Embodiment No. 1. A compound of Formula (I′):

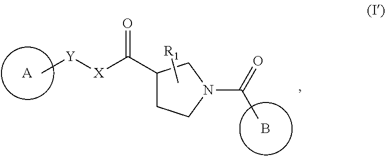

or a prodrug, solvate, or pharmaceutically acceptable salt thereof, wherein:
X is —N(Rₓ)— or —O—;
Y is absent or —C(R_{Y})₂—
Rₓ is H, C₁-C₆ alkyl, C₂-C₆ alkenyl, C₂-C₆ alkynyl, or C₁-C₆ haloalkyl;
each R_{Y} independently is H, C₁-C₆ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl;
Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A};
Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B};
R₁ is H, C₁-C₆ alkyl, C₂-C₆ alkenyl, C₂-C₆ alkynyl, or C₁-C₆ haloalkyl;
each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₁-C₇ cycloalkyl;
each R_{B} independently is halogen, —CN, —(CH₂)ₙ—OR_{B1}, —(CH₂)ₙ—N(R_{B1})(R_{B2}), —(CH₂)ₙ—S(R_{B1}), C₁-C₆ alkyl, C₂-C₆ alkenyl, C₁-C₆ haloalkyl, C₁-C₆ alkoxy, —(CH₂)ₙ—(C₆-C₁₀ aryl), (5- to 10-membered heteroaryl), (C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4};
each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or
R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₁-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′};
each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy;
each R_{B3} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₘ—C(O)R_{B4″}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4″}, or —N(R_{B4″})(R_{B4″});
each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH;
n is 0, 1, 2, 3, 4, or 5; and
m is 0, 1, 2, 3, 4, or 5,
provided that when R_{B} is a substituted or unsubstituted alkyl, Ring A is substituted by at least one R_{A}.

Exemplary Embodiment No. 2. The compound of Exemplary Embodiment 1, wherein:
X is —N(R)—;
Y is absent;
Rₓ is H or C₁-C₆ alkyl;
Ring A is C₆-C₁₀ aryl or 5- to 10-membered heteroaryl, wherein the aryl and heteroaryl are optionally substituted with one or more R_{A};
Ring B is 5- to 10-membered heteroaryl optionally substituted with one or more R_{B}; and
R₁ is H or C₁-C₆ alkyl;
provided that when R_{B} is a substituted or unsubstituted alkyl, Ring A is substituted by at least on R_{A}.

Exemplary Embodiment No. 3. The compound of Exemplary Embodiment 1, wherein X is —N(Rₓ)—.

Exemplary Embodiment No. 4. The compound of Exemplary Embodiment 1, wherein Y is absent or —CH₂—.

Exemplary Embodiment No. 5. The compound of Exemplary Embodiment 1, wherein Rₓ is H.

Exemplary Embodiment No. 6. The compound of Exemplary Embodiment 1, wherein Rₓ is C₁-C₆ alkyl, C₂-C₆ alkenyl, or C₂-C₆ alkynyl.

Exemplary Embodiment No. 7. The compound of Exemplary Embodiment 1, wherein Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{A}.

Exemplary Embodiment No. 8. The compound of Exemplary Embodiment 1, wherein Ring A is C₆-C₁₀ aryl substituted with one or more R_{A}.

Exemplary Embodiment No. 9. The compound of Exemplary Embodiment 1, wherein Ring A is phenyl substituted with two R_{A}.

Exemplary Embodiment No. 10. The compound of Exemplary Embodiment 1, wherein Ring A is phenyl substituted with three R_{A}.

Exemplary Embodiment No. 11. The compound of Exemplary Embodiment 1, wherein Ring B is 5- to 10-membered heteroaryl optionally substituted with one or more R_{B}.

Exemplary Embodiment No. 12. The compound of Exemplary Embodiment 1, wherein Ring B is 5- to 10-membered heteroaryl substituted with one or more R_{B}.

Exemplary Embodiment No. 13. The compound of Exemplary Embodiment 1, wherein R₁ is H.

Exemplary Embodiment No. 14. The compound of Exemplary Embodiment 1, wherein R₁ is C₁-C₆ alkyl, C₂-C₆ alkenyl, C₂-C₆ alkynyl, or C₁-C₆ haloalkyl.

Exemplary Embodiment No. 15. The compound of Exemplary Embodiment 1, wherein each R_{A} independently is halogen, —CN, —OH, —NH₂, —NH(C₁-C₆ alkyl), or —N(C₁-C₆ alkyl)₂.

Exemplary Embodiment No. 16. The compound of Exemplary Embodiment 1, wherein each R_{A} independently is C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₆ cycloalkyl.

Exemplary Embodiment No. 17. The compound of Exemplary Embodiment 1, wherein each R_{A} independently is halogen, —CN, C₁-C₆ alkyl, or C₃-C₇ cycloalkyl.

Exemplary Embodiment No. 18. The compound of Exemplary Embodiment 1, wherein each R_{B} independently is halogen, —CN, —(CH₂)ₙ—OR_{B1}, —(CH₂)ₙ—N(R_{B1})(R_{B2}), —(CH₂)ₙ—S(R_{B1}), —C(O)R_{B1}, —C(O)OR_{B1}, or —C(O)N(R_{B1})(R_{B2}).

Exemplary Embodiment No. 19. The compound of Exemplary Embodiment 1, wherein each R_{B} independently is C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₁-C₇ cycloalkyl), or —(CH₂)ₙ-(3- to 7-membered heterocycloalkyl), wherein the alkyl, alkenyl, alkynyl, aryl, heteroaryl, cycloalkyl, and heterocycloalkyl are optionally substituted with one or more R_{B4}.

Exemplary Embodiment No. 20. The compound of Exemplary Embodiment 1, wherein each R_{B1} and R_{B2} is independently H.

Exemplary Embodiment No. 21. The compound of Exemplary Embodiment 1, wherein each R_{B1} and R_{B2} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₁-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

Exemplary Embodiment No. 22. The compound of Exemplary Embodiment 1, wherein each R_{B3} is independently C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more R_{B4}.

Exemplary Embodiment No. 23. The compound of Exemplary Embodiment 1, wherein each R_{B} is independently C₆-C₁₀ aryl, 5- to 10-membered heteroaryl, C₃-C₆ cycloalkyl, or 3- to 7-membered heterocycloalkyl, wherein the aryl, heteroaryl, cycloalkyl, and heterocycloalkyl are optionally substituted with one or more R_{B3′}.

Exemplary Embodiment No. 24. The compound of Exemplary Embodiment 1, wherein each R_{B3′} is independently halogen, —CN, —OH, —NH₂, —NH(C₁-C₆ alkyl), —NH(C₁-C₆ alkyl)-OH, or —N(C₁-C₆ alkyl)₂.

Exemplary Embodiment No. 25. The compound of Exemplary Embodiment 1, wherein each R_{B3′} is independently C₁-C₆ alkyl, C₂-C₆ alkenyl, C₂-C₆ alkynyl, C₁-C₆ haloalkyl, or C₁-C₆ alkoxy.

Exemplary Embodiment No. 26. The compound of Exemplary Embodiment 1, wherein each R_{B4} is independently oxo, halogen, —CN, —OH, —NH₂, —NH(C₁-C₆ alkyl), —NH(C₁-C₆ alkyl)-OH, —N(C₁-C₆ alkyl)₂, or, —(CH₂)ₙ—C(O)R_{B4′} wherein the alkyl is optionally substituted with one or more halogen, —CN, —OR_{B4″}, or —N(R_{B4″})(R_{B4″}).

Exemplary Embodiment No. 27. The compound of Exemplary Embodiment 1, wherein each R_{B4} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4″}, or —N(R_{B4″})(R_{B4″}).

Exemplary Embodiment No. 28. The compound of Exemplary Embodiment 1, wherein R_{B4} is H or —OH.

Exemplary Embodiment No. 29. The compound of Exemplary Embodiment 1, wherein R_{B4″} is C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

Exemplary Embodiment No. 30. The compound of Exemplary Embodiment 1, wherein R_{B4″} is H or —OH.

Exemplary Embodiment No. 31. The compound of Exemplary Embodiment 1, wherein R_{B4″} is C₁-C₆ alkyl, C₂-C₆ alkenyl, C₂-C₆ alkynyl, C₁-C₆ haloalkyl, C₁-C₆ alkoxy, C₁-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH.

Exemplary Embodiment No. 32. The compound of Exemplary Embodiment 1, wherein n is 0, 1, or 2.

Exemplary Embodiment No. 33. The compound of Exemplary Embodiment 1, wherein the compound is of Formula (I′-c) or (I′-d):

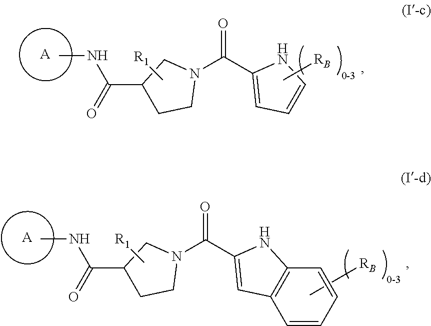

or a prodrug, solvate, or pharmaceutically acceptable salt thereof.

Exemplary Embodiment No. 34. The compound of Exemplary Embodiment 1, wherein the compound is of Formula (I′-c1):

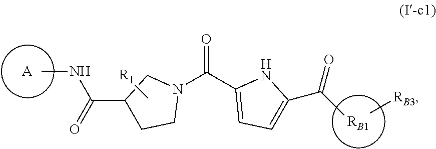

or a prodrug, solvate, or pharmaceutically acceptable salt thereof.

Exemplary Embodiment No. 35. The compound of Exemplary Embodiment 1, wherein the compound is of Formula (I-a′) or (1-b′):

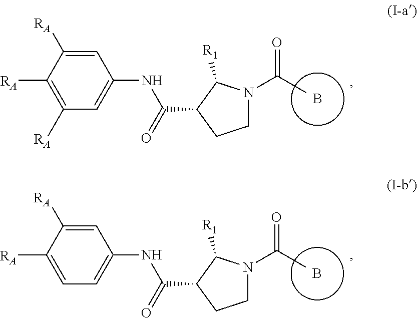

or a prodrug, solvate, or pharmaceutically acceptable salt thereof.

Exemplary Embodiment No. 36. The compound of Exemplary Embodiment 1, wherein the compound is of Formula (I-c′) or (I-d′):

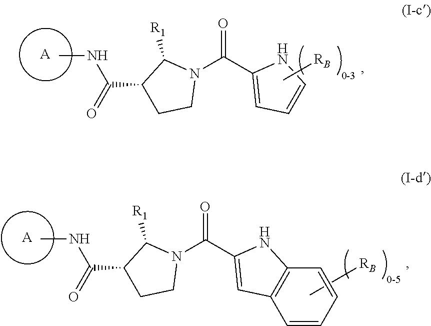

or a prodrug, solvate, or pharmaceutically acceptable salt thereof.

Exemplary Embodiment No. 37. The compound of Exemplary Embodiment 1, wherein the compound is of Formula (I-c1′);

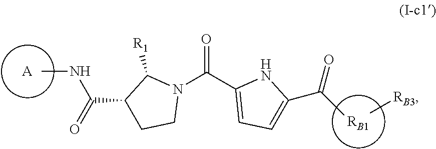

or a prodrug, solvate, or pharmaceutically acceptable salt thereof.

Exemplary Embodiment No. 38. The compound of any one of the preceding Exemplary Embodiments, being selected from Compound Nos. 1-175 and prodrugs and pharmaceutically acceptable salts thereof.

Exemplary Embodiment No. 39. The compound of any one of the preceding Exemplary Embodiments, being selected from Compound Nos. 1-175 and pharmaceutically acceptable salts thereof.

Exemplary Embodiment No. 40. The compound of any one of the preceding Exemplary Embodiments, being selected from Compound Nos. 1-175.

Exemplary Embodiment No. 41. A compound obtainable by, or obtained by, a method described herein; optionally, the method comprises one or more steps described in Schemes I-V.

Exemplary Embodiment No. 42. A pharmaceutical composition comprising the compound of any one of Exemplary Embodiments 1-41 or a pharmaceutically acceptable salt thereof, and a pharmaceutically acceptable diluent or carrier.

Exemplary Embodiment No. 43. The pharmaceutical composition of Exemplary Embodiment 42, wherein the compound is selected from Compound Nos. 1-175.

Exemplary Embodiment No. 44. A method of treating or preventing a disease or disorder in a subject in need thereof, comprising administering to the subject a therapeutically effective amount of the compound of any one of Exemplary Embodiments 1-41 or a pharmaceutically acceptable salt thereof, or the pharmaceutical composition of Exemplary Embodiment 42 or Exemplary Embodiment 43.

Exemplary Embodiment No. 45. The compound of any one of Exemplary Embodiments 1-41, or the pharmaceutical composition of Exemplary Embodiment 42 or Exemplary Embodiment 43, for use in treating or preventing a disease or disorder.

Exemplary Embodiment No. 46. Use of the compound of any one of Exemplary Embodiments 1-41 or a pharmaceutically acceptable salt thereof in the manufacture of a medicament for treating or preventing a disease or disorder.

Exemplary Embodiment No. 47. The method, compound, pharmaceutical composition, or use of any one of the preceding Exemplary Embodiments, wherein the disease or disorder is a viral infection.

Exemplary Embodiment No. 48. The method, compound, pharmaceutical composition, or use of Exemplary Embodiment 47, wherein the viral infection is hepatitis B virus

Exemplary Embodiment No. 49. A method of modulating the HBV replication cycle in a subject in need thereof, comprising administering to the subject a therapeutically effective amount of the compound of any one of Exemplary Embodiments 1-41 or a pharmaceutically acceptable salt thereof, or the pharmaceutical composition of Exemplary Embodiment 42 or Exemplary Embodiment 43.

Exemplary Embodiment No. 50. The compound of any one of Exemplary Embodiments 1-41, or the pharmaceutical composition of Exemplary Embodiment 42 or Exemplary Embodiment 43, for use modulating the HBV replication cycle.

Exemplary Embodiment No. 51. Use of the compound of any one of Exemplary Embodiments 1-41 or a pharmaceutically acceptable salt thereof in the manufacture of a medicament for modulating the HBV replication cycle.

Exemplary Embodiment No. 52. The method, compound, pharmaceutical composition, or use of any one of the preceding Exemplary Embodiments, in combination with one or more additional therapeutic agent.

Exemplary Embodiment No. 53. The method, compound, pharmaceutical composition, or use of Exemplary Embodiment 52, wherein the one or more additional therapeutic agent is useful for treating virus infection.

Exemplary Embodiment No. 54. The method, compound, pharmaceutical composition, or use of Exemplary Embodiment 52 or Exemplary Embodiment 53, wherein the at least one or more additional therapeutic agent comprises a medicament for treatment of HBV.

Exemplary Embodiment No. 55. The method, compound, pharmaceutical composition, or use of any one of Exemplary Embodiments 52-54, wherein the one or more additional therapeutic agent is administered simultaneously, separately, or sequentially.

### EXAMPLES

For exemplary purpose, neutral compounds of Formula (I) or Formula (I′) are synthesized and tested in the examples. It is understood that the neutral compounds of Formula (I) or Formula (I′) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Nuclear magnetic resonance (NMR) spectra were recorded at 400 MHz or 300 MHz as stated and at 300.3 K unless otherwise stated; the chemical shifts (6) are reported in parts per million (ppm). Spectra were recorded using a JEOL, JNM-ECZ500R instrument with 8, 16 or 32 scans.

LC-MS chromatograms and spectra were recorded using a LC pump, a diode-array (DAD), or a UV detector and a column as specified in the respective methods. If necessary additional detectors were included (See, Table of methods below).

### LCMS Methods

| | | | Flow | Run | |
|---|---|---|---|---|---|
| | | | (mL/min) | time | |
| Method | Column | Gradient | (Col T (º C.)) | (min) | Instrument |
| A | Kinetic C-18, | 80% A held for 0.5 min. From | 0.2 (45) | 10 | Sciex: |
| | 1.3 μm, 50 × | 80% A to 10% A in 6.0 min, | | | ExionLC TM |
| | 2.1 mm | held for 1.0 min. From 10% A | | | and SQD |
| | (Phenonenex) | to 80% A in 0.5 min, held for | | | (QTRAP |
| | | 2.0 min, | | | 6500⁺) |
| B | Eclipse | 90% A held for 1.0 min, From | 0.3 (45) | 12 | Agilent: |
| | XDB-Phenyl, | 90% A to 30% A in 1.0 min, | | | 1260 |
| | 3.5 μm, 100 × | From 30% A to 15% A in 4.0 | | | Infinity II, |
| | 3.0 mm | min, held for 1.0 min, From | | | — |
| | (Agilent) | 15% A to 30% A in 2.0 min, | | | DAD, SQD |
| | | From 30% A to 90% A in 1.0 | | | (LC/MSD) |
| | | min, held for 2.0 min. | | | |
| C | Kinetic C-18, | 98% A held for 0.5 min, From | 0.3 (45) | 7 | Sciex: |
| | 1.3 μm, 50 × | 98% A to 35% A in 4.0 min, | | | ExionLC ^{™} |
| | 2.1 mm | From 35% A to 5% A in 0.5 | | | and Q-Tof |
| | (Phenonenex) | min, held for 1.0 min, From | | | (X500B) |
| | | 5% A to 98% A in 0.1 min, | | | |
| | | held for 0.9 min. | | | |

*Col T = Column temperature;
Mobile phase A = 0.1% HCOOH + H₂O, Mobile phase B = 0.1% HCOOH + CH₃CN

Flow from the column was brought to the Mass Spectrometer (MS) which was configured with an atmospheric pressure ion source. The tune parameters (e.g. scanning range, dwell time, and collision energy) were set within the knowledge of the skilled person for obtaining ions allowing for the identification of the compounds monoisotopic molecular weight (MW). Data acquisition was perform with appropriate software.

The compound characterization is presented by experimental retention times (Rt) and ions. The reported molecular ion corresponds to the [M+H]+(protonated molecule) and/or [M−H]− (deprotonated molecule). In case the compound was not directly ionizable the type of adduct is specified (i.e. [M+NH₄+]+, [M+Na]+, [M+HCOO]−, etc.). All result were obtained with experimental uncertainties that are commonly associated with the method used.

Hereinafter, “SQD” means Single Quadrupole Detector; “Q-Tof” Quadrupole Time-of-flight mass spectrometers; “DAD” Diode Array Detector, “RT” room temperature; and injection volumes were 0.7-8.0 μl with flow rates typically at 0.8 or 1.2 mi/min.

Detection methods were diode array (DAD) or evaporative light scattering (ELSD), as well as positive ion electrospray ionization. MS range was 100-1000 Da. Solvents were gradients of water and acetonitrile both containing a modifier (typically 0.01-0.04%) such as trifluoroacetic acid or ammonium carbonate.

### Abbreviations

ACN acetonitrile
AcOH acetic acid
DCE 1,2-dichloroethane
DCM dichloromethane
DIAD diisopropyl azodicarboxylate
DIPEA N,N-diisopropylethylamine
DMAP N,N-dimethylaminopyridine
DMF N,N-dimethylformamide
DPPA diphenylphosphoryl azide
dppf 1,1′-bis(diphenylphosphino)ferrocene
EDCI N-(3-dimethylaminopropyl)-N′-ethylcarbodiimide hydrochloride
ESI electrospray ionization
EtOAc ethyl acetate
EtOH ethanol
h hour(s)
HATU N-[(dimethylamino)-1H-1,2,3-triazolo-[4,5-b]pyridin-1-ylmethylene]-N-methylmethanaminium hexafluorophosphate N-oxide
HMPA hexamethylphosphoramide
HOBt hydroxybenzotriazole
Jones reagent CrO₃ in aqueous H₂SO₄
Lawesson's reagent 2,4-Bis(4-methoxyphenyl)-1,3,2,4-dithiadiphosphetane-2,4-dithione
LCMS Liquid Chromatography—Mass Spectrometry
MeCN acetonitrile
MeOD methanol-d₄
MeOH methanol
Me₂S dimethylsulfide
min minute(s)
m/z mass/charge
NBS N-bromosuccinimide
NCS N-chlorosuccinimide
Pd/C palladium on carbon
Pd₂(dba)₃ tris(dibenzylideneacetone)dipalladium(0)
Pd(PPh₃)₄ tetrakis(triphenylphosphine)palladium(0)
PPh₃ triphenylphosphine
prep-HPLC preparative high-performance liquid chromatography
prep-TLC preparative thin-layer chromatography
psi pound-force per square inch
Pt/C platinum on carbon
RM reaction mixture
rt room temperature
Rt retention time
TFA trifluoroacetic acid
THF tetrahydrofuran
XPhos 2-dicyclohexylphosphino-2′,4′,6′-triisopropylbiphenyl
Y yield

### Synthesis of Intermediate III of Schemes I, II, III, IV, and V

### Intermediate III-A: Synthesis of (S)—N-(3,4,5-trifluorophenyl)pyrrolidine-3-carboxamide hydrochloride

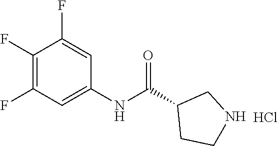 [SMILES: O=C(Nc1cc(F)c(F)c(F)c1)[C@H]1CCNC1 ; Cl]

HATU 2.80 g (6 mmol) and N,N-diisopropylethylamine (DIPEA) 2 mL were added to a solution of (S)-1-(tert-butoxycarbonyl)pyrrolidine-3-carboxylic acid 0.86 g (4 mmol), 3,4,5-trifluoroaniline 0.66 g (4.5 mmol) in dimethylformamide (DMF) (12 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAc:Hexanes. The desired fractions were pooled and the solvent was removed under reduced pressure to afford the Boc-protected title compound (3.4 mmol) as a pale-yellow oil.

Subsequent Boc deprotection HCl (4M in dioxane, 30 minutes at room temperature) afforded (S)—N-(3,4,5-trifluorophenyl)pyrrolidine-3-carboxamide hydrochloride that was used as such in the next step without further purification.

### Intermediate III-B: Synthesis of(S)—N-(4-fluoro-3-methylphenyl)pyrrolidine-3-carboxamide hydrochloride

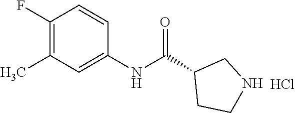 [SMILES: Cc1cc(NC(=O)[C@H]2CCNC2)ccc1F ; Cl]

HATU 1.71 g (4.5 mmol) and N,N-diisopropylethylamine (DIPEA) 2 mL were added to a solution of (S)-1-(tert-butoxycarbonyl)pyrrolidine-3-carboxylic acid 0.65 g (3 mmol), 4-fluoro-3-methylaniline 0.44 g (3.5 mmol) in dimethylformamide (DMF) (7 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC:Hexanes. The desired fractions were pooled and the solvent was removed under reduced pressure to afford the Boc-protected title compound (2.8 mmol) as a pale-yellow oil.

Subsequent Boc deprotection HCl (4M in dioxane, 30 minutes at room temperature) afforded (S)—N-(4-fluoro-3-methylphenyl)pyrrolidine-3-carboxamide hydrochloride that was used as such in the next step without further purification.

### Intermediate III-C: Synthesis of(S)—N-(3-bromo-4,5-difluorophenyl)pyrrolidine-3-carboxamide hydrochloride

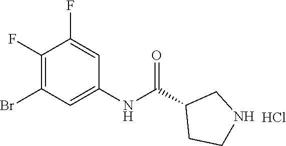 [SMILES: O=C(Nc1cc(F)c(F)c(Br)c1)[C@H]1CCNC1 ; Cl]

HATU 570 mg (1.5 mmol) and N,N-diisopropylethylamine (DIPEA) 1 mL were added to a solution of (S)-1-(tert-butoxycarbonyl)pyrrolidine-3-carboxylic acid 215 g (1 mmol), 3-bromo-4,5-difluoroaniline 250 mg (1.2 mmol) in dimethylformamide (DMF) (5 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford the Boc-protected title compound (0.69 mmol) as a pale-yellow oil.

Subsequent Boc deprotection HCl (4M in dioxane, 30 minutes at room temperature) afford (S)—N-(3-bromo-4,5-difluorophenyl)pyrrolidine-3-carboxamide hydrochloride that was used as such in the next step without further purification.

### Intermediate III-D: Synthesis of (2S,3S)—N-(3,4,5-trifluorophenyl)-2-methylpyrrolidine-3-carboxamide hydrochloride

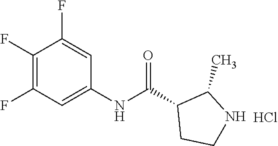 [SMILES: C[C@@H]1NCC[C@@H]1C(=O)Nc1cc(F)c(F)c(F)c1 ; Cl]

HATU 570 mg (7.5 mmol) and N,N-diisopropylethylamine (DIPEA) 1 mL were added to a solution of (2S,3S)-1-(tert-butoxycarbonyl)-2-methylpyrrolidine-3-carboxylic acid 229 mg (1 mmol), 3,4,5-trifluoroaniline 221 mg (1.5 mmol) in dimethylformamide (DMF) (5 mL) and stirred at room temperature overnight. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC:Hexanes. The desired fractions were pooled and the solvent was removed under reduced pressure to afford the Boc-protected title compound (0.68 mmol) as a pale-yellow oil.

Subsequent Boc deprotection HCl (4M in dioxane, 30 minutes at room temperature) afforded (2S,3S)-2-methyl-N-(3,4,5-trifluorophenyl)pyrrolidine-3-carboxamide hydrochloride that was used as such in the next step without further purification.

### Intermediate III-E: Synthesis of(S)—N-(3-cyano-4-fluorophenyl)pyrrolidine-3-carboxamide hydrochloride

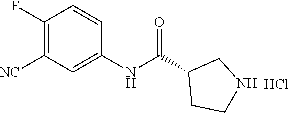 [SMILES: N#Cc1cc(NC(=O)[C@H]2CCNC2)ccc1F ; Cl]

HATU 2.80 g (7.5 mmol) and N,N-diisopropylethylamine (DIPEA) 3 mL (17.2 mmol) were added to a solution of (S)-1-(tert-butoxycarbonyl)pyrrolidine-3-carboxylic acid 1.1 g (5.1 mmol), 5-amino-2-fluorobenzonitrile 0.68 g (5 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford the Boc-protected title compound (3.25 mmol) as a pale-yellow oil.

Subsequent Boc deprotection HCl (4M in dioxane, 30 minutes at room temperature) afford (S)—N-(3-cyano-4-fluorophenyl)pyrrolidine-3-carboxamide hydrochloride that was used as such in the next step without further purification.

### Intermediate III-F: Synthesis of (2S,3S)—N-(3-chloro-4,5-difluorophenyl)-2-methylpyrrolidine-3-carboxamide hydrochloride

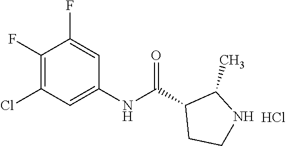 [SMILES: C[C@@H]1NCC[C@@H]1C(=O)Nc1cc(F)c(F)c(Cl)c1 ; Cl]

HATU 0.76 g (2 mmol) and N,N-diisopropylethylamine (DIPEA) 1 mL were added to a solution of (2S,3S)-1-tert-butyl 3-methyl 2-methylpyrrolidine-1,3-dicarboxylate 229 g (1 mmol), 3-chloro-4,5-difluoroaniline 326 g (2 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford the Boc-protected title compound (0.73 mmol) as a pale-yellow oil.

Subsequent Boc deprotection HCl (4M in dioxane, 30 minutes at room temperature) afford (2S,3S)—N-(3-chloro-4,5-difluorophenyl)-2-methylpyrrolidine-3-carboxamide hydrochloride that was used as such in the next step without further purification.

### Intermediate III-G: Synthesis of (2S,3S)—N-(3-cyano-4-fluorophenyl)-2-methylpyrrolidine-3-carboxamide hydrochloride

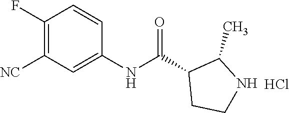 [SMILES: C[C@@H]1NCC[C@@H]1C(=O)Nc1ccc(F)c(C#N)c1 ; Cl]

HATU 570 mg (1.5 mmol) and N,N-diisopropylethylamine (DIPEA) 1 mL were added to a solution of (2S,3S)-1-(tert-butoxycarbonyl)-2-methylpyrrolidine-3-carboxylic acid 229 mg (1.0 mmol), 3-cyano-4-fluoroaniline 204 mg (1.5 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC. Hexanes. The desired fractions were pooled and the solvent was removed under reduced pressure to afford the Boc-protected title compound (0.55 mmol) as a pale-yellow oil.

Subsequent Boc deprotection HCl (4M in dioxane, 30 minutes at room temperature) afforded (2S,3S)—N-(3-cyano-4-fluorophenyl)-2-methylpyrrolidine-3-carboxamide hydrochloride that was used as such in the next step without further purification.

### Intermediate III-H: Synthesis of (2S,3S)-2-methyl-N-(4-fluoro-3-methylphenyl)pyrrolidine-3-carboxamide hydrochloride

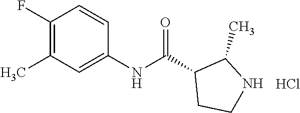 [SMILES: Cc1cc(NC(=O)[C@H]2CCN[C@H]2C)ccc1F ; Cl]

HATU 570 g (1.5 mmol) and N,N-diisopropylethylamine (DIPEA) 1 mL were added to a solution of (2S,3S)-1-(tert-butoxycarbonyl)-2-methylpyrrolidine-3-carboxylic acid 229 mg (1 mmol), 4-fluoro-3-methylaniline 187 mg (1.5 mmol) in dimethylformamide (DMF) (5 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC:Hexanes. The desired fractions were pooled and the solvent was removed under reduced pressure to afford the Boc-protected title compound (0.90 mmol) as a pale-yellow oil.

Subsequent Boc deprotection HCl (4M in dioxane, 30 minutes at room temperature) afforded (2S,3S)-2-methyl-N-(4-fluoro-3-methylphenyl)pyrrolidine-3-carboxamide hydrochloride that was used as such in the next step without further purification.

### Intermediate III-I: Synthesis of(S)—N-(3-chloro-4-fluorophenyl)pyrrolidine-3-carboxamide hydrochloride

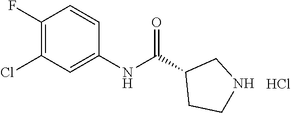 [SMILES: O=C(Nc1ccc(F)c(Cl)c1)[C@H]1CCNC1 ; Cl]

HATU 2.28 g (6 mmol) and N,N-diisopropylethylamine (DIPEA) 2 mL were added to a solution of (S)-1-(tert-butoxycarbonyl)pyrrolidine-3-carboxylic acid 0.86 g (4 mmol), 3-chloro-4-fluoroaniline 0.99 g (4.5 mmol) in dimethylformamide (DMF) (12 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC/hexanes. The desired fractions were pooled and the solvent was removed under reduced pressure to afford the Boc-protected title compound (3.56 mmol) as a pale-yellow oil.

Subsequent Boc deprotection HCl (4M in dioxane, 30 minutes at room temperature) afforded (S)—N-(3-chloro-4-fluorophenyl)pyrrolidine-3-carboxamide hydrochloride that was used as such in the next step without further purification.

### Intermediate III-J: Synthesis of (2S,3S)—N-(3-bromo-4,5-difluorophenyl)-2-methylpyrrolidine-3-carboxamide hydrochloride

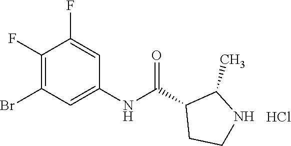 [SMILES: C[C@@H]1NCC[C@@H]1C(=O)Nc1cc(F)c(F)c(Br)c1 ; Cl]

HATU 2.80 g (7.5 mmol) and N,N-diisopropylethylamine (DIPEA) 3 mL (17.2 mmol) were added to a solution of (2S,3S)-1-(tert-butoxycarbonyl)-2-methylpyrrolidine-3-carboxylic acid 1.2 g (5.1 mmol), 3-bromo-4,5-difluoroaniline 0.74 g (5 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford the Boc-protected title compound (2.5 mmol) as a pale-yellow oil.

Subsequent Boc deprotection HCl (4M in dioxane, 30 minutes at room temperature) afford (S)—N-(3-bromo-4,5-difluorophenyl)pyrrolidine-3-carboxamide hydrochloride that was used as such in the next step without further purification.

### Intermediate III-K: Synthesis of (S)—N-(3-chloro-4,5-difluorophenyl)pyrrolidine-3-carboxamide hydrochloride

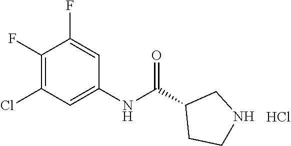 [SMILES: O=C(Nc1cc(F)c(F)c(Cl)c1)[C@H]1CCNC1 ; Cl]

HATU 2.80 g (7.5 mmol) and N,N-diisopropylethylamine (DIPEA) 3 mL (17.2 mmol) were added to a solution of (S)-1-(tert-butoxycarbonyl)pyrrolidine-3-carboxylic acid 1.1 g (5.1 mmol), 3-chloro-4,5-difluoroaniline 0.74 g (5 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford the Boc-protected title compound (4 mmol) as a pale-yellow oil.

Subsequent Boc deprotection HCl (4M in dioxane, 30 minutes at room temperature) afford (S)—N-(3-bromo-4,5-difluorophenyl)pyrrolidine-3-carboxamide hydrochloride that was used as such in the next step without further purification.

### Intermediate III-L: Synthesis of (S)—N-((2-chlorothiazol-5-yl)methyl)pyrrolidine-3-carboxamide hydrochloride

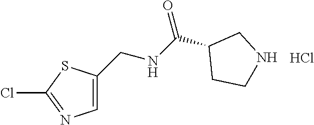 [SMILES: O=C(NCc1cnc(Cl)s1)[C@H]1CCNC1 ; Cl]

EDCI-HCl 1.91 g (10.0 mmol), HOBt 1.35 g (10.0 mmol) and N,N-diisopropylethylamine (DIPEA) 3 mL (17.2 mmol) were added to a solution of (S)-1-(tert-butoxycarbonyl)pyrrolidine-3-carboxylic acid 1.1 g (5.1 mmol), (2-chlorothiazol-5-yl)methanamine 0.74 g (5 mmol) in dimethylformamide (DMF) (15 mL) at 0° C. and stirred for overnight at room temperature. After completion of the reaction, ice was added and the reaction was partitioned with EtOAc. The organic layers were dried over sodium sulfate anhydrous, the solids were removed by filtration, and the solvent was removed under reduced pressure and the crude was purified via silica gel column chromatography by using 70% EtOAc/Hexanes. The desired fractions were pooled and the solvent was removed under reduced pressure to afford the Boc-protected title compound (2.85 mmol) as a pale-yellow oil.

Subsequent Boc deprotection HCl (4M in dioxane, 10 minutes at room temperature) afforded (S)—N-((2-chlorothiazol-5-yl)methyl)pyrrolidine-3-carboxamide hydrochloride that was used as such in the next step without further purification.

### Intermediate III-M: Synthesis of (2S,3S)—N-(3-chloro-4-fluorophenyl)-2-methylpyrrolidine-3-carboxamide hydrochloride

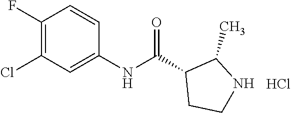 [SMILES: C[C@@H]1NCC[C@@H]1C(=O)Nc1ccc(F)c(Cl)c1 ; Cl]

HATU 2.80 g (7.5 mmol) and N,N-diisopropylethylamine (DIPEA) 3 mL (17.2 mmol) were added to a solution of (2S,3S)-1-(tert-butoxycarbonyl)-2-methylpyrrolidine-3-carboxylic acid 1.2 g (5.1 mmol), 3-chloro-4-fluoroaniline 0.74 g (5 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC:Hexanes. The desired fractions were pooled and the solvent was removed under reduced pressure to afford the Boc-protected title compound (4.45 mmol) as a pale-yellow oil.

Subsequent Boc deprotection HCl (4M in dioxane, 30 minutes at room temperature) afforded (2S,3S)—N-(3-chloro-4-fluorophenyl)-2-methylpyrrolidine-3-carboxamide hydrochloride that was used as such in the next step without further purification.

### Intermediate III-N. Synthesis of (2S,3S)—N-(3,4-difluorophenyl)-2-methylpyrrolidine-3-carboxamide hydrochloride

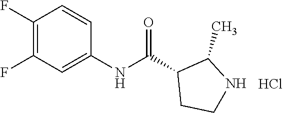 [SMILES: C[C@@H]1NCC[C@@H]1C(=O)Nc1ccc(F)c(F)c1 ; Cl]

HATU 2.80 g (7.5 mmol) and N,N-diisopropylethylamine (DIPEA) 3 mL (17.2 mmol) were added to a solution of (2S,3S)-1-(tert-butoxycarbonyl)-2-methylpyrrolidine-3-carboxylic acid, 3,4-difluoroaniline 0.74 g (5 mmol) in dimethylformamide (DMF) (15 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC:Hexanes. The desired fractions were pooled and the solvent was removed under reduced pressure to afford the Boc-protected title compound (4.2 mmol) as pale-yellow oil.

Subsequent Boc deprotection HCl (4M in dioxane, 30 minutes at room temperature) afforded (2S,3S)—N-(3,4-difluorophenyl)-2-methylpyrrolidine-3-carboxamide hydrochloride that was used as such in the next step without further purification.

### Intermediate III-O: Synthesis of (S)—N-(3,4-difluorophenyl)pyrrolidine-3-carboxamide hydrochloride

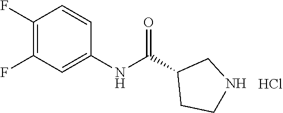 [SMILES: O=C(Nc1ccc(F)c(F)c1)[C@H]1CCNC1 ; Cl]

HATU 570 mg (1.5 mmol) and N,N-diisopropylethylamine (DIPEA) 1 mL were added to a solution of (S)-1-(tert-butoxycarbonyl)pyrrolidine-3-carboxylic acid 215 mg (1 mmol), 3,4-difluoroaniline 155 mg (1.2 mmol) in dimethylformamide (DMF) (5 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 20% EtOAC:Hexanes. The desired fractions were pooled and the solvent was removed under reduced pressure to afford the Boc-protected title compound (0.84 mmol) as pale-yellow oil.

Subsequent Boc deprotection HCl (4M in dioxane, 30 minutes at room temperature) afforded (S)—N-(3,4-difluorophenyl)pyrrolidine-3-carboxamide hydrochloride that was used as such in the next step without further purification.

### Intermediate III-P: Synthesis of (2S,3S)—N-(3-cyclopropyl-4-fluorophenyl)-2-methylpyrrolidine-3-carboxamide hydrochloride

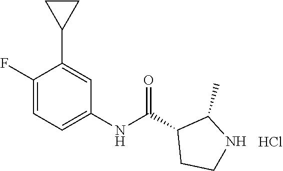 [SMILES: C[C@@H]1NCC[C@@H]1C(=O)Nc1ccc(F)c(C2CC2)c1 ; Cl]

HATU 1.67 g (4.4 mmol) and N,N-diisopropylethylamine (DIPEA) 1.15 mL (6.6 mmol) were added to a solution of (S)-1-(tert-butoxycarbonyl)pyrrolidine-3-carboxylic acid 477 mg (2.21 mmol), 3-chloro-4,5-difluoroaniline 400 mg (2.65 mmol) in dimethylformamide (DMF) (10 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford as a pale-yellow oil.

Subsequent Boc deprotection HCl (4M in dioxane, 30 minutes at room temperature) afford (2S,3S)—N-(3-cyclopropyl-4-fluorophenyl)-2-methylpyrrolidine-3-carboxamide hydrochloride that was used as such in the next step without further purification.

### Intermediate III-Q: Synthesis of (3S,4R)—N-(4-fluoro-3-methylphenyl)-4-methylpyrrolidine-3-carboxamide

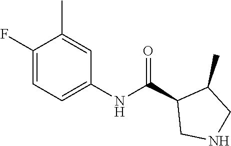 [SMILES: Cc1cc(NC(=O)[C@@H]2CNC[C@@H]2C)ccc1F]

(3S,4R)-1-((benzyloxy)carbonyl)-4-methylpyrrolidine-3-carboxylic acid 62.8 mg (0.5 mmol) was dissolved in 5 mL of DMF. Then, 346.8 g of HATU (0.912 mmol), and 218 μL of DIPEA (2.28 mmol) were added to the solution. The reaction mixture was stirred for 10 mins. and followed by adding 62.8 mg of p-toluidine. The reaction was kept stirring for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 30% EtOAC:Hexanes. The desired fractions were pooled and the solvent was removed under reduced pressure.

To remove the protecting group, 180 mg of benzyl (3S,4R)-3-((4-fluoro-3-methylphenyl) carbamoyl)-4-methylpyrrolidine-1-carboxylate (0.49 mmol) was dissolved in 5 mL of MeOH. Then, 10 mol % of Pd/C and 18.6 mg of NaBH₄ (0.49 mmol) were added. The reaction was stirred at room temperature for 30 mins. The desired product was obtained by filtering the reaction mixture through celite. The solvent was removed by rotavapor.

### Intermediate III-R: Synthesis of (3S,4S)—N-(4-fluoro-3-methylphenyl)-4-methylpyrrolidine-3-carboxamide, hydrochloride

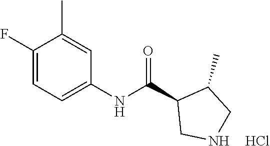 [SMILES: Cc1cc(NC(=O)[C@@H]2CNC[C@H]2C)ccc1F ; Cl]

HATU 933.3 mg (2 mmol) and N,N-diisopropylethylamine (DIPEA) 2 mL were added to a solution of (3S,4S)-1-(tert-butoxycarbonyl)-4-methylpyrrolidine-3-carboxylic acid 229 mg (1 mmol), p-toluidine 187.7 mg (1.5 mmol) in dimethylformamide (DMF) (5 mL) and stirred for 4 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography by using 30% EtOAC/Hexanes. The desired fractions were pooled and the solvent was removed under reduced pressure to afford product as a colorless oil.

Subsequent Boc deprotection HCl (4M in dioxane, 30 minutes at room temperature) afforded (3S,4S)—N-(4-fluoro-3-methylphenyl)-4-methylpyrrolidine-3-carboxamide, hydrochloride that was used as such in the next step without further purification.

### Synthesis of Intermediate V of Schemes III

### Intermediate V-A: Synthesis of methyl 5-(4,4,5,5-tetramethyl-1,3,2-dioxaborolan-2-yl)-1H-pyrrole-2-carboxylate

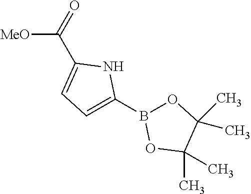 [SMILES: COC(=O)c1ccc(B2OC(C)(C)C(C)(C)O2)[nH]1]

1.02 g (5 mmol) of methyl 5-bromo-1H-pyrrole-2-carboxylate and 1.9 g (7.5 mmol) of bis(pinacolato)diboron was dissolved in 1,4-dioxane. The reaction mixture was bubbled under argon for 10 mins. Then 10 mol % of Pd(dppf)Cl₂ and 0.98 g (10 mmol) of KOAc was added to the reaction. Then, the reaction was heated at 10° C. for 4 hours. The product was obtained by filtering through celite. The celite was washed by EtOAc to remove the remaining product. The organic product was concentrated by rotavapor. Concentrated filtrate was used in the next step.

### Synthesis of Intermediate V of Schemes IV

### Intermediate V-B: Synthesis of methyl 6-(4,4,5,5-tetramethyl-1,3,2-dioxaborolan-2-yl)-1H-indole-2-carboxylate

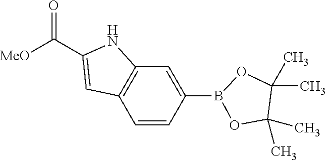 [SMILES: COC(=O)c1cc2ccc(B3OC(C)(C)C(C)(C)O3)cc2[nH]1]

670 mg (2.5 mmol) of methyl 6-bromo-1H-indole-2-carboxylate was dissolved in 1,4-dioxane. Consequently, 762 mg (3 mmol) bis(pinacolato)diboron and 736 mg (7.5 mmol) of KOAc were added to the flask. The reaction mixture was bubbled under argon for 15 mins. Then 10 mol % of Pd(dppf)Cl₂-DCM was added to the reaction. Then, the reaction was heated at 110° C. for 4 hours. The product was obtained by filtering through celite. The celite was washed by EtOAc to remove the remaining product. The organic product was concentrated by rotavapor. Concentrated filtrate was used in the next step.

### Example 1. (S)-1-(5-methyl-1H-pyrrole-2-carbonyl)-N-(3,4,5-trifluorophenyl)pyrrolidine-3-carboxamide

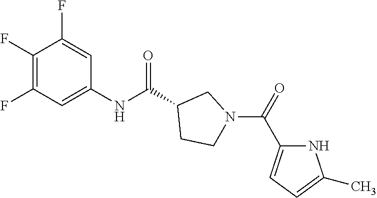 [SMILES: Cc1ccc(C(=O)N2CC[C@H](C(=O)Nc3cc(F)c(F)c(F)c3)C2)[nH]1]

The mixture of Intermediate III-A (1 eq.) and 5-methyl-1H-pyrrole-2-carboxylic acid (1.2 eq.) was dissolved in DMF (2 mL). Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 50-70% EtOAc/Hexanes. The desired fractions were pooled and the solvent was removed under reduced pressure to afford Example 1 (20 mg, 100% purity by UV) as a white solid. LCMS Method A, Rt=4.52 min, m/z=351.8 [M+H]+, LCMS Method C, Rt=4.42 min, m/z=352.1271 [M+H]+, exact mass: 351.1195.

### Example 2. (S)-1-(3-chloro-1H-indole-2-carbonyl)-N-(4-fluoro-3-methylphenyl)pyrrolidine-3-carboxamide

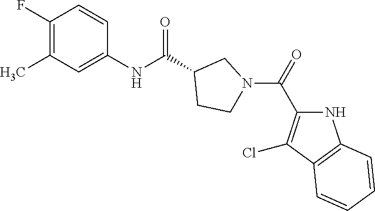 [SMILES: Cc1cc(NC(=O)[C@H]2CCN(C(=O)c3[nH]c4ccccc4c3Cl)C2)ccc1F]

The mixture of Intermediate III-B (1 eq.) and 3-chloro-1H-indole-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 50-70% EtOAc/hexanes to afford Example 2 (38 mg, 93% purity by UV) as a white solid. LCMS Method B, Rt=4.84 min, m/z=400.1 [M+H]+, LCMS Method C, Rt=4.81 min, m/z=400.1214 [M+H]+, exact mass: 399.1150.

### Example 3. (S)-1-(3-chlorothiophene-2-carbonyl)-N-(3,4,5-trifluorophenyl)pyrrolidine-3-carboxamide

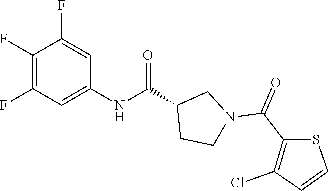 [SMILES: O=C(Nc1cc(F)c(F)c(F)c1)[C@H]1CCN(C(=O)c2sccc2Cl)C1]

The mixture of Intermediate III-A (1 eq.) and 3-chlorothiophene-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 60-70% EtOAc/hexanes to afford Example 3 (36 mg, 99% purity by UV) as a white solid. LCMS Method B, Rt=4.75 min, m/z=389.0 [M+H]+, LCMS Method C, Rt=4.61 min, m/z=389.0330 [M+H]+, exact mass: 388.0260.

### Example 4. (S)-1-(1H-indole-2-carbonyl)-N-(3,4,5-trifluorophenyl)pyrrolidine-3-carboxamide

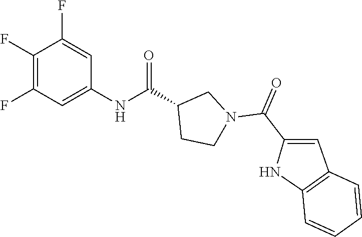 [SMILES: O=C(Nc1cc(F)c(F)c(F)c1)[C@H]1CCN(C(=O)c2cc3ccccc3[nH]2)C1]

The mixture of Intermediate III-A (1 eq.) and 1H-indole-2-carboxylic acid (1.2 eq.) was dissolved in DMF (3 mL). Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 50-80% EtOAc/Hexanes. The desired fractions were pooled and the solvent was removed under reduced pressure to afford Example 4 (28 mg, 100% purity by UV) as a white solid. LCMS Method B, Rt=4.45 min, m/z=388.1 [M+H]+, LCMS Method C, Rt=4.78 min, m/z=388.1252 [M+H]+, exact mass; 387.1195.

### Example 5. (S)-1-(3-chloro-5-methylthiophene-2-carbonyl)-N-(3,4,5-trifluorophenyl)pyrolidine-3-carboxamide

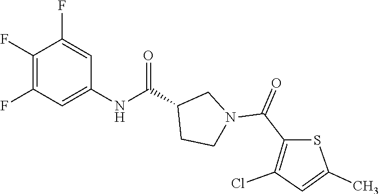 [SMILES: Cc1cc(Cl)c(C(=O)N2CC[C@H](C(=O)Nc3cc(F)c(F)c(F)c3)C2)s1]

The mixture of Intermediate III-A (1 eq.) and 3-chloro-5-methylthiophene-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 5 (5 mg, 100% purity by UV) as a white solid. LCMS Method B, Rt=4.86 min, m/z=403.1 [M+H]+, 425.1 [M+Na]+ exact mass: 402.0417.

### Example 6. (S)-1-(1H-indole-7-carbonyl)-N-(3,4,5-trifluorophenyl)pyrrolidine-3-carboxamide

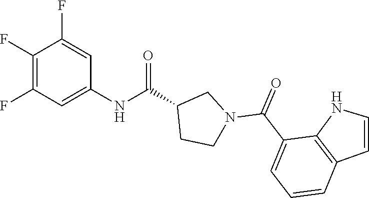 [SMILES: O=C(Nc1cc(F)c(F)c(F)c1)[C@H]1CCN(C(=O)c2cccc3cc[nH]c23)C1]

The mixture of Intermediate III-A (1 eq.) and 1H-indole-7-carboxylic acid was dissolved (1.2 eq.) in DMF (3 mL). Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 50-80% EtOAc/Hexanes. The desired fractions were pooled and the solvent was removed under reduced pressure to afford Example 6 (8 mg, 93 purity by UV) as a white solid. LCMS Method B, Rt=4.71 min, m/z=388.1 [M+H]+, 410.1 [M+Na]+ exact mass: 387.1195.

### Example 7. (S)-1-(4H-thieno[3,2-b]pyrrole-5-carbonyl)-N-(3,4,5-trifluoro-phenyl)pyrrolidine-3-carboxamide

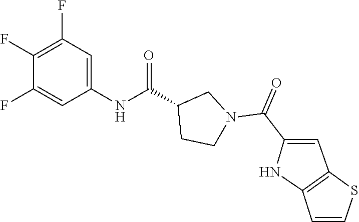 [SMILES: O=C(Nc1cc(F)c(F)c(F)c1)[C@H]1CCN(C(=O)c2cc3sccc3[nH]2)C1]

The mixture of Intermediate III-A (1 eq.) and 4H-thieno[3,2-b]pyrrole-5-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 7 (29 mg, 100% purity by UV) as a white solid. LCMS Method B, Rt=4.72 min, m/z=394.1 [M+H]+, 416.1 [M+Na]+, LCMS Method C, Rt=4.70 min, m/z=394.0814 [M+H]+, exact mass: 393.0759.

### Example 8. (S)-1-(5-cyano-1H-indole-2-carbonyl)-N-(3,4,5-trifluorophenyl)pyrrolidine-3-carboxamide

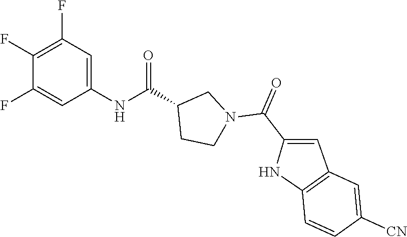 [SMILES: N#Cc1ccc2[nH]c(C(=O)N3CC[C@H](C(=O)Nc4cc(F)c(F)c(F)c4)C3)cc2c1]

The mixture of Intermediate III-A (1 eq.) and 5-cyano-1H-indole-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 8 (21 mg, 97% purity by UV) as a white solid. LCMS Method B, Rt=4.66 min, m/z=413.1 [M+H]+, 435.1 [M+Na]+, LCMS Method C, Rt=4.62 min, m/z=413.1221 [M+H]+, exact mass: 412.1147.

### Example 9. (S)-1-(4H-furo[3,2-b]pyrrole-5-carbonyl)-N-(3,4,5-trifluorophenyl)pyrrolidine-3-carboxamide

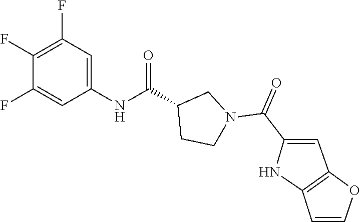 [SMILES: O=C(Nc1cc(F)c(F)c(F)c1)[C@H]1CCN(C(=O)c2cc3occc3[nH]2)C1]

The mixture of Intermediate III-A (1 eq.) and 4H-furo[3,2-b]pyrrole-5-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 9 (11 mg, 98% purity by UV) as a white solid. LCMS Method B, Rt=4.56 min, m/z=378.1 [M+H]+, 400.1 [M+Na]+ exact mass: 377.0987.

### Example 10. (S)—N-(3-bromo-4,5-difluorophenyl)-1-(1H-indole-2-carbonyl)pyrrolidine-3-carboxamide

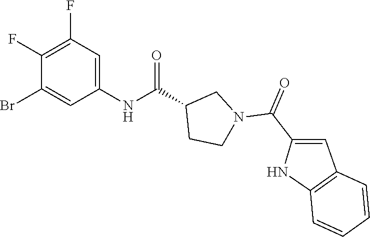 [SMILES: O=C(Nc1cc(F)c(F)c(Br)c1)[C@H]1CCN(C(=O)c2cc3ccccc3[nH]2)C1]

The mixture of Intermediate III-C (1 eq.) and 1H-indole-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 10 (19 mg, 100% purity by UV) as a white solid. LCMS Method B, Rt=4.90 min, m/z=448.1 [M+H]+, 470.1 [M+Na]+ exact mass: 447.0394.

### Example 11. (S)-1-(5-(difluoromethyl)-1H-pyrrole-2-carbonyl)-N-(3,4,5-trifluorophenyl)pyrrolidine-3-carboxamide

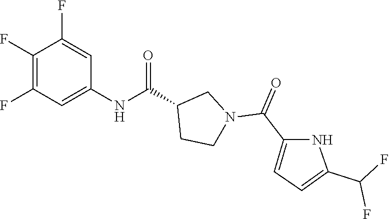 [SMILES: O=C(Nc1cc(F)c(F)c(F)c1)[C@H]1CCN(C(=O)c2ccc(C(F)F)[nH]2)C1]

The mixture of Intermediate III-A (1 eq.) and 5-formyl-1H-pyrrole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford (S)-1-(5-formyl-1H-pyrrole-2-carbonyl)-N-(3,4,5-trifluorophenyl)pyrrolidine-3-carboxamide as a white solid.

The solution of (S)-1-(5-formyl-1H-pyrrole-2-carbonyl)-N-(3,4,5-trifluorophenyl)pyrrolidine-3-carboxamide (1 eq.) in DCM (5 mL) was cooled to 0° C. and treated dropwise with diethylaminosulfur trifluoride (DAST) (5 eq.). The reaction was stirred for 12 hours. Brine was added to quench the reaction. The mixture was extracted with EtOAc (2×30 mL). The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 11 (18 mg, 88% purity by UV) as a white solid. LCMS Method B, Rt=4.61 min, m/z=388.1 [M+H]+, 410.1 [M+Na]+ exact mass: 387.1006.

### Example 12. (S)-1-(5-methoxy-1H-pyrrolo[2,3-c]pyridine-2-carbonyl)-N-(3,4,5-trifluorophenyl)pyrrolidine-3-carboxamide

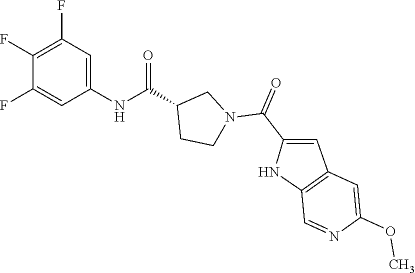 [SMILES: COc1cc2cc(C(=O)N3CC[C@H](C(=O)Nc4cc(F)c(F)c(F)c4)C3)[nH]c2cn1]

The mixture of Intermediate III-A (1 eq.) and 5-methoxy-1H-pyrrolo[2,3-c]pyridine-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 12 (3 mg, 98% purity by UV) as a white solid. LCMS Method B, Rt=4.18 min, m/z=419.1 [M+H]+ exact mass: 418.1253.

### Example 13. (S)-1-(6-methyl-1H-indole-2-carbonyl)-N-(3,4,5-trifluorophenyl)pyrrolidine-3-carboxamide

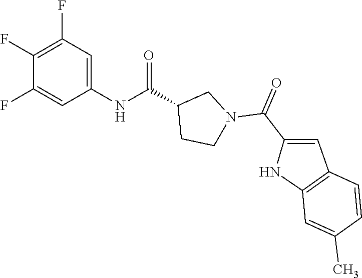 [SMILES: Cc1ccc2cc(C(=O)N3CC[C@H](C(=O)Nc4cc(F)c(F)c(F)c4)C3)[nH]c2c1]

The mixture of Intermediate III-A (1 eq.) and 6-methyl-1H-indole-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 13 (51 mg, 100% purity by UV) as a white solid. LCMS Method B, Rt=4.89 min., m/z=402.1 [M+H]+ 424.1 [M+Na]+, exact mass: 401.1351.

### Example 14. (S)-1-(1H-pyrrolo[3,2-b]pyridine-2-carbonyl)-N-(3,4,5-trifluorophenyl)pyrrolidine-3-carboxamide

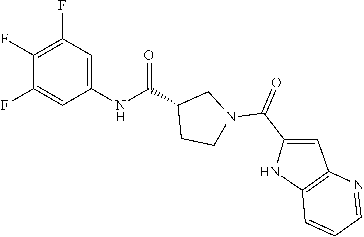 [SMILES: O=C(Nc1cc(F)c(F)c(F)c1)[C@H]1CCN(C(=O)c2cc3ncccc3[nH]2)C1]

The mixture of Intermediate III-A (1 eq.) and 1H-pyrrolo[3,2-b]pyridine-2-carboxylic acid (1 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 14 (2 mg, 98% purity by UV) as a white solid. LCMS Method B, Rt=4.00 min, m/z=389.1 [M+H]+, exact mass: 388.1147.

### Example 15. (S)-1-(4,5-dimethyl-1H-pyrrole-2-carbonyl)-N-(3,4,5-trifluorophenyl)pyrrolidine-3-carboxamide

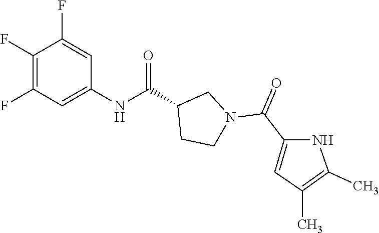 [SMILES: Cc1cc(C(=O)N2CC[C@H](C(=O)Nc3cc(F)c(F)c(F)c3)C2)[nH]c1C]

The mixture of Intermediate III-A (1 eq.) and 4,5-dimethyl-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 15 (5 mg, 89% purity by UV) as a white solid. LCMS Method B, Rt=4.67 min, m/z=366.1 [M+H]+, LCMS Method C, Rt=4.65 min, m/z=366.1439 [M+H]+, exact mass: 365.1351.

### Example 16. (S)-1-(3-chloro-1H-pyrrole-2-carbonyl)-N-(3,4,5-trifluorophenyl)pyrrolidine-3-carboxamide

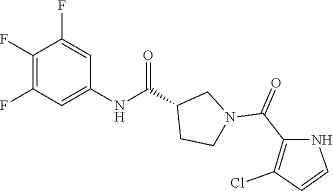 [SMILES: O=C(Nc1cc(F)c(F)c(F)c1)[C@H]1CCN(C(=O)c2[nH]ccc2Cl)C1]

The mixture of Intermediate III-A (1 eq.) and 3-chloro-1H-pyrrole-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 16 (24 mg, 99% purity by UV) as a white solid. LCMS Method B, Rt=4.55 min, m/z=372.1 [M+H]+ 394.1 [M+Na]+, LCMS Method C, Rt=4.41 min, m/z=372.0733 [M+H]+, exact mass: 371.0648.

### Example 17. (2S,3S)-2-methyl-1-(1H-pyrrolo[2,3-b]pyridine-2-carbonyl)-N-(3,4,5-trifluorophenyl) pyrrolidine-3-carboxamide

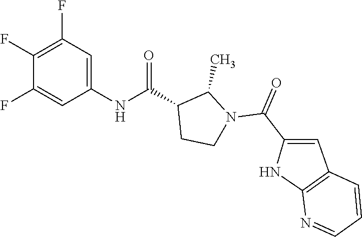 [SMILES: C[C@H]1[C@@H](C(=O)Nc2cc(F)c(F)c(F)c2)CCN1C(=O)c1cc2cccnc2[nH]1]

The mixture of Intermediate III-D (1 eq.) and 1H-pyrrolo[2,3-b]pyridine-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 17 (46 mg, 100% purity by UV) as a white solid. LCMS Method B, Rt=4.60 min, m/z=403.1 [M+H]+, LCMS Method C, Rt=4.41 min, m/z=403.1368 [M+H]+, exact mass: 402.1304.

### Example 18. (S)—N-(3-cyano-4-fluorophenyl)-1-(1H-pyrrolo[2,3-b]pyridine-2-carbonyl)pyrrolidine-3-carboxamide

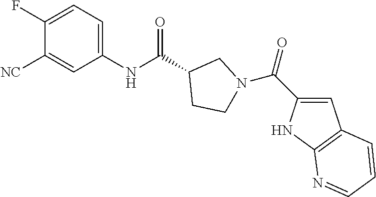 [SMILES: N#Cc1cc(NC(=O)[C@H]2CCN(C(=O)c3cc4cccnc4[nH]3)C2)ccc1F]

The mixture of Intermediate III-E (1 eq.) and 1H-pyrrolo[2,3-b]pyridine-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to give Example 18 (8 mg, 100% purity by UV) as a white solid. LCMS Method B, Rt=4.30 min, m/z=378.1 [M+H]+, LCMS Method C, Rt=3.70 min, m/z=378.1360 [M+H]+, exact mass: 377.1288.

### Example 19. (2S,3S)-1-(5-cyano-1H-pyrrole-2-carbonyl)-2-methyl-N-(3,4,5-trifluorophenyl)pyrrolidine-3-carboxamide

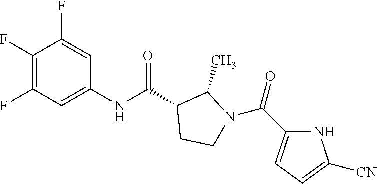 [SMILES: C[C@H]1[C@@H](C(=O)Nc2cc(F)c(F)c(F)c2)CCN1C(=O)c1ccc(C#N)[nH]1]

The mixture Intermediate III-D (1 eq.) and 5-cyano-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 50-100% EtOAc/Hexanes. The desired fractions were pooled and the solvent was removed under reduced pressure to afford Example 19 (12 mg, 96% purity by UV) as a white solid. LCMS Method B, Rt=4.68 min, m/z=377.1 [M+H]+ 399 [M+Na]+, LCMS Method C, Rt=4.57 min, m/z=377.1237 [M+H]+, exact mass: 376.1147.

### Example 20. (2S,3S)—N-(3-chloro-4,5-difluorophenyl)-2-methyl-1-(5-methyl-1H-pyrrole-2-carbonyl) pyrrolidine-3-carboxamide

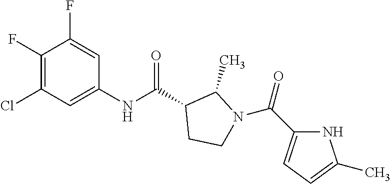 [SMILES: Cc1ccc(C(=O)N2CC[C@H](C(=O)Nc3cc(F)c(F)c(Cl)c3)[C@@H]2C)[nH]1]

The mixture of Intermediate III-F (1 eq.) and 5-methyl-1H-pyrrole-2-carboxylic acid (1.1 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to give Example 20 (4 mg, 99% purity by UV) as a white solid. LCMS Method B, Rt=4.82 min, m/z=382.1 [M+H]+, LCMS Method C, Rt=4.87 min, m/z=382.1156 [M+H]+, exact mass: 381.1056.

### Example 21. (2S,3S)—N-(3-cyano-4-fluorophenyl)-2-methyl-1-(1H-pyrrolo[2,3-b]pyridine-2-carbonyl) pyrrolidine-3-carboxamide

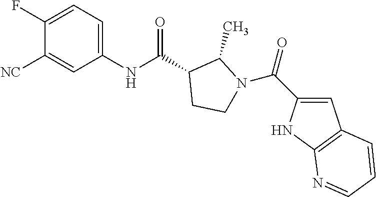 [SMILES: C[C@H]1[C@@H](C(=O)Nc2ccc(F)c(C#N)c2)CCN1C(=O)c1cc2cccnc2[nH]1]

The mixture Intermediate III-G (1 eq.) and 1H-pyrrolo[2,3-b]pyridine-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 21 (12 mg, 100% purity by UV) as a white solid. LCMS Method B, Rt=4.41 min, m/z=392.2 [M+H]+, exact mass: 391.1444.

### Example 22. (S)-1-(5-chloro-1H-pyrrole-2-carbonyl)-N-(3-cyano-4-fluorophenyl)pyrrolidine-3-carboxamide

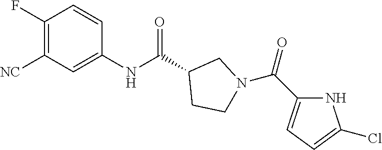 [SMILES: N#Cc1cc(NC(=O)[C@H]2CCN(C(=O)c3ccc(Cl)[nH]3)C2)ccc1F]

The mixture of Intermediate III-E (1.2 eq.) and 5-chloro-1H-pyrrole-2-carboxylic acid (1 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to give afford Example 22 (5 mg, 100% purity by UV) as a white solid. LCMS Method B, Rt=4.52 min, m/z=361.1 [M+H]+, LCMS Method C, Rt=4.10 min, m/z=361.0884 [M+H]+, exact mass: 360.0789.

### Example 23. (S)-1-(5H-pyrrolo[2,3-b]pyrazine-6-carbonyl)-N-(3,4,5-trifluorophenyl)pyrrolidine-3-carboxamide

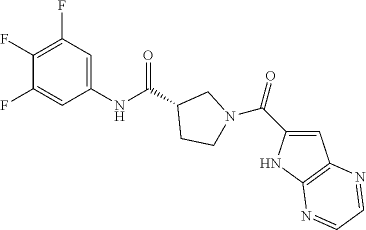 [SMILES: O=C(Nc1cc(F)c(F)c(F)c1)[C@H]1CCN(C(=O)c2cc3nccnc3[nH]2)C1]

The mixture of Intermediate III-A (1 eq.) and 5H-pyrrolo[2,3-b]pyrazine-6-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 23 (10 mg, 90% purity by UV) as a white solid. LCMS Method B, Rt=4.29 min, m/z=390.1 [M+H]+, exact mass: 389.1100. ¹H-NMR (500 MHz, DMSO-d₆) δ 10.53 (s, 1H), 10.46 (s, 1H), 8.49 (d, J=2.4 Hz, 1H), 8.37 (d, J=2.4 Hz, 1H), 7.61-7.42 (m, 2H), 7.14 (d, J=3.4 Hz, 1H), 4.04-3.83 (m, 2H), 3.81-3.68 (m, 1H), 3.31-3.19 (m, 2H), 2.37-2.07 (m, 2H).

### Example 24. (S)—N-(4-fluoro-3-methylphenyl)-1-(1H-pyrrolo[2,3-b]pyridine-2-carbonyl)pyrrolidine-3-carboxamide

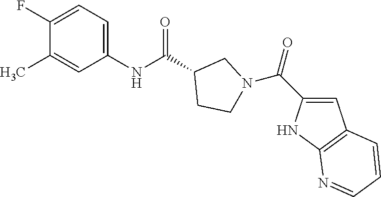 [SMILES: Cc1cc(NC(=O)[C@H]2CCN(C(=O)c3cc4cccnc4[nH]3)C2)ccc1F]

The mixture of Intermediate III-B (1 eq.) and 1H-pyrrolo[2,3-b]pyridine-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 24 (32 mg, 99% purity by UV) as a white solid. LCMS Method B, Rt=4.37 min, m/z=367.1 [M+H]+, LCMS Method C, Rt=3.90 min, m/z=367.1575 [M+H]+, exact mass: 366.1492.

### Example 25. (S)-1-(1H-benzo[d]imidazole-2-carbonyl)-N-(3,4,5-trifluorophenyl)pyrrolidine-3-carboxamide

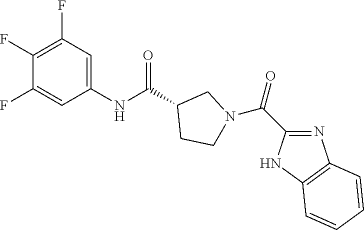 [SMILES: O=C(Nc1cc(F)c(F)c(F)c1)[C@H]1CCN(C(=O)c2nc3ccccc3[nH]2)C1]

The mixture of Intermediate III-A (1 eq.) and 1H-benzo[d]imidazole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 25 (15 mg, 100% purity by UV) as a white solid. LCMS Method B, Rt=4.61 min, m/z=389.1 [M+H]+, exact mass: 388.1147. ¹H-NMR (500 MHz, Acetone-d₆) δ 12.12 (s, 1H), 9.82 (s, 1H), 7.76 (d, J=8.1 Hz, 1H), 7.63 (d, J=8.0 Hz, 1H), 7.58-7.50 (m, 2H), 7.34 (dd, J=7.9, 7.3 Hz, 1H), 7.30-7.24 (m, 1H), 3.97 (dd, J=12.0, 8.4 Hz, 1H), 3.93-3.82 (m, 1H), 3.71-3.65 (m, 1H), 3.40 (p, J=7.4, 1H), 3.31 (p, J=7.5 Hz, 1H), 2.43-2.20 (m, 2H).

### Example 26. (S)-1-(1H-indole-3-carbonyl)-N-(3,4,5-trifluorophenyl)pyrrolidine-3-carboxamide

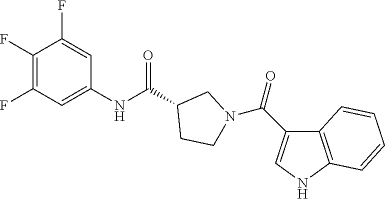 [SMILES: O=C(Nc1cc(F)c(F)c(F)c1)[C@H]1CCN(C(=O)c2c[nH]c3ccccc23)C1]

The mixture of Intermediate III-A (1 eq. and added 1H-indole-3-carboxylic acid (1 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 80% EtOAc/hexanes to afford Example 26 (34 mg, 96% purity by UV) as a white solid. LCMS Method A, Rt=4.35 min, m/z=387.9 [M+H]+, exact mass: 387.1195.

### Example 27. (S)-1-(1H-imidazole-2-carbonyl)-N-(3,4,5-trifluorophenyl)pyrrolidine-3-carboxamide

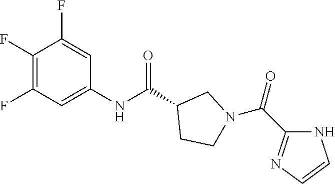 [SMILES: O=C(Nc1cc(F)c(F)c(F)c1)[C@H]1CCN(C(=O)c2ncc[nH]2)C1]

The mixture of Intermediate III-A (1 eq.) and 1H-imidazole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 27 (41 mg, 98% purity by UV) as a white solid. LCMS Method A, Rt=3.34 min, m/z=338.7 [M+H]+, exact mass; 338.0991.

### Example 28. (S)-1-(thiazole-2-carbonyl)-N-(3,4,5-trifluorophenyl)pyrrolidine-3-carboxamide

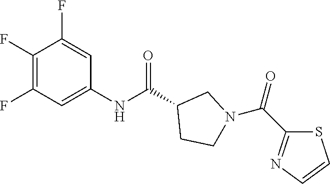 [SMILES: O=C(Nc1cc(F)c(F)c(F)c1)[C@H]1CCN(C(=O)c2nccs2)C1]

The mixture of Intermediate III-A (1 eq.) and thiazole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 28 (22 mg, 98% purity by UV) as a white solid. LCMS Method A, Rt=4.40 min, m/z=355.7 [M+H]+, LCMS Method C, Rt=4.35 min, m/z=356.0669 [M+H]+, exact mass: 355.0602.

### Example 29. (S)-1-(4-methyl-1H-pyrazole-3-carbonyl)-N-(3,4,5-trifluorophenyl)pyrrolidine-3-carboxamide

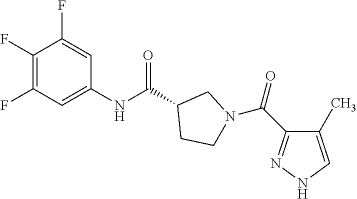 [SMILES: Cc1c[nH]nc1C(=O)N1CC[C@H](C(=O)Nc2cc(F)c(F)c(F)c2)C1]

The mixture of Intermediate III-A (1 eq.) and 4-methylpyrazole-3-carboxylic acid (1 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 80% EtOAc/hexanes to afford Example 29 (28 mg, 100% purity by UV) as a white solid. LCMS Method A, Rt=3.74 min, m/z=352.7 [M+H]+, exact mass: 352.1147.

### Example 30. (S)-1-(1H-imidazole-5-carbonyl)-N-(3,4,5-trifluorophenyl)pyrrolidine-3-carboxamide

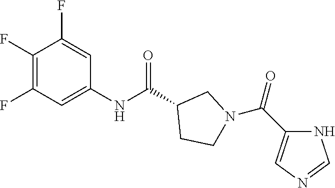 [SMILES: O=C(Nc1cc(F)c(F)c(F)c1)[C@H]1CCN(C(=O)c2cnc[nH]2)C1]

The mixture of Intermediate III-A (1 eq.) and 1H-imidazole-5-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 30 (25 mg, 100% purity by UV) as a white solid. LCMS Method A, Rt=3.41 min, m/z=338.7 [M+H]+, exact mass: 338.0991.

### Example 31. (S)-1-(3-methyl-1H-pyrazole-4-carbonyl)-N-(3,4,5-trifluorophenyl)pyrrolidine-3-carboxamide

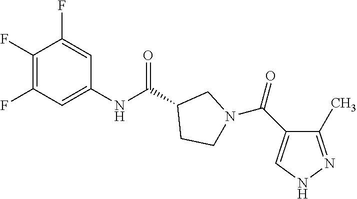 [SMILES: Cc1n[nH]cc1C(=O)N1CC[C@H](C(=O)Nc2cc(F)c(F)c(F)c2)C1]

The mixture of Intermediate III-A (1.2 eq.) and added 3-methyl-1H-pyrazole-4-carboxylic acid (1 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with EtOAc to afford Example 31 (10 mg, 100% purity) as a white solid. LCMS Method A, Rt=3.45 min, m/z=352.7 [M+H]+, exact mass: 352.1147.

### Example 32. (S)—N-(3-chloro-4-fluorophenyl)-1-(1H-imidazole-2-carbonyl)pyrrolidine-3-carboxamide

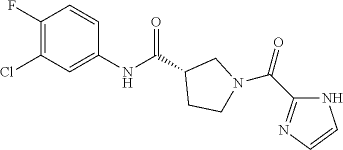 [SMILES: O=C(Nc1ccc(F)c(Cl)c1)[C@H]1CCN(C(=O)c2ncc[nH]2)C1]

The mixture of Intermediate III-I (1 eq.) and 1H-imidazole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 32 (16 mg, 100% purity by UV) as a white solid. LCMS Method A, Rt=3.25 min, m/z=336.8 [M+H]+, exact mass: 336.0789.

### Example 33. (S)—N-(3-chloro-4-fluorophenyl)-1-(5-methyl-1H-pyrrole-2-carbonyl)pyrrolidine-3-carboxamide

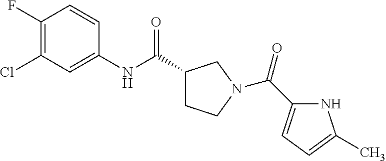 [SMILES: Cc1ccc(C(=O)N2CC[C@H](C(=O)Nc3ccc(F)c(Cl)c3)C2)[nH]1]

The mixture of Intermediate III-I (1 eq.) and 5-methyl-1H-pyrrole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 33 (9 mg, 100% purity by UV) as a white solid. LCMS Method A, Rt=4.47 min, m/z=349.8 [M+H]+, LCMS Method C, Rt=4.40 min, m/z=350.1077 [M+H]+, exact mass: 349.0993.

### Example 34. (S)-1-(3,5-dimethyl-1H-pyrrole-2-carbonyl)-N-(3,4,5-trifluorophenyl)pyrrolidine-3-carboxamide

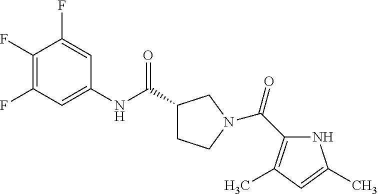 [SMILES: Cc1cc(C)c(C(=O)N2CC[C@H](C(=O)Nc3cc(F)c(F)c(F)c3)C2)[nH]1]

The mixture of Intermediate III-A (1 eq.) and 3,5-dimethyl-1H-pyrrole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 34 (8 mg, 94% purity by UV) as a white solid. LCMS Method B, Rt=4.43 min, m/z=366.2 [M+H]+, LCMS Method C, Rt=4.51 min, m/z=366.1430 [M+H]+, exact mass: 365.1351.

### Example 35. (S)-1-(1H-indole-6-carbonyl)-N-(3,4,5-trifluorophenyl)pyrrolidine-3-carboxamide

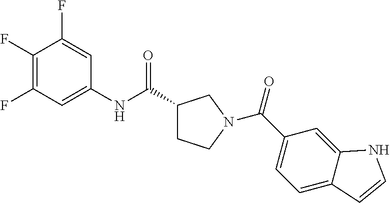 [SMILES: O=C(Nc1cc(F)c(F)c(F)c1)[C@H]1CCN(C(=O)c2ccc3cc[nH]c3c2)C1]

The mixture of Intermediate III-A (1 eq.) and 1H-indole-6-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 35 (13 mg, 98% purity) as a white solid. LCMS Method B, Rt=4.36 min, m/z=388.2 [M+H]+, LCMS Method C, Rt=4.42 min, m/z=388.1285 [M+H]+, exact mass: 387.1195.

### Example 36. (S)-1-(3-formyl-1H-indole-6-carbonyl)-N-(3,4,5-trifluorophenyl)pyrrolidine-3-carboxamide

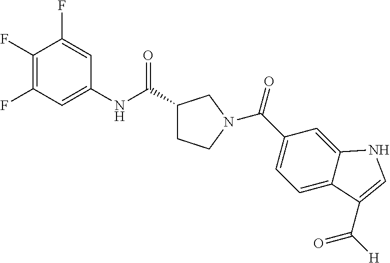 [SMILES: O=Cc1c[nH]c2cc(C(=O)N3CC[C@H](C(=O)Nc4cc(F)c(F)c(F)c4)C3)ccc12]

The mixture of Intermediate III-A (1 eq.) and 3-formyl-1H-indole-6-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 36 (41 mg, 99% purity by UV) as a white solid. LCMS Method B, Rt=4.21 min, m/z=416.2 [M+H]+, LCMS Method C, Rt=4.03 min, m/z=416.1251 [M+H]+, exact mass: 415.1144.

### Example 37. (S)-1-(3-(((2-hydroxyethyl)amino)methyl)-1H-indole-6-carbonyl)-N-(3,4,5-trifluorophenyl) pyrrolidine-3-carboxamide

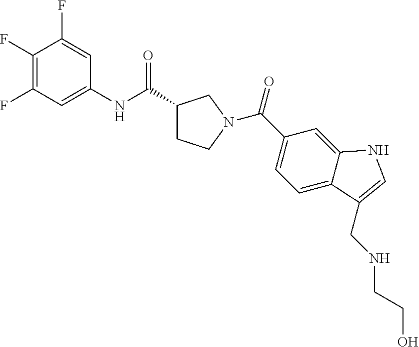 [SMILES: O=C(Nc1cc(F)c(F)c(F)c1)[C@H]1CCN(C(=O)c2ccc3c(CNCCO)c[nH]c3c2)C1]

To a stirred solution of Example 36 (1 eq.), ethanolamine (4 eq.) and acetic acid (cat.) in DCM:MeOH (1:1, 5 mL) at 0° C. under N2, was added sodiumtriacetoxyborohydride (2.1 eq.) portion wise over 10 minutes. The reaction mixture was allowed to warm to room temperature over 12 hours and stirred for an additional 24 to 72 hours at room temperature. The reaction mixture was quenched by 1 M NaOH and was allowed to stir at room temperature for 30 minutes. The mixture was then extracted with dichloromethane (3×100 mL), collected the organic layer, and washed with brine (2×50 mL), then dried over anhydrous sodium sulfate. The solvent was removed in vacuo to give the crude product. The product was purified by sephadex afforded the corresponding alcohol 37 (13 mg, 99% purity by UV) as colorless oil. LCMS Method B, Rt=3.98 min, m/z=461.2 [M+H]+, exact mass: 460.1722.

### Example 38. (S)-1-(4-acetyl-1H-pyrrole-2-carbonyl)-N-(3,4,5-trifluorophenyl)pyrrolidine-3-carboxamide

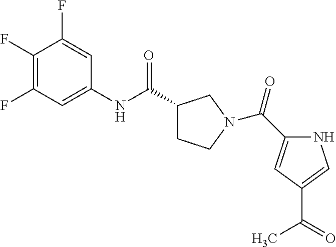 [SMILES: CC(=O)c1c[nH]c(C(=O)N2CC[C@H](C(=O)Nc3cc(F)c(F)c(F)c3)C2)c1]

The mixture of Intermediate III-A (1 eq.) and 4-acetyl-1H-pyrrole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layers was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 38 (23 mg, 100% purity by UV) as a white solid. LCMS Method B, Rt=4.19 min, m/z=380.1 [M+H]+, exact mass: 379.1144.

### Example 39. (3S)-1-(4-(1-hydroxyethyl)-1H-pyrrole-2-carbonyl)-N-(3,4,5-trifluorophenyl)pyrrolidine-3-carboxamide

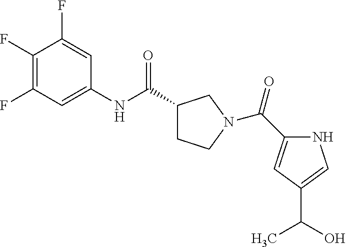 [SMILES: CC(O)c1c[nH]c(C(=O)N2CC[C@H](C(=O)Nc3cc(F)c(F)c(F)c3)C2)c1]

Example 39 could be achieved by treating Example 38 (1 eq.) with sodiumborohydride (2 eq.) in MeOH. The reaction mixture was stirred for 30 to 60 minutes at room temperature. The mixture was removed under reduced pressure and then extracted with EtOAc (3×25 mL) and collected the organic layer. The organic phase was washed with brine (2×30 mL), and dried over anhydrous sodium sulfate. The solvent was removed in vacuo to give the crude amine. Further purification by sephadex afforded the corresponding alcohol 39 (8 mg, 100% purity by UV) as liquid/oil. LCMS Method B, Rt=4.10 min, m/z=382.1 [M+H]+ 404.1 [M+Na]+, exact mass: 381.1300.

### Example 40. (S)—N-(3-chloro-4-fluorophenyl)-1-(4-cyano-1H-pyrrole-2-carbonyl)pyrrolidine-3-carboxamide

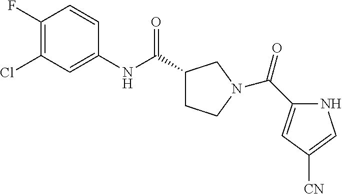 [SMILES: N#Cc1c[nH]c(C(=O)N2CC[C@H](C(=O)Nc3ccc(F)c(Cl)c3)C2)c1]

The mixture of Intermediate III-I (1 eq.) and 4-cyano-1H-pyrrole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 40 (21 mg, 100% purity by UV as a white solid. LCMS Method B, Rt=4.26 min, m/z=361.1 [M+H]+, exact mass: 360.0789.

### Example 41. (S)-1-(3-chloro-1H-indole-2-carbonyl)-N-(3-chloro-4-fluoro-phenyl)pyrrolidine-3-carboxamide

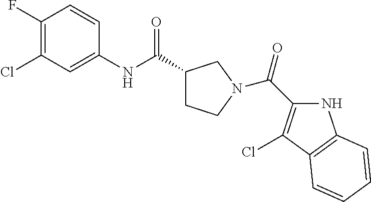 [SMILES: O=C(Nc1ccc(F)c(Cl)c1)[C@H]1CCN(C(=O)c2[nH]c3ccccc3c2Cl)C1]

The mixture of Intermediate III-I (1 eq.) and 3-chloro-1H-indole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 70-100% EtOAc/Hexanes. The desired fractions were pooled and the solvent was removed under reduced pressure to afford Example 41 (12 mg, 91% purity by UV) as a white solid. LCMS Method B, Rt=4.60 min, m/z=420.0 [M+H]+, LCMS Method C, Rt=4.93 min, m/z=420.0678 [M+H]+, exact mass: 419.0604.

### Example 42. (2S,3S)-1-(3-chloro-1H-indole-2-carbonyl)-2-methyl-N-(3,4,5-trifluorophenyl)pyrrolidine-3-carboxamide

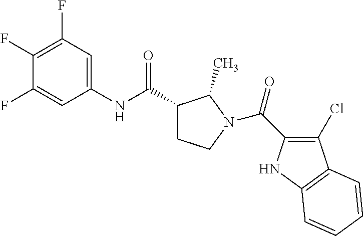 [SMILES: C[C@H]1[C@@H](C(=O)Nc2cc(F)c(F)c(F)c2)CCN1C(=O)c1[nH]c2ccccc2c1Cl]

The mixture of Intermediate III-D (1 eq.) and 3-chloro-1H-indole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 42 (22 mg, 99% purity by UV) as a white solid. LCMS Method B, Rt=5.11 min, m/z=436.1 [M+H]+, LCMS Method C, Rt=5.23 min, m/z=436.1039 [M+H]+, exact mass: 435.0961. ¹H-NMR (500 MHz, DMSO-d₆) δ11.9 (s, 1H), 10.4 (s, 1H), 7.53-7.49 (m, 3H), 7.41 (d, J=8.2 Hz, 1H), 7.25 (t, J=7.6 Hz, 1H), 7.15 (t, J=7.5 Hz, 1H), 4.71 (m, 1H), 3.80 (m, 1H), 3.23 (m, 2H), 2.30 (m, 1H), 1.98 (m, 1H), 1.12 (d, J=5.8 Hz, 3H).

### Example 43. (S)-1-(3-chloro-1H-indole-2-carbonyl)-N-(3-cyano-4-fluorophenyl)pyrrolidine-3-carboxamide

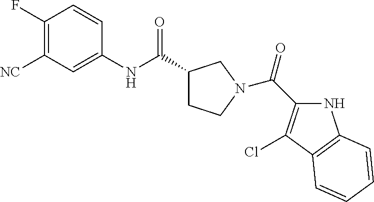 [SMILES: N#Cc1cc(NC(=O)[C@H]2CCN(C(=O)c3[nH]c4ccccc4c3Cl)C2)ccc1F]

The mixture of Intermediate III-E (1 eq.) and 3-chloro-1H-indole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 43 (12 mg, 93% purity by UV) as a white solid. LCMS Method B, Rt=4.67 min, m/z=409.1 [M−H]−, LCMS Method C, Rt=4.59 min, m/z=411.1016 [M+H]+, exact mass: 410.0946.

### Example 44. (S)-1-(5-methylthiophene-2-carbonyl)-N-(3,4,5-trifluorophenyl)pyrrolidine-3-carboxamide

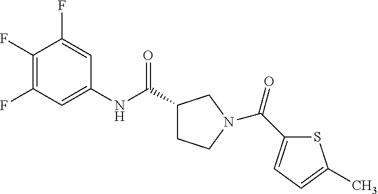 [SMILES: Cc1ccc(C(=O)N2CC[C@H](C(=O)Nc3cc(F)c(F)c(F)c3)C2)s1]

The mixture of Intermediate III-A (1 eq.) and 5-methylthiophene-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 44 (10 mg, 100% purity by UV) as a white solid. LCMS Method B, Rt=4.76 min., m/z=369.1 [M+H]+, LCMS Method C, Rt=4.66 min, m/z=369.0868 [M+H]+, exact mass: 368.0806.

### Example 45. (S)-1-(3-cyano-1H-indole-2-carbonyl)-N-(3,4,5-trifluorophenyl)pyrrolidine-3-carboxamide

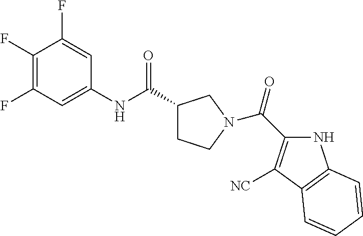 [SMILES: N#Cc1c(C(=O)N2CC[C@H](C(=O)Nc3cc(F)c(F)c(F)c3)C2)[nH]c2ccccc12]

The mixture of Intermediate III-A (1 eq.) and 3-cyano-1H-indole-2-carboxylic acid (1.2 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 45 (15 mg, 93% purity by UV as a white solid. LCMS Method B, Rt=4.78 min, m/z=413.1 [M+H]+, LCMS Method C, Rt=4.62 min, m/z=413.1214 [M+H]+, exact mass: 412.1147.

### Example 46. (S)-1-(1H-pyrrolo[3,2-c]pyridine-2-carbonyl)-N-(3,4,5-trifluorophenyl)pyrrolidine-3-carboxamide

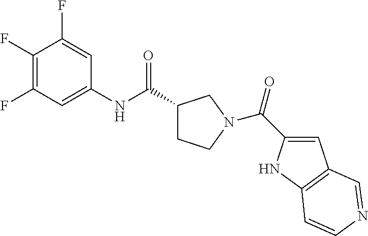 [SMILES: O=C(Nc1cc(F)c(F)c(F)c1)[C@H]1CCN(C(=O)c2cc3cnccc3[nH]2)C1]

The mixture of Intermediate III-A (1 eq.) and 5-azaindole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 46 (11 mg, 87% purity by UV) as a white solid. LCMS Method B, Rt=4.01 min, m/z=389.1 [M+H]+ 411.1 [M+Na]+, LCMS Method C, Rt=3.29 min, m/z=389.1207 [M+H]+, exact mass: 388.1147.

### Example 47. (S)-1-(1H-pyrrolo[2,3-c]pyridine-2-carbonyl)-N-(3,4,5-trifluorophenyl)pyrrolidine-3-carboxamide

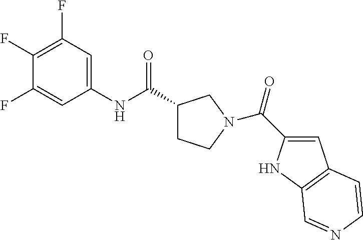 [SMILES: O=C(Nc1cc(F)c(F)c(F)c1)[C@H]1CCN(C(=O)c2cc3ccncc3[nH]2)C1]

The mixture of Intermediate III-A (1 eq.) and 1H-pyrrolo[2,3-c]pyridine-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 47 (11 mg, 100% purity by UV) as a white solid. LCMS Method B, Rt=4.01 min, m/z=389.1 [M+H]+, LCMS Method C, Rt=3.28 min, m/z=389.1215 [M+H]+, exact mass: 388.1147.

### Example 48. (S)-1-(1H-pyrrole-2-carbonyl)-N-(3,4,5-trifluorophenyl)pyrrolidine-3-carboxamide

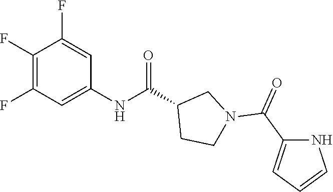 [SMILES: O=C(Nc1cc(F)c(F)c(F)c1)[C@H]1CCN(C(=O)c2ccc[nH]2)C1]

The mixture of Intermediate III-A (1 eq.) and 1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 48 (18 mg, 100% purity by UV) as a white solid. LCMS Method B, Rt=4.50 min, m/z=338.1 [M+H]+, 360.1 [M+Na]+, LCMS Method C, Rt=4.26 min, m/z=338.1126 [M+H]+, exact mass: 337.1038.

### Example 49. (S)-1-(5,6-difluoro-1H-indole-2-carbonyl)-N-(3,4,5-trifluorophenyl)pyrrolidine-3-carboxamide

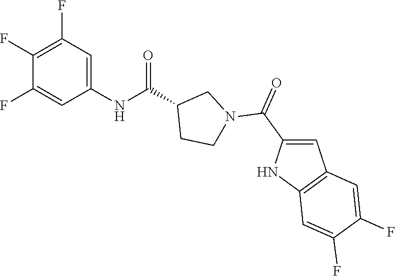 [SMILES: O=C(Nc1cc(F)c(F)c(F)c1)[C@H]1CCN(C(=O)c2cc3cc(F)c(F)cc3[nH]2)C1]

The mixture of Intermediate III-A (1 eq.) and 5,6-difluoro-1H-indole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 49 (36 mg, 98% purity by UV) as a white solid. LCMS Method B, Rt=4.91 min, m/z=424.1 [M+H]+ 446.1 [M+Na]+, exact mass: 423.1006.

### Example 50. (S)-1-(5-cyano-1H-pyrrole-2-carbonyl)-N-(3,4,5-trifluorophenyl)pyrrolidine-3-carboxamide

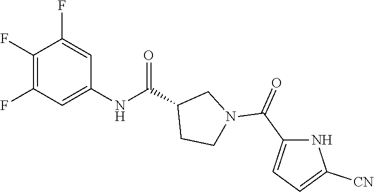 [SMILES: N#Cc1ccc(C(=O)N2CC[C@H](C(=O)Nc3cc(F)c(F)c(F)c3)C2)[nH]1]

The mixture of Intermediate III-A (1 eq.) and 5-cyano-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 50 (35 mg, 100% purity by UV) as a white solid. LCMS Method B, Rt=4.50 min, m/z=363.1 [M+H]+ 385.1 [M+Na]+, exact mass: 362.0991.

### Example 51. (S)—N-(3-bromo-4,5-difluorophenyl)-1-(5-methyl-1H-pyrrole-2-carbonyl)pyrrolidine-3-carboxamide

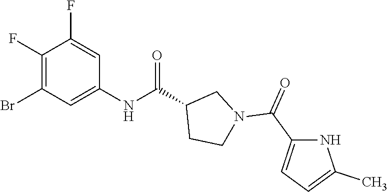 [SMILES: Cc1ccc(C(=O)N2CC[C@H](C(=O)Nc3cc(F)c(F)c(Br)c3)C2)[nH]1]

The mixture of Intermediate III-C (1 eq.) and 5-methyl-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer as dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 51 (28 mg, 100% purity by UV) as a white solid. LCMS Method B. Rt=4.73 min., m/z=412.1 [M+H]+, LCMS Method C, Rt=4.64 min, m/z=412.0506 [M+H]+, exact mass: 411.0394.

### Example 52. (S)-1-(5-chloro-1H-pyrrole-2-carbonyl)-N-(3,4,5-trifluorophenyl)pyrrolidine-3-carboxamide

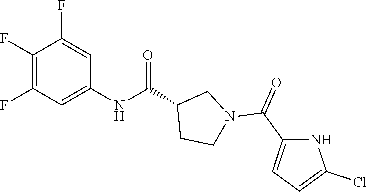 [SMILES: O=C(Nc1cc(F)c(F)c(F)c1)[C@H]1CCN(C(=O)c2ccc(Cl)[nH]2)C1]

The mixture of Intermediate III-A (1 eq.) and 5-chloro-1H-pyrrole-2-carboxylic acid (1.3 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 52 (3 mg, 97% purity by UV) as a white solid. LCMS Method B, Rt=4.61 min, m/z=372.1 [M+H]+ 394.0 [M+Na]+, LCMS Method C, Rt=4.56 min, m/z=372.0736 [M+H]+, exact mass: 371.0648.

### Example 53. (S)—N-(3-bromo-4,5-difluorophenyl)-1-(1H-pyrrolo[3,2-c]pyridine-2-carbonyl)pyrrolidine-3-carboxamide

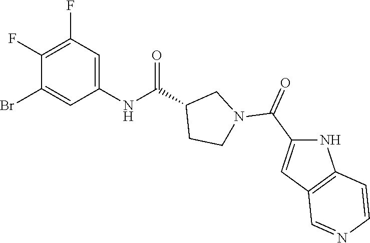 [SMILES: O=C(Nc1cc(F)c(F)c(Br)c1)[C@H]1CCN(C(=O)c2cc3cnccc3[nH]2)C1]

The mixture of Intermediate III-C (1 eq.) and 1H-pyrrolo[3,2-c]pyridine-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 53 (22 mg, 100% purity by UV) as a white solid. LCMS Method B, Rt=4.11 min, m/z=449.1 [M+H]+ 451.1 [M+Na]+, exact mass: 448.0346.

### Example 54. (S)-1-(1H-pyrrolo[2,3-b]pyridine-2-carbonyl)-N-(3,4,5-trifluorophenyl)pyrrolidine-3-carboxamide

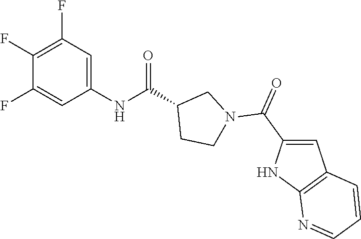 [SMILES: O=C(Nc1cc(F)c(F)c(F)c1)[C@H]1CCN(C(=O)c2cc3cccnc3[nH]2)C1]

The mixture of Intermediate III-A (1 eq.) and 1H-pyrrolo[2,3-b]pyridine-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 54 (16 mg, 98% purity by UV) as a white solid. LCMS Method B, Rt=4.41 min, m/z=389.1 [M+H]+, LCMS Method C, Rt=4.13 min, m/z=389.1224 [M+H]+, exact mass: 388.1147.

### Example 55. (S)-1-(5-methyl-1H-indole-2-carbonyl)-N-(3,4,5-trifluorphenyl)pyrrolidine-3-carboxamide

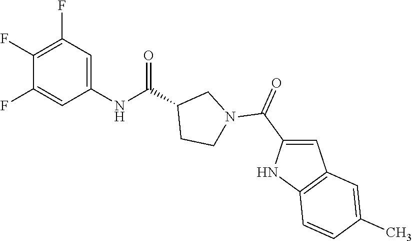 [SMILES: Cc1ccc2[nH]c(C(=O)N3CC[C@H](C(=O)Nc4cc(F)c(F)c(F)c4)C3)cc2c1]

The mixture of Intermediate III-A (1 eq.) and 5-methyl-1H-indole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 55 (16 mg, 100% purity by UV) as a white solid. LCMS Method B, Rt=4.94 min, m/z=402.1 [M+H]+, exact mass: 401.1351.

### Example 56. (S)-1-(5-(hydroxymethyl)-1H-pyrrole-2-carbonyl)-N-(3,4,5-trifluorophenyl)pyrrolidine-3-carboxamide

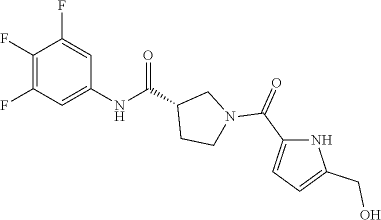 [SMILES: O=C(Nc1cc(F)c(F)c(F)c1)[C@H]1CCN(C(=O)c2ccc(CO)[nH]2)C1]

The mixture of Intermediate III-A (1 eq.) and 5-(hydroxymethyl)-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 56 (26 mg, 99% purity by UV) as a white solid. LCMS Method B, Rt=4.24 min, m/z=368.1 [M+H]+ 390.1 [M+Na]+, exact mass: 367.1144.

### Example 57. (S)-1-(5-fluoro-1H-indole-2-carbonyl)-N-(3,4,5-trifluorophenyl)pyrrolidine-3-carboxamide

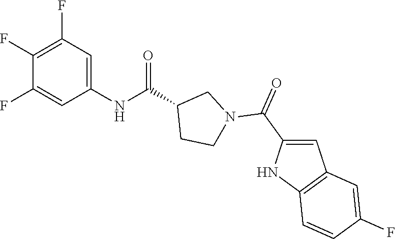 [SMILES: O=C(Nc1cc(F)c(F)c(F)c1)[C@H]1CCN(C(=O)c2cc3cc(F)ccc3[nH]2)C1]

The mixture of Intermediate III-A (1 eq.) and 5-fluoro-1H-indole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 57 (7 mg, 98% purity by UV) as a white solid. LCMS Method B, Rt=4.83 min, m/z=406.1 [M+H]+ 428.1 [M+Na]+, exact mass: 405.1100.

### Example 58. (2S,3S)—N-(3-bromo-4,5-difluorophenyl)-2-methyl-1-(5-methyl-1H-pyrrole-2-carbonyl)pyrroline-3-carboxamide

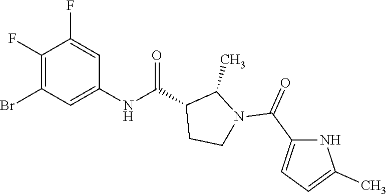 [SMILES: Cc1ccc(C(=O)N2CC[C@H](C(=O)Nc3cc(F)c(F)c(Br)c3)[C@@H]2C)[nH]1]

The mixture Intermediate III-J (1 eq.) and 5-methyl-1H-pyrrole-2-carboxylic acid (1.5 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 60-100% EtOAc/Hexanes. The desired fractions were pooled and the solvent was removed under reduced pressure to afford Example 58 (16 mg, 100% purity by UV) as a white solid. LCMS Method B, Rt=4.91 min, m/z=426.1 [M+H]+, exact mass: 425.0550.

### Example 59. (S)-1-(3-chloro-1H-pyrrolo[2,3-b]pyridine-2-carbonyl)-N-(3,4,5-trifluorophenyl)pyrrolidine-3-carboxamide

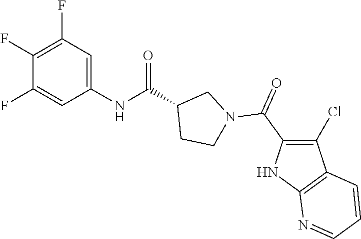 [SMILES: O=C(Nc1cc(F)c(F)c(F)c1)[C@H]1CCN(C(=O)c2[nH]c3ncccc3c2Cl)C1]

The mixture of Intermediate IU-A (1 eq.) and 3-chloro-1H-pyrrolo[2,3-b]pyridine-2-carboxylic acid (1.2 eq.) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 59 (16 mg, 100% purity by UV) as a white solid. LCMS Method B, Rt=4.55 min, m/z=423.1 [M+H]+, LCMS Method C, Rt=4.49 min, m/z=423.0827 [M+H]+, exact mass: 422.0757.

### Example 60. (2S,3S)-1-(5-chloro-1H-pyrrole-2-carbonyl)-2-methyl-N-(3,4,5-trifluorophenyl)pyrrolidine-3-carboxamide

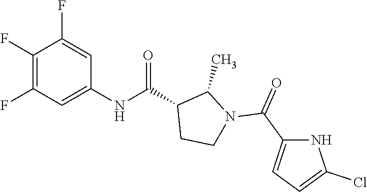 [SMILES: C[C@H]1[C@@H](C(=O)Nc2cc(F)c(F)c(F)c2)CCN1C(=O)c1ccc(Cl)[nH]1]

The mixture Intermediate III-D (1 eq.) and 5-chloro-1H-pyrrole-2-carboxylic acid (1.5 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 50-100% EtOAc/Hexanes. The desired fractions were pooled and the solvent was removed under reduced pressure to afford Example 60 (13 mg, 95% purity by UV) as a white solid. LCMS Method B, Rt=4.83 min, m/z=386.1 [M+H]+, LCMS Method C, Rt=4.82 min, m/z=386.0876 [M+H]+, exact mass: 385.0805.

### Example 61. (S)-1-(5-isopropyl-1H-pyrrole-2-carbonyl)-N-(3,4,5-trifluorophenyl)pyrrolidine-3-carboxamide

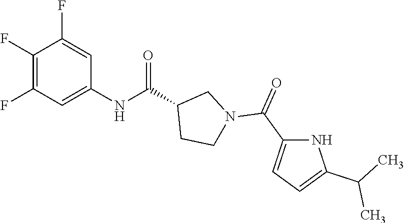 [SMILES: CC(C)c1ccc(C(=O)N2CC[C@H](C(=O)Nc3cc(F)c(F)c(F)c3)C2)[nH]1]

The mixture of Intermediate III-A (1 eq.) and 5-isopropyl-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 61 (28 mg, 100% purity by UV) as a white solid. LCMS Method B, Rt=4.87 min, m/z=380.1 [M+H]+, LCMS Method C, Rt=4.93 min, m/z=380.1579 [M+H]+, exact mass: 379.1508.

### Example 62. (S)—N-(3-chloro-4,5-difluorophenyl)-1-(5-methyl-1H-pyrrole-2-carbonyl)pyrrolidine-3-carboxamide

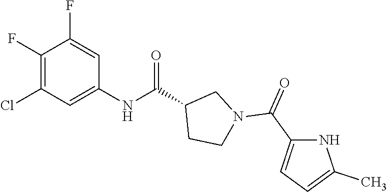 [SMILES: Cc1ccc(C(=O)N2CC[C@H](C(=O)Nc3cc(F)c(F)c(Cl)c3)C2)[nH]1]

The mixture of Intermediate III-K (1 eq.) and 5-methyl-1H-pyrrole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 62 (36 mg, 100% purity by UV) as a white solid. LCMS Method B, Rt=4.68 min, m/z=368.1 [M+H]+, LCMS Method C, Rt=4.63 min, m/z=368.0967 [M+H]+, exact mass: 367.0899.

### Example 63. (S)-1-(3-cyano-1H-pyrrolo[2,3-b]pyridine-2-carbonyl)-N-(3,4,5-trifluorophenyl)pyrrolidine-3-carboxamide

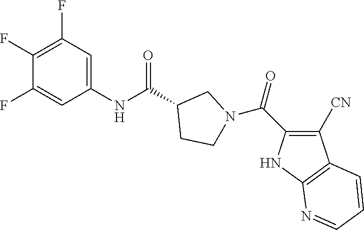 [SMILES: N#Cc1c(C(=O)N2CC[C@H](C(=O)Nc3cc(F)c(F)c(F)c3)C2)[nH]c2ncccc12]

### Step 1: ('S)-1-(3-bromo-1H-pyrrolo[2,3-b]pyridine-2-carbonyl)-N-(3,4,5-trifluorophenyl)pyrrolidine-3-carboxamide

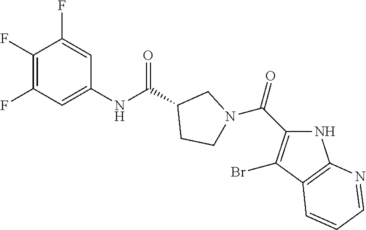 [SMILES: O=C(Nc1cc(F)c(F)c(F)c1)[C@H]1CCN(C(=O)c2[nH]c3ncccc3c2Br)C1]

The mixture of Intermediate III-A (1 eq.) and 3-bromo-1H-pyrrolo[2,3-b]pyridine-2-carboxylic acid (1.1 eq.) was dissolved in DMF 5 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford the product (0.19 mmol).

### Step 2: (S)-1-(3-cyano-1H-pyrrolo[2,3-b]pyridine-2-carbonyl)-N-(3,4,5-trifluorophenyl)pyrrolidine-3-carboxamide

A mixture of (S)-1-(3-bromo-1H-pyrrolo[2,3-b]pyridine-2-carbonyl)-N-(3,4,5-trifluorophenyl) pyrrolidine-3-carboxamide (1 eq.) and Copper(I)cyanide (1 eq.) in NMP (2 mL) was heated at 120° C. in a microwave reactor for 20 mins. Then the mixture was diluted with water and extracted the product with EtOAc for 3 times. The organic layer was washed with brine. The crude reaction was purified by column chromatography with 80% EtOAc to give Example 63 (3 mg, 96% purity by UV). LCMS Method B, Rt=4.49 min, m/z=414.1 [M+H]+ 436.1 [M+Na]+, LCMS Method C, Rt=4.24 min, m/z=414.1195 [M+H]+, exact mass: 413.1100.

### Example 64. (2S,3S)-2-methyl-1-(1H-pyrrolo[3,2-b]pyridine-2-carbonyl)-N-(3,4,5-trifluorophenyl) pyrrolidine-3-carboxamide

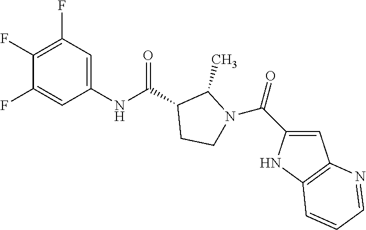 [SMILES: C[C@H]1[C@@H](C(=O)Nc2cc(F)c(F)c(F)c2)CCN1C(=O)c1cc2ncccc2[nH]1]

The mixture Intermediate III-D (1 eq.) and 1H-pyrrolo[3,2-b]pyridine-2-carboxylic acid (1.5 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 64 (20 mg, 100% purity by UV) as a white solid. LCMS Method B, Rt=4.07 min, m/z=403.1 [M+H]+, exact mass: 402.1304.

### Example 65. (2S,3S)-1-(5-chloro-1H-pyrrole-2-carbonyl)-N-(3-cyano-4-fluorophenyl)-2-methylpyrrolidine-3-carboxamide

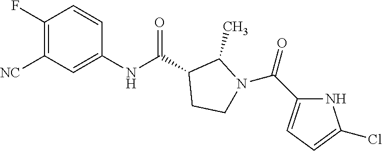 [SMILES: C[C@H]1[C@@H](C(=O)Nc2ccc(F)c(C#N)c2)CCN1C(=O)c1ccc(Cl)[nH]1]

The mixture Intermediate III-G (1 eq.) and 5-chloro-1H-pyrrole-2-carboxylic acid (1.5 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 65 (9 mg, 100% purity by UV) as a white solid. LCMS Method B, Rt=4.65 min, m/z=375.1 [M+H]+, LCMS Method C, Rt=4.36 min, m/z=375.1824 [M+H]+, exact mass: 374.0946.

### Example 66. (2S,3S)—N-(3-cyano-4-fluorophenyl)-2-methyl-1-(5-methyl-1H-pyrrole-2-carbonyl)pyrrolidine-3-carboxamide

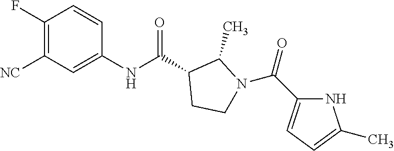 [SMILES: Cc1ccc(C(=O)N2CC[C@H](C(=O)Nc3ccc(F)c(C#N)c3)[C@@H]2C)[nH]1]

The mixture of Intermediate III-G (1 eq.) and 5-methyl-1H-pyrrole-2-carboxylic acid (1.5 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography with 50-100% EtOAc/Hexanes. The desired fractions were pooled and the solvent was removed under reduced pressure to afford Example 66 (17 mg, 100% purity by UV) as a white solid. LCMS Method B, Rt=4.56 min, m/z=355.1 [M+H]+, LCMS Method C, Rt=4.27 min, m/z=355.1583 [M+H]+, exact mass: 354.1492.

### Example 67. (S)-1-(3H-imidazo[4,5-b]pyridine-2-carbonyl)-N-(3,4,5-trifluorophenyl)pyrrolidine-3-carboxamide

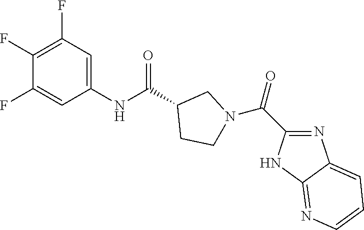 [SMILES: O=C(Nc1cc(F)c(F)c(F)c1)[C@H]1CCN(C(=O)c2nc3cccnc3[nH]2)C1]

The mixture of Intermediate III-A (1 eq.) and 3H-imidazo[4,5-b]pyridine-2-carboxylic acid (1 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 67 (2 mg, 92% purity by UV) as a white solid. LCMS Method B, Rt=4.28 min, m/z=390.1 [M+H]+ 412.1 [M+Na]+, LCMS Method C, Rt=3.91 min, m/z=390.1180 [M+H]+, exact mass: 389.1100.

### Example 68. (S)-1-(1H-benzo[d]imidazole-2-carbonyl)-N-(4-fluoro-3-methylphenyl)pyrrolidine-3-carboxamide

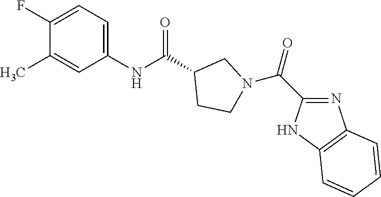 [SMILES: Cc1cc(NC(=O)[C@H]2CCN(C(=O)c3nc4ccccc4[nH]3)C2)ccc1F]

The mixture of Intermediate III-B (1 eq.) and 1H-benzo[d]imidazole-2-carboxylic acid (1.1 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 68 (11 mg, 98% purity by UV) as a white solid. LCMS Method B, Rt=4.51 min, m/z=367.2 [M+H]+ 389.1 [M+Na]+, exact mass: 366.1492.

### Example 69. (S)—N-(3-chloro-4-fluorophenyl)-1-(3H-imidazo[4,5-b]pyridine-2-carbonyl)pyrrolidine-3-carboxamide

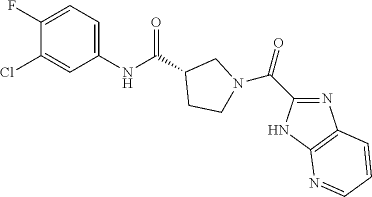 [SMILES: O=C(Nc1ccc(F)c(Cl)c1)[C@H]1CCN(C(=O)c2nc3cccnc3[nH]2)C1]

The mixture of Intermediate III-I (1 eq.) and 3H-imidazo[4,5-b]pyridine-2-carboxylic acid (1.1 eq.) was dissolved in DMF 3 mL. Then 1.5 eq. of HATU, and 1 mL of DIPEA were added. The reaction mixture was stirred for 12 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 69 (17 mg, 100% purity by UV) as a white solid. LCMS Method B, Rt=4.31 min, m/z=388.1 [M+H]+ 412.1 [M+Na]+, exact mass: 387.0898.

### Example 70. (2S,3S)-1-(3H-imidazo[4,5-b]pyridine-2-carbonyl)-2-methyl-N-(3,4,5-trifluorophenyl)pyrrolidine-3-carboxamide

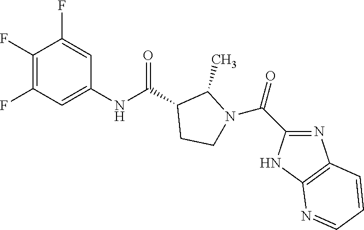 [SMILES: C[C@H]1[C@@H](C(=O)Nc2cc(F)c(F)c(F)c2)CCN1C(=O)c1nc2cccnc2[nH]1]

The mixture of Intermediate III-D (1 eq.) and 3H-imidazo[4,5-b]pyridine-2-carboxylic acid (1.3 eq.) was dissolved in DMF 3 mL. Then 2 eq. of EDCI-HCl, HOBt, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 70 (10 mg, 100% purity by UV) as a white solid. LCMS Method B, Rt=4.45 min, m/z=404.1 [M+H]+ 426.1 [M+Na]+, LCMS Method C, Rt=4.22 min, m/z=404.1312 [M+H]+, exact mass: 403.1256.

### Example 71. (S)-1-(3-chloro-1H-indole-2-carbonyl)-N-((2-chlorothiazol-5-yl)methyl)pyrrolidine-3-carboxamide

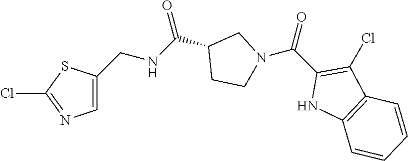 [SMILES: O=C(NCc1cnc(Cl)s1)[C@H]1CCN(C(=O)c2[nH]c3ccccc3c2Cl)C1]

The mixture of Intermediate III-L (1 eq.) and 3-chloro-1H-indole-2-carboxylic acid (1.2 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 71 (13 mg, 96% purity by UV) as a white solid. LCMS Method B, Rt=4.51 min, m/z=423.1 [M+H]+ 445.0 [M+Na]+, LCMS Method C, Rt=4.28 min, m/z=423.0453 [M+H]+, exact mass: 422.0371. ¹H-NMR (500 MHz, Acetone-d₆) δ 10.88 (s, 1H), 8.08 (d, J=44.8 Hz, 1H), 7.59 (d, J=8.0 Hz, 1H), 7.50 (d, J=8.3 Hz, 1H), 7.47 (d, J=25.0 Hz, 1H), 7.29 (t, J=7.5 Hz, 1H), 7.19 (dd, J=7.8, 7.3 Hz, 1H), 4.54 (d, J=31.1 Hz, 2H), 4.09-3.51 (m, 4H), 3.20-3.14 (m, 1H), 2.30-2.10 (m, 2H).

### Example 72. (2S,3S)-1-(3-(hydroxymethyl)-1H-indole-2-carbonyl)-2-methyl-N-(3,4,5-trifluorophenyl)pyrrolidine-3-carboxamide

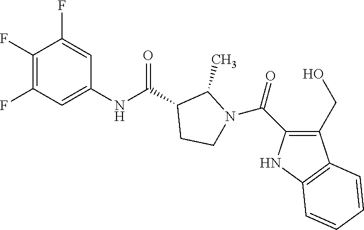 [SMILES: C[C@H]1[C@@H](C(=O)Nc2cc(F)c(F)c(F)c2)CCN1C(=O)c1[nH]c2ccccc2c1CO]

The mixture of Intermediate III-D (1 eq.) and 3-(hydroxymethyl)-1H-indole-2-carboxylic acid (1.4 eq.) was dissolved in DMF 3 mL. Then 1.5 eq. of HATU, and 1 mL of DIPEA were added. The reaction mixture was stirred for 8 hours at room temperature. Brine was added and the mixture was partitioned with EtOAc. The organic layer was dried over sodium sulfate anhydrous, the solids were removed by filtration, and the solvent was removed under reduced pressure and the crude was purified via silica gel column chromatography. The desired fractions were pooled and the solvent was removed under reduced pressure to afford Example 72 (21 mg, 86% purity by UV) as a white solid. LCMS Method B, Rt=4.59 min, m/z=430.1 [M+H]+, exact mass: 431.1457.

### Example 73. (S)-1-(5-cyclopropyl-1H-pyrrole-2-carbonyl)-N-(3,4,5-trifluorophenyl)pyrrolidine-3-carboxamide

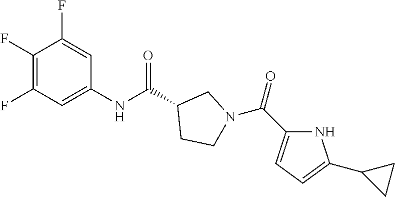 [SMILES: O=C(Nc1cc(F)c(F)c(F)c1)[C@H]1CCN(C(=O)c2ccc(C3CC3)[nH]2)C1]

### Step 1: Synthesis of methyl 5-cyclopropyl-1H-pyrrole-2-carboxylate

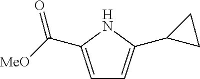 [SMILES: COC(=O)c1ccc(C2CC2)[nH]1]

1.2 eq. of methyl 5-bromo-1H-pyrrole-2-carboxylate was dissolved in H₂O:1,4-dioxane (1:5). Then, 1 eq. of cyclopropylboronic acid and 3 eq. of K₂CO₃ were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/hexane.

### Step 2: Deprotection of methyl 5-cyclopropyl-1H-pyrrole-2-carboxylate

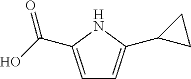 [SMILES: O=C(O)c1ccc(C2CC2)[nH]1]

Methyl 5-cyclopropyl-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-cyclopropyl-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 73 (17 mg, 96% purity by UV) as a white solid. LCMS Method A, Rt=4.71 min, m/z=378.1 [M+H]+, 400.1 [M+Na]+, LCMS Method C, Rt=4.70 min, m/z=378.1465 [M+H]+, exact mass: 377.1351.

### Example 74. (2S,3S)—N-(3-cyano-4-fluorophenyl)-1-(5-cyclopropyl-1H-pyrrole-2-carbonyl)-2-methylpyrrolidine-3-carboxamide

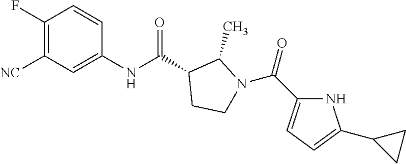 [SMILES: C[C@H]1[C@@H](C(=O)Nc2ccc(F)c(C#N)c2)CCN1C(=O)c1ccc(C2CC2)[nH]1]

### Step 1: Synthesis of methyl 5-cyclopropyl-1H-pyrrole-2-carboxylate

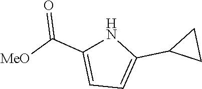 [SMILES: COC(=O)c1ccc(C2CC2)[nH]1]

1.2 eq. of methyl 5-bromo-1H-pyrrole-2-carboxylate was dissolved in H₂O:1,4-dioxane (1:5). Then, 1 eq. of cyclopropylboronic acid and 3 eq. of K₂CO₃ were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/hexane.

### Step 2: Deprotection of methyl 5-cyclopropyl-1H-pyrrole-2-carboxylate

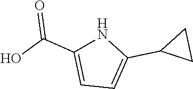 [SMILES: O=C(O)c1ccc(C2CC2)[nH]1]

Methyl 5-cyclopropyl-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-cyclopropyl-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. of Intermediate III-G was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 74 (20 mg, 86% purity by UV) as a white solid. LCMS Method A, Rt=4.74 min, m/z=381.2 [M+H]+, LCMS Method C, Rt=4.55 min, m/z=381.1748 [M+H]+, exact mass: 380.1649.

### Example 75. (S)—N-(3-cyano-4-fluorophenyl)-1-(5-(pyridin-3-yl)-1H-pyrrole-2-carbonyl)pyrrolidine-3-carboxamide

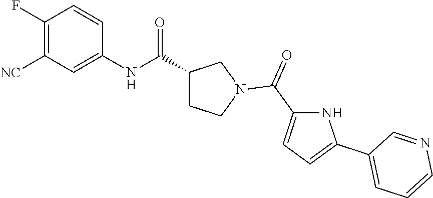 [SMILES: N#Cc1cc(NC(=O)[C@H]2CCN(C(=O)c3ccc(-c4cccnc4)[nH]3)C2)ccc1F]

### Step 1: Synthesis of methyl 5-(pyridin-3-yl)-1H-pyrrole-2-carboxylate

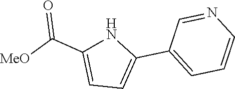 [SMILES: COC(=O)c1ccc(-c2cccnc2)[nH]1]

1.2 eq. of methyl 5-bromo-1H-pyrrole-2-carboxylate was dissolved in H₂O:1,4-dioxane (1:5). Then, 1 eq. of pyridin-3-ylboronic acid and 3 eq. of K₂CO₃ were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/hexane.

### Step 2: Deprotection of methyl 5-(pyridin-3-yl)-1H-pyrrole-2-carboxylate

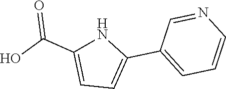 [SMILES: O=C(O)c1ccc(-c2cccnc2)[nH]1]

Methyl 5-(pyridin-3-yl)-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(pyridin-3-yl)-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. of Intermediate III-E was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 75 (13 mg, 86% purity by UV) as a white solid. LCMS Method A, Rt=4.12 min, m/z=404.2 [M+H]+, exact mass: 403.1445.

### Example 76. (S)—N-(3-cyano-4-fluorophenyl)-1-(5-(4-fluorophenyl)-1H-pyrrole-2-carbonyl)pyrrolidine-3-carboxamide

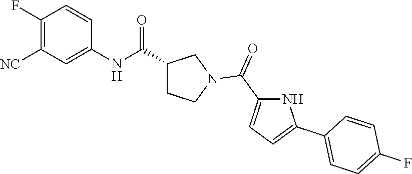 [SMILES: N#Cc1cc(NC(=O)[C@H]2CCN(C(=O)c3ccc(-c4ccc(F)cc4)[nH]3)C2)ccc1F]

### Step 1: Synthesis of methyl 5-(4-fluorophenyl)-1H-pyrrole-2-carboxylate

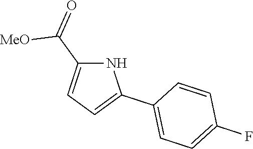 [SMILES: COC(=O)c1ccc(-c2ccc(F)cc2)[nH]1]

1.2 eq. of methyl 5-bromo-1H-pyrrole-2-carboxylate was dissolved in H₂O:1,4-dioxane (1:5). Then, 1 eq. of 4-fluorophenylboronic acid and 3 eq. of K₂COM were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/hexane.

### Step 2: Deprotection of methyl 5-(4-fluorophenyl)-1H-pyrrole-2-carboxylate

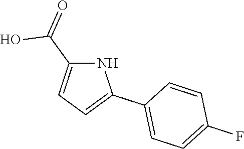 [SMILES: O=C(O)c1ccc(-c2ccc(F)cc2)[nH]1]

Methyl 5-(4-fluorophenyl)-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4-fluorophenyl)-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. of Intermediate III-E was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 76 (12 mg, 98% purity by UV) as a white solid. LCMS Method A, Rt=4.80 min, m/z=421.2 [M+H]+, exact mass: 420.1398.

### Example 77. (S)-1-(5-(pyridin-4-yl)-1H-pyrrole-2-carbonyl)-N-(3,4,5-trifluorophenyl)pyrrolidine-3-carboxamide

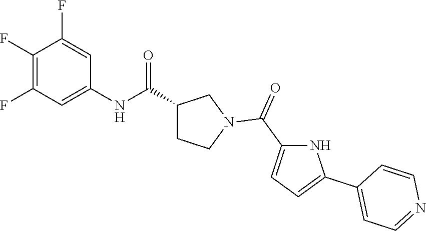 [SMILES: O=C(Nc1cc(F)c(F)c(F)c1)[C@H]1CCN(C(=O)c2ccc(-c3ccncc3)[nH]2)C1]

### Step 1: (S)-1-(5-bromo-1H-pyrrole-2-carbonyl)-N-(3,4,5-trifluorophenyl)pyrrolidine-3-carboxamide

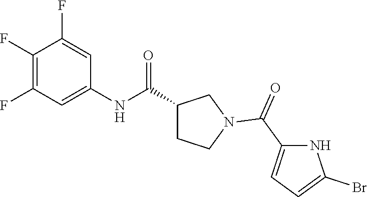 [SMILES: O=C(Nc1cc(F)c(F)c(F)c1)[C@H]1CCN(C(=O)c2ccc(Br)[nH]2)C1]

A mixture of 5-bromo-1H-pyrrole-2-carboxylic acid (1 eq.), Intermediate III-A (1.14 eq.) was dissolved in DMF (5 mL). Then added 1.5 eq. of HATU and 1 mL of DIPEA. The mixture was stirred overnight at room temperature. Then the mixture was diluted with water. The product was extracted with EtOAc for 3 times. The organic layer was washed by brine and purified by column chromatography with 60% EtOAc/hexanes. The desired fractions were pooled and the solvent was removed under reduced pressure to afford the product.

### Step 2: Suzuki Coupling Reaction

A mixture of (S)-1-(5-bromo-1H-pyrrole-2-carbonyl)-N-(3,4,5-trifluorophenyl)pyrrolidine-3-carboxamide (1 eq.) and pyridin-4-yl boronic acid (3 eq.) in DME/H₂O (4:1, 0.1 M) was bubbled by argon for 10 mins. The mixture was then added with Pd(PPh₃)₂Cl₂ (10 mol %) and sodium hydrogen carbonate (3 eq.). The reaction was heated at 110° C. overnight in sealed tube and then cooled to room temperature, filtered, and concentrated in vacuo. The crude product was purified by liquid column chromatography using 5% MeOH/EtOAc to give Example 77 (4 mg, 90% purity by UV). LCMS Method B, Rt=4.12 min, m/z=415.2 [M+H]+, LCMS Method C, Rt=3.54 min, m/z=415.1375 [M+H]+, exact mass: 414.1304.

### Example 78. (S)—N-(3-cyano-4-fluorophenyl)-1-(5-(pyridin-4-yl)-1H-pyrrole-2-carbonyl)pyrrolidine-3-carboxamide

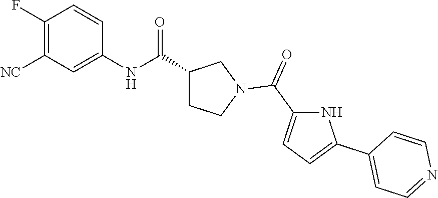 [SMILES: N#Cc1cc(NC(=O)[C@H]2CCN(C(=O)c3ccc(-c4ccncc4)[nH]3)C2)ccc1F]

### Step 1: (S)-1-(5-bromo-1H-pyrrole-2-carbonyl)-N-(3-cyano-4-fluorophenyl)pyrrolidine-3-carboxamide

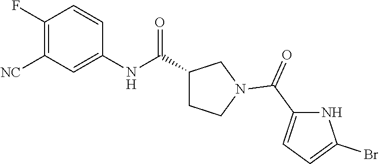 [SMILES: N#Cc1cc(NC(=O)[C@H]2CCN(C(=O)c3ccc(Br)[nH]3)C2)ccc1F]

A mixture of 5-bromo-1H-pyrrole-2-carboxylic acid (1 eq.), Intermediate III-E (1.2 eq.) was dissolved in DMF (5 mL). Then added 1.5 eq. of HATU and 1 mL of DIPEA. The mixture was stirred overnight at room temperature. Then the mixture was diluted with water. The product was extracted with EtOAc for 3 times. The organic layer was washed by brine and purified by column chromatography with 60% EtOAc/hexanes. The desired fractions were pooled and the solvent was removed under reduced pressure to afford the product.

### Step 2: Suzuki Coupling Reaction

A mixture of (S)-1-(5-bromo-1H-pyrrole-2-carbonyl)-N-(3-cyano-4-fluorophenyl)pyrrolidine-3-carboxamide (1 eq.) and pyridin-4-ylboronic acid (3 eq.) in DME/H₂O (4:1, 0.1 M) was bubbled by argon for 10 mins. The mixture was then added with Pd(PPh₃)₂Cl₂ (10 mol %) and sodium hydrogen carbonate (3 eq.). The reaction was heated at 110° C. overnight in sealed tube and then cooled to room temperature, filtered, and concentrated in vacuo. The crude product was purified by liquid column chromatography using 5% MeOH/EtOAc to give Example 78 (14 mg, 100% purity by UV) as a white solid. LCMS Method B, Rt=4.01 min, m/z=404.1 [M+H]+, exact mass: 403.1445.

### Example 79. (S)-1-(5-(pyrimidin-5-yl)-1H-pyrrole-2-carbonyl)-N-(3,4,5-trifluorophenyl)pyrrolidine-3-carboxamide

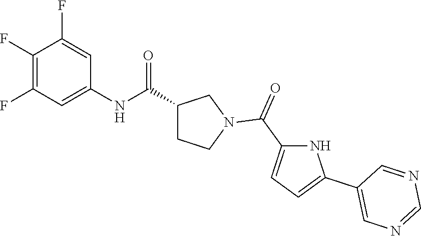 [SMILES: O=C(Nc1cc(F)c(F)c(F)c1)[C@H]1CCN(C(=O)c2ccc(-c3cncnc3)[nH]2)C1]

### Step 1: Synthesis of methyl 5-(pyrimidin-5-yl)-1H-pyrrole-2-carboxylate

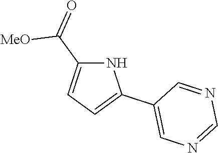 [SMILES: COC(=O)c1ccc(-c2cncnc2)[nH]1]

1.2 eq. of methyl 5-bromo-1H-pyrrole-2-carboxylate was dissolved in H₂O:1,4-dioxane (1:5). Then, 1 eq. of pyrimidin-5-ylboronic acid and 3 eq. of K₂CO₃ were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/hexane.

### Step 2: Deprotection of methyl 5-(pyrimidin-5-yl)-1H-pyrrole-2-carboxylate

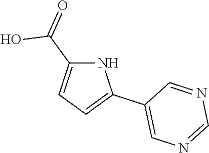 [SMILES: O=C(O)c1ccc(-c2cncnc2)[nH]1]

Methyl 5-(pyrimidin-5-yl)-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(pyrimidin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 79 (5 mg, 90% purity by UV) as a white solid. LCMS Method A, Rt=4.44 min, m/z=416.1 [M+H]+, exact mass: 415.1256.

### Example 80. (S)-1-(5-(2-methylpyridin-3-yl)-1H-pyrrole-2-carbonyl)-N-(3,4,5-trifluorophenyl) pyrrolidine-3-carboxamide

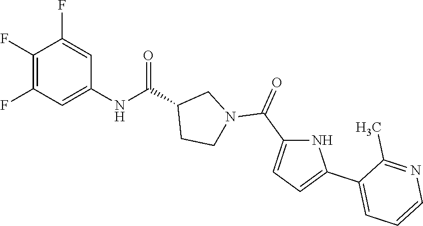 [SMILES: Cc1ncccc1-c1ccc(C(=O)N2CC[C@H](C(=O)Nc3cc(F)c(F)c(F)c3)C2)[nH]1]

### Step 1: Synthesis of methyl 5-(2-methylpyridin-3-yl)-1H-pyrrole-2-carboxylate

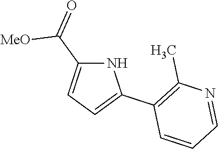 [SMILES: COC(=O)c1ccc(-c2cccnc2C)[nH]1]

1.2 eq. of methyl 5-bromo-1H-pyrrole-2-carboxylate was dissolved in H₂O:1,4-dioxane (1:5). Then, 1 eq. of (2-methylpyridin-3-yl)boronic acid and 3 eq. of K₂CO₃ were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/Hexanes.

### Step 2: Deprotection of methyl 5-(2-methylpyridin-3-yl)-1H-pyrrole-2-carboxylate

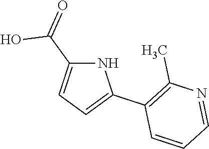

Methyl 5-(2-methylpyridin-3-yl)-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(2-methylpyridin-3-yl)-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. Intermediate III-A was added to the solution. The mixture was stirred overnight at room temperature. Then the mixture was diluted with water. The product was extracted with EtOAc for 3 times. The organic layer was washed by brine and purified by column chromatography with 1-10% MeOH/EtOAc. The desired fractions were pooled and the solvent was removed under reduced pressure to afford Example 80 (10 mg, 100% purity by UV) as a white solid. LCMS Method A, Rt=4.15 min, m/z=429.2 [M+H]+, LCMS Method C, Rt=3.61 min, m/z=429.1543 [M+H]+, exact mass: 428.1460.

### Example 81. (S)—N-(3-cyano-4-fluorophenyl)-1-(5-(2-methylpyridin-3-yl)-1H-pyrrole-2-carbonyl) pyrrolidine-3-carboxamide

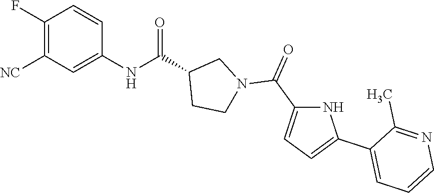 [SMILES: Cc1ncccc1-c1ccc(C(=O)N2CC[C@H](C(=O)Nc3ccc(F)c(C#N)c3)C2)[nH]1]

### Step 1: Synthesis of methyl 5-(2-methylpyridin-3-yl)-1H-pyrrole-2-carboxylate

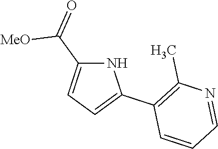 [SMILES: COC(=O)c1ccc(-c2cccnc2C)[nH]1]

1.2 eq. of methyl 5-bromo-1H-pyrrole-2-carboxylate was dissolved in H₂O:1,4-dioxane (1:5). Then, 1 eq. of (2-methylpyridin-3-yl)boronic acid and 3 eq. of K₂CO₃ were added to the flask. The reaction was bubbled by argon gas for 5-15 mins. Then 10 mol % Pd(PPh₃)₄ was added and heated the reaction under argon. Reaction was heated under argon gas at 110° C. for an hour to overnight. To achieve pure product, crude product was purified by liquid column chromatography with EtOAc/hexane.

### Step 2: Deprotection of methyl 5-(2-methylpyridin-3-yl)-1H-pyrrole-2-carboxylate

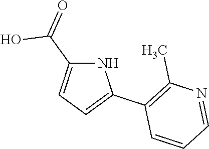

Methyl 5-(2-methylpyridin-3-yl)-1H-pyrrole-2-carboxylate (1 eq.) was dissolved in 4 mL of THF. Then, 5 eq. of LiOH was dissolved in 4 mL of H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(2-methylpyridin-3-yl)-1H-pyrrole-2-carboxylic acid was dissolved in DMF. Then 1.5 eq. of HATU and 3 eq. of DIPEA were added. The reaction mixture was stirred at room temperature for 10 mins. Then, 1.1 eq. of Intermediate III-E was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 81 (13 mg, 96% purity by UV) as a white solid. LCMS Method A, Rt=4.03 min, m/z=418.2 [M+H]+, exact mass: 417.1601.

### Example 82. (S)-)-5)-1pyridin-3yl)-1H-pyrrole-2carbonyl)-N-(3,4,-5 trifluorophenyl)pyrrolidine-3carboxamide

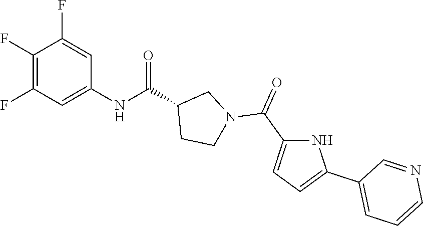 [SMILES: O=C(Nc1cc(F)c(F)c(F)c1)[C@H]1CCN(C(=O)c2ccc(-c3cccnc3)[nH]2)C1]

### Step 1: (S)-1-(5-bromo-1H-pyrrole-2-carbonyl)-N-(3,4,5-trifluorophenyl)pyrrolidine-3-carboxamide

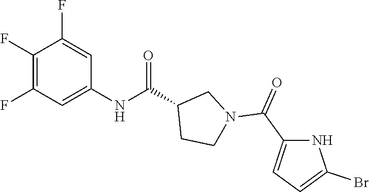 [SMILES: O=C(Nc1cc(F)c(F)c(F)c1)[C@H]1CCN(C(=O)c2ccc(Br)[nH]2)C1]

A mixture of 5-bromo-1H-pyrrole-2-carboxylic acid (1 eq.), Intermediate III-A (1.14 eq.) was dissolved in DMF (5 mL). Then added 1.5 eq. of HATU and 1 mL of DIPEA. The mixture was stirred overnight at room temperature. Then the mixture was diluted with water. The product was extracted with EtOAc for 3 times. The organic layer was washed by brine and purified by column chromatography with 60% EtOAc/hexanes. The desired fractions were pooled and the solvent was removed under reduced pressure to afford the product.

### Step 2: Suzuki Coupling Reaction

A mixture of (S)-1-(5-bromo-1H-pyrrole-2-carbonyl)-N-(3,4,5-trifluorophenyl)pyrrolidine-3-carboxamide (1 eq.) and pyridin-3-yl boronic acid (3 eq.) in DME/H₂O (4:1, 0.1 M) was bubbled by argon for 10 mins. The mixture was then added with Pd(PPh₃)₂Cl₂ (10 mol %) and sodium hydrogen carbonate (3 eq.). The reaction was heated at 110° C. overnight in sealed tube and then cooled to room temperature, filtered, and concentrated in vacuo. The crude product was purified by liquid column chromatography using 5% MeOH/EtOAc to give Example 82 (8 mg, 99% purity by UV). LCMS Method B, Rt=4.20 min, m/z=415.1 [M+H]+, LCMS Method C, Rt=3.80 min, m/z=415.1372 [M+H]+, exact mass: 414.1304.

### Example 83. (2S,3S)-2-methyl-1-(5-(pyridazin-4-yl)-1H-pyrrole-2-carbonyl)-N-(3,4,5-trifluorophenyl)pyrrolidine-3-carboxamide

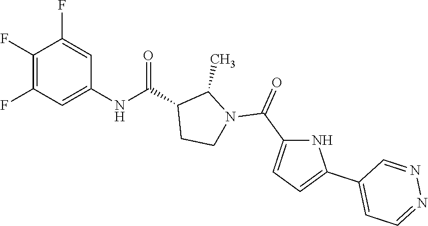 [SMILES: C[C@H]1[C@@H](C(=O)Nc2cc(F)c(F)c(F)c2)CCN1C(=O)c1ccc(-c2ccnnc2)[nH]1]

### Step 1: Synthesis of methyl 5-(pyridazin-4-yl)-1H-pyrrole-2-carboxylate via Suzuki cross coupling

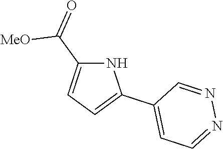 [SMILES: COC(=O)c1ccc(-c2ccnnc2)[nH]1]

1 eq. of pyridazin-4-amine was dissolved in 1,2-DME. Then, 0.5 eq. of iodine, 1 eq. of potassium iodide, 0.3 eq. of copper(I)iodide and 4 eq. of isoamyl nitrite were added to the previous solution. The reaction mixture was heated at 70° C. for 2 hours. Crude reaction was evaporated by rotavapor. Diluted with water and extracted by EtOAc for 3 times. The organic layer was concentrated. The product was purified by liquid column chromatography with EtOAc/hexane to achieve the pure product of 4-iodopyridazine.

A mixture of 1 eq. of 4-iodopyridazine and 1.11 eq. of Intermediate V-A in 1,4-dioxane:H₂O (6:1) was bubbled by argon for 10 mins. The mixture was then added with 10 mol % of Pd(PPh₃)₄ and 1.11 eq. of potassium carbonate. The reaction was heated at 80° C. for 3 hours in sealed tube and then cooled to room temperature, filtered, and concentrated in vacuo. The crude product was purified by liquid column chromatography using 60% EtOAc/hexane to give yellow oil.

### Step 2: Hydrolysis of methyl 5-(pyridazin-4-yl)-H-pyrrole-2-carboxylate

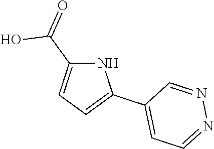 [SMILES: O=C(O)c1ccc(-c2ccnnc2)[nH]1]

1 eq. of Methyl 5-(pyridazin-4-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

The mixture of 1.05 eq. of Intermediate III-D (0.25 mmol) and 1 eq. of 5-(pyridazin-4-yl)-1H-pyrrole-2-carboxylic acid (43 mg, 0.23 mmol) was dissolved in DMF 3 mL. Then 1.5 eq. of HATU, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 70% EtOAc/hexanes to afford Example 83 (4.4 mg, 98.0% purity). LCMS Method A, Rt=4.41 min, m/z=430.2 [M+H]+, LCMS Method C, Rt=4.30 min, m/z=430.1476 [M+H]+, exact mass: 429.1413.

### Example 84. (2S,3S)-2-methyl-1-(5-(pyrazin-2-yl)-1H-pyrrole-2-carbonyl)-N-(3,4,5-trifluorophenyl) pyrrolidine-3-carboxamide

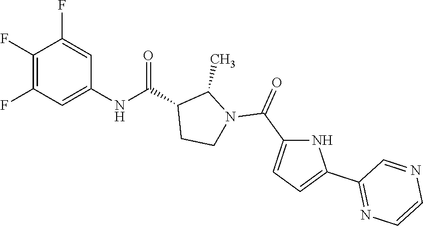 [SMILES: C[C@H]1[C@@H](C(=O)Nc2cc(F)c(F)c(F)c2)CCN1C(=O)c1ccc(-c2cnccn2)[nH]1]

### Step 1: Synthesis of methyl 5-(pyrazin-2-yl)-1H-pyrrole-2-carboxylate via Suzuki cross coupling

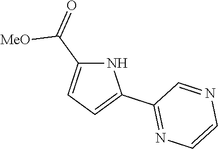 [SMILES: COC(=O)c1ccc(-c2cnccn2)[nH]1]

1.2 eq. of 2-bromopyrazine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(pyrazin-2-yl)-1H-pyrrole-2-carboxylate

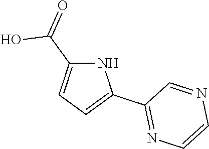 [SMILES: O=C(O)c1ccc(-c2cnccn2)[nH]1]

1 eq. of Methyl 5-(pyrazin-2-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(pyrazin-2-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-D was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 84 (14 mg, 99% purity by UV). LCMS Method A, Rt=4.68 min, m/z=430.2 [M+H]+ 452.2 [M+Na]+, LCMS Method C, Rt=4.56 min, m/z=430.1488 [M+H]+, exact mass: 429.1413.

### Example 85. (S)-1-(5-(4,6-dimethylpyrimidin-5-yl)-1H-pyrrole-2-carbonyl)-N-(3,4,5-trifluorophenyl) pyrrolidine-3-carboxamide

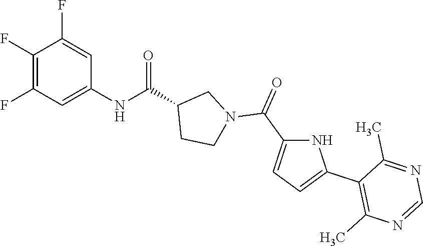 [SMILES: Cc1ncnc(C)c1-c1ccc(C(=O)N2CC[C@H](C(=O)Nc3cc(F)c(F)c(F)c3)C2)[nH]1]

### Step 1: Synthesis of methyl 5-(4,6-dimethylpyrimidin-5-yl)-1H-pyrrole-2-carboxylate via Suzuki cross coupling

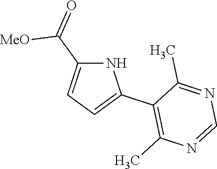 [SMILES: COC(=O)c1ccc(-c2c(C)ncnc2C)[nH]1]

A mixture of 1 eq. of 5-bromo-4,6-dimethylpyrimidine and 1.05 eq. of Intermediate V-A in 1,4-dioxane:H₂O (6:1) was bubbled by argon for 10 mins. The mixture was then added with 10 mol % of Pd(PPh₃)₄ and 3 eq. of potassium carbonate. The reaction was heated at 110° C. for 1 hours in sealed tube and then cooled to room temperature, filtered, and concentrated in vacuo. The crude product was purified by liquid column chromatography using 70% EtOAc/hexane to give yellow oil. ¹H-NMR (500 MHz, Acetone-d₆) δ 11.14 (s, 1H), 8.83 (s, 1H), 6.97 (dd, J=3.5, 2.7 Hz, 1H), 6.29 (dd, J=3.5, 2.5 Hz, 1H), 3.80 (s, 3H), 2.30 (s, 6H).

### Step 2: Hydrolysis of methyl 5-(4,6-dimethylpyrimidin-5-yl)-1H-pyrrole-2-carboxylate

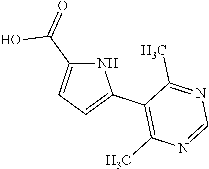 [SMILES: Cc1ncnc(C)c1-c1ccc(C(=O)O)[nH]1]

1 eq. of methyl 5-(pyridazin-4-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(pyridazin-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 85 (5 mg, 98% purity). LCMS Method B, Rt=4.48 min, m/z=444.2 [M+H]+, LCMS Method C, Rt=4.33 min, m/z=444.1634 [M+H]+, exact mass: 443.1569. ¹H-NMR (500 MHz, MeOD-d₄) δ 8.84 (s, 1H), 7.43 (dd, J=10.1, 6.3 Hz, 2H), 6.86 (d, J=3.7 Hz, 1H), 6.28 (d, J=4.5 Hz, 1H), 4.18-3.56 (m, 4H), 3.35-3.16 (m, 1H), 2.41-2.14 (m, 8H).

### Example 86. (S)-1-(5-(2-methylpyrimidin-5-yl)-1H-pyrrole-2-carbonyl)-N-(3,4,5-trifluorophenyl)pyrrolidine-3-carboxamide

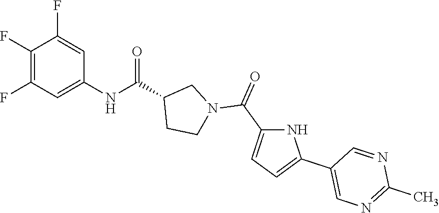 [SMILES: Cc1ncc(-c2ccc(C(=O)N3CC[C@H](C(=O)Nc4cc(F)c(F)c(F)c4)C3)[nH]2)cn1]

Step 1: Synthesis of methyl 5-(2-methylpyrimidin-5-yl)-1H-pyrrole-2-carboxylate via Suzuki cross coupling

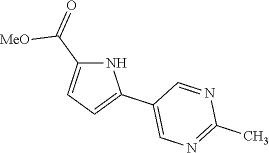 [SMILES: COC(=O)c1ccc(-c2cnc(C)nc2)[nH]1]

1.2 eq. of 5-bromo-2-methylpyrimidine was dissolved in H₂O:1,4-dioxane (1:5). Then 1 eq. Intermediate V-A and 3 eq. of K₂C₀M were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(2-methylpyrimidin-5-yl)-1H-pyrrole-2-carboxylate

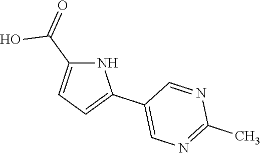

1 eq. of Methyl 5-(2-methylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(2-methylpyrimidin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 86 (9 mg, 97% purity by UV). LCMS Method A, Rt=4.45 min, m/z=430.2 [M+H]+, exact mass: 429.1413.

### Example 87. (S)-1-(5-(3,5-dimethyl-1H-pyrazol-4-yl)-1H-pyrrole-2-carbonyl)-N-(3,4,5-trifluorophenyl) pyrrolidine-3-carboxamide

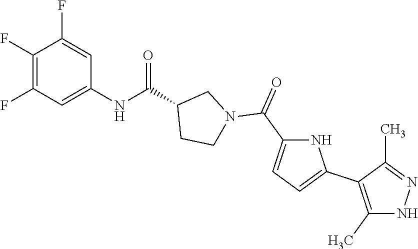 [SMILES: Cc1n[nH]c(C)c1-c1ccc(C(=O)N2CC[C@H](C(=O)Nc3cc(F)c(F)c(F)c3)C2)[nH]1]

### Step 1: Synthesis of methyl 5-(3,5-dimethyl-1H-pyrazol-4-yl)-1H-pyrrole-2-carboxylate via

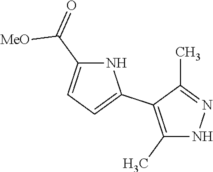 [SMILES: COC(=O)c1ccc(-c2c(C)n[nH]c2C)[nH]1]

1.2 eq. of 4-iodo-3,5-dimethyl-1H-pyrazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(3,5-dimethyl-1H-pyrazole-4-yl)-1H-pyrrole-2-carboxylate

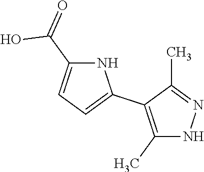

1 eq. of Methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3,5-dimethyl-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 87 (18 mg, 92% purity by UV). LCMS Method A, Rt=4.39 min, m/z=432.2 [M+H]+, exact mass: 431.1569.

### Example 88. (S)—N-(3,4,5-trifluorophenyl)-1-(5-(1,3,5-trimethyl-1H-pyrazol-4-yl)-1H-pyrrole-2-carbonyl) pyrrolidine-3-carboxamide

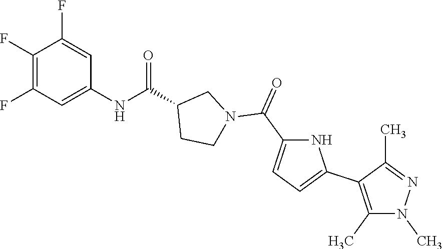 [SMILES: Cc1nn(C)c(C)c1-c1ccc(C(=O)N2CC[C@H](C(=O)Nc3cc(F)c(F)c(F)c3)C2)[nH]1]

### Step 1: Synthesis of methyl 5-(1,3,5-trimethyl-1H-pyrazol-4-yl)-1H-pyrrole-2-carboxylate via Suzuki cross coupling

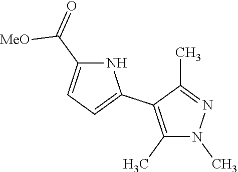 [SMILES: COC(=O)c1ccc(-c2c(C)nn(C)c2C)[nH]1]

A mixture of 1 eq. of 4-bromo-1,3,5-trimethyl-1H-pyrazole and 1.25 eq. of Intermediate V-A in 1,4-dioxane:H₂O (6:1) was bubbled by argon for 10 mins. The mixture was then added 10 mol % of Pd(PPh₃)₄ and 3 eq. of potassium carbonate. The reaction was heated at 105° C. overnight in sealed tube and then cooled to room temperature, filtered, and concentrated in vacuo. The crude product was purified by liquid column chromatography using 50% EtOAc/hexane to give yellow oil.

### Step 2: Hydrolysis of methyl 5-(1,3,5-trimethyl-1H-pyrazol-4-yl)-1H-pyrrole-2-carboxylate

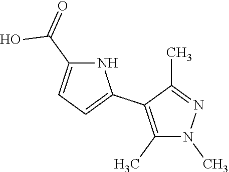 [SMILES: Cc1nn(C)c(C)c1-c1ccc(C(=O)O)[nH]1]

1 eq. of Methyl 5-(1,3,5-trimethyl-1H-pyrazol-4-yl)-1H-pyrrole-2-carboxylate was dissolved in THF. Then, 5 eq. of LiOH was dissolved in H₂O and added to the THF solution. The reaction mixture was stirred at 95° C. for 30 min. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(1,3,5-trimethyl-1H-pyrazol-4-yl)-1H-pyrrole-2-carboxylic acid was dissolved in DMF. Then added 1.5 eq. of HATU and 5 eq. of DIPEA. The reaction mixture was stirred at room temperature for 10 mins. Then, 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 88 (18 mg, 96% purity by UV). LCMS Method B, Rt=4.54 min, m/z=446.2 [M+H]+, exact mass: 445.1726.

### Example 89. (S)-1-(5-(4-methylpyrimidin-5-yl)-1H-pyrrole-2-carbonyl)-N-(3,4,5-trifluorophenyl) pyrrolidine-3-carboxamide

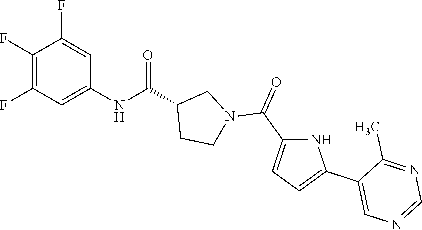 [SMILES: Cc1ncncc1-c1ccc(C(=O)N2CC[C@H](C(=O)Nc3cc(F)c(F)c(F)c3)C2)[nH]1]

### Step 1: Synthesis of methyl 5-(4-methylpyrimidin-5-yl)-1H-pyrrole-2-carboxylate via Suzuki cross coupling

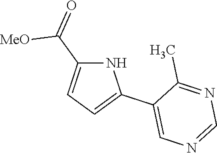 [SMILES: COC(=O)c1ccc(-c2cncnc2C)[nH]1]

A mixture of 1 eq. of 5-bromo-4-methylpyrimidine and 1.5 eq. of Intermediate V-A in 1,4-dioxane:H₂O (6:1) was bubbled by argon for 10 mins. The mixture was then added 10 mol % of Pd(PPh₃)₄ and 3 eq. of potassium carbonate. The reaction was heated at 105° C. overnight in sealed tube and then cooled to room temperature, filtered, and concentrated in vacuo. The crude product was purified by liquid column chromatography using 50% EtOAc/hexane to give yellow oil.

### Step 2: Hydrolysis of methyl 5-(4-methylpyrimidin-5-yl)-1H-pyrrole-2-carboxylate

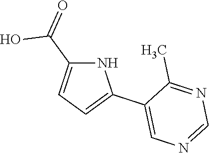

1 eq. of Methyl 5-(4-methylpyrimidin-5-yl)-1H-pyrrole-2-carboxylate was dissolved in THF. Then, 5 eq. of LiOH was dissolved in H₂O and added to the THF solution. The reaction mixture was stirred at 95° C. for 30 mins. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(4-methylpyrimidin-5-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 89 (13 mg, 100% purity by UV). LCMS Method B, Rt=4.40 min, m/z=430.2 [M+H]+, exact mass: 429.1413.

### Example 90. (S)-1-(5-(pyridin-2-yl)-1H-pyrrole-2-carbonyl)-N-(3,4,5-trifluorophenyl)pyrrolidine-3-carboxamide

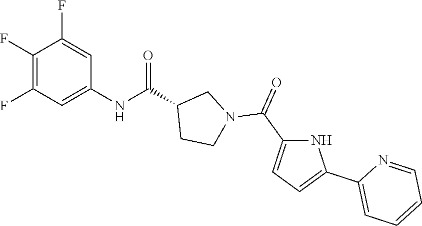 [SMILES: O=C(Nc1cc(F)c(F)c(F)c1)[C@H]1CCN(C(=O)c2ccc(-c3ccccn3)[nH]2)C1]

### Step 1: Synthesis of methyl 5-(pyridin-2-yl)-1H-pyrrole-2-carboxylate via Suzuki cross coupling

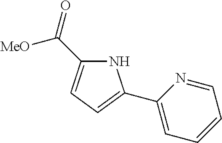 [SMILES: COC(=O)c1ccc(-c2ccccn2)[nH]1]

1.2 eq. of 2-bromopyridine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(pyridin-2-yl)-1H-pyrrole-2-carboxylate

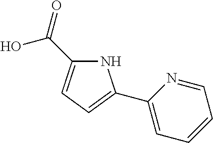 [SMILES: O=C(O)c1ccc(-c2ccccn2)[nH]1]

1 eq. of Methyl 5-(pyridin-2-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(pyridin-2-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 90 (8 mg, 100% purity by UV). LCMS Method A, Rt=4.57 min, m/z=415.2 [M+H]+, exact mass: 414.1304.

### Example 91. (S)-1-(5-(1-methyl-1H-imidazol-2-yl)-1H-pyrrole-2-carbonyl)-N-(3,4,5-trifluorophenyl)pyrrolidine-3-carboxamide

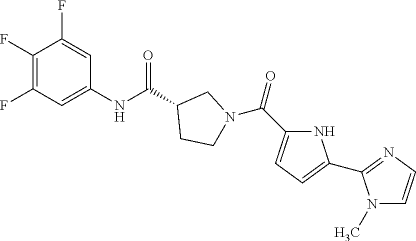 [SMILES: Cn1ccnc1-c1ccc(C(=O)N2CC[C@H](C(=O)Nc3cc(F)c(F)c(F)c3)C2)[nH]1]

### Step 1: Synthesis of methyl 5-(1-methyl-1H-imidazol-2-yl)-1H-pyrrole-2-carboxylate via Suzuki cross coupling

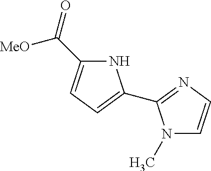

1.2 eq. of 2-bromo-1-methyl-1H-imidazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(1-methyl-1H-imidazol-2-yl)-1H-pyrrole-2-carboxylate

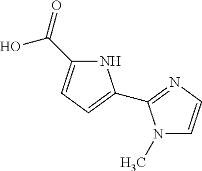

1 eq. of methyl 5-(1-methyl-1H-imidazol-2-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(1-methyl-1H-imidazol-2-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 91 (12 mg, 87% purity by UV). LCMS Method A, Rt=4.10 min, m/z=418.2 [M+H]+, LCMS Method C, Rt=3.44 min, m/z=418.1486 [M+H]+, exact mass: 417.1413.

### Example 92. (S)-1-(5-(3-methylpyrazin-2-yl)-1H-pyrrole-2-carbonyl)-N-(3,4,5-trifluorophenyl)pyrrolidine-3-carboxamide

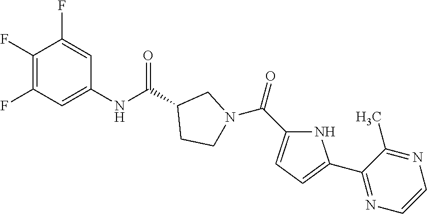 [SMILES: Cc1nccnc1-c1ccc(C(=O)N2CC[C@H](C(=O)Nc3cc(F)c(F)c(F)c3)C2)[nH]1]

### Step 1: Synthesis of methyl 5-(3-methylpyrazin-2-yl)-1H-pyrrole-2-carboxylate via Suzuki cross coupling

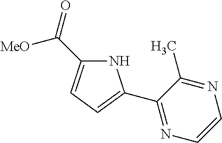 [SMILES: COC(=O)c1ccc(-c2nccnc2C)[nH]1]

1.2 eq. of 2-bromo-3-methylpyrazine was dissolved in H₂O:1,4-dioxane (1:5). Then 1 eq. Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(3-methylpyrazin-2-yl)-1H-pyrrole-2-carboxylate

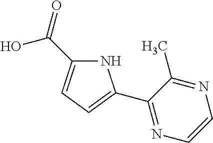

1 eq. of methyl 5-(3-methylpyrazin-2-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3-methylpyrazin-2-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 92 (14 mg, 96% purity by UV). LCMS Method A, Rt=4.62 min, m/z=430.2 [M+H]+, LCMS Method C, Rt=4.51 min, m/z=430.1479 [M+H]+, exact mass: 429.1413.

### Example 93. (2S,3S)-2-methyl-1-(5-(3-methylpyrazin-2-yl)-1H-pyrrole-2-carbonyl)-N-(3,4,5-trifluorophenyl) pyrrolidine-3-carboxamide

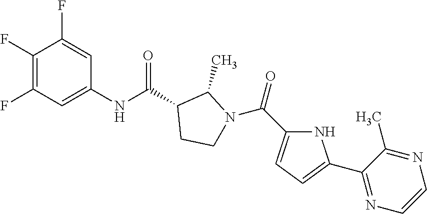 [SMILES: Cc1nccnc1-c1ccc(C(=O)N2CC[C@H](C(=O)Nc3cc(F)c(F)c(F)c3)[C@@H]2C)[nH]1]

### Step 1: Synthesis of methyl 5-(3-methylpyrazin-2-yl)-1H-pyrrole-2-carboxylate via Suzuki cross coupling

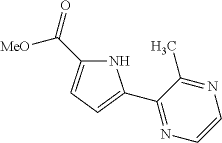 [SMILES: COC(=O)c1ccc(-c2nccnc2C)[nH]1]

1.2 eq. of 2-bromo-3-methylpyrazine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(3-methylpyrazin-2-yl)-1H-pyrrole-2-carboxylate

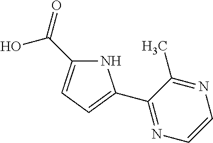

1 eq. of methyl 5-(3-methylpyrazin-2-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3-methylpyrazin-2-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 93(10 mg, 89% purity by UV). LCMS Method A, Rt=4.76 min, m/z=444.2 [M+H]+, LCMS Method C, Rt=4.81 min, m/z=444.1736 [M+H]+, exact mass: 443.1569.

### Example 94. (S)—N-(3-cyano-4-fluorophenyl)-1-(5-(4,6-dimethylpyrimidin-5-yl)-1H-pyrrole-2-carbonyl)pyrrolidine-3-carboxamide

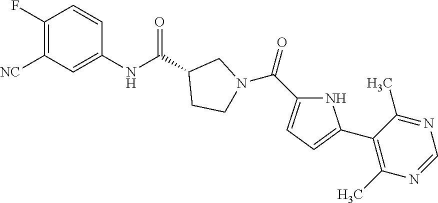 [SMILES: Cc1ncnc(C)c1-c1ccc(C(=O)N2CC[C@H](C(=O)Nc3ccc(F)c(C#N)c3)C2)[nH]1]

### Step 1: Synthesis of methyl 5-(4,6-dimethylpyrimidin-5-yl)-1H-pyrrole-2-carboxylate via Suzuki cross coupling

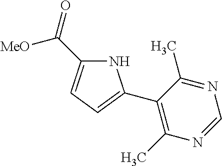 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(4,6-dimethylpyrimidin-5-yl)-1H-pyrrole-2-carboxylate

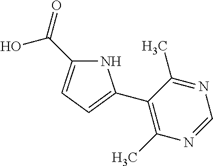 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4,6-dimethylpyrimidin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-E was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 94 (6 mg, 86% purity by UV). LCMS Method A, Rt=4.33 min, m/z=433.2 [M+H]+, LCMS Method C, Rt=3.90 min, m/z=433.1784 [M+H]+, exact mass: 432.1710.

### Example 95. (S)-1-(5-(3-fluoropyridin-4-yl)-1H-pyrrole-2-carbonyl)-N-(3,4,5-trifluorophenyl) pyrrolidine-3-carboxamide

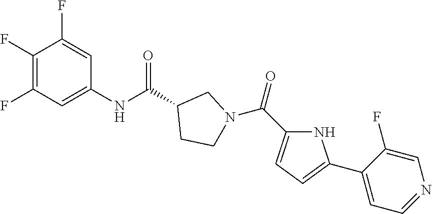 [SMILES: O=C(Nc1cc(F)c(F)c(F)c1)[C@H]1CCN(C(=O)c2ccc(-c3ccncc3F)[nH]2)C1]

### Step 1: Synthesis of methyl 5-(3-fluoropyridin-4-yl)-1H-pyrrole-2-carboxylate via Suzuki cross coupling

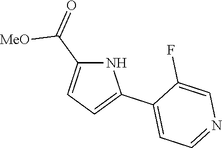 [SMILES: COC(=O)c1ccc(-c2ccncc2F)[nH]1]

A mixture of 1 eq. of 4-bromo-3-fluoropyridine and 1.5 eq. of Intermediate V-A in 1,4-dioxane:H₂O (6:1) was bubbled by argon for 10 mins. The mixture was then added with 10 mol % of Pd(PPh₃)₄ and 3 eq. of potassium carbonate. The reaction was heated at 110° C. for 2 hour in sealed tube and then cooled to room temperature, filtered, and concentrated in vacuo. The crude product was purified by liquid column chromatography using 50% EtOAc/hexane to give yellow oil.

### Step 2: Hydrolysis of methyl 5-(3-fluoropyridin-4-yl)-1H-pyrrole-2-carboxylate

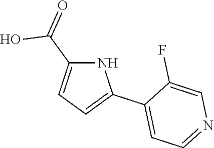 [SMILES: O=C(O)c1ccc(-c2ccncc2F)[nH]1]

1 eq. of methyl 5-(3-fluoropyridin-4-yl)-1H-pyrrole-2-carboxylate was dissolved in THF. Then, 5 eq. of LiOH was dissolved in H₂O and added to the THF solution. The reaction mixture was stirred at 95° C. for 30 mins. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(3-fluoropyridin-4-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 95 (12 mg, 94% purity by UV). LCMS Method B, Rt=4.59 min, m/z=433.1 [M+H]+, exact mass: 432.1209.

### Example 96. (S)-1-(5-(pyridazin-3-yl)-1H-pyrrole-2-carbonyl)-N-(3,4,5-trifluorophenyl)pyrrolidine-3-carboxamide

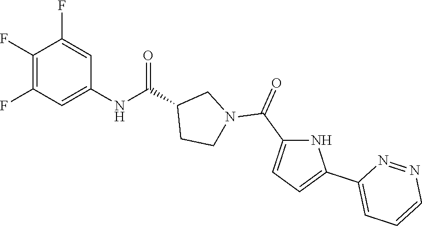 [SMILES: O=C(Nc1cc(F)c(F)c(F)c1)[C@H]1CCN(C(=O)c2ccc(-c3cccnn3)[nH]2)C1]

### Step 1: Synthesis of methyl 5-(pyridazin-3-yl)-1H-pyrrole-2-carboxylate via Suzuki cross coupling

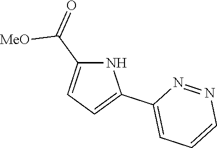 [SMILES: COC(=O)c1ccc(-c2cccnn2)[nH]1]

A mixture of 1 eq. of 3-bromopyridazine and 1.0 eq. of Intermediate V-A in 1,4-dioxane:H₂O (6:1) was bubbled by argon for 10 mins. The mixture was then added with 10 mol % of Pd(PPh₃)₄ and 3 eq. of potassium carbonate. The reaction was heated at 110° C. for 1 hour in sealed tube and then cooled to room temperature, filtered, and concentrated in vacuo. The crude product was purified by liquid column chromatography using 50% EtOAc/hexane to give yellow oil.

### Step 2: Hydrolysis of methyl 5-(pyridazin-3-yl)-1H-pyrrole-2-carboxylate

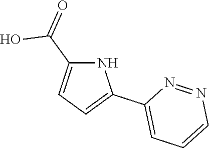 [SMILES: O=C(O)c1ccc(-c2cccnn2)[nH]1]

1 eq. of methyl 5-(pyridazin-3-yl)-1H-pyrrole-2-carboxylate was dissolved in THF. Then, 5 eq. of LiOH was dissolved in H₂O and added to the THF solution. The reaction mixture was stirred at 95° C. for 30 mins. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(pyridazin-3-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 96 (3 mg, 100% purity by UV). LCMS Method B, Rt=4.45 min, m/z=416.1 [M+H]+, exact mass: 415.1256.

### Example 97. (S)-1-(5-(2-cyanopyridin-3-yl)-1H-pyrrole-2-carbonyl)-N-(3,4,5-trifluorophenyl) pyrrolidine-3-carboxamide

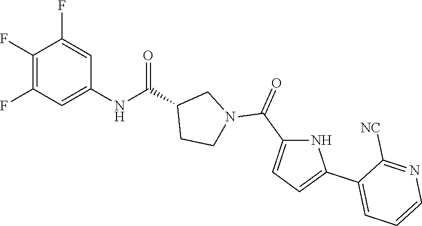 [SMILES: N#Cc1ncccc1-c1ccc(C(=O)N2CC[C@H](C(=O)Nc3cc(F)c(F)c(F)c3)C2)[nH]1]

### Step 1: Synthesis of methyl 5-(2-cyanopyridin-3-yl)-1H-pyrrole-2-carboxylate via Suzuki cross coupling

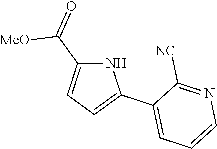 [SMILES: COC(=O)c1ccc(-c2cccnc2C#N)[nH]1]

1.2 eq. of 3-bromopicolinontrile was dissolved in H₂O:1,4-dioxane (1:5). Then 1 eq. of intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(2-cyanopyridin-3-yl)-1H-pyrrole-2-carboxylate

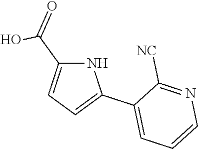 [SMILES: N#Cc1ncccc1-c1ccc(C(=O)O)[nH]1]

1 eq. of Methyl 5-(2-cyanopyridin-3-yl)-1H-pyrrole-2-carboxylate was dissolved in THF. Then, 5 eq. of LiOH was dissolved in H₂O and added to the THF solution. The reaction mixture was stirred overnight at room temperature. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(2-cyanopyridin-3-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 97 (5 mg, 99% purity by UV). LCMS Method A, Rt=4.72 min, m/z=440.2 [M+H]+, exact mass: 439.1256.

### Example 98. (S)-1-(5-(4-methylpyridazin-3-yl)-1H-pyrrole-2-carbonyl)-N-(3,4,5-trifluorophenyl) pyrrolidine-3-carboxamide

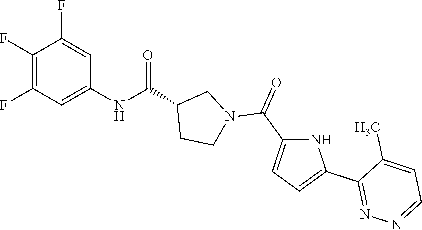 [SMILES: Cc1ccnnc1-c1ccc(C(=O)N2CC[C@H](C(=O)Nc3cc(F)c(F)c(F)c3)C2)[nH]1]

### Step 1: Synthesis of methyl 5-(4-methylpyridazin-3-yl)-1H-pyrrole-2-carboxylate via Suzuki cross coupling

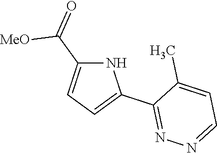 [SMILES: COC(=O)c1ccc(-c2nnccc2C)[nH]1]

1.2 eq. of 3-chloro-4-methylpyridazine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(4-methylpyridazin-3-yl)-1H-pyrrole-2-carboxylate

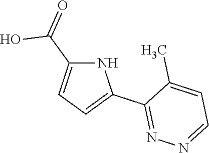

1 eq. of methyl 5-(4-methylpyridazin-3-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4-methylpyridazin-3-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 98 (8 mg, 95% purity by UV). LCMS Method A, Rt=4.50 min, m/z=430.2 [M+H]+, exact mass: 429.1413.

### Example 99. (S)—N-(3-chloro-4-fluorophenyl)-1-(5-(4,6-dimethylpyrimidin-5-yl)-1H-pyrrole-2-carbonyl) pyrrolidine-3-carboxamide

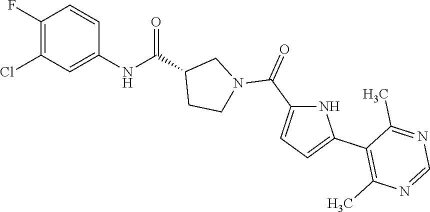 [SMILES: Cc1ncnc(C)c1-c1ccc(C(=O)N2CC[C@H](C(=O)Nc3ccc(F)c(Cl)c3)C2)[nH]1]

### Step 1: Synthesis of methyl 5-(4,6-dimethylpyrimidin-5-yl)-H-pyrrole-2-carboxylate via Suzuki cross coupling

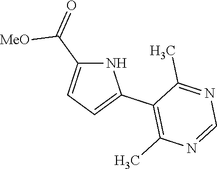 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(4,6-dimethylpyrimidin-5-yl)-1H-pyrrole-2-carboxylate

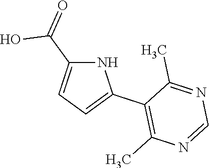 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4,6-dimethylpyrimidin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-I was added to the solution. The mixture was stirred overnight at room temperature. Then the mixture was diluted with water. The product was extracted with EtOAc for 3 times. The organic layer was washed by brine and purified by column chromatography with 1-10% MeOH/EtOAc. The desired fractions were pooled and the solvent was removed under reduced pressure to afford Example 99 (23 mg, 100% purity by UV). LCMS Method B, Rt=4.51 min, m/z=442.1 [M+H]+, LCMS Method C, Rt=4.27 min, m/z=442.1424 [M+H]+, exact mass: 441.1368. ¹H-NMR (500 MHz, Acetone-d₆) δ 11.22 (s, 1H), 9.63 (s, 1H), 8.73 (s, 1H), 7.97 (s, 1H), 7.50 (s, 1H), 7.19 (t, J=9.0 Hz, 1H), 6.73 (s, 1H), 6.19 (dd, J=3.6, 2.7 Hz, 1H), 3.99-3.95 (m, 1H), 3.61-3.49 (m, 2H), 3.31 (br, 1H), 3.15 (m, 1H), 2.21 (s, 6H), 1.98-1.96 (overlap, 2H).

### Example 100. (S)—N-(3-chloro-4,5-difluorophenyl)-1-(5-(4,6-dimethylpyrimidin-5-yl)-1H-pyrrole-2-carbonyl)pyrrolidine-3-carboxamide

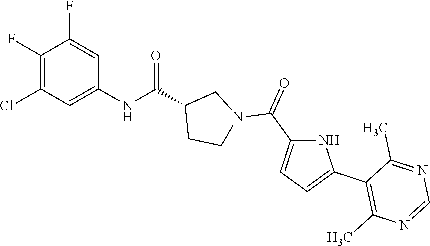 [SMILES: Cc1ncnc(C)c1-c1ccc(C(=O)N2CC[C@H](C(=O)Nc3cc(F)c(F)c(Cl)c3)C2)[nH]1]

### Step 1: Synthesis of methyl 5-(4,6-dimethylpyrimidin-5-yl)-H-pyrrole-2-carboxylate via Suzuki

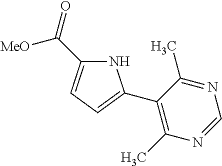 [SMILES: COC(=O)c1ccc(-c2c(C)ncnc2C)[nH]1]

A mixture of 1 eq. of 5-bromo-4,6-dimethylpyrimidine and 1.5 eq. of Intermediate V-A in 1,4-dioxane:H₂O (6:1) was bubbled by argon for 10 mins. The mixture was then added with 10 mol % of Pd(PPh₃)₄ and 3 eq. of potassium carbonate (400 mg, 3 mmol). The reaction was heated at 110° C. for 1 hour in sealed tube and then cooled to room temperature, filtered, and concentrated in vacuo. Silica gel chromatography (40% EtOAc/Hexanes) gave the product. The crude product was purified by liquid column chromatography using 70% EtOAc/hexane to give a yellow oil.

### Step 2: Hydrolysis of methyl 5-(4,6-dimethylpyrimidin-5-yl)-1H-pyrrole-2-carboxylate

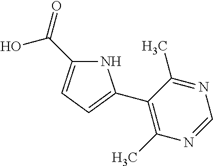 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid. Yield: 28 mg, 0.27 mmol, 57% yield.

### Step 3: Amide Formation

1 eq. of 5-(4,6-dimethylpyrimidin-5-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-K was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 100 (6 mg, 96% purity by UV). LCMS Method B, Rt=4.61 min, m/z=460.1 [M+H]+, LCMS Method C, Rt=4.48 min, m/z=460.1345 [M+H]+, exact mass: 459.1274.

### Example 101. (S)-1-(5-(3,5-dimethyl-1H-pyrazol-4-yl)-1H-pyrrole-2-carbonyl)-N-(4-fluoro-3-methylphenyl)pyrrolidine-3-carboxamide

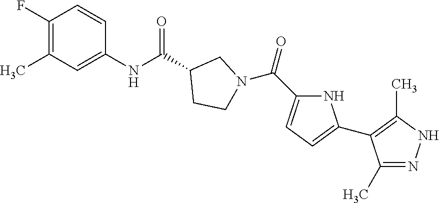 [SMILES: Cc1cc(NC(=O)[C@H]2CCN(C(=O)c3ccc(-c4c(C)n[nH]c4C)[nH]3)C2)ccc1F]

### Step 1: Synthesis of methyl 5-(3,5-dimethyl-1H-pyrazol-4-yl)-1H-pyrrole-2-carboxylate via Suzuki cross coupling

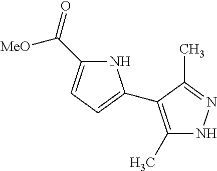 [SMILES: COC(=O)c1ccc(-c2c(C)n[nH]c2C)[nH]1]

1.2 eq. of 4-iodo-3,5-dimethyl-1H-pyrazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(3,5-dimethyl-1H-pyrazol-4-yl)-1H-pyrrole-2-carboxylate

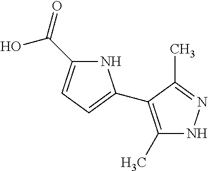

1 eq. of methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3,5-dimethyl-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-B was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 101 (10 mg, 98% purity by UV). LCMS Method B, Rt=4.33 min, m/z=410.2 [M+H]+, exact mass: 409.1914.

### Example 102. (2S,3S)—N-(4-fluoro-3-methylphenyl)-2-methyl-1-(5-(pyridazin-4-yl)-1H-pyrrole-2-carbonyl) pyrrolidine-3-carboxamide

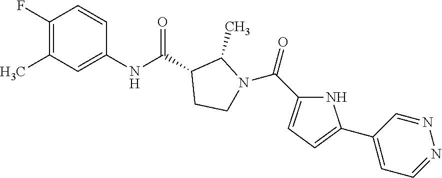 [SMILES: Cc1cc(NC(=O)[C@H]2CCN(C(=O)c3ccc(-c4ccnnc4)[nH]3)[C@H]2C)ccc1F]

### Step 1: Synthesis of methyl 5-(pyridazin-4-yl)-1H-pyrrole-2-carboxylate via Suzuki cross coupling

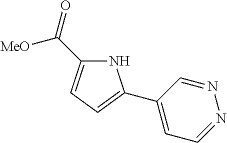 [SMILES: COC(=O)c1ccc(-c2ccnnc2)[nH]1]

1 eq. of pyridazin-4-amine was dissolved in 1,2-DME. Then, 0.5 eq. of iodine, 1 eq. of potassium iodide, 0.3 eq. of copper(I)iodide and 4 eq. of isoamyl nitrite were added to the previous solution. The reaction mixture was heated at 70° C. for 2 hours. Crude reaction was evaporated by rotavapor. Diluted with water and extracted by EtOAc for 3 times. The organic layer was concentrated. The product was purified by liquid column chromatography with EtOAc/hexane to achieve the pure product of 4-iodopyridazine.

A mixture of 1 eq. of 4-iodopyridazine and 1.11 eq. of Intermediate V-A in 1,4-dioxane:H₂O (6:1) was bubbled by argon for 10 mins. The mixture was then added with 10 mol % of Pd(PPh₃)₄ and 1.11 eq. of potassium carbonate. The reaction was heated at 80° C. for 3 hours in sealed tube and then cooled to room temperature, filtered, and concentrated in vacuo. The crude product was purified by liquid column chromatography using 60% EtOAc/hexane to give yellow oil.

### Step 2: Hydrolysis of methyl 5-(pyridazin-4-yl)-1H-pyrrole-2-carboxylate

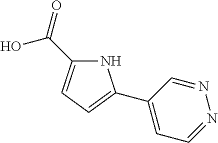 [SMILES: O=C(O)c1ccc(-c2ccnnc2)[nH]1]

1 eq. of methyl 5-(pyridazin-4-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(pyridazin-4-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-H was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 102 (10 mg, 100% purity by UV). LCMS Method B, Rt=4.37 min, m/z=408.2 [M+H]+, LCMS Method C, Rt=4.02 min, m/z=408.1827 [M+H]+, exact mass: 407.1758. ¹H-NMR (500 MHz, Acetone-d₆) δ 11.2 (s, 1H), 9.67 (s, 1H), 9.29 (s, 1H), 9.12 (d, J=5.8 Hz, 1H), 8.02 (dd, J=6.5, 8.3 Hz, 1H), 7.58 (d, J=9.3 Hz, 1H), 7.53-7.45 (m, 1H), 7.13-7.06 (m, 1H), 6.99 (t, J=9.5 Hz, 1H), 6.89-6.82 (m, 1H), 3.83 (m, 1H), 3.28 (m, 1H), 2.61 (m, 2H), 2.61 (m, 1H), 1.92 (m, 1H), 1.20 (d, J=4.4 Hz, 3H).

### Example 103. (2S,3S)-1-(5-(3,5-dimethyl-1H-pyrazol-4-yl)-1H-pyrrole-2-carbonyl)-N-(4-fluoro-3-methyl phenyl)-2-methylpyrrolidine-3-carboxamide

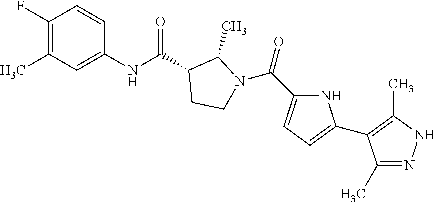 [SMILES: Cc1cc(NC(=O)[C@H]2CCN(C(=O)c3ccc(-c4c(C)n[nH]c4C)[nH]3)[C@H]2C)ccc1F]

### Step 1: Synthesis of methyl 5-(3,5-dimethyl-1H-pyrazol-4-yl)-1H-pyrrole-2-carboxylate via Suzuki cross coupling

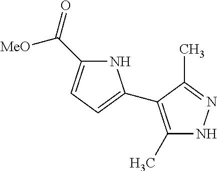 [SMILES: COC(=O)c1ccc(-c2c(C)n[nH]c2C)[nH]1]

1.2 eq. of 4-iodo-3,5-dimethyl-1H-pyrazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(3,5-dimethyl-1H-pyrazol-4-yl)-1H-pyrrole-2-carboxylate

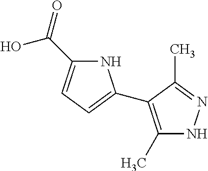

1 eq. of methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3,5-dimethyl-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-H was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 103 (11 mg, 97% purity by UV). LCMS Method B, Rt=4.51 min, m/z=424.2 [M+H]+, LCMS Method C, Rt=4.27 min, m/z=424.1249 [M+H]+, exact mass: 423.2071. ¹H-NMR (500 MHz, DMSO-d₆) δ12.2 (s, 1H), 11.0 (s, 1H), 10.0 (s, 1H), 7.52 (dd, J=7.0, 2.2 Hz, 1H), 7.44-7.36 (m, 1H), 7.07 (t, J=9.2 Hz, 1H), 6.67 (t, J=5.0 Hz, 1H), 6.06 (t, J=3.0 Hz, 1H), 3.69 (m, 1H), 3.46 (m, 1H), 3.11 (m, 2H), 2.37 (m, 1H), 2.21 (s, 6H), 2.08 (m, 1H), 1.07 (d, J=5.2 Hz, 3H).

### Example 104. (2S,3S)—N-(3-chloro-4-fluorophenyl)-1-(5-(4,6-dimethylpyrimidin-5-yl)-1H-pyrrole-2-carbonyl)-2-methylpyrrolidine-3-carboxamide

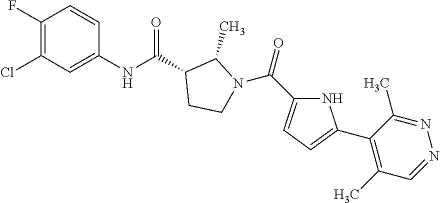 [SMILES: Cc1cnnc(C)c1-c1ccc(C(=O)N2CC[C@H](C(=O)Nc3ccc(F)c(Cl)c3)[C@@H]2C)[nH]1]

### Step 1: Synthesis of methyl 5-(4,6-dimethylpyrimidin-5-yl)-1H-pyrrole-2-carboxylate via Suzuki cross coupling

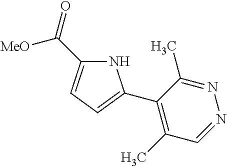 [SMILES: COC(=O)c1ccc(-c2c(C)cnnc2C)[nH]1]

1.2 eq. of 5-bromo-4,6-dimethylpyrimidine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexane.

### Step 2: Hydrolysis of methyl 5-(4,6-dimethylpyrimidin-5-yl)-1H-pyrrole-2-carboxylate

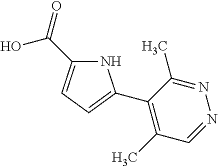 [SMILES: Cc1cnnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was heated to 90° C. and kept heating for an hour. The reaction was acidified by adding 2N HCl until pH of solution approximated to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4,6-dimethylpyrimidin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-M was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 104 (18 mg, 93% purity by UV). LCMS Method B, Rt=4.61 min, m/z=456.2 [M+H]+, LCMS Method C, Rt=4.45 min, m/z=456.1587 [M+H]+, exact mass: 455.1524. ¹H-NMR (500 MHz, DMSO-d₆) δ 11.7 (s, 1H), 10.3 (s, 1H), 8.84 (s, 1H), 7.95 (dd, J=6.8, 2.5 Hz, 1H), 7.52-7.18 (m, 1H), 7.38 (t, J=9.1 Hz, 1H), 6.76 (m, 1H), 6.24 (s, 1H), 3.97 (m, 1H), 3.73 (m, 1H), 3.17 (m, 2H), 2.43 (m, 1H), 2.28 (s, 6H), 2.10 (m, 1H), 1.07 (brs, 3H).

### Example 105. (2S,3S)—N-(3-chloro-4-fluorophenyl)-1-(5-(3,5-dimethyl-1H-pyrazol-4-yl)-1H-pyrrole-2-carbonyl)-2-methylpyrrolidine-3-carboxamide

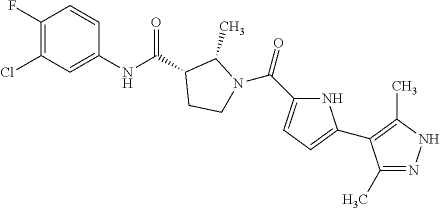 [SMILES: Cc1n[nH]c(C)c1-c1ccc(C(=O)N2CC[C@H](C(=O)Nc3ccc(F)c(Cl)c3)[C@@H]2C)[nH]1]

### Step 1: Synthesis of methyl 5-(3,5-dimethyl-1H-pyrazol-4-yl)-1H-pyrrole-2-carboxylate via Suzuki cross coupling

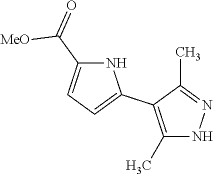 [SMILES: COC(=O)c1ccc(-c2c(C)n[nH]c2C)[nH]1]

1.2 eq. of 4-iodo-3,5-dimethyl-1H-pyrazole was dissolved in H-20:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(3,5-dimethyl-1H-pyrazol-4-yl)-1H-pyrrole-2-carboxylate

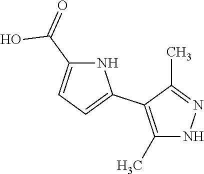

1 eq. of methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3,5-dimethyl-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-M was added to the solution. The mixture was stirred overnight at room temperature. Then the mixture was diluted with water. The product was extracted with EtOAc for 3 times. The organic layer was washed by brine and purified by column chromatography with 1-10% MeOH/EtOAc. The desired fractions were pooled and the solvent was removed under reduced pressure to afford Example 105 (5 mg, 99% purity by UV). LCMS Method B, Rt=4.54 min, m/z=444.1 [M+H]+, LCMS Method C, Rt=4.39 min, m/z=444.1604 [M+H]+, exact mass: 443.1524.

### Example 106. (S)—N-(3-chloro-4-fluorophenyl)-1-(5-(4-methoxy-6-methylpyrimidin-5-yl)-1H-pyrrole-2-carbonyl)pyrrolidine-3-carboxamide

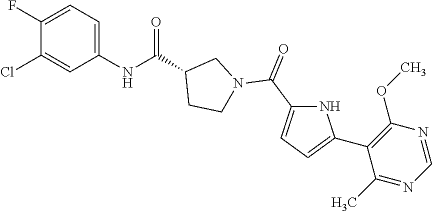 [SMILES: COc1ncnc(C)c1-c1ccc(C(=O)N2CC[C@H](C(=O)Nc3ccc(F)c(Cl)c3)C2)[nH]1]

### Step 1: Synthesis of methyl 5-(4-methoxy-6-methylpyrimidin-5-yl)-1H-pyrrole-2-carboxylate via Suzuki cross coupling

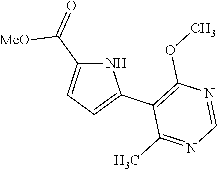 [SMILES: COC(=O)c1ccc(-c2c(C)ncnc2OC)[nH]1]

A mixture of 1 eq. of 5-bromo-4-methoxy-6-methylpyrimidine and 1 eq. of Intermediate V-A in 1,4-dioxane:H₂O (6:1) was bubbled by argon for 10 mins. The mixture was then added with 10 mol % of Pd(PPh₃)₄ and 3 eq. of potassium carbonate. The reaction was heated at 110° C. for 1 hour in sealed tube and then cooled to room temperature, filtered, and concentrated in vacuo. The crude product was purified by liquid column chromatography using 70% EtOAc/hexane to give yellow oil.

### Step 2: Hydrolysis of methyl 5-(4-methoxy-6-methylpyrimidin-5-yl)-1H-pyrrole-2-carboxylate

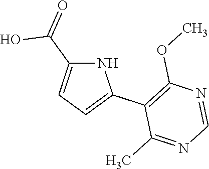 [SMILES: COc1ncnc(C)c1-c1ccc(C(=O)O)[nH]1]

1 eq. of methyl 5-(4-methoxy-6-methylpyrimidin-5-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(4-methoxy-6-methylpyrimidin-5-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-I was added to the solution. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 106 (7 mg, 98% purity by UV). LCMS Method B, Rt=4.57 min, m/z=458.1 [M+H]+, exact mass: 457.1317.

### Example 107. (S)-1-(5-(1,4-dimethyl-1H-imidazol-5-yl)-1H-pyrrole-2-carbonyl)-N-(3,4,5-trifluorophenyl) pyrrolidine-3-carboxamide

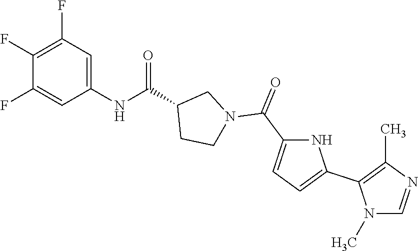 [SMILES: Cc1ncn(C)c1-c1ccc(C(=O)N2CC[C@H](C(=O)Nc3cc(F)c(F)c(F)c3)C2)[nH]1]

### Step 1: Synthesis of methyl 5-(1,4-dimethyl-1H-imidazol-5-yl)-1H-pyrrole-2-carboxylate via Suzuki cross coupling

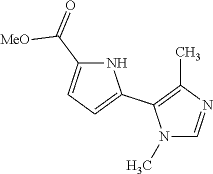 [SMILES: COC(=O)c1ccc(-c2c(C)ncn2C)[nH]1]

1.2 eq. of 5-bromo-1,4-dimethyl-1H-imidazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(1,4-dimethyl-1H-imidazol-5-yl)-1H-pyrrole-2-carboxylate

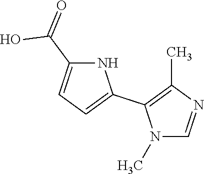

1 eq. of methyl 5-(1,4-dimethyl-1H-imidazol-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(1,4-dimethyl-1H-imidazol-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 107 (13 mg, 93% purity by UV). LCMS Method A, Rt=4.20 min, m/z=432.2 [M+H]+, exact mass: 431.1569.

### Example 108. (S)-1-(5-(4-methylthiazol-5-yl)-1H-pyrrole-2-carbonyl)-N-(3,4,5-trifluorophenyl) pyrrolidine-3-carboxamide

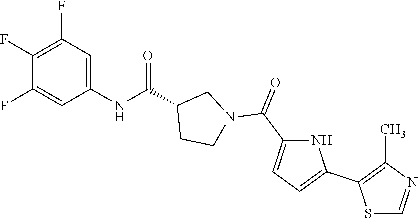 [SMILES: Cc1ncsc1-c1ccc(C(=O)N2CC[C@H](C(=O)Nc3cc(F)c(F)c(F)c3)C2)[nH]1]

### Step 1: Synthesis of methyl 5-(4-methylthiazol-5-yl)-1H-pyrrole-2-carboxylate via Suzuki cross coupling

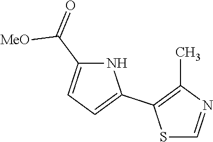

1.2 eq. of 5-bromo-4-methylthiazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(4-methylthiazol-5-yl)-1H-pyrrole-2-carboxylate

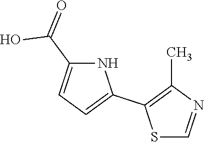

1 eq. of methyl 5-(4-methylthiazol-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4-methylthiazol-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 108 (17 mg, 100% purity by UV). LCMS Method A, Rt=4.56 min, m/z=435.1 [M+H]+, exact mass: 434.1024.

### Example 109. (S)-1-(5-(3,5-dimethylisoxazol-4-yl)-1H-pyrrole-2-carbonyl)-N-(3,4,5-trifluorophenyl) pyrrolidine-3-carboxamide

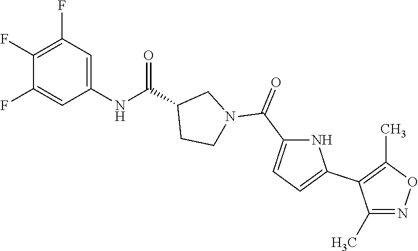 [SMILES: Cc1noc(C)c1-c1ccc(C(=O)N2CC[C@H](C(=O)Nc3cc(F)c(F)c(F)c3)C2)[nH]1]

### Step 1: Synthesis of methyl 5-(3,5-dimethylisoxazol-4-yl)-1H-pyrrole-2-carboxylate via Suzuki cross coupling

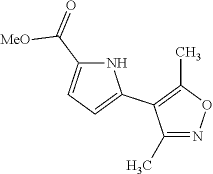 [SMILES: COC(=O)c1ccc(-c2c(C)noc2C)[nH]1]

A mixture of 1 eq. of 4-bromo-3,5-dimethylisoxazole, 1 eq. of Intermediate V-A, 10 mol % of tris(dibenzylideneacetone) dipalladium(0), 20 mol % of Xphos and 3 eq. of potassium phosphate monohydrate in 1,4-dioxane was heated at 105° C. for 3 hours in sealed tube and then cooled to room temperature, filtered, and concentrated in vacuo. Crude product was purified via column chromatography by using 50% EtOAc/Hexanes.

### Step 2: Hydrolysis of methyl 5-(3,5-dimethylisoxazol-4-yl)-1H-pyrrole-2-carboxylate

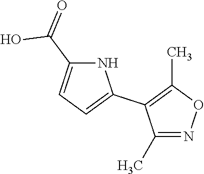

1 eq. of methyl 5-(3,5-dimethylisoxazol-4-yl)-1H-pyrrole-2-carboxylate was dissolved in THF. Then, 5 eq. of LiOH was dissolved in H₂O and added to the THF solution. The reaction mixture was heated until it reached to 80° C. and kept heating for 2 hours. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3,5-dimethylisoxazol-4-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-A was added to the solution. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with EtOAc to give Example 109 (6 mg, 96% purity by UV). LCMS Method B, Rt 4.71 min, m/z=433.1 [M+H]+, 455.1 [M+Na]+, LCMS Method C, Rt=4.68 min, m/z=433.1478 [M+H]+, exact mass: 432.1409.

### Example 110. (2S,3S)—N-(3,4-difluorophenyl)-2-methyl-1-(5-(pyridazin-4-yl)-1H-pyrrole-2-carbonyl) pyrrolidine-3-carboxamide

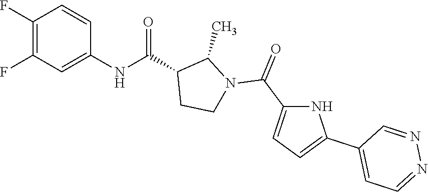 [SMILES: C[C@H]1[C@@H](C(=O)Nc2ccc(F)c(F)c2)CCN1C(=O)c1ccc(-c2ccnnc2)[nH]1]

### Step 1: Synthesis of methyl 5-(pyridazin-4-yl)-1H-pyrrole-2-carboxylate via Suzuki cross coupling

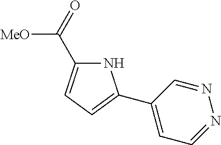 [SMILES: COC(=O)c1ccc(-c2ccnnc2)[nH]1]

1 eq. of pyridazin-4-amine was dissolved in 1,2-DME. Then, 0.5 eq. of iodine, 1 eq. of potassium iodide, 0.3 eq. of copper(I)iodide and 4 eq. of isoamyl nitrite were added to the previous solution. The reaction mixture was heated at 70° C. for 2 hours. Crude reaction was evaporated by rotavapor. Diluted with water and extracted by EtOAc for 3 times. The organic layer was concentrated. The product was purified by liquid column chromatography with EtOAc/hexane to achieve the pure product of 4-iodopyridazine.

A mixture of 1 eq. of 4-iodopyridazine and 1.11 eq. of Intermediate V-A in 1,4-dioxane:H₂O (6:1) was bubbled by argon for 10 mins. The mixture was then added with 10 mol % of Pd(PPh₃)₄ and 1.11 eq. of potassium carbonate. The reaction was heated at 80° C. for 3 hours in sealed tube and then cooled to room temperature, filtered, and concentrated in vacuo. The crude product was purified by liquid column chromatography using 60% EtOAc/hexane to give yellow oil.

### Step 2: Hydrolysis of methyl 5-(pyridazin-4-yl)-1H-pyrrole-2-carboxylate

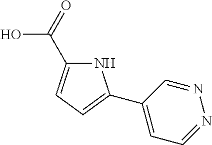 [SMILES: O=C(O)c1ccc(-c2ccnnc2)[nH]1]

1 eq. of methyl 5-(pyridazin-4-yl)-1H-pyrrole-2-carboxylate was dissolved in THE. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(pyridazin-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-N was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 110 (12 mg, 99% purity by UV). LCMS Method A, Rt=4.38 min, m/z=412.1 [M+H]+, exact mass: 411.1507.

### Example 111. (2S,3S)-1-(5-(4,6-dimethylpyrimidin-5-yl)-1H-pyrrole-2-carbonyl)-2-methyl-N-(3,4,5-trifluorophenyl)pyrrolidine-3-carboxamide

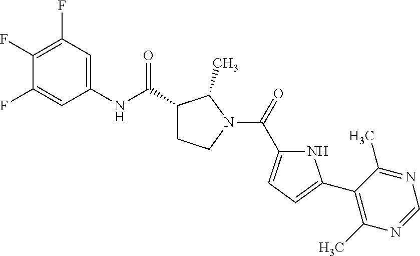 [SMILES: Cc1ncnc(C)c1-c1ccc(C(=O)N2CC[C@H](C(=O)Nc3cc(F)c(F)c(F)c3)[C@@H]2C)[nH]1]

### Step 1: Synthesis of methyl 5-(4,6-dimethylpyrimidin-5-yl)-1H-pyrrole-2-carboxylate via Suzuki cross coupling

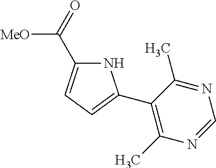 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(4,6-dimethylpyrimidin-5-yl)-1H-pyrrole-2-carboxylate

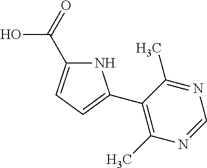 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(4,6-dimethylpyrimidin-5-yl)-1H-pyrrole-2-carboxylic acid (0.27 mmol) was dissolved in DMF. Then 1.5 eq. of HATU and 5 eq. of DIPEA were added. The reaction mixture was stirred at room temperature for 10 mins. Then, 1.1 eq. of Intermediate III-D was added to the solution. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 111 (2 mg, 98% purity by UV). LCMS Method B, Rt=4.64 min, m/z=458.2 [M+H]+, LCMS Method C, Rt=4.52 min, m/z=458.1791 [M+H]+, exact mass: 457.1726.

### Example 112. (S)-1-(5-(1-(2-(3-hydroxyazetidin-1-yl)-2-oxoethyl)-3,5-dimethyl-1H-pyrazol-4-yl)-1H-pyrrole-2-carbonyl)-N-(3,4,5-trifluorophenyl)pyrrolidine-3-carboxamide

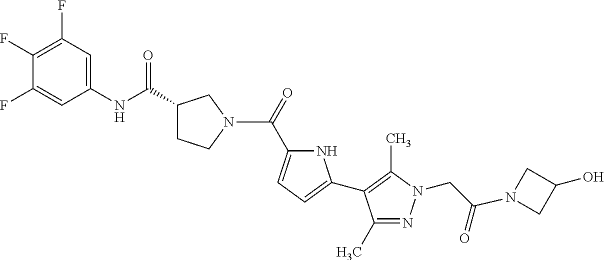 [SMILES: Cc1nn(CC(=O)N2CC(O)C2)c(C)c1-c1ccc(C(=O)N2CC[C@H](C(=O)Nc3cc(F)c(F)c(F)c3)C2)[nH]1]

### Step 1: Synthesis of methyl 5-(1-(2-(3-hyroxyazetidin-1-yl)-2-oxoethyl)-3,5-dimethyl-1H-pyrazol-4-yl)-1H-pyrrole-2-carboxylate

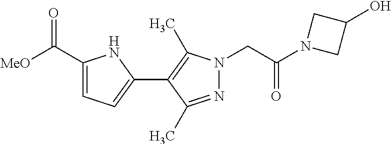 [SMILES: COC(=O)c1ccc(-c2c(C)nn(CC(=O)N3CC(O)C3)c2C)[nH]1]

1 eq. of 4-iodo-3,5-dimethyl-1H-pyrazole was dissolved in THF. Then 60% sodium hydride solution (1.05 eq.) was added to the mixture. The mixture was stirred for 10 mins. at 0° C. 1.16 eq. of tert-butyl 3-bromopropanoate was added. The solution was heated at 60° C. for 2 hours. Then water was added to quench the reaction. The product was extracted by EtOAc and the crude was purified via silica gel column chromatography by using EtOAC:Hexanes.

Subsequent t-butyl ester deprotection HCl (4M in dioxane, 30 minutes at room temperature) afforded 3-(4-iodo-3,5-dimethyl-1H-pyrazol-1-yl)propanoic acid that was used as such in the next step without further purification.

1 eq. of 3-(4-iodo-3,5-dimethyl-1H-pyrazol-1-yl)propanoic acid in DMF was treated with 5 eq. of DIPEA. Then 1.5 eq. of HATU was added and stirred for 5 mins. 1.2 eq. of azetidin-3-ol was added to the solution and stirred it at room temperature overnight. Brine was added and the mixture was partitioned with EtOAc. The organic layers were dried over sodium sulfate anhydrous, the solids were removed by filtration, and the solvent was removed under reduced pressure and the crude was purified via silica gel column chromatography by using MeOH:EtOAC.

1.2 eq. of 1-(3-hydroxyazetidin-1-yl)-2-(4-iodo-3,5-dimethyl-1H-pyrazol-1-yl)ethan-1-one was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(1-(2-(3-hydroxyazetidin-1-yl)-2-oxoethyl)-3,5-dimethyl-1H-pyrazol-4-yl)-1H-pyrrole-2-carboxylate

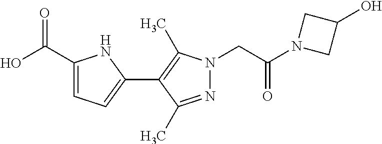 [SMILES: Cc1nn(CC(=O)N2CC(O)C2)c(C)c1-c1ccc(C(=O)O)[nH]1]

1 eq. of methyl 5-(1-(2-(3-hydroxyazetidin-1-yl)-2-oxoethyl)-3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(1-(2-(3-hydroxyazetidin-1-yl)-2-oxoethyl)-3,5-dimethyl-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 112 (5 mg, 96% purity by UV). LCMS Method A, Rt=4.28 min, m/z=545.2 [M+H]+, LCMS Method C, Rt=4.09 min, m/z=545.2107 [M+H]+, exact mass: 544.2046.

### Example 113. (2S,3S)—N-(3-cyano-4-fluorophenyl)-2-methyl-1-(5-(5-methyl-3-(trifluoromethyl)-1H-pyrazol-4-yl)-1H-pyrrole-2-carbonyl)pyrrolidine-3-carboxamide

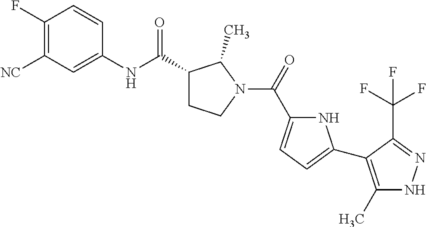 [SMILES: Cc1[nH]nc(C(F)(F)F)c1-c1ccc(C(=O)N2CC[C@H](C(=O)Nc3ccc(F)c(C#N)c3)[C@@H]2C)[nH]1]

### Step 1: Synthesis of methyl 5-(5-methyl-3-(trifluoromethyl)-1H-pyrazol-4-yl)-1H-pyrrole-2-carboxylate via Suzuki cross coupling

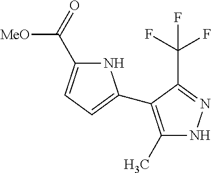 [SMILES: COC(=O)c1ccc(-c2c(C(F)(F)F)n[nH]c2C)[nH]1]

1.2 eq. of 4-bromo-5-methyl-3-(trifluoromethyl)-1H-pyrazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(5-methyl-3-(trifluoromethyl)-1H-pyrazol-4-yl)-1H-pyrrole-2-carboxylate

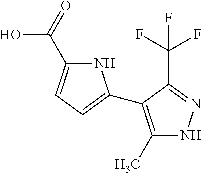 [SMILES: Cc1[nH]nc(C(F)(F)F)c1-c1ccc(C(=O)O)[nH]1]

1 eq. of methyl 5-(5-methyl-3-(trifluoromethyl)-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(5-methyl-3-(trifluoromethyl)-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-G was added to the solution. The mixture was stirred overnight at room temperature. Then the mixture was diluted with water. The product was extracted with EtOAc for 3 times. The organic layer was washed by brine and purified by column chromatography with 1-10% MeOH/EtOAc. The desired fractions were pooled and the solvent was removed under reduced pressure to afford Example 113 (11 mg, 96% purity by UV). LCMS Method A, Rt=4.64 min, m/z=489.1 [M+H]+, 511.1 [M+Na]+, LCMS Method C, Rt=4.58 min, m/z=489.1673 [M+H]+, exact mass: 488.1584.

### Example 114. (S)—N-(4-fluoro-3-methylphenyl)-1-(5-(5-methyl-3-(trifluoromethyl)-1H-pyrazole-4-carbonyl)-1H-pyrrole-2-carbonyl)pyrrolidine-3-carboxamide

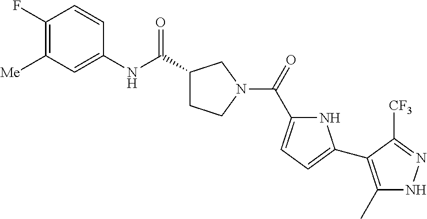 [SMILES: Cc1cc(NC(=O)[C@H]2CCN(C(=O)c3ccc(-c4c(C(F)(F)F)n[nH]c4C)[nH]3)C2)ccc1F]

### Step 1: Synthesis of methyl 5-(5-methyl-3-(trifluoromethyl)-1H-pyrazol-4-yl)-1H-pyrrole-2-carboxylate via Suzuki cross coupling

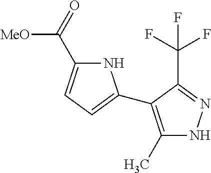 [SMILES: COC(=O)c1ccc(-c2c(C(F)(F)F)n[nH]c2C)[nH]1]

1.2 eq. of 4-bromo-5-methyl-3-(trifluoromethyl)-1H-pyrazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(5-methyl-3-(trifluoromethyl)-1H-pyrazol-4-yl)-1H-pyrrole-2-carboxylate

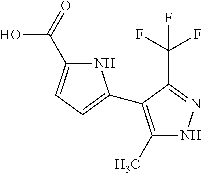 [SMILES: Cc1[nH]nc(C(F)(F)F)c1-c1ccc(C(=O)O)[nH]1]

1 eq. of methyl 5-(5-methyl-3-(trifluoromethyl)-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(5-methyl-3-(trifluoromethyl)-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-B was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 114 (6 mg, 98% purity by UV). LCMS Method A, Rt=4.57 min, m/z=464.1 [M+H]+ 486.2 [M+Na]+, exact mass: 463.1581. ¹H-NMR (500 MHz, DMSO-d₆) δ11.3 (s, 1H), 10.1 (s, 1H), 7.95 (s, 1H), 7.54 (dd, J=7.9, 2.4 Hz, 1H), 7.44-7.37 (m, 1H), 7.07 (t, J=9.2 Hz, 1H), 6.68 (t, J=5.0 Hz, 1H), 6.12 (t, J=5.0 Hz, 1H), 3.87 (m, 1H), 3.78 (m, 1H), 3.66 (m, 1H), 3.52 (m, 1H), 3.16 (m, 1H), 2.20 (s, 3H) 2.08 (brs, 2H).

### Example 115. (S)—N-(3,4-difluorophenyl)-1-(5-(3,5-dimethylpyridazin-4-yl)-1H-pyrrole-2-carbonyl)pyrrolidine-3-carboxamide

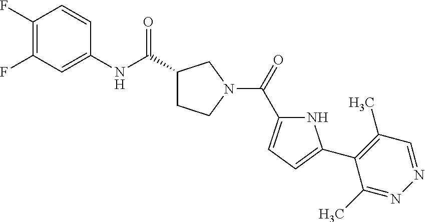 [SMILES: Cc1cnnc(C)c1-c1ccc(C(=O)N2CC[C@H](C(=O)Nc3ccc(F)c(F)c3)C2)[nH]1]

### Step 1: Synthesis of methyl 5-(3,5-dimethylpyridazin-4-yl)-1H-pyrrole-2-carboxylate

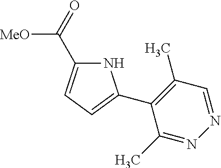 [SMILES: COC(=O)c1ccc(-c2c(C)cnnc2C)[nH]1]

A mixture of 1 eq. of 4,5-dichloropyridazin-3-ol, 8 eq. of 3,4-dihydro-2H-pyran and 0.2 eq. of para-toluenesulfonic acid in THF (200 ML) was refluxed for 1 days. After cooling to room temperature, the mixture was concentrated under reduced pressure. The residue was purified via column chromatography by using ethyl acetate/hexanes to afford the product as a white solid.

A mixture of 1 eq. of 4,5-dichloro-2-(tetrahydro-2H-pyran-2-yl)pyridazin-3(2H)-one, 1 eq. of methylboronic acid and 3 eq. of cesium carbonate was stirred in 1,4-dioxane:water (10:1). Then 44 eq. of 1,1′-bis(diphenylphosphino)ferrocene]dichloro palladium(11) was added to the mixture. The reaction mixture was stirred at 110° C. for 2 hours, then concentrated under reduced pressure. Crude product was purified via column chromatography by using ethyl acetate:hexanes to obtain pure product as a yellow solid.

A mixture of 1 eq. of 4-chloro-5-methyl-2-(tetrahydro-2H-pyran-2-yl)pyridazin-3(2H)-one, 1.25 eq. of Intermediate V-A, 10 mol % of tris(dibenzylideneacetone)dipalladium(0), 20 mol % of tricyclohexylphosphine and 3 eq. of potassium phosphate monohydrate in 1,4-dioxane was heated at 105° C. for 3 hours in sealed tube and then cooled to room temperature, filtered, and concentrated in vacuo. The crude product was purified by liquid column chromatography using 20% EtOAc/hexane to obtain yellow oil.

1 eq. of methyl 5-(5-methyl-3-oxo-2-(tetrahydro-2H-pyran-2-yl)-2,3-dihydropyridazin-4-yl)-1H-pyrrole-2-carboxylate was dissolved in methanol, treated with 20 eq. of 4M hydrogen chloride in 1,4-dioxane, and allowed to stir at room temperature for 3 hours. Removal of solvent under reduced pressure provided the product as a pale yellow solid, presumed to be the hydrochloride salt that was used as such in the next step without further purification.

1 eq. of methyl 5-(5-methyl-3-oxo-2,3-dihydropyridazin-4-yl)-1H-pyrrole-2-carboxylate hydrochloride was suspended in 50 eq. of phosphorus oxychloride. The reaction mixture was heated at 90° C. for 2 hours. After removal of phosphorus oxychloride under reduced pressure, the residue was partitioned between Ethyl acetate, water, and saturated aqueous sodium bicarbonate solution. The organic layer was dried over sodium sulfate, filtered, and concentrated in vacuo. The crude product was purified by liquid column chromatography using 20% EtOAc/hexane to obtain yellow oil.

A mixture of 1 eq. of methyl 5-(3-chloro-5-methylpyridazin-4-yl)-1H-pyrrole-2-carboxylate, 5 eq. of methylboronic acid, 10 mol % of 1,1′-bis(diphenylphosphino)ferrocene]dichloropalladium(11), and 3 eq. of cesium carbonate in 1,4-dioxane was heated at 105° C. for 2.5 hours in sealed tube and then cooled to room temperature, filtered, and concentrated in vacuo. The crude product was purified by liquid column chromatography using 25% EtOAc/hexane to obtain a yellow solid

### Step 2: Hydrolysis of methyl 5-(3,5-dimethylpyridazin-4-yl)-1H-pyrrole-2-carboxylate

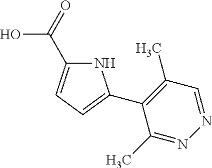 [SMILES: Cc1cnnc(C)c1-c1ccc(C(=O)O)[nH]1]

1 eq. of methyl 5-(3,5-dimethylpyridazin-4-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(3,5-dimethylpyridazin-4-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-O was added to the solution. The mixture was stirred overnight at room temperature. Then the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 115 (20 mg, 97% purity by UV). LCMS Method B, Rt=4.40 min, m/z=426.2 [M+H]+, LCMS Method C, Rt=3.95 min, m/z=426.1743 [M+H]+, exact mass: 425.1663.

### Example 116. (2S,3S)—N-(3-cyano-4-fluorophenyl)-1-(5-(3,5-dimethylpyridazin-4-yl)-1H-pyrrole-2-carbonyl)-2-methylpyrrolidine-3-carboxamide

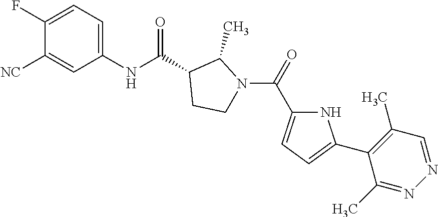 [SMILES: Cc1cnnc(C)c1-c1ccc(C(=O)N2CC[C@H](C(=O)Nc3ccc(F)c(C#N)c3)[C@@H]2C)[nH]1]

### Step 1: Synthesis of methyl 5-(3,5-dimethylpyridazin-4-yl)-1H-pyrrole-2-carboxylate

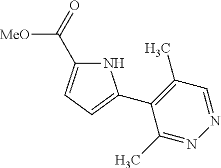 [SMILES: COC(=O)c1ccc(-c2c(C)cnnc2C)[nH]1]

A mixture of 1 eq. of 4,5-dichloropyridazin-3-ol, 8 eq. of 3,4-dihydro-2H-pyran and 0.2 eq. of para-toluenesulfonic acid in THF (200 ML) was refluxed for 1 days. After cooling to room temperature, the mixture was concentrated under reduced pressure. The residue was purified via column chromatography by using ethyl acetate/hexanes to afford the product as a white solid.

A mixture of 1 eq. of 4,5-dichloro-2-(tetrahydro-2H-pyran-2-yl)pyridazin-3(2H)-one, 1 eq. of methylboronic acid and 3 eq. of cesium carbonate was stirred in 1,4-dioxane:water (10:1). Then 44 eq. of 1,1′-bis(diphenylphosphino)ferrocene]dichloro palladium(11) was added to the mixture. The reaction mixture was stirred at 110° C. for 2 hours, then concentrated under reduced pressure. Crude product was purified via column chromatography by using ethyl acetate:hexane to obtain pure product as a yellow solid.

A mixture of 1 eq. of 4-chloro-5-methyl-2-(tetrahydro-2H-pyran-2-yl)pyridazin-3(2H)-one, 1.25 eq. of Intermediate V-A, 10 mol % of tris(dibenzylideneacetone)dipalladium(0), 20 mol % of tricyclohexylphosphine and 3 eq. of potassium phosphate monohydrate in 1,4-dioxane was heated at 105° C. for 3 hours in sealed tube and then cooled to room temperature, filtered, and concentrated in vacuo. The crude product was purified by liquid column chromatography using 20% EtOAc/hexane to obtain yellow oil.

1 eq. of methyl 5-(5-methyl-3-oxo-2-(tetrahydro-2H-pyran-2-yl)-2,3-dihydropyridazin-4-yl)-1H-pyrrole-2-carboxylate was dissolved in methanol, treated with 20 eq. of 4M hydrogen chloride in 1,4-dioxane, and allowed to stir at room temperature for 3 hours. Removal of solvent under reduced pressure provided the product as a pale-yellow solid, presumed to be the hydrochloride salt that was used as such in the next step without further purification.

1 eq. of methyl 5-(5-methyl-3-oxo-2,3-dihydropyridazin-4-yl)-1H-pyrrole-2-carboxylate hydrochloride was suspended in 50 eq. of phosphorus oxychloride. The reaction mixture was heated at 90° C. for 2 hours. After removal of phosphorus oxychloride under reduced pressure, the residue was partitioned between ethyl acetate, water, and saturated aqueous sodium bicarbonate solution. The organic layer was dried over sodium sulfate, filtered, and concentrated in vacuo. The crude product was purified by liquid column chromatography using 20% EtOAc/hexane to obtain yellow oil.

A mixture of 1 eq. of methyl 5-(3-chloro-5-methylpyridazin-4-yl)-1H-pyrrole-2-carboxylate, 5 eq. of methylboronic acid, 10 mol % of 1,1′-bis(diphenylphosphino)ferrocene]dichloropalladium(11), and 3 eq. of cesium carbonate in 1,4-dioxane was heated at 105° C. for 2.5 hours in sealed tube and then cooled to room temperature, filtered, and concentrated in vacuo. The crude product was purified by liquid column chromatography using 25% EtOAc/hexane to obtain a yellow solid

### Step 2: Hydrolysis of methyl 5-(3,5-dimethylpyridazin-4-yl)-1H-pyrrole-2-carboxylate

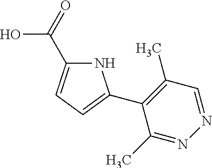 [SMILES: Cc1cnnc(C)c1-c1ccc(C(=O)O)[nH]1]

1 eq. of methyl 5-(3,5-dimethylpyridazin-4-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(3,5-dimethylpyridazin-4-yl)-1H-pyrrole-2-carboxylic acid and Intermediate III-G (1 eq.) was dissolved in 3 mL DMF. Then 2.0 eq. of EDCI-HCl, HOBt and 1 mL DIPEA were added. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 116 (4 mg, 93% purity by UV). LCMS Method B, Rt=4.37 min, m/z=447.2 [M+H]+, LCMS Method C, Rt=4.03 min, m/z=447.1956 [M+H]+, exact mass: 446.1867.

### Example 117. (S)-1-(5-(3,5-dimethylpyridazin-4-yl)-1H-pyrrole-2-carbonyl)-N-(4-fluoro-3-methylphenyl)pyrrolidine-3carboxamide

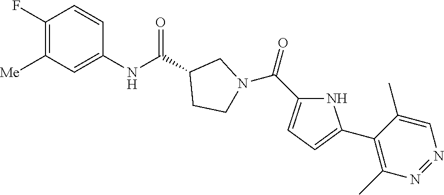 [SMILES: Cc1cc(NC(=O)[C@H]2CCN(C(=O)c3ccc(-c4c(C)cnnc4C)[nH]3)C2)ccc1F]

### Step 1: Synthesis of methyl 5-(3,5-dimethylpyridazin-4-yl)-1H-pyrrole-2-carboxylate

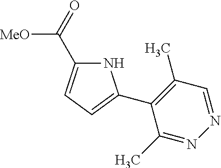 [SMILES: COC(=O)c1ccc(-c2c(C)cnnc2C)[nH]1]

A mixture of 1 eq. of 4,5-dichloropyridazin-3-ol, 8 eq. of 3,4-dihydro-2H-pyran and 0.2 eq. of para-toluenesulfonic acid in THF (200 ML) was refluxed for 1 day. After cooling to room temperature, the mixture was concentrated under reduced pressure. The residue was purified via column chromatography by using ethyl acetate/hexanes to afford the product as a white solid.

A mixture of 1 eq. of 4,5-dichloro-2-(tetrahydro-2H-pyran-2-yl)pyridazin-3(2H)-one, 1 eq. of methylboronic acid and 3 eq. of cesium carbonate was stirred in 1,4-dioxane:water (10:1). Then 44 eq. of 1,1′-bis(diphenylphosphino)ferrocene]dichloro palladium(11) was added to the mixture. The reaction mixture was stirred at 110° C. for 2 hours, then concentrated under reduced pressure. Crude product was purified via column chromatography by using ethyl acetate:hexanes to obtain pure product as a yellow solid.

A mixture of 1 eq. of 4-chloro-5-methyl-2-(tetrahydro-2H-pyran-2-yl)pyridazin-3(2H)-one, 1.25 eq. of Intermediate V-A, 10 mol % of tris(dibenzylideneacetone)dipalladium(0), 20 mol % of tricyclohexylphosphine and 3 eq. of potassium phosphate monohydrate in 1,4-dioxane was heated at 105° C. for 3 hours in sealed tube and then cooled to room temperature, filtered, and concentrated in vacuo. The crude product was purified by liquid column chromatography using 20% EtOAc/hexane to obtain yellow oil.

1 eq. of methyl 5-(5-methyl-3-oxo-2-(tetrahydro-2H-pyran-2-yl)-2,3-dihydropyridazin-4-yl)-1H-pyrrole-2-carboxylate was dissolved in methanol, treated with 20 eq. of 4M hydrogen chloride in 1,4-dioxane, and allowed to stir at room temperature for 3 hours. Removal of solvent under reduced pressure provided the product as a pale-yellow solid, presumed to be the hydrochloride salt that was used as such in the next step without further purification.

1 eq. of methyl 5-(5-methyl-3-oxo-2,3-dihydropyridazin-4-yl)-1H-pyrrole-2-carboxylate hydrochloride was suspended in 50 eq. of phosphorus oxychloride. The reaction mixture was heated at 90° C. for 2 hours. After removal of phosphorus oxychloride under reduced pressure, the residue was partitioned between ethyl acetate, water, and saturated aqueous sodium bicarbonate solution. The organic layer was dried over sodium sulfate, filtered, and concentrated in vacuo. The crude product was purified by liquid column chromatography using 20% EtOAc/hexane to obtain yellow oil.

A mixture of 1 eq. of methyl 5-(3-chloro-5-methylpyridazin-4-yl)-1H-pyrrole-2-carboxylate, 5 eq. of methylboronic acid, 10 mol % of 1,1′-bis(diphenylphosphino)ferrocene]dichloropalladium(11), and 3 eq. of cesium carbonate in 1,4-dioxane was heated at 105° C. for 2.5 hours in sealed tube and then cooled to room temperature, filtered, and concentrated in vacuo. The crude product was purified by liquid column chromatography using 25% EtOAc/hexane to obtain a yellow solid

### Step 2: Hydrolysis of methyl 5-(3,5-dimethylpyridazin-4-yl)-1H-pyrrole-2-carboxylate

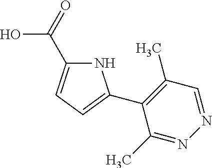 [SMILES: Cc1cnnc(C)c1-c1ccc(C(=O)O)[nH]1]

1 eq. of methyl 5-(3,5-dimethylpyridazin-4-yl)-1H-pyrrole-2-carboxylate was dissolved in THF. Then, 5 eq. of LiOH was dissolved in H₂O and added to the THF solution. The reaction mixture was stirred at room temperature for overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(3,5-dimethylpyridazin-4-yl)-1H-pyrrole-2-carboxylic acid and Intermediate III-B (1 eq.) were dissolved in 3 mL DMF. Then 2 eq. of EDCI, HOBt, and 10 eq. of DIPEA were added. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 117 (5 mg, 97% purity by UV). LCMS Method B, Rt=4.39 min, m/z=422.2 [M+H]+, LCMS Method C, Rt=3.91 min, m/z=422.2000 [M+H]+, exact mass: 421.1914.

### Example 118. (S)-1-(5-(4-methyl-2-oxo-1,2-dihydropyridin-3-yl)-1H-pyrrole-2-carbonyl)-N-(3,4,5-trifluorophenyl)pyrrolidine-3-carboxamide

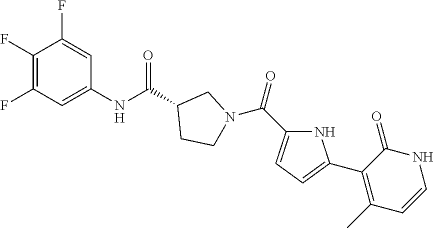 [SMILES: Cc1cc[nH]c(=O)c1-c1ccc(C(=O)N2CC[C@H](C(=O)Nc3cc(F)c(F)c(F)c3)C2)[nH]1]

### Step 1: Synthesis of methyl 5-(4-methyl-2-oxo-1,2-dihydropyridin-3-yl)-1H-pyrrole-2-carboxylate via Suzuki cross coupling

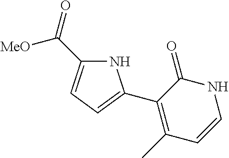 [SMILES: COC(=O)c1ccc(-c2c(C)cc[nH]c2=O)[nH]1]

A mixture of 1 eq. of 3-bromo-4-methylpyridin-2(1H)-one and 1.5 eq. of Intermediate V-A in 1,4-dioxane was bubbled under argon for 10 mins. The mixture was then added with 10 mol % of tris(dibenzylideneacetone)dipalladium(0), 20 mol % of tricyclohexylphosphine and 3 eq. of potassium phosphate monohydrate. The reaction was heated at 105° C. for 2 hours in sealed tube and then cooled to room temperature, filtered, and concentrated in vacuo. The crude product was purified by liquid column chromatography using 50% EtOAc/hexane to obtain yellow oil.

### Step 2: Hydrolysis of methyl 5-(4-methyl-2-oxo-1,2-dihydropyridin-3-yl)-1H-pyrrole-2-carboxylate

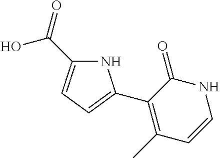 [SMILES: Cc1cc[nH]c(=O)c1-c1ccc(C(=O)O)[nH]1]

1 eq. of methyl 5-(4-methyl-2-oxo-1,2-dihydropyridin-3-yl)-1H-pyrrole-2-carboxylate was dissolved in THF. Then, 10 eq. of LiOH was dissolved in H₂O and added to the THF solution. The reaction mixture was stirred at room temperature for overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(4-methyl-2-oxo-1,2-dihydropyridin-3-yl)-1H-pyrrole-2-carboxylic acid and Intermediate III-A (1 eq.) was dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 118 (12 mg, 100% purity by UV). LCMS Method B, Rt=4.51 min, m/z=445.1 [M+H]+, LCMS Method C, Rt=4.39 min, m/z=445.1536 [M+H]+, exact mass: 444.1409.

### Example 119. (2S,3S)—N-(3-chloro-4-fluorophenyl)-2-methyl-1-(5-(4-methyl-2-oxo-1,2-dihydropyridin-3-yl)-1H-pyrrole-2-carbonyl)pyrrolidine-3-carboxamide

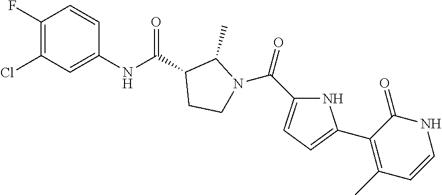 [SMILES: Cc1cc[nH]c(=O)c1-c1ccc(C(=O)N2CC[C@H](C(=O)Nc3ccc(F)c(Cl)c3)[C@@H]2C)[nH]1]

### Step 1: Synthesis of methyl 5-(4-methyl-2-oxo-1,2-dihydropyridin-3-yl)-1H-pyrrole-2-carboxylate via Suzuki cross coupling

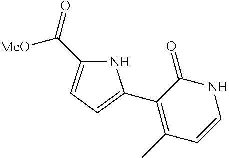 [SMILES: COC(=O)c1ccc(-c2c(C)cc[nH]c2=O)[nH]1]

A mixture of 1 eq. of 3-bromo-4-methylpyridin-2(1H)-one and 1.2 eq. of Intermediate V-A in 1,4-dioxane was bubbled under argon for 10 mins. The mixture was then added with 10 mol % of tris(dibenzylideneacetone)dipalladium(0), 20 mol % of tricyclohexylphosphine and 3 eq. of potassium phosphate monohydrate. The reaction was heated at 105° C. for 2 hours in sealed tube and then cooled to room temperature, filtered, and concentrated in vacuo. The crude product was purified by liquid column chromatography using 50% EtOAc/hexane to give yellow oil.

### Step 2: Hydrolysis of methyl 5-(4-methyl-2-oxo-1,2-dihydropyridin-3-yl)-1H-pyrrole-2-carboxylate

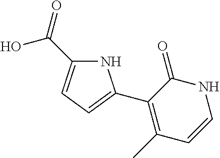 [SMILES: Cc1cc[nH]c(=O)c1-c1ccc(C(=O)O)[nH]1]

1 eq. of methyl 5-(4-methyl-2-oxo-1,2-dihydropyridin-3-yl)-1H-pyrrole-2-carboxylate was dissolved in THF. Then, 10 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(4-methyl-2-oxo-1,2-dihydropyridin-3-yl)-1H-pyrrole-2-carboxylic acid and Intermediate III-M (1 eq.) was dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 119 (6 mg, 100% purity by UV). LCMS Method B, Rt=4.62 min, m/z=457.1 [M+H]+, LCMS Method C, Rt=4.56 min, m/z=457.1423 [M+H]+, exact mass: 456.1364.

### Example 120. (2S,3S)—N-(4-fluoro-3-methylphenyl)-2-methyl-1-(5-(4-methyl-2-oxo-1,2-dihydropyridin-3-yl)-1H-pyrrole-2-carbonyl)pyrrolidine-3-carboxamide

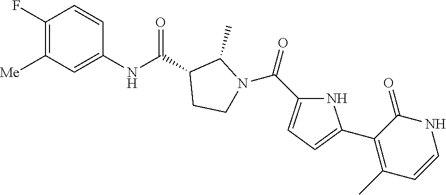 [SMILES: Cc1cc(NC(=O)[C@H]2CCN(C(=O)c3ccc(-c4c(C)cc[nH]c4=O)[nH]3)[C@H]2C)ccc1F]

### Step 1: Synthesis of methyl 5-(4-methyl-2-oxo-1,2-dihydropyridin-3-yl)-1H-pyrrole-2-carboxylate via Suzuki cross coupling

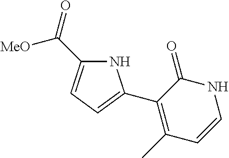 [SMILES: COC(=O)c1ccc(-c2c(C)cc[nH]c2=O)[nH]1]

A mixture of 1 eq. of 3-bromo-4-methylpyridin-2(1H)-one and 1.2 eq. of Intermediate V-A in 1,4-dioxane was bubbled under argon for 10 mins. The mixture was then added with 10 mol % of tris(dibenzylideneacetone)dipalladium(0), 20 mol % of tricyclohexylphosphine and 3 eq. of potassium phosphate monohydrate. The reaction was heated at 105° C. for 2 hours in sealed tube and then cooled to room temperature, filtered, and concentrated in vacuo. The crude product was purified by liquid column chromatography using 50% EtOAc/hexane to give yellow oil.

### Step 2: Hydrolysis of methyl 5-(4-methyl-2-oxo-1,2-dihydropyridin-3-yl)-1H-pyrrole-2-carboxylate

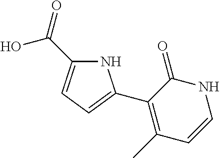 [SMILES: Cc1cc[nH]c(=O)c1-c1ccc(C(=O)O)[nH]1]

1 eq. of methyl 5-(4-methyl-2-oxo-1,2-dihydropyridin-3-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(4-methyl-2-oxo-1,2-dihydropyridin-3-yl)-1H-pyrrole-2-carboxylic acid Intermediate III-H (1 eq.) was dissolved in DMF 3 mL. Then 2 eq. of EDCI-HCl, HOBt, and 1 mL of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 120 (17 mg, 100% purity by UV). LCMS Method B, Rt=4.54 min, m/z=437.2 [M+H]+, LCMS Method C, Rt=4.39 min, m/z=437.2015 [M+H]+, exact mass: 436.1911.

### Example 121. (S)-1-(5-(6-oxo-1,6-dihydropyridazin-4-yl)-1H-pyrrole-2-carbonyl)-N-(3,4,5-trifluorophenyl)pyrrolidine-3-carboxamide

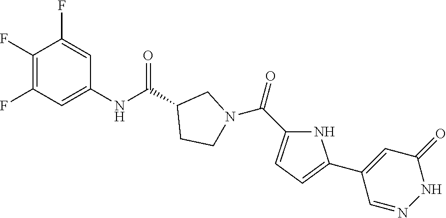 [SMILES: O=C(Nc1cc(F)c(F)c(F)c1)[C@H]1CCN(C(=O)c2ccc(-c3cn[nH]c(=O)c3)[nH]2)C1]

### Step 1: Synthesis of methyl 5-(6-oxo-1,6-dihydropyridazin-4-yl)-1H-pyrrole-2-carboxylate via Suzuki cross coupling

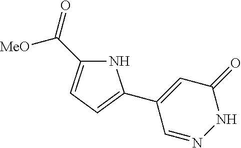 [SMILES: COC(=O)c1ccc(-c2cn[nH]c(=O)c2)[nH]1]

1.2 eq. of 5-chloropyridazin-3(2H)-one was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(6-oxo-1,6-dihydropyridazin-4-yl)-1H-pyrrole-2-carboxylate

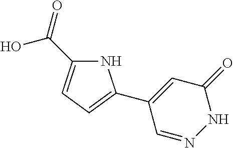 [SMILES: O=C(O)c1ccc(-c2cn[nH]c(=O)c2)[nH]1]

1 eq. of methyl 5-(6-oxo-1,6-dihydropyridazin-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(6-oxo-1,6-dihydropyridazin-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 121 (16 mg, 92% purity by UV). LCMS Method B, Rt=4.37 min, m/z=432.0 [M+H]+, exact mass: 431.1205.

### Example 122. (S)-1-(5-(6-methyl-1H-indazol-5-yl)-1H-pyrrole-2-carbonyl)-N-(3,4,5-trifluorophenyl) pyrrolidine-3-carboxamide

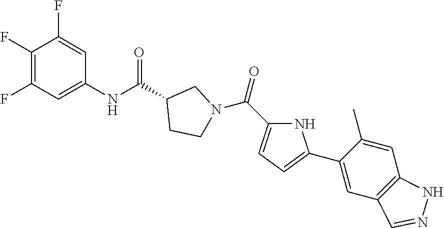 [SMILES: Cc1cc2[nH]ncc2cc1-c1ccc(C(=O)N2CC[C@H](C(=O)Nc3cc(F)c(F)c(F)c3)C2)[nH]1]

### Step 1: Synthesis of methyl 5-(6-methyl-1H-indazol-5-yl)-1H-pyrrole-2-carboxylate via Suzuki cross coupling

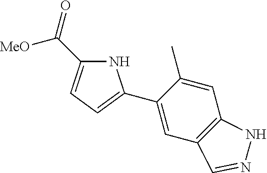 [SMILES: COC(=O)c1ccc(-c2cc3cn[nH]c3cc2C)[nH]1]

1.2 eq. of 5-bromo-6-methyl-1H-indazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(6-methyl-1H-indazol-5-yl)-1H-pyrrole-2-carboxylate

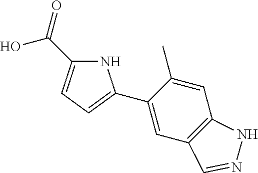 [SMILES: Cc1cc2[nH]ncc2cc1-c1ccc(C(=O)O)[nH]1]

1 eq. of methyl 5-(6-methyl-1H-indazol-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(6-methyl-1H-indazol-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 122 (18 mg, 100% purity by UV). LCMS Method B, Rt=3.89 min, m/z=467.80 [M+H]+, exact mass: 467.1569.

### Example 123. (S)—N-(3-cyano-4-fluorophenyl)-1-(5-(6-methyl-1H-indazol-5-yl)-1H-pyrrole-2-carbonyl) pyrrolidine-3-carboxamide

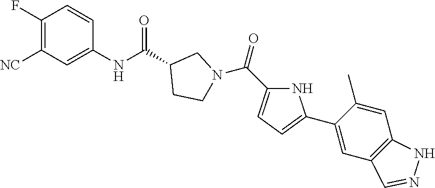 [SMILES: Cc1cc2[nH]ncc2cc1-c1ccc(C(=O)N2CC[C@H](C(=O)Nc3ccc(F)c(C#N)c3)C2)[nH]1]

### Step 1: Synthesis of methyl 5-(6-methyl-1H-indazol-5-yl)-1H-pyrrole-2-carboxylate via Suzuki cross coupling

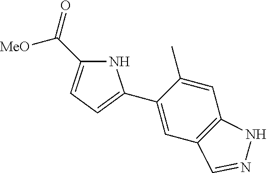 [SMILES: COC(=O)c1ccc(-c2cc3cn[nH]c3cc2C)[nH]1]

1.2 eq. of 5-bromo-6-methyl-1H-indazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(6-methyl-1H-indazol-5-yl)-1H-pyrrole-2-carboxylate

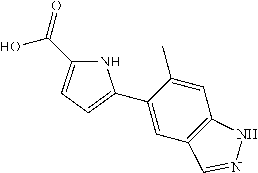 [SMILES: Cc1cc2[nH]ncc2cc1-c1ccc(C(=O)O)[nH]1]

1 eq. of methyl 5-(6-methyl-1H-indazol-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(6-methyl-1H-indazol-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-E was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 123 (11 mg, 99% purity by UV). LCMS Method B, Rt=4.47 min, m/z=457.2 [M+H]+, LCMS Method C, Rt=4.30 min, m/z=457.1793 [M+H]+, exact mass: 456.1710.

### Example 124. (S)-1-(5-(5-methyl-3-oxo-2,3-dihydropyridazin-4-yl)-1H-pyrrole-2-carbonyl)-N-(3,4,5-trifluorophenyl)pyrrolidine-3-carboxamide

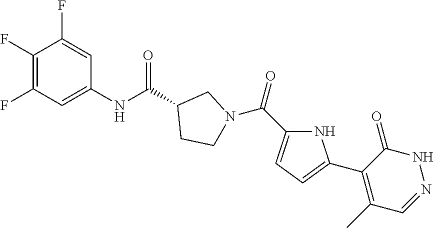 [SMILES: Cc1cn[nH]c(=O)c1-c1ccc(C(=O)N2CC[C@H](C(=O)Nc3cc(F)c(F)c(F)c3)C2)[nH]1]

### Step 1: Synthesis of methyl 5-(3,5-dimethylpyridazin-4-yl)-1H-pyrrole-2-carboxylate

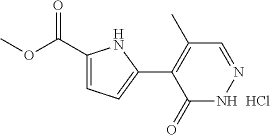 [SMILES: COC(=O)c1ccc(-c2c(C)cn[nH]c2=O)[nH]1 ; Cl]

A mixture of 1 eq. of 4,5-dichloropyridazin-3-ol, 8 eq. of 3,4-dihydro-2H-pyran and 0.2 eq. of para-toluenesulfonic acid in THF was refluxed for 1 day. After cooling to room temperature, the mixture was concentrated under reduced pressure. The residue was purified via column chromatography by using 3% to 5% ethyl acetate/hexanes to afford the product as a white solid.

5 mol % of [1,1′-bis(diphenylphosphino)ferrocene]dichloropalladium(11) was added to a mixture of 1 eq. of 4,5-dichloro-2-(tetrahydro-2H-pyran-2-yl)pyridazin-3(2H)-one, 1.03 eq. of methylboronic acid and 3 eq. of cesium carbonate in 1,4-dioxane:water (10:1). The reaction mixture was stirred at 110° C. for 2 hours, then concentrated under reduced pressure. The crude product was purified by silica gel chromatography (5% ethyl acetate in hexanes) to provide product as a pale-yellow solid.

A mixture of 1 eq. of 4-chloro-5-methyl-2-(tetrahydro-2H-pyran-2-yl)pyridazin-3(2H)-one, 1.2 eq. of Intermediate V-A in 1,4-dioxane was bubbled by argon for 10 mins. The mixture was then added with 10 mol % of tris(dibenzylideneacetone)dipalladium(0), 20 mol % of tricyclohexylphosphine and 3 eq. of potassium phosphate monohydrate. The reaction was heated at 105° C. for 3 hours in sealed tube and then cooled to room temperature, filtered, and concentrated in vacuo. The crude product was purified by liquid column chromatography using 20% EtOAc/hexanes to give yellow oil.

1 eq. of methyl 5-(5-methyl-3-oxo-2-(tetrahydro-2H-pyran-2-yl)-2,3-dihydropyridazin-4-yl)-1H-pyrrole-2-carboxylate was dissolved in dioxane (2.5 mL), treated with 2.5 eq. of 4M hydrogen chloride in 1,4-dioxane and allowed to stir at room temperature for 3 hours. The solvent was removed under vacuum. The product was purified via silica gel column chromatography treated with 1% triethylamine in 20% EtOAc/hexanes to obtain a pale-yellow solid.

### Step 2: Hydrolysis of methyl 5-(5-methyl-3-oxo-2,3-dihydropyridazin-4-yl)-1H-pyrrole-2-carboxylate

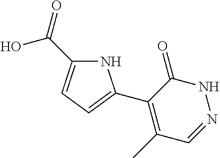 [SMILES: Cc1cn[nH]c(=O)c1-c1ccc(C(=O)O)[nH]1]

1 eq. of methyl 5-(5-methyl-3-oxo-2,3-dihydropyridazin-4-yl)-1H-pyrrole-2-carboxylate was dissolved in THF. Then, 10 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(5-methyl-3-oxo-2,3-dihydropyridazin-4-yl)-1H-pyrrole-2-carboxylic acid and Intermediate III-A (1 eq.) was dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 3% MeOH/EtOAc to give Example 124 (30 mg, 100% purity by UV). LCMS Method B, Rt=4.50 min, m/z=446.1 [M+H]+, exact mass: 445.1362.

### Example 125. (2S,3S)—N-(4-fluoro-3-methylphenyl)-2-methyl-1-(5-(5-methyl-3-oxo-2,3-dihydro pyridazin-4-yl)-1H-pyrrole-2-carbonyl)pyrrolidine-3-carboxamide

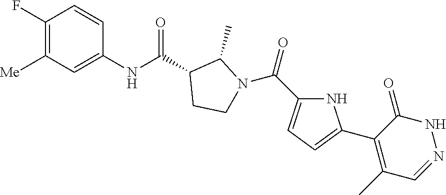 [SMILES: Cc1cc(NC(=O)[C@H]2CCN(C(=O)c3ccc(-c4c(C)cn[nH]c4=O)[nH]3)[C@H]2C)ccc1F]

### Step 1: Synthesis of methyl 5-(3,5-dimethylpyridazin-4-yl)-1H-pyrrole-2-carboxylate

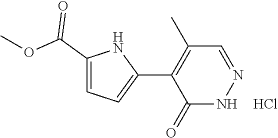 [SMILES: COC(=O)c1ccc(-c2c(C)cn[nH]c2=O)[nH]1 ; Cl]

A mixture of 1 eq. of 4,5-dichloropyridazin-3-ol, 8 eq. of 3,4-dihydro-2H-pyran and 0.2 eq. of para-toluenesulfonic acid in THF was refluxed for 1 day. After cooling to room temperature, the mixture was concentrated under reduced pressure. The residue was purified via column chromatography by using 3% to 5% ethyl acetate/hexanes to afford the product as a white solid.

5 mol % of [1,1′-bis(diphenylphosphino)ferrocene]dichloropalladium(11) was added to a mixture of 1 eq. of 4,5-dichloro-2-(tetrahydro-2H-pyran-2-yl)pyridazin-3(2H)-one, 1.03 eq. of methylboronic acid and 3 eq. of cesium carbonate in 1,4-dioxane:water (10:1). The reaction mixture was stirred at 110° C. for 2 hours, then concentrated under reduced pressure. The crude product was purified by silica gel chromatography (5% ethyl acetate in hexanes) to provide product as a pale-yellow solid.

A mixture of 1 eq. of 4-chloro-5-methyl-2-(tetrahydro-2H-pyran-2-yl)pyridazin-3(2H)-one, 1.2 eq. of Intermediate V-A in 1,4-dioxane was bubbled by argon for 10 mins. The mixture was then added with 10 mol % of tris(dibenzylideneacetone)dipalladium(0), 20 mol % of tricyclohexylphosphine and 3 eq. of potassium phosphate monohydrate. The reaction was heated at 105° C. for 3 hours in sealed tube and then cooled to room temperature, filtered, and concentrated in vacuo. The crude product was purified by liquid column chromatography using 20% EtOAc/hexanes to give yellow oil.

1 eq. of methyl 5-(5-methyl-3-oxo-2-(tetrahydro-2H-pyran-2-yl)-2,3-dihydropyridazin-4-yl)-1H-pyrrole-2-carboxylate was dissolved in dioxane (2.5 mL), treated with 2.5 eq. of 4M hydrogen chloride in 1,4-dioxane and allowed to stir at room temperature for 3 hours. The solvent was removed under vacuum. The product was purified via silica gel column chromatography treated with 1% triethylamine in 20% EtOAc/hexanes to obtain a pale-yellow solid.

### Step 2: Hydrolysis of methyl 5-(5-methyl-3-oxo-2,3-dihydropyridazin-4-yl)-1H-pyrrole-2-carboxylate

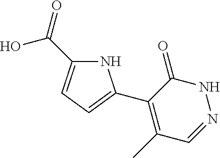 [SMILES: Cc1cn[nH]c(=O)c1-c1ccc(C(=O)O)[nH]1]

1 eq. of methyl 5-(5-methyl-3-oxo-2,3-dihydropyridazin-4-yl)-1H-pyrrole-2-carboxylate was dissolved in THF. Then, 10 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(5-methyl-3-oxo-2,3-dihydropyridazin-4-yl)-1H-pyrrole-2-carboxylic acid and Intermediate III-H (1 eq.) was dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude reaction was purified by column chromatography with 2% MeOH/EtOAc to give Example 125 (10 mg, 96% purity by UV). LCMS Method B, Rt=4.54 min, m/z=438.2 [M+H]+, LCMS Method C, Rt=4.37 min, m/z=438.1918 [M+H]+, exact mass: 437.1863.

### Example 126. (2S,3S)—N-(4-fluoro-3-methylphenyl)-1-(5-(1-(2-hydroxyethyl)-3,5-dimethyl-1H-pyrazol-4-yl)-1H-pyrrole-2-carbonyl)-2-methylpyrrolidine-3-carboxamide

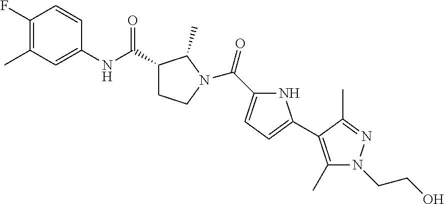 [SMILES: Cc1cc(NC(=O)[C@H]2CCN(C(=O)c3ccc(-c4c(C)nn(CCO)c4C)[nH]3)[C@H]2C)ccc1F]

### Step 1: Synthesis of methyl-H-pyrazol-4-yl)-1H-pyrrole-2-carboxylate via Suzuki cross coupling

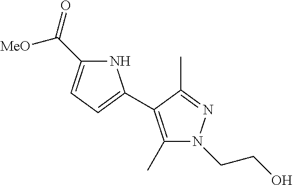 [SMILES: COC(=O)c1ccc(-c2c(C)nn(CCO)c2C)[nH]1]

1 eq. of 4-iodo-3,5-dimethyl-1H-pyrazole was dissolved in THF. The solution was cooled to 0oC. Then 60% of sodium hydride solution in THF (1.05 eq) was dropped to the cooled solution. The reaction mixture was stirred for 10 mins. at 0° C. Then, 1.16 eq. of tert-butyl 2-bromoacetate was dropped to the previous solution slowly. Then the reaction was heated for 2 h at 60° C. The reaction was quenched by adding water. The aqueous solution was extracted by EtOAc for 3 times to obtain the product. The organic layer was dried by anhydrous sodium sulfate and removed solvent by rotavapor. The product was used in the next step without purification.

1 eq. of tert-butyl 2-(4-iodo-3,5-dimethyl-1H-pyrazol-1-yl)acetate was dissolved in THF. The solution was cooled to 0° C. under N₂. Then, 2.5M of lithium aluminium hydride in THF (5 eq.) was added to the previous solution slowly. The reaction was let to reach room temperature and further stirred for 30 mins. Then the reaction was cooled to 0° C. and added methanol to quench the reaction. The mixture was removed solvent by rotavapor. The crude product was washed by EtOAc for 3 times to get the pure product. The product was dried by rotavapor and used for the next step.

1.2 eq. of 2-(4-iodo-3,5-dimethyl-1H-pyrazol-1-yl)ethanol was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(1-(2-hydroxyethyl)-3,5-dimethyl-1H-pyrazol-4-yl)-1H-pyrrole-2-carboxylate

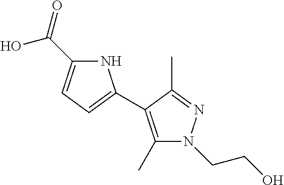 [SMILES: Cc1nn(CCO)c(C)c1-c1ccc(C(=O)O)[nH]1]

1 eq. of methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(1-(2-hydroxyethyl)-3,5-dimethyl-1H-pyrazol-4-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-H was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 126 (9 mg, 97% purity by UV). LCMS Method B, Rt=4.40 min, m/z=468.2 [M+H]+, exact mass: 467.2333.

### Example 127. (2S,3S)—N-(3-chloro-4-fluorophenyl)-2-methyl-1-(5-(2-methylpyrimidin-5-yl)-1H-pyrrole-2-carbonyl)pyrrolidine-3-carboxamide

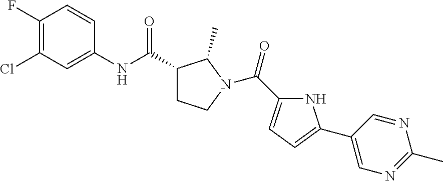 [SMILES: Cc1ncc(-c2ccc(C(=O)N3CC[C@H](C(=O)Nc4ccc(F)c(Cl)c4)[C@@H]3C)[nH]2)cn1]

### Step 1: Synthesis of methyl 5-(2-methylpyrimidin-5-yl)-1H-pyrrole-2-carboxylate via Suzuki cross coupling

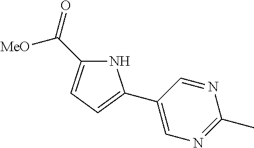 [SMILES: COC(=O)c1ccc(-c2cnc(C)nc2)[nH]1]

1.2 eq. of 5-bromo-2-methylpyrimidine was dissolved in H₂O:1,4-dioxane (1:5). Then 1 eq. of Intermediate V-A and 3 eq. of K₂COM were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexane.

### Step 2: Hydrolysis of methyl 5-(2-methylpyrimidin-5-yl)-1H-pyrrole-2-carboxylate

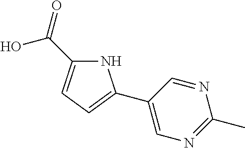

1 eq. of methyl 5-(2-methylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(2-methylpyrimidin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-M was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 127 (14 mg, 93% purity by UV). LCMS Method B, Rt=4.56 min, m/z=442.1 [M+H]+, exact mass: 441.1368.

### Example 128. (S)-1-(5-(4-methyl-1H-indazol-5-yl)-1H-pyrrole-2-carbonyl)-N-(3,4,5-trifluorophenyl) pyrrolidine-3-carboxamide

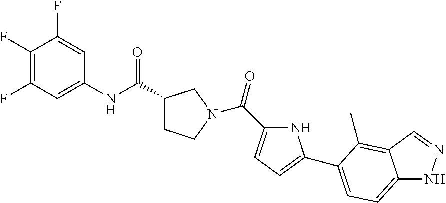 [SMILES: Cc1c(-c2ccc(C(=O)N3CC[C@H](C(=O)Nc4cc(F)c(F)c(F)c4)C3)[nH]2)ccc2[nH]ncc12]

### Step 1: Synthesis of methyl 5-(4-methyl-1H-indazol-5-yl)-1H-pyrrole-2-carboxylate via Suzuki cross coupling

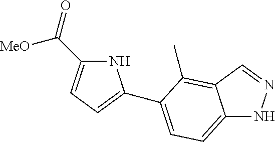 [SMILES: COC(=O)c1ccc(-c2ccc3[nH]ncc3c2C)[nH]1]

1.2 eq. of 5-bromo-4-methyl-1H-indazole was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hours. To achieve pure product, crude product was purified by liquid column chromatography with EtOAc/hexanes.

### Step 2: Hydrolysis of methyl 5-(4-methyl-1H-indazol-5-yl)-1H-pyrrole-2-carboxylate

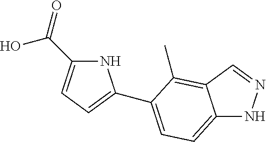 [SMILES: Cc1c(-c2ccc(C(=O)O)[nH]2)ccc2[nH]ncc12]

1 eq. of 5-(4-methyl-1H-indazol-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4-methyl-1H-indazol-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 128 (12 mg, 93% purity by UV). LCMS Method B, Rt=3.64 min, m/z=468.2 [M+H]+, exact mass: 467.1569.

### Example 129. (S)-1-(5-(4-methyl-1H-pyrrolo[2,3-b]pyridin-5-yl)-1H-pyrrole-2-carbonyl)-N-(3,4,5-trifluorophenyl)pyrrolidine-3-carboxamide

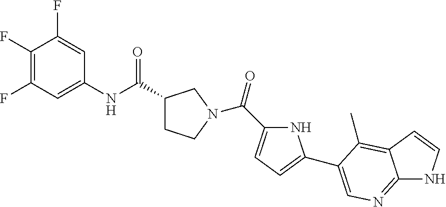 [SMILES: Cc1c(-c2ccc(C(=O)N3CC[C@H](C(=O)Nc4cc(F)c(F)c(F)c4)C3)[nH]2)cnc2[nH]ccc12]

### Step 1: Synthesis of methyl 5-(4-methyl-1H-pyrrolo[2,3-b]pyridin-5-yl)-1H-pyrrole-2-carboxylate via Suzuki cross coupling

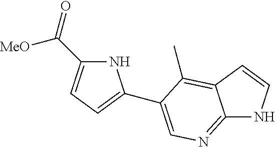 [SMILES: COC(=O)c1ccc(-c2cnc3[nH]ccc3c2C)[nH]1]

1.2 eq. of 5-bromo-4-methyl-1H-pyrrolo[2,3-b]pyridine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(4-methyl-1H-pyrrolo[2,3-b]pyridin-5-yl)-1H-pyrrole-2-carboxylate

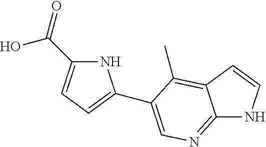 [SMILES: Cc1c(-c2ccc(C(=O)O)[nH]2)cnc2[nH]ccc12]

1 eq. of methyl 5-(4-methyl-1H-pyrrolo[2,3-b]pyridin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(4-methyl-1H-pyrrolo[2,3-b]pyridin-5-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 129 (7 mg, 93% purity by UV). LCMS Method B, Rt=4.52 min, m/z=468.2 [M+H]+, exact mass: 467.1569.

### Example 130. (S)—N-(4-fluoro-3-methylphenyl)-1-(5-(pyridazin-4-yl)-1H-pyrrole-2-carbonyl)pyrrolidine-3-carboxamide

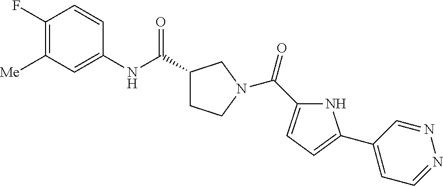 [SMILES: Cc1cc(NC(=O)[C@H]2CCN(C(=O)c3ccc(-c4ccnnc4)[nH]3)C2)ccc1F]

### Step 1: Synthesis of methyl 5-(pyridazin-4-yl)-1H-pyrrole-2-carboxylate via Suzuki cross coupling

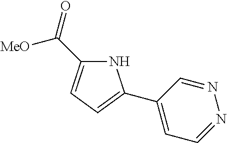 [SMILES: COC(=O)c1ccc(-c2ccnnc2)[nH]1]

1 eq. of pyridazin-4-amine was dissolved in 1,2-DME. Then, 0.5 eq. of iodine, 1 eq. of potassium iodide, 0.3 eq. of copper(I)iodide and 4 eq. of isoamyl nitrite were added to the previous solution. The reaction mixture was heated at 70° C. for 2 hours. Crude reaction was evaporated by rotavapor. Diluted with water and extracted by EtOAc for 3 times. The organic layer was concentrated. The product was purified by liquid column chromatography with EtOAc/hexane to achieve the pure product of 4-iodopyridazine.

A mixture of 1 eq. of 4-iodopyridazine and 1.11 eq. of Intermediate V-A in 1,4-dioxane:H₂O (6:1) was bubbled by argon for 10 mins. The mixture was then added with 10 mol % of Pd(PPh₃)₄ and 1.11 eq. of potassium carbonate. The reaction was heated at 80° C. for 3 hours in sealed tube and then cooled to room temperature, filtered, and concentrated in vacuo. The crude product was purified by liquid column chromatography using 60% EtOAc/hexane to give a yellow oil.

### Step 2: Hydrolysis of methyl 5-(pyridazin-4-yl)-1H-pyrrole-2-carboxylate

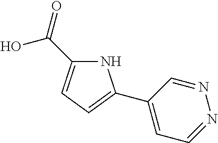 [SMILES: O=C(O)c1ccc(-c2ccnnc2)[nH]1]

1 eq. of methyl 5-(pyridazin-4-yl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(pyridazin-4-yl)-1H-pyrrole-2-carboxylic acid was dissolved in DMF. Then 1.5 eq. of HATU and 5 eq. of DIPEA were added. The reaction mixture was stirred at room temperature for 10 mins. Then, 1.1 eq. of Intermediate III-B was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 130 (10 mg, 100% purity by UV). LCMS Method B, Rt=4.28 min, m/z=394.2 [M+H]+, exact mass: 393.1601. ¹H-NMR (500 MHz, Acetone-d₆) δ 11.30 (1H), 9.67 (d, J=1.2 Hz, 1H), 9.39 (s, 1H), 9.17-9.08 (dd, J=5.5, 0.9 Hz, 1H), 8.02 (dd, J=5.5, 2.4 Hz, 1H), 7.58 (d, J=5.4 Hz, 1H), 7.52-7.44 (m, 1H), 7.14-7.05 (m, 1H), 6.99 (t, J=9.2 Hz, 1H), 6.84 (brd, 1H), 4.05 (dd, J=14.2, 7.0 Hz, 1H), 3.97-3.76 (m, 2H), 3.40-3.37 (m, 1H), 3.30-3.23 (m, 1H), 2.22 (d, J=1.1 Hz, 3H), 2.05-2.04 (overlap, 2H).

### Example 131. (S)-1-(5-(pyrazin-2-yl)-1H-pyrrole-2-carbonyl)-N-(3,4,5-trifluorophenyl)pyrrolidine-3-carboxamide

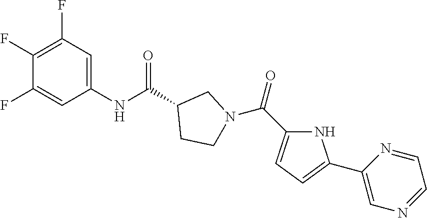 [SMILES: O=C(Nc1cc(F)c(F)c(F)c1)[C@H]1CCN(C(=O)c2ccc(-c3cnccn3)[nH]2)C1]

### Step 1: Synthesis of methyl 5-(pyrazin-2-yl)-1H-pyrrole-2-carboxylate via Suzuki cross coupling

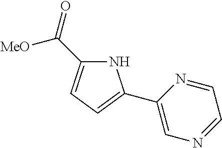 [SMILES: COC(=O)c1ccc(-c2cnccn2)[nH]1]

1.2 eq. of 2-bromopyrazine was dissolved in H₂O:1,4-dioxane (1:5). Then 1 eq. of Intermediate V-A and 3 eq. of K₂CO₃ were added to the flask. The reaction mixture was bubbled by argon gas for 5-15 mins. Then 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(pyrazin-2-yl)-1H-pyrrole-2-carboxylate

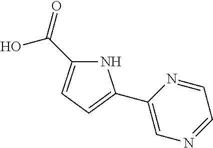 [SMILES: O=C(O)c1ccc(-c2cnccn2)[nH]1]

1 eq. of methyl 5-(pyrazin-2-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution equals to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(pyrazin-2-yl)-1H-pyrrole-2-carboxylic acid was dissolved in DMF. Then 1.5 eq. of HATU and 3 eq. of DIPEA were added. The reaction mixture was stirred at room temperature for 10 mins. Then 1.1 eq. of Intermediate III-A was added to the solution. The mixture was stirred overnight at room temperature. Then the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 131 (10 mg, 95% purity by UV). LCMS Method A, Rt=4.53 min, m/z=416.1 [M+H]+, LCMS Method C, Rt=4.32 min, m/z=416.1358 [M+H]+, exact mass: 415.1256.

### Example 132. (S)-1-(6-(4,6-dimethylpyrimidin-5-yl)-1H-indole-2-carbonyl)-N-(3,4,5-trifluorophenyl) pyrrolidine-3-carboxamide

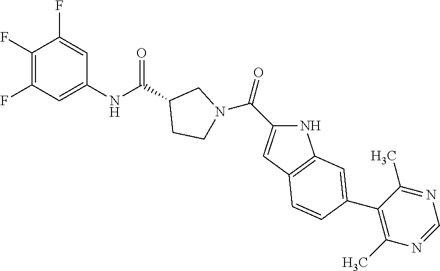 [SMILES: Cc1ncnc(C)c1-c1ccc2cc(C(=O)N3CC[C@H](C(=O)Nc4cc(F)c(F)c(F)c4)C3)[nH]c2c1]

### Step 1: Synthesis of methyl 6-(4,6-dimethylpyrimidin-5-yl)-1H-indole-2-carboxylate via Suzuki cross coupling

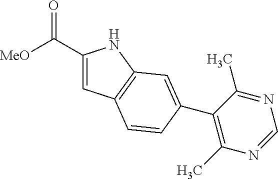 [SMILES: COC(=O)c1cc2ccc(-c3c(C)ncnc3C)cc2[nH]1]

1.0 eq. of 5-bromo-4,6-dimethylpyrimidine and 1.5 eq. of Intermediate V-B was dissolved in 1,4-dioxane. Then bubbled argon gas for 10 mins. The mixture was added 3 eq. of K₂C₀M and 10 mol % Pd(dppf)Cl₂ under argon. Reaction was heated under argon gas at 110° C. for 1.5 hours. The crude product was purified by liquid column chromatography with 50% EtOAc/hexanes to give a pale-yellow solid.

### Step 2: Hydrolysis of methyl 6-(4,6-dimethylpyrimidin-5-yl)-1H-indole-2-carboxylate

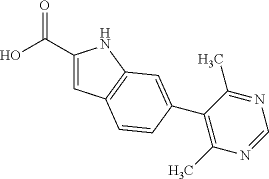 [SMILES: Cc1ncnc(C)c1-c1ccc2cc(C(=O)O)[nH]c2c1]

1 eq. of methyl 6-(4,6-dimethylpyrimidin-5-yl)-1H-indole-2-carboxylate was dissolved in THF. Then, 10 eq. of LiOH was dissolved in H₂O and added to the THF solution. The reaction mixture was stirred at room temperature overnight and concentrated by rotavapor. Then, the reaction was acidified with 2N HCl until pH of solution equals to 3. During this acidification, a white precipitate formed. The solid was collected via filtration and washed with water, providing the product as a white solid.

### Step 3: Amide Formation

1 eq. of 6-(4,6-dimethylpyrimidin-5-yl)-1H-indole-2-carboxylic acid and Intermediate III-A (1 eq.) were dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude product was purified by column chromatography using 2% MeOH/EtOAc to afford Example 132 (120 mg, 99% purity by UV). LCMS Method B, Rt=4.70 min, m/z=494.2 [M+H]+, exact mass: 493.1726.

### Example 133. (S)-1-(3-chloro-6-(4,6-dimethylpyrimidin-5-yl)-1H-indole-2-carbonyl)-N-(3,4,5-trifluorophenyl)pyrrolidine-3-carboxamide

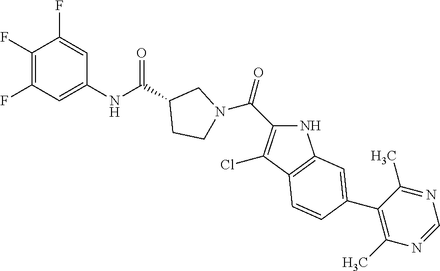 [SMILES: Cc1ncnc(C)c1-c1ccc2c(Cl)c(C(=O)N3CC[C@H](C(=O)Nc4cc(F)c(F)c(F)c4)C3)[nH]c2c1]

1.1 eq. of N-chlorosuccinimide was added to a solution of Example 132 (1 eq.) in 3 mL of DCM. The reaction mixture was stirred at room temperature overnight, added N-chlorosuccinimide 1.5 eq. and stirred at room temperature until the reaction was completed. The reaction was quenched by water and extracted with EtOAc. The organic layer was washed with brine, dried over anhydrous sodium sulfate, and concentrated under reduced pressure. The crude product was purified by column chromatography using 2% MeOH/EtOAc to afford Example 133 (5 mg, 88.0% purity). LCMS Method A. Rt=4.73 min, m/z=528.2 [M+H]+, exact mass: 527.1336 Example 134. (S)-1-(6-(3,5-dimethyl-1H-pyrazol-4-yl)-1H-indole-2-carbonyl)-N-(3,4,5-trifluorophenyl)pyrrolidine-3-carboxamide

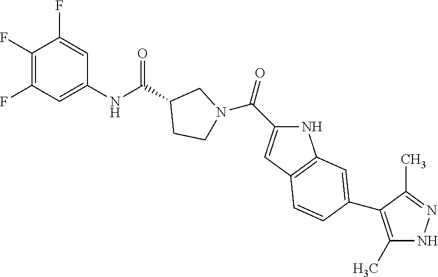 [SMILES: Cc1n[nH]c(C)c1-c1ccc2cc(C(=O)N3CC[C@H](C(=O)Nc4cc(F)c(F)c(F)c4)C3)[nH]c2c1]

### Step 1: Synthesis of methyl 6-(3,5-dimethyl-1H-pyrazol-4-yl)-1H-indole-2-carboxylate via Suzuki cross coupling

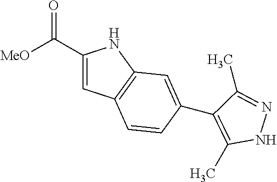 [SMILES: COC(=O)c1cc2ccc(-c3c(C)n[nH]c3C)cc2[nH]1]

1.2 eq. of 4-iodo-3,5-dimethyl-1H-pyrazole and 1 eq. of Intermediate V-B were dissolved in H₂O:1,4-dioxane (1:5) and then bubbled argon gas for 10 mins. The mixture was added 3 eq. of K₂CO₃ and 10 mol % Pd(dppf)Cl₂ under argon. Reaction was heated under argon gas at 110° C. for 1 hour. To achieve pure product, crude product was purified by liquid column chromatography with EtOAc/hexanes.

### Step 2: Hydrolysis of methyl 6-(3,5-dimethyl-1H-pyrazol-4-yl)-1H-indole-2-carboxylate

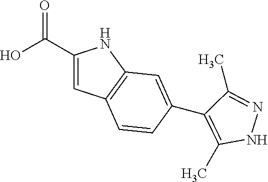 [SMILES: Cc1n[nH]c(C)c1-c1ccc2cc(C(=O)O)[nH]c2c1]

1 eq. of methyl 6-(3,5-dimethyl-1H-pyrazol-4-yl)-1H-indole-2-carboxylate was dissolved in THF. Then, 5 eq. of LiOH was dissolved in H₂O and added to the THF solution. The reaction mixture was stirred at room temperature overnight and concentrated by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 3: Amide Formation

1 eq. of 6-(3,5-dimethyl-1H-pyrazol-4-yl)-1H-indole-2-carboxylic acid was dissolved in DMF. Then 2 eq. of EDCI-HCl, HOBt, and 10 eq. of DIPEA were added. The reaction mixture was stirred at room temperature for 10 mins. and added 1.1 eq. of Intermediate III-A. The mixture was stirred overnight at room temperature. Then the mixture was diluted with water and extracted the product with EtOAc for 3 times. The organic layer was washed with brine. The crude reaction was purified by column chromatography with MeOH/EtOAc to afford Example 134 (21 mg, 95% purity by UV). LCMS Method A, Rt=4.48 min, m/z=482.2 [M+H]+, exact mass: 481.1726

### Example 135. (S)-1-(3-chloro-6-(4,6-dimethylpyrimidin-5-yl)-1H-indole-2-carbonyl)-N-(3-cyano-4-fluorophenyl)pyrrolidine-3-carboxamide

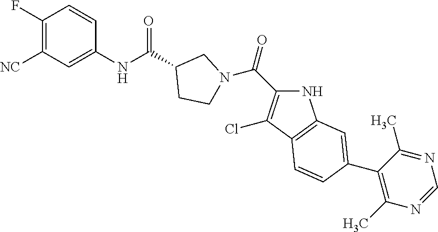 [SMILES: Cc1ncnc(C)c1-c1ccc2c(Cl)c(C(=O)N3CC[C@H](C(=O)Nc4ccc(F)c(C#N)c4)C3)[nH]c2c1]

### Step 1: Synthesis of methyl 6-(4,6-dimethylpyrimidin-5-yl)-1H-indole-2-carboxylate via Suzuki cross coupling

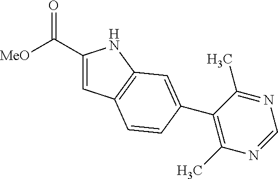 [SMILES: COC(=O)c1cc2ccc(-c3c(C)ncnc3C)cc2[nH]1]

1.2 eq. of 5-bromo-4,6-dimethylpyrimidine and 1 eq. of Intermediate V-B was dissolved in H₂O:1,4-dioxane (1:5) and then bubbled argon gas for 10 mins. The mixture was added 3 eq. of K₂CO₃ and 10 mol % Pd(dppf)Cl₂ under argon. Reaction was heated under argon gas at 110° C. for 1 hour. To achieve pure product, crude product was purified by liquid column chromatography with EtOAc/hexanes.

### Step 2: Synthesis of methyl 3-chloro-6-(4,6-dimethylpyrimidin-5-yl)-1H-indole-2-carboxylate

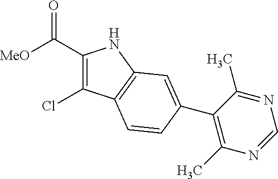 [SMILES: COC(=O)c1[nH]c2cc(-c3c(C)ncnc3C)ccc2c1Cl]

A solution of 1 eq. of methyl 6-(4,6-dimethylpyrimidin-5-yl)-1H-indole-2-carboxylate in DCM were added to 1.05 eq. of N-chlorosuccinimide. The reaction mixture was stirred at room temperature overnight. The reaction was quenched by water and extracted with EtOAc. The organic layer was washed with brine, anhydrous sodium sulfate, and concentrated. The crude reaction was purified by column chromatography with EtOAc/Hexanes.

### Step 3: Hydrolysis of methyl 3-chloro-6-(4,6-dimethylpyrimidin-5-yl)-1H-indole-2-carboxylate

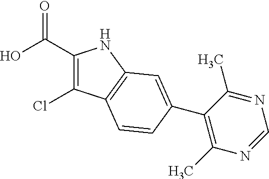 [SMILES: Cc1ncnc(C)c1-c1ccc2c(Cl)c(C(=O)O)[nH]c2c1]

1 eq. of methyl 3-chloro-6-(4,6-dimethylpyrimidin-5-yl)-1H-indole-2-carboxylate was dissolved in THF. Then 5 eq. of LiOH was dissolved in H₂O and added to the THF solution. The reaction mixture was stirred at room temperature overnight and dried the reaction by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 4: Amide Formation

1 eq. of 3-chloro-6-(4,6-dimethylpyrimidin-5-yl)-1H-indole-2-carboxylic acid was dissolved in DMF. Then 2 eq. of EDCI-HCl, HOBt, and 10 eq. of DIPEA were added. The reaction mixture was stirred at room temperature for 10 mins. and added Intermediate III-E. The mixture was stirred overnight at room temperature. Then the mixture was diluted with water and extracted the product with EtOAc for 3 times. The organic layer was washed with brine. The crude reaction was purified by column chromatography with MeOH/EtOAc to afford Example 135 (26 mg, 93% purity by UV). LCMS Method A, Rt=4.61 min, m/z=517.2 [M+H]+, LCMS Method C, Rt=4.46 min, m/z=517.1546 [M+H]+, exact mass: 516.1477.

### Example 136. (S)-1-(3-chloro-6-(4,6-dimethylpyrimidin-5-yl)-1H-indole-2-carbonyl)-N-((2-chlorothiazol-5-yl)methyl)pyrrolidine-3-carboxamide

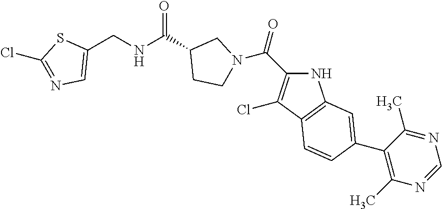 [SMILES: Cc1ncnc(C)c1-c1ccc2c(Cl)c(C(=O)N3CC[C@H](C(=O)NCc4cnc(Cl)s4)C3)[nH]c2c1]

### Step 1: Synthesis of methyl 6-(4,6-dimethylpyrimidin-5-yl)-1H-indole-2-carboxylate via Suzuki cross coupling

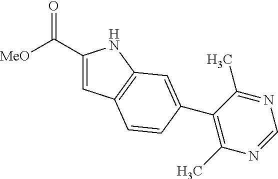 [SMILES: COC(=O)c1cc2ccc(-c3c(C)ncnc3C)cc2[nH]1]

1.2 eq. of 5-bromo-4,6-dimethylpyrimidine and 1 eq. of Intermediate V-B was dissolved in H₂O:1,4-dioxane (1:5) and then bubbled argon gas for 10 mins. The mixture was added 3 eq. of K₂CO₃ and 10 mol % Pd(dppf)Cl₂ under argon. Reaction was heated under argon gas at 110° C. for 1 hour. To achieve pure product, crude product was purified by liquid column chromatography with EtOAc/hexane.

### Step 2: Synthesis of methyl 3-chloro-6-(4,6-dimethylpyrimidin-5-yl)-1H-indole-2-carboxylate

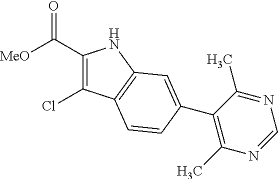 [SMILES: COC(=O)c1[nH]c2cc(-c3c(C)ncnc3C)ccc2c1Cl]

A solution of 1 eq. of methyl 6-(4,6-dimethylpyrimidin-5-yl)-1H-indole-2-carboxylate in DCM were added 1.05 eq. of N-chlorosuccinimide. The reaction mixture was stirred at room temperature overnight. The reaction was quenched by water and extracted with EtOAc. The organic layer was washed with brine, anhydrous sodium sulfate, and concentrated. Crude reaction was purified by column chromatography with EtOAc/Hexanes.

### Step 3: Hydrolysis of methyl 3-chloro-6-(4,6-dimethylpyrimidin-5-yl)-1H′-indole-2-carboxylate

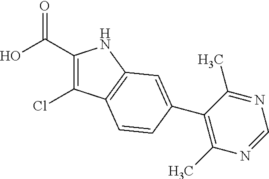 [SMILES: Cc1ncnc(C)c1-c1ccc2c(Cl)c(C(=O)O)[nH]c2c1]

1 eq. of methyl 3-chloro-6-(4,6-dimethylpyrimidin-5-yl)-1H-indole-2-carboxylate was dissolved in 4 mL of TH. Then, 5 eq. of LiOH was dissolved in 8 mL of H₂O and added to the THF solution. The reaction mixture was stirred at room temperature overnight and concentrated by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 4: Amide Formation

1 eq. of 3-chloro-6-(4,6-dimethylpyrimidin-5-yl)-1H-indole-2-carboxylic acid was dissolved in DMF. Then 2 eq. of EDCI-HCl, HOBt, and 10 eq. of DIPEA were added. The reaction mixture was stirred at room temperature for 10 mins. then 1.1 eq. of Intermediate III-L was added. The mixture was stirred overnight at room temperature. Then the mixture was diluted with water and extracted the product with EtOAc for 3 times. The organic layer was washed with brine. The crude reaction was purified by column chromatography with MeOH/EtOAc to afford Example 136 (14 mg, 95% purity by UV). LCMS Method A, Rt=2.93 min, m/z=529.1 [M+H]+, LCMS Method C, Rt=4.14 min, m/z=529.1006 [M+H]+, exact mass: 528.0902.

### Example 137. (S)-1-(3-chloro-7-(4,6-dimethylpyrimidin-5-yl)-1H-indole-2-carbonyl)-N-(3-cyano-4-fluorophenyl)pyrrolidine-3-carboxamide

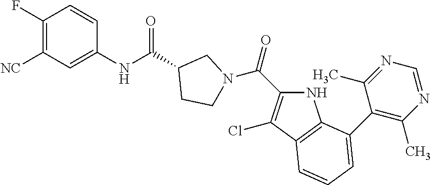 [SMILES: Cc1ncnc(C)c1-c1cccc2c(Cl)c(C(=O)N3CC[C@H](C(=O)Nc4ccc(F)c(C#N)c4)C3)[nH]c12]

### Step 1: Synthesis of methyl 7-(4,4,5,5-tetramethyl-1,3,2-dioxaborolan-2-yl)-1H-indole-2-carboxylate

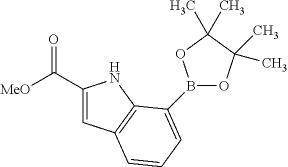 [SMILES: COC(=O)c1cc2cccc(B3OC(C)(C)C(C)(C)O3)c2[nH]1]

1 eq. of methyl 7-bromo-1H-indole-2-carboxylate was dissolved in 1,4-dioxane. Consequently, 1.2 eq. bis(pinacolato)diboron and 3 eq. of KOAc were added to the flask. The reaction mixture was bubbled under argon for 15 mins. Then 10 mol % of Pd(dppf)Cl₂·DCM was added to the reaction. Then, the reaction was heated at 110° C. for 4 hours. The product was obtained by filtering through celite. The celite was washed by EtOAc to remove the remaining product. The organic product was concentrated by rotavapor. Concentrated filtrate was used in the next step.

### Step 2: Synthesis of methyl 7-(4,6-dimethylpyrimidin-5-yl)-1H-indole-2-carboxylate via Suzuki cross coupling

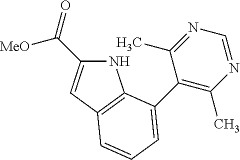 [SMILES: COC(=O)c1cc2cccc(-c3c(C)ncnc3C)c2[nH]1]

1.2 eq. of 5-bromo-4,6-dimethylpyrimidine and 1 eq. of methyl 7-(4,4,5,5-tetramethyl-1,3,2-dioxaborolan-2-yl)-1H-indole-2-carboxylate were dissolved in H₂O:1,4-dioxane (1:5) and then bubbled argon gas for 10 mins. The mixture was added 3 eq. of K₂CO₃ and 10 mol % Pd(dppf)Cl₂ under argon. Reaction was heated under argon gas at 110° C. for 1 hour. To achieve pure product, crude product was purified by liquid column chromatography with EtOAc/hexane.

### Step 3: Synthesis of methyl 3-chloro-7-(4,6-dimethylpyrimidin-5-yl)-1H-indole-2-carboxylate

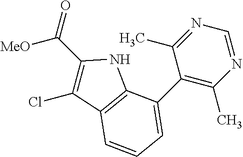 [SMILES: COC(=O)c1[nH]c2c(-c3c(C)ncnc3C)cccc2c1Cl]

A solution of 1 eq. of methyl 7-(4,6-dimethylpyrimidin-5-yl)-1H-indole-2-carboxylate in DCM was added to 1.05 eq. of N-chlorosuccinimide. The reaction mixture was stirred at room temperature overnight. The reaction was quenched by water and extracted with EtOAc. The organic layer was washed with brine, anhydrous sodium sulfate, and concentrated. Crude reaction was purified by column chromatography with EtOAc/Hexanes.

### Step 4: Hydrolysis of methyl 3-chloro-7-(4,6-dimethylpyrimidin-5-yl)-1H-indole-2-carboxylate

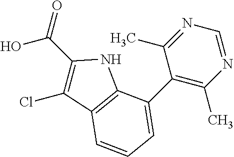 [SMILES: Cc1ncnc(C)c1-c1cccc2c(Cl)c(C(=O)O)[nH]c12]

1 eq. of methyl 3-chloro-7-(4,6-dimethylpyrimidin-5-yl)-1H-indole-2-carboxylate was dissolved in THF. Then, 5 eq. of LiOH was dissolved in H₂O and added to the THF solution. The reaction mixture was stirred at room temperature overnight and concentrated by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 5: Amide Formation

1 eq. of 3-chloro-7-(4,6-dimethylpyrimidin-5-yl)-1H-indole-2-carboxylic acid was dissolved in DMF. Then 2 eq. of EDCI-HCl, HOBt, and 10 eq. of DIPEA were added. The reaction mixture was stirred at room temperature for 10 mins. then 1.1 eq. of Intermediate III-E was added. The mixture was stirred overnight at room temperature. Then the mixture was diluted with water and extracted the product with EtOAc for 3 times. The organic layer was washed with brine. The crude reaction was purified by column chromatography with MeOH/EtOAc to afford Example 137 (21 mg, 97% purity by UV). LCMS Method A, Rt=4.65 min, m/z=517.2 [M+H]+, LCMS Method C, Rt=4.63 min, m/z=517.1552 [M+H]+, exact mass: 516.1477.

### Example 138. (2S,3S)-1-(3-chloro-6-(4,6-dimethylpyrimidin-5-yl)-1H-indole-2-carbonyl)-N-(3-cyano-4-fluorophenyl)-2-methylpyrrolidine-3-carboxamide

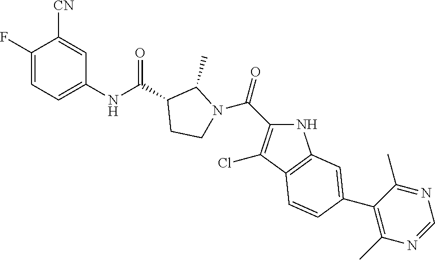 [SMILES: Cc1ncnc(C)c1-c1ccc2c(Cl)c(C(=O)N3CC[C@H](C(=O)Nc4ccc(F)c(C#N)c4)[C@@H]3C)[nH]c2c1]

### Step 1: Synthesis of methyl 6-(4,6-dimethylpyrimidin-5-yl)-1H-indole-2-carboxylate via Suzuki cross coupling

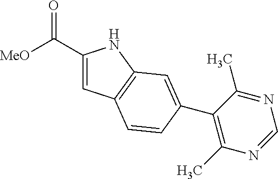 [SMILES: COC(=O)c1cc2ccc(-c3c(C)ncnc3C)cc2[nH]1]

1.2 eq. of 5-bromo-4,6-dimethylpyrimidine and 1 eq. of Intermediate V-B was dissolved in H₂O:1,4-dioxane (1:5) and then bubbled argon gas for 10 mins. The mixture was added 3 eq. of K₂CO₃ and 10 mol % Pd(dppf)Cl₂ under argon. Reaction was heated under argon gas at 110° C. for 1 hour. To achieve pure product, crude product was purified by liquid column chromatography with EtOAc/hexanes.

### Step 2: Synthesis of methyl 3-chloro-6-(4,6-dimethylpyrimidin-5-yl)-1H-indole-2-carboxylate

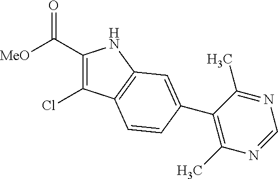 [SMILES: COC(=O)c1[nH]c2cc(-c3c(C)ncnc3C)ccc2c1Cl]

A solution of methyl 6-(4,6-dimethylpyrimidin-5-yl)-1H-indole-2-carboxylate in DCM were added to 1.05 eq. of N-chlorosuccinimide. The reaction mixture was stirred at room temperature overnight. The reaction was quenched by water and extracted with EtOAc. The organic layer was washed with brine, anhydrous sodium sulfate, and concentrated. The crude reaction was purified by column chromatography with EtOAc/Hexanes.

### Step 3: Hydrolysis of methyl 3-chloro-6-(4,6-dimethylpyrimidin-5-yl-1H-indole-2-carboxylate

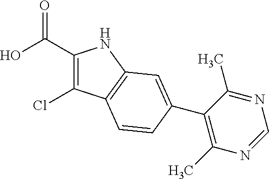 [SMILES: Cc1ncnc(C)c1-c1ccc2c(Cl)c(C(=O)O)[nH]c2c1]

1 eq. of 3-chloro-6-(4,6-dimethylpyrimidin-5-yl)-1H-indole-2-carboxylate was dissolved in THF. Then 5 eq. of LiOH was dissolved in H₂O and added to the THF solution. The reaction mixture was stirred at room temperature overnight and dried the reaction by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 4: Amide Formation

1 eq. of 3-chloro-6-(4,6-dimethylpyrimidin-5-yl)-1H-indole-2-carboxylic acid was dissolved in DMF. Then 2 eq. of EDCI, HOBt, and 10 eq. of DIPEA were added. The reaction mixture was stirred at room temperature for 10 mins, then Intermediate MI-G was added. The mixture was stirred overnight at room temperature. Then the mixture was diluted with water and extracted the product with EtOAc for 3 times. The organic layer was washed with brine. The crude reaction was purified by column chromatography with 1-10% MeOH/EtOAc to afford Example 138 (31 mg, 100% purity by UV). LCMS Method A, Rt=4.70 min, m/z=531.2 [M+H]+, exact mass: 530.1633.

### Example 139. (S)-1-(3-chloro-6-(3-methylpyrazin-2-yl)-1H-indole-2-carbonyl)-N-(3-cyano-4-fluorophenyl) pyrrolidine-3-carboxamide

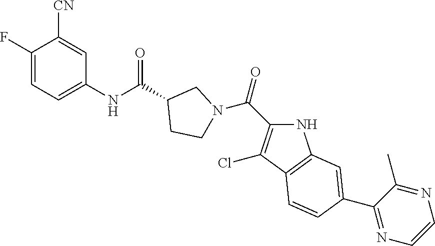 [SMILES: Cc1nccnc1-c1ccc2c(Cl)c(C(=O)N3CC[C@H](C(=O)Nc4ccc(F)c(C#N)c4)C3)[nH]c2c1]

### Step 1: Synthesis of methyl 6-(3-methylpyrazin-2-yl)-1H-indole-2-carboxylate via Suzuki cross coupling

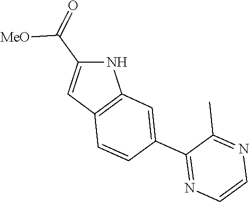 [SMILES: COC(=O)c1cc2ccc(-c3nccnc3C)cc2[nH]1]

1.2 eq. of 2-bromo-3-methylpyrazine and 1 eq. of Intermediate V-B was dissolved in H₂O:1,4-dioxane (1:5) and then bubbled argon gas for 10 mins. The mixture was added 3 eq. of K₂C₀M and 10 mol % Pd(dppf)Cl₂ under argon. Reaction was heated under argon gas at 110° C. for 1 hour. To achieve pure product, crude product was purified by liquid column chromatography with EtOAc/hexanes.

### Step 2: Synthesis of methyl 3-chloro-6-(3-methylpyrazin-2-yl)-1H-indole-2-carboxylate

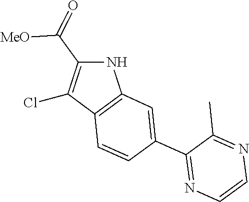 [SMILES: COC(=O)c1[nH]c2cc(-c3nccnc3C)ccc2c1Cl]

A solution of 1 eq. of methyl 6-(3-methylpyrazin-2-yl)-1H-indole-2-carboxylate in DCM was added to 1.05 eq. of N-chlorosuccinimide. The reaction mixture was stirred at room temperature overnight. The reaction was quenched by water and extracted with EtOAc. The organic layer was washed with brine, anhydrous sodium sulfate, and concentrated. The crude reaction was purified by column chromatography with EtOAc/Hexanes.

### Step 3: Hydrolysis of methyl 3-chloro-6-(3-methylpyrazin-2-yl)-1H-indole-2-carboxylate

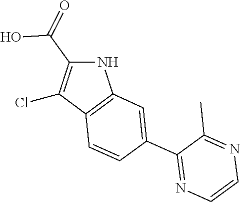 [SMILES: Cc1nccnc1-c1ccc2c(Cl)c(C(=O)O)[nH]c2c1]

1 eq. of methyl 3-chloro-6-(4,6-dimethylpyrimidin-5-yl)-1H-indole-2-carboxylate was dissolved in THF. Then 5 eq. of LiOH was dissolved in H₂O and added to the THF solution. The reaction mixture was stirred at room temperature overnight and dried the reaction by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 4: Amide Formation

1 eq. of 3-chloro-6-(3-methylpyrazin-2-yl)-1H-indole-2-carboxylic acid was dissolved in DMF. Then 2 eq. of EDCI-HCl, HOBt, and 10 eq. of DIPEA were added. The reaction mixture was stirred at room temperature for 10 mins, then Intermediate III-E was added. The mixture was stirred overnight at room temperature. Then the mixture was diluted with water and extracted the product with EtOAc for 3 times. The organic layer was washed with brine. The crude reaction was purified by column chromatography with MeOH/EtOAc to afford Example 139 (15 mg, 96% purity by UV). LCMS Method A, Rt=4.61 min, m/z=503.1 [M+H]+, LCMS Method C, Rt=4.47 min, m/z=503.1399 [M+H]+, exact mass: 502.1320.

### Example 140. (S)-1-(3-(1H-pyrazol-4-yl)-1H-indole-2-carbonyl)-N-(3,4,5-trifluorophenyl)pyrrolidine-3-carboxamide

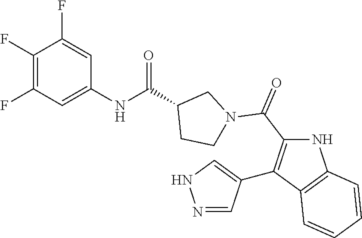 [SMILES: O=C(Nc1cc(F)c(F)c(F)c1)[C@H]1CCN(C(=O)c2[nH]c3ccccc3c2-c2cn[nH]c2)C1]

### Step 1: Synthesis of methyl 3-(1H-pyrazol-4-yl)-1H-indole-2-carboxylate via Suzuki cross coupling

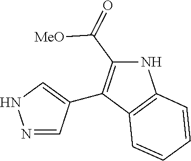 [SMILES: COC(=O)c1[nH]c2ccccc2c1-c1cn[nH]c1]

1.5 eq. of pyrazole-4-boronic acid was dissolved in H₂O:1,4 dioxane (1:5). Then added 1 eq. of methyl 3-bromo-1H-indole-2-carboxylate and 3 eq. of K₂COM to the flask. Bubbled argon gas for 15 mins. Then added 10 mol % Pd(PPh₃)₄ and heated the reaction under argon. Reaction was heated under argon gas at 110° C. for 1.5 hours. To achieve pure product, crude product was purified by liquid column chromatography with EtOAc/hexane.

### Step 2: Hydrolysis of methyl 3-(1H-pyrazol-4-yl)-1H-indole-2-carboxylate

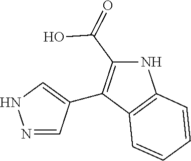 [SMILES: O=C(O)c1[nH]c2ccccc2c1-c1cn[nH]c1]

1 eq. of methyl 3-(1H-pyrazol-4-yl)-1H-indole-2-carboxylate was dissolved in THF. Then dissolved 5 eq. LiOH in H₂O and added to the THF solution. Reaction mixture was stirred at room temperature overnight. Then, acidified the reaction with 2N HCl until pH of solution equals to 2. Dried the reaction mixture with rotavapor. Washed the solid with 15% MeOH/EtOAc. Transferred the solution into another flask and dried. Dried crude product was used in further synthesis.

### Step 3: Amide Formation

1 eq. 3-(1H-pyrazol-4-yl)-1H-indole-2-carboxylic acid was dissolved in DMF. Then 1.5 eq. of HATU and 3 eq. of DIPEA were added. The reaction mixture was stirred at room temperature for 10 mins followed by adding 1.1 eq. of Intermediate III-A. Stirred mixture overnight at room temperature. Then diluted the mixture with water. Then the mixture was diluted with water and extracted the product with EtOAc for 3 times. The organic layer was washed with brine. The crude reaction was purified by column chromatography with MeOH/EtOAc to afford Example 140 (25 mg, 96% purity by UV). LCMS Method A, Rt=2.80 min, m/z=454.1 [M+H]+, exact mass: 453.1413.

### Example 141. (2S,3S)-1-(3-chloro-6-(1H-pyrazol-4-yl)-1H-indole-2-carbonyl)-N-(3-cyano-4-fluorophenyl)-2-methylpyrrolidine-3-carboxamide

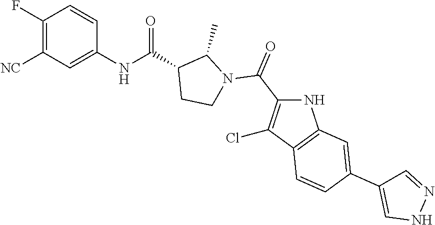 [SMILES: C[C@H]1[C@@H](C(=O)Nc2ccc(F)c(C#N)c2)CCN1C(=O)c1[nH]c2cc(-c3cn[nH]c3)ccc2c1Cl]

### Step 1: Synthesis of methyl 6-bromo-3-chloro-1H-indole-2-carboxylate

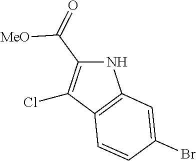 [SMILES: COC(=O)c1[nH]c2cc(Br)ccc2c1Cl]

A solution of 1 eq. of methyl 6-bromo-1H-indole-2-carboxylate in DCM was added to 1.05 eq. of N-chlorosuccinimide. The reaction mixture was stirred at room temperature overnight. The reaction was quenched by water and extracted with EtOAc. The organic layer was washed with brine, anhydrous sodium sulfate, and concentrated. The crude reaction was purified by column chromatography with EtOAc/Hexanes.

### Step 2: Synthesis of methyl 3-chloro-6-(1H-pyrazol-4-yl)-1H-indole-2-carboxylate via Suzuki cross coupling

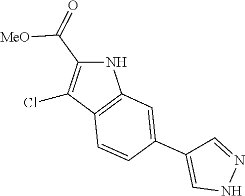 [SMILES: COC(=O)c1[nH]c2cc(-c3cn[nH]c3)ccc2c1Cl]

1.5 eq. of Pyrazole-4-boronic acid and 1 eq. of methyl 6-bromo-3-chloro-1H-indole-2-carboxylate was dissolved in H₂O:1,4-dioxane (1:5) and then bubbled argon gas for 10 mins. The mixture was added 3 eq. of K₂CO₃ and 10 mol % Pd(dppf)Cl₂ under argon. Reaction was heated under argon gas at 110° C. for 1 hour. To achieve pure product, crude product was purified by liquid column chromatography with EtOAc/hexanes.

### Step 3: Hydrolysis of methyl 3-chloro-6-(1H-pyrazol-4-yl)-1H-indole-2-carboxylate

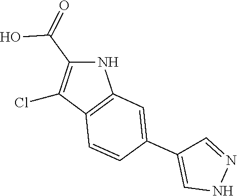 [SMILES: O=C(O)c1[nH]c2cc(-c3cn[nH]c3)ccc2c1Cl]

1 eq. of methyl 3-chloro-6-(1H-pyrazol-4-yl)-1H-indole-2-carboxylate was dissolved in THF. Then 5 eq. of LiOH was dissolved in H₂O and added to the THF solution. The reaction mixture was stirred at room temperature overnight and dried the reaction by rotavapor. Then, the reaction was acidified with 2N HCl until pH of solution equals to 3-4. The solid was washed with 15% MeOH/EtOAc. The solution was transferred into another flask and dried. Dried crude product was used in further synthesis.

### Step 4: Amide Formation

1 eq. of 3-chloro-6-(1H-pyrazol-4-yl)-1H-indole-2-carboxylic acid was dissolved in DMF. Then 2 eq. of EDCI-HCl, HOBt, and 10 eq. of DIPEA were added. The reaction mixture was stirred at room temperature for 10 mins and 1.2 eq. of Intermediate III-G was added. The mixture was stirred overnight at room temperature. Then the mixture was diluted with water and extracted the product with EtOAc for 3 times. The organic layer was washed with brine. The crude reaction was purified by column chromatography with MeOH/EtOAc to afford Example 141 (21 mg, 93% purity by UV). LCMS Method A, Rt=4.49 min, m/z=491.2 [M+H]+, LCMS Method C, Rt=4.43 min, m/z=491.1378 [M+H]+, exact mass: 490.1320.

### Example 142. (S)-5)-1nicotinoyl-1H-pyrrole-2carbonyl)-N-(3,4,-5 trifluorophenyl)pyrrolidine-3carboxamide

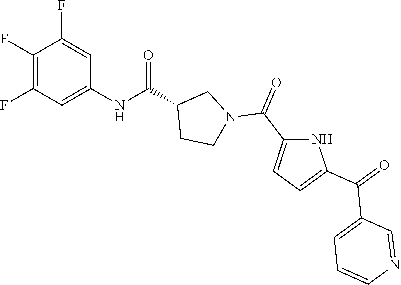 [SMILES: O=C(c1cccnc1)c1ccc(C(=O)N2CC[C@H](C(=O)Nc3cc(F)c(F)c(F)c3)C2)[nH]1]

### Step 1: Synthesis of methyl 5-nicotinoyl-1H-pyrrole-2-carboxylate

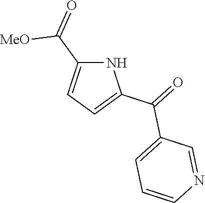 [SMILES: COC(=O)c1ccc(C(=O)c2cccnc2)[nH]1]

1 eq. of nicotinoyl chloride from previous step was dissolved in DCE at 0° C. under argon. Then, 3 eq. of aluminium chloride was added. The mixture was stirred 10 mins. at 0° C. under argon. Consequently, 1.1 eq. of methyl 1H-pyrrole-2-carboxylate was added to the reaction. The mixture was kept stirring overnight at room temperature. To quench the reaction, water was added to the reaction. The aqueous phase was washed by ether to remove excess reactant. The aqueous layer was neutralized by 1M NaOH solution until the pH of solution was around 10-12. Then, the aqueous layer was extracted by EtOAc for 3 times to obtain methyl 5-(2-methylnicotinoyl)-1H-pyrrole-2-carboxylate. The product was used in next step without purification.

### Step 2: Hydrolysis of methyl 5-nicotinoyl-1H-pyrrole-2-carboxylate

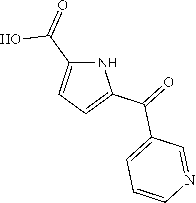 [SMILES: O=C(O)c1ccc(C(=O)c2cccnc2)[nH]1]

1 eq. of methyl 5-nicotinoyl-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water.

### Step 3: Amide Formation

1 eq. of 5-nicotinoyl-1H-pyrrole-2-carboxylic acid and Intermediate IIM-A were dissolved in DMF 3 mL. Then 2 eq. of EDCI·HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude product was purified by column chromatography using 2% MeOH/EtOAc to afford Example 142 (15 mg, 98% purity by UV). LCMS Method A, Rt=4.34 min, m/z=443.1 [M+H]+, exact mass: 442.1253.

### Example 143. (S)-1-(5-(pyridin-3-ylmethyl)-1H-pyrrole-2-carbonyl)-N-(3,4,5-trifluorophenyl)pyrrolidine-3-carboxamide

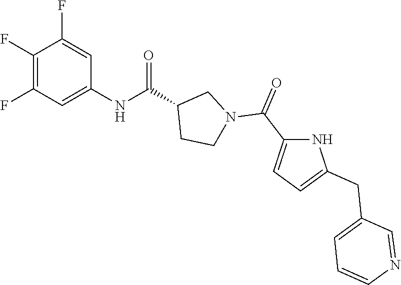 [SMILES: O=C(Nc1cc(F)c(F)c(F)c1)[C@H]1CCN(C(=O)c2ccc(Cc3cccnc3)[nH]2)C1]

1 eq. of (S)-5)-1nicotinoyl-1H-pyrrole-2carbonyl)-N-(3,4,5trifluorophenyl)pyrrolidine-3carboxamide was dissolved in EtOH. The mixture was cooled to 0° C. Then, 1.05 eq. of NaBH₄ was added. The reaction mixture was stirred for an hour at room temperature. Brine was added to stop the reaction. The aqueous layer was extracted by DCM to product.

1 eq. of crude product from previous step was dissolved in TFA at 0° C. Then, 1.05 eq. of triethylsilane was added to the solution. The reaction was stirred overnight at room temperature under argon. To quench the reaction, saturated NaHCO₃ was added to the reaction. The aqueous layer was washed by EtOAc. The crude product was purified by column chromatography with MeOH/EtOAc to obtain Example 143 (12 mg, 93% purity by UV). LCMS Method A, Rt=4.15 min, m/z=429.1 [M+H]+, exact mass: 428.1460.

### Example 144. (S)-1-(5-(2-methylnicotinoyl)-1H-pyrrole-2-carbonyl)-N-(3,4,5-trifluorophenyl)pyrrolidine-3-carboxamide

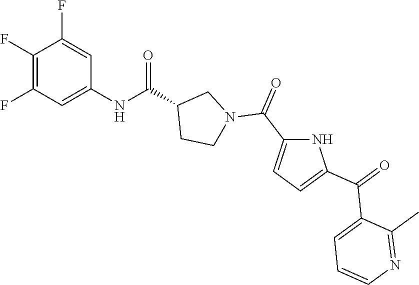 [SMILES: Cc1ncccc1C(=O)c1ccc(C(=O)N2CC[C@H](C(=O)Nc3cc(F)c(F)c(F)c3)C2)[nH]1]

### Step 1: Synthesis of methyl 5-(2-methylnicotinoyl)-1H-pyrrole-2-carboxylate

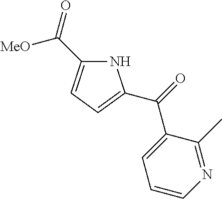 [SMILES: COC(=O)c1ccc(C(=O)c2cccnc2C)[nH]1]

2-methylnicotinic acid was dissolved in oxalyl chloride at 0° C. Then DMF was added to the reaction at 0° C. as a catalyst. The reaction was warmed up to room temperature. The reaction mixture was kept stirring for an hour. Then, crude reaction was dried by rotavapor to obtain the acyl chloride product.

1 eq. of acyl chloride from previous step was dissolved in DCE at 0° C. under argon. Then, 3 eq. of aluminium chloride was added. The mixture was stirred 10 mins. at 0° C. under argon. Consequently, 1.1 eq. of methyl 1H-pyrrole-2-carboxylate was added to the reaction. The mixture was kept stirring overnight at room temperature. To quench the reaction, water was added to the reaction. The aqueous phase was washed by ether to remove excess reactant. The aqueous layer was neutralized by 1M NaOH solution until the pH of solution was around 10-12. Then, the aqueous layer was extracted by EtOAc for 3 times to obtain methyl 5-(2-methylnicotinoyl)-1H-pyrrole-2-carboxylate. The product was used in next step without purification.

### Step 2: Hydrolysis of methyl 5-(2-methylnicotinoyl)-1H-pyrrole-2-carboxylate

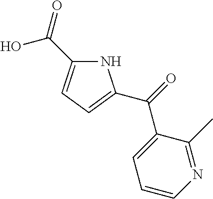 [SMILES: Cc1ncccc1C(=O)c1ccc(C(=O)O)[nH]1]

1 eq. of methyl 5-(2-methylnicotinoyl)-1H-pyrrole-2-carboxylate was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water.

### Step 3: Amide Formation

1 eq. of 5-(2-methylnicotinoyl)-1H-pyrrole-2-carboxylic acid and Intermediate III-A were dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude product was purified by column chromatography using 2% MeOH/EtOAc to afford Example 144 (16 mg, 98% purity by UV). LCMS Method A, Rt=4.21 min, m/z=457.1 [M+H]+, exact mass: 456.1409. ¹H-NMR (500 MHz, Acetone-d₆) δ 11.32 (s, 1H), 9.79 (s, 1H), 8.55 (d, J=4.2 Hz, 1H), 7.79 (d, J=7.7 Hz, 1H), 7.57-7.48 (m, 2H), 7.37 (s, 1H), 7.29 (t, J=5.0 Hz, 1H), 7.04 (d, J=15.1 Hz, 1H), 4.13-3.86 (br, 2H), 3.70-3.65 (m, 1H), 3.39 (br, 1H), 3.24 (br, 1H), 2.49 (s, 3H), 2.41-2.24 (m, 1H), 2.09-2.04 (overlap, 2H), 1.19-1.09 (m, 4H).

### Example 145. (S)-1-(5-((2-methylpyridin-3-yl)methyl)-1H-pyrrole-2-carbonyl)-N-(3,4,5-trifluorophenyl)pyrrolidine-3-carboxamide

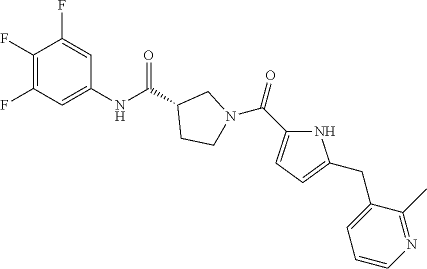 [SMILES: Cc1ncccc1Cc1ccc(C(=O)N2CC[C@H](C(=O)Nc3cc(F)c(F)c(F)c3)C2)[nH]1]

1 eq. of (S)-1-(5-(2-methylnicotinoyl)-1H-pyrrole-2-carbonyl)-N-(3,4,5-trifluorophenyl)pyrrolidine-3-carboxamide was dissolved in EtOH. The mixture was cooled to 0° C. Then, 1.05 eq. of NaBH₄ was added. The reaction mixture was stirred for an hour at room temperature. Brine was added to stop the reaction. The aqueous layer was extracted by DCM to product.

1 eq. of crude product from previous step was dissolved in TFA at 0° C. Then, 1.05 eq. of triethylsilane was added to the solution. The reaction was stirred overnight at room temperature under argon. To quench the reaction, saturated NaHCO₃ was added to the reaction. The aqueous layer was washed by EtOAc. The crude product was purified by column chromatography with MeOH/EtOAc to obtain Example 145 (10 mg, 99% purity by UV). LCMS Method A, Rt=4.16 min, m/z=443.2 [M+H]+, LCMS Method C, Rt=3.51 min, m/z=443.1714 [M+H]+, exact mass: 442.1617. ¹H-NMR (500 MHz, Acetone-d₆) δ 10.71 (s, 1H), 9.99 (d, J=16.2 Hz, 1H), 8.61 (d, J=4.3 Hz, 1H), 8.13 (d, J=7.7 Hz, 1H), 7.68 (t, J=1.7 Hz, 1H), 7.59-7.48 (m, 2H), 6.89 (s, 1H), 6.57 (s, 1H), 4.04 (s, 2H), 3.91-3.14 (m(br), 5H), 2.74 (s, 3H), 2.39-2.10 (m, 2H).

### Example 146. (S)-1-(5-((1,3-dimethyl-1H-pyrazol-4-yl)methyl)-1H-pyrrole-2-carbonyl)-N-(3,4,5-trifluorophenyl)pyrrolidine-3-carboxamide

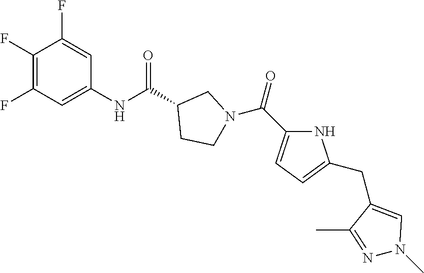 [SMILES: Cc1nn(C)cc1Cc1ccc(C(=O)N2CC[C@H](C(=O)Nc3cc(F)c(F)c(F)c3)C2)[nH]1]

### Step 1: Synthesis of methyl 5-(1,3-dimethyl-1H-pyrrazole-4-carbonyl)-1H-pyrrole-2-carboxylate

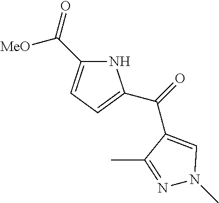 [SMILES: COC(=O)c1ccc(C(=O)c2cn(C)nc2C)[nH]1]

Ethyl 3-methyl-1H-pyrazole-4-carboxylate (1 eq.) was dissolved in THF. The solution was cooled to 0° C. Then 60% of sodium hydride solution in THF (1.05 eq) was dropped to the cooled solution. The reaction mixture was stirred for 10 mins. at 0° C. Then, 1.16 eq. of tert-butyl 2-bromoacetate was dropped to the previous solution slowly. Then the reaction was heated for 2 hours at 60° C. The reaction was quenched by adding water. The aqueous solution was extracted by EtOAc for 3 times to obtain the product. The organic layer was dried by anhydrous sodium sulfate and removed solvent by rotavapor. The product was used in the next step without purification.

1 eq. of crude product was dissolved in THF. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

Then, 1,3-dimethyl-1H-pyrazole-4-carboxylic acid was dissolved in oxalyl chloride at 0° C. Then DMF was added to the reaction at 0° C. as a catalyst. The reaction was warmed up to room temperature. The reaction mixture was kept stirring for an hour. Then, crude reaction was dried by rotavapor to obtain the acyl chloride product.

1 eq. of acyl chloride from previous step was dissolved in DCE at 0° C. under argon. Then, 3 eq. of aluminium chloride was added. The mixture was stirred 10 mins. at 0° C. under argon. Consequently, 1.1 eq. of methyl 1H-pyrrole-2-carboxylate was added to the reaction. The mixture was kept stirring overnight at room temperature. To quench the reaction, water was added to the reaction. The aqueous phase was washed by ether to remove excess reactant. The aqueous layer was neutralized by 1M NaOH solution until the pH of solution was around 10-12. Then, the aqueous layer was extracted by EtOAc for 3 times to obtain methyl 5-(2-methylnicotinoyl)-1H-pyrrole-2-carboxylate. The product was used in next step without purification.

### Step 2: Hydrolysis of methyl 5-(1,3-dimethyl-1H-pyrazole-4-carbonyl)-1H-pyrrole-2-carboxylate

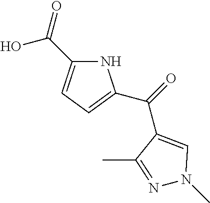 [SMILES: Cc1nn(C)cc1C(=O)c1ccc(C(=O)O)[nH]1]

1 eq. of Methyl 5-(1,3-dimethyl-1H-pyrazole-4-carbonyl)-1H-pyrrole-2-carboxylate was dissolved in THE. Then, 5 eq. of LiOH was dissolved in H₂O and added to the THF solution. The reaction mixture was stirred at room temperature overnight. The reaction was concentrated in vacuo. The reaction was acidified by 2N HCl until pH of solution equals to 3. During this acidification, a white precipitate formed. The solid was collected via filtration and washed with water, providing the product as a white solid.

### Step 3: Amide Formation

1 eq. of 5-(1,3-dimethyl-1H-pyrazole-4-carbonyl)-1H-pyrrole-2-carboxylic acid and Intermediate III-A (1 eq.) were dissolved in DMF 3 mL. Then 2 eq. of EDCI-HCl, HOBt, and 10 eq. of DIPEA were added. The reaction mixture was stirred at room temperature. The mixture was stirred overnight at room temperature. Then the mixture was diluted with water and extracted the product with EtOAc for 3 times. The organic layer was washed with brine. The crude product was purified by column chromatography using MeOH/EtOAc to afford Example 146 (18 mg, 87% purity by UV). LCMS Method A, Rt=4.41 min, m/z=446.2 [M+H]⁺, exact mass: 445.1726.

### Example 147. 2-(4-(5-((2S,3S)-3-((4-fluoro-3-methylphenyl)carbamoyl)-2-methylpyrrolidine-1-carbonyl)-1H-pyrrol-2-yl)-3,5-dimethyl-1H-pyrazol-1-yl)acetic acid

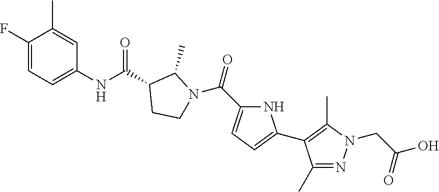 [SMILES: Cc1cc(NC(=O)[C@H]2CCN(C(=O)c3ccc(-c4c(C)nn(CC(=O)O)c4C)[nH]3)[C@H]2C)ccc1F]

### Step 1: Synthesis of (2S,3S)-1-(5-bromo-1H-pyrrole-2-carbonyl)-N-(4-fluoro-3-methylphenyl)-2-methylpyrrolidine-3-carboxamide

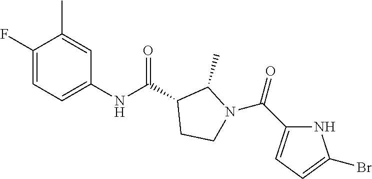 [SMILES: Cc1cc(NC(=O)[C@H]2CCN(C(=O)c3ccc(Br)[nH]3)[C@H]2C)ccc1F]

A mixture of 5-bromo-1H-pyrrole-2-carboxylic acid (1 eq.), Intermediate III-H (1.14 eq.) was dissolved in DMF (5 mL). Then, added 1.5 eq. of HATU and 1 mL of DIPEA. The mixture was stirred for overnight at room temperature. Then, the mixture was diluted with water. The product was extracted with EtOAc for 3 times. The organic layer was washed by brine and purified by column chromatography with 60% EtOAc/hexanes. The desired fractions were pooled and the solvent was removed under reduced pressure to afford the product.

### Step 2: Suzuki Coupling Reaction

1 eq. of 4-iodo-3,5-dimethyl-1H-pyrazole was dissolved in THF. Then, 60% sodium hydride solution (1.05 eq.) was added to the mixture. The mixture was stirred for 10 mins. at 0° C. 1.16 eq. of tert-butyl 3-bromopropanoate was added. The solution was heated at 60° C. for 2 hours. Then, water was added to quench the reaction. The product was extracted by EtOAc and the crude was purified via silica gel column chromatography by using EtOAC:Hexanes.

1 eq. of tert-butyl 2-(4-iodo-3,5-dimethyl-1H-pyrazol-1-yl)acetate and 1.5 eq. of bis(pinacolato)diboron were dissolved in 1,4-dioxane. The reaction mixture was bubbled under argon for 10 mins. Then, 10 mol % of Pd(dppf)Cl₂ and 2 eq. of KOAc were added to the reaction. Then, the reaction was heated at 80° C. for 2 hours. The product was obtained by filtering through celite. The celite was washed by EtOAc to remove the remaining product. The organic product was concentrated by rotavapor. Concentrated filtrate was used in the next step.

A mixture of (2S,3S)-1-(5-bromo-1H-pyrrole-2-carbonyl)-N-(4-fluoro-3-methylphenyl)-2-methylpyrrolidine-3-carboxamide and tert-butyl 2-(3,5-dimethyl-4-(4,4,5,5-tetramethyl-1,3,2-dioxaborolan-2-yl)-1H-pyrazol-1-yl)acetate (3 eq.) in 1,4-dioxane/H₂O (4:1, 0.1 M) was bubbled by argon for 10 mins. The mixture was added with Pd(PPh₃)₂Cl₂ (10 mol %) and potassium carbonate (3 eq.). The reaction was heated at 80° C. for 2 hours in sealed tube and then cooled to room temperature, filtered, and concentrated in vacuo. The crude product was purified by liquid column chromatography using 5% MeOH/EtOAc.

1 eq. of tert-butyl 2-(4-(5-((2S,3S)-3-((4-fluoro-3-methylphenyl)carbamoyl)-2-methylpyrrolidine-1-carbonyl)-1H-pyrrol-2-yl)-3,5-dimethyl-1H-pyrazol-1-yl)acetate dissolved in THF. Then, 5 eq. of LiOH was dissolved in H₂O and added to the THF solution. The reaction mixture was stirred for overnight. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was purified by sephadex LH₂O column chromatography to afford Example 147 (1.01 mg, 94.4% purity by UV). LCMS Method B, Rt=4.40 min, m/z=482.2 [M+H]+, exact mass: 481.2125.

### Example 148. (S)—N-(3-chloro-4-fluorophenyl)-1-(5-(2-cyanopyridin-3-yl)-1H-pyrrole-2-carbonyl)pyrrolidine-3-carboxamide

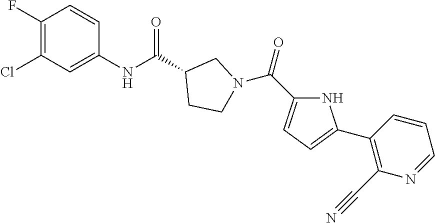 [SMILES: N#Cc1ncccc1-c1ccc(C(=O)N2CC[C@H](C(=O)Nc3ccc(F)c(Cl)c3)C2)[nH]1]

### Step 1: Synthesis of methyl 5-(2-cyanopyridin-3-yl)-1H-pyrrole-2-carboxylate via Suzuki cross coupling

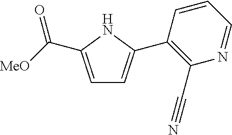 [SMILES: COC(=O)c1ccc(-c2cccnc2C#N)[nH]1]

1.2 eq. of 3-bromopicolinonitrile was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(2-cyanopyridin-3-yl)-1H-pyrrole-2-carboxylate

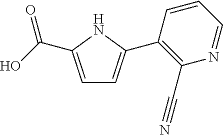 [SMILES: N#Cc1ncccc1-c1ccc(C(=O)O)[nH]1]

1 eq. of methyl 5-(2-cyanopyridin-3-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(2-cyanopyridin-3-yl)-1H-pyrrole-2-carboxylic acid was dissolved in DMF. Then, ² eq. of EDCI-HCl, HOBt, and 10 eq. of DIPEA were added. The reaction mixture was stirred at room temperature for 10 mins. and added 1.1 eq. of Intermediate III-I. The mixture was stirred for overnight at room temperature. Then, the mixture was diluted with water and extracted the product with EtOAc for 3 times. The organic layer was washed with brine. The crude reaction was purified by column chromatography with MeOH/EtOAc to afford Example 148 (4 mg, 94% purity by UV). LCMS Method A, Rt=4.60 min, m/z=438.1 [M+H]*, exact mass: 437.1055.

### Example 149 (2S,3S)-1-(5-(4,6-dimethylpyrimidin-5-yl)-1H-pyrrole-2-carbonyl)-N-(4-fluoro-3-methylphenyl)-2-methylpyrrolidine-3-carboxamide

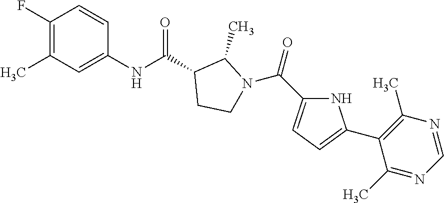 [SMILES: Cc1cc(NC(=O)[C@H]2CCN(C(=O)c3ccc(-c4c(C)ncnc4C)[nH]3)[C@H]2C)ccc1F]

Step 1: Synthesis of methyl 5-(4,6-dimethylpyrimidin-5-yl)-1H-pyrrole-2-carboxylate via Suzuki cross coupling

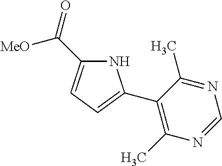 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes. ¹H-NMR (500 MHz, Acetone-d₆) δ 11.14 (s, 1H), 8.83 (s, 1H), 6.97 (dd, J=3.5, 2.7 Hz, 1H), 6.29 (dd, J=3.5, 2.5 Hz, 1H), 3.80 (s, 3H), 2.30 (s, 6H).

### Step 2: Hydrolysis of methyl 5-(4,6-dimethylpyrimidin-5-yl)-1H-pyrrole-2-carboxylate

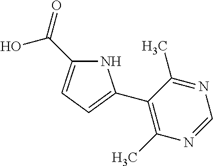 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was stirred at room temperature for overnight. The reaction was concentrated in vacuo. Then, the mixture was acidified by 2N HCl until pH of solution approximate to 3. During this acidification, a white precipitate formed. The solid was collected via filtration and washed with water, providing the product as a white solid.

### Step 3: Amide Formation

1 eq. of 5-(4,6-dimethylpyrimidin-5-yl)-1H-pyrrole-2-carboxylic acid and Intermediate III-H (1 eq.) was dissolved in DMF 3 mL. Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 2% MeOH/EtOAc to give Example 149 (12 mg, 99% purity by UV). LCMS Method A, Rt=4.53 min, m/z=436.2 [M+H]+, LCMS Method C, Rt=4.33 min, m/z=436.2138 [M+H]+, exact mass: 435.2071. ¹H-NMR (500 MHz, DMSO-d₆) δ 611.67 (s, 1H), 9.99 (s, 1H), 8.84 (s, 1H), 7.52 (dd, J=7.0, 2.2 Hz, 1H), 7.44-7.37 (m, 1H), 7.08 (t, J=9.2 Hz, 1H), 6.76 (s, 1H), 6.24 (s, 1H), 3.97 (br, 1H), 3.73 (br, 1H), 3.21-3.03 (m, 2H), 2.27 (s, 6H), 2.21 (d, J=1.2 Hz, 3H), 1.08 (dd, J=9.0, 5.0 Hz, 3H).

### Example 150. (S)-1-(5-(2-cyanopyridin-3-yl)-1H-pyrrole-2-carbonyl)-N-(4-fluoro-3-methylphenyl)pyrrolidine-3-carboxamide

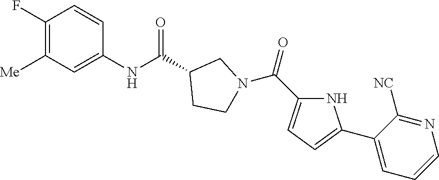 [SMILES: Cc1cc(NC(=O)[C@H]2CCN(C(=O)c3ccc(-c4cccnc4C#N)[nH]3)C2)ccc1F]

### Step 1: Synthesis of methyl 5-(2-cyanopyridin-3-yl)-1H-pyrrole-2-carboxylate via Suzuki cross coupling

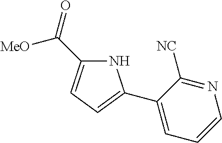 [SMILES: COC(=O)c1ccc(-c2cccnc2C#N)[nH]1]

1.2 eq. of 3-bromopicolinonitrile was dissolved in H₂O:1,4-dioxane (1:5). Then, 1 eq. of Intermediate V-AV-A and 3 eq. of K₂COM were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(2-cyanopyridin-3-yl)-1H-pyrrole-2-carboxylate

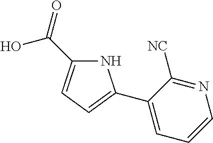 [SMILES: N#Cc1ncccc1-c1ccc(C(=O)O)[nH]1]

1 eq. of Methyl 5-(2-cyanopyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(2-cyanopyridin-3-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-B was added to the solution. The mixture was stirred for overnight at room temperature. Then, the mixture was diluted with water. The product was extracted with EtOAc for 3 times. The organic layer was washed by brine and purified by column chromatography with 1-5% MeOH/EtOAc. The desired fractions were pooled and the solvent was removed under reduced pressure to afford Example 150 (4 mg, 98% purity by UV) LCMS Method A, Rt=4.58 min, m/z=418.1 [M+H]+ 440.1 [M+Na]+ exact mass:417.1601. 1H-NMR (500 MHz, Acetone-d₆) δ 11.21 (s, 1H), 9.37 (s, 1H), 8.59 (dd, J=4.6, 0.9 Hz, 1H), 8.38-8.28 (m, 1H), 7.71 (dd, J=8.3, 4.6 Hz, 1H), 7.56 (d, J=6.3 Hz, 1H), 7.47 (dd, J=8.2, 3.9 Hz, 1H), 7.04-6.92 (m, 2H), 6.82 (s, 1H), 4.04 (s, 1H), 3.84 (br, 1H), 3.80-3.68 (m, 1H), 3.37 (brs, 1H), 3.22 (brs, 1H), 2.20 (s, 3H), 2.03-2.01 (overlap, 2H).

### Example 151. (S)-1-(5-(1-methyl-1H-pyrrolo[3,2-b]pyridin-3-yl)-1H-pyrrole-2-carbonyl)-N-(3,4,5-trifluorophenyl)pyrrolidine-3-carboxamide

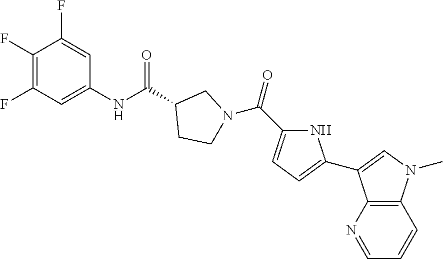 [SMILES: Cn1cc(-c2ccc(C(=O)N3CC[C@H](C(=O)Nc4cc(F)c(F)c(F)c4)C3)[nH]2)c2ncccc21]

### Step 1: Synthesis of methyl 5-(1-methyl-1H-pyrrolo[3,2-b]pyridin-3-yl)-1H-pyrrole-2-carboxylate via Suzuki cross coupling

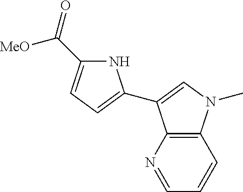 [SMILES: COC(=O)c1ccc(-c2cn(C)c3cccnc23)[nH]1]

1 eq. of 3-bromo-1H-pyrrolo[3,2-b]pyridine was dissolved in DMF at 0° C. under N₂. Then, 1.6 eq. of sodium hydride in DMF was added to the previous solution. The reaction mixture was stirred for 10 mins. In next step, 1.05 eq. of methyl iodide was added. The reaction was left to reach room temperature and stirred for 2 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was used in the next step.

1.2 eq. of 3-bromo-1-methyl-1H-pyrrolo[3,2-b]pyridine was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1-methyl-1H-pyrrolo[3,2-b]pyridin-3-yl)-1H-pyrrole-2-carboxylate

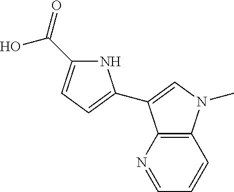 [SMILES: Cn1cc(-c2ccc(C(=O)O)[nH]2)c2ncccc21]

1 eq. of methyl 5-(1-methyl-1H-pyrrolo[3,2-b]pyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(1-methyl-1H-pyrrolo[3,2-b]pyridin-3-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-A was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 151 (6 mg, 93% purity by UV). LCMS Method A, Rt=4.38 min, m/z=468.2 [M+H]*, exact mass: 467.1569.

### Example 152. (S)-1-(5-(1,2-dimethyl-1H-pyrrolo[3,2-b]pyridin-3-yl)-1H-pyrrole-2-carbonyl)-N-(3,4,5-trifluorophenyl)pyrrolidine-3-carboxamide

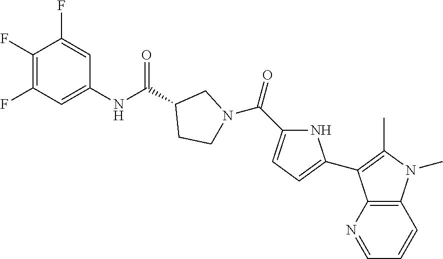 [SMILES: Cc1c(-c2ccc(C(=O)N3CC[C@H](C(=O)Nc4cc(F)c(F)c(F)c4)C3)[nH]2)c2ncccc2n1C]

### Step 1: Synthesis of methyl 5-(1,2-dimethyl-1H-pyrrolo[3,2-b]pyridin-3-yl)-1H-pyrrole-2-carboxylate via Suzuki cross coupling

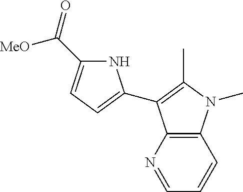 [SMILES: COC(=O)c1ccc(-c2c(C)n(C)c3cccnc23)[nH]1]

1 eq. of 3-bromo-1H-pyrrolo[3,2-b]pyridine was dissolved in DMF at 0° C. under N₂. Then, 1.6 eq. of sodium hydride in DMF was added to the previous solution. The reaction mixture was stirred for 10 mins. In next step, 1.05 eq. of methyl iodide was added. The reaction was left to reach room temperature and stirred for 2 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was used in the next step.

1 eq. of crude product from previous was dissolved in dried THF. The reaction mixture was cooled to −10° C. under N₂. Then, 1.5 eq. of 2M lithium diisopropylamide in THF/heptane/ethylbenzene was added to the reaction. Mixture was further stirred at −10° C. for 2 hours. 1.2 eq. of methyl iodide was added to the reaction and continued stirring for 2 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product purified by liquid column chromatography with EtOAc/hexanes.

1.2 eq. of 3-bromo-1,2-dimethyl-1H-pyrrolo[3,2-b]pyridine was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1,2-dimethyl-1H-pyrrolo[3,2-b]pyridin-3-yl)-1H-pyrrole-2-carboxylate

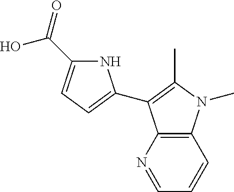 [SMILES: Cc1c(-c2ccc(C(=O)O)[nH]2)c2ncccc2n1C]

1 eq. of methyl 5-(1,2-dimethyl-1H-pyrrolo[3,2-b]pyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1,2-dimethyl-1H-pyrrolo[3,2-b]pyridin-3-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-A was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 152 (8 mg, 96% purity by UV). LCMS Method A, Rt=4.40 min, m/z=482.2 [M+H]+, exact mass: 481.1726.

### Example 153. (S)-1-(5-(1,2-dimethyl-1H-pyrrolo[2,3-b]pyridin-3-yl)-1H-pyrrole-2-carbonyl)-N-(3,4,5-trifluorophenyl)pyrrolidine-3-carboxamide

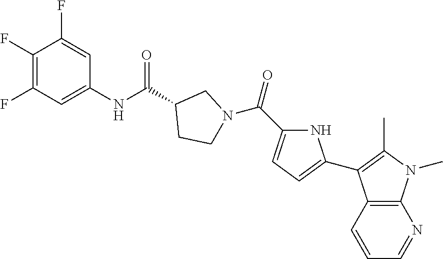 [SMILES: Cc1c(-c2ccc(C(=O)N3CC[C@H](C(=O)Nc4cc(F)c(F)c(F)c4)C3)[nH]2)c2cccnc2n1C]

Step 1: Synthesis of methyl 5-(1,2-dimethyl-1H-pyrrolo[2,3-b]pyridin-3-yl)-1H-pyrrole-2-carboxylate via Suzuki cross coupling

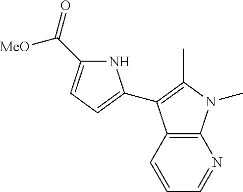 [SMILES: COC(=O)c1ccc(-c2c(C)n(C)c3ncccc23)[nH]1]

1 eq. of 2-methyl-1H-pyrrolo[2,3-b]pyridine was dissolved in MeCN. 3 eq. of CuBr₂ was added to the flask under N₂. The reaction was stirred at room temperature for 2 hours. Then, ammonia solution was added to the reaction to quench. The product was obtained by extracting with EtOAc. The organic layer was washed by H₂O. The product was used in the next step.

1 eq. of 3-bromo-2-methyl-1H-pyrrolo[2,3-b]pyridine was dissolved in DMF at 0° C. under N₂. Then, 1.6 eq. of sodium hydride in DMF was added to the previous solution. The reaction mixture was stirred for 10 mins. In next step, 1.05 eq. of methyl iodide was added. The reaction was left to reach RT and stirred for 2 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was purified by liquid column chromatography with EtOAc/hexanes.

1.2 eq. of 3-bromo-1,2-dimethyl-1H-pyrrolo[2,3-b]pyridine was dissolved in H₂O:1,4-dioxane) 1:5. (Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1,2-dimethyl-1H-pyrrolo[2,3-b]pyridin-3-yl)-1H-pyrrole-2-carboxylate

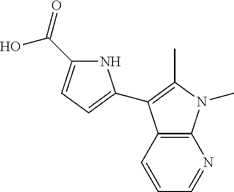 [SMILES: Cc1c(-c2ccc(C(=O)O)[nH]2)c2cccnc2n1C]

1 eq. of methyl 5-(1,2-dimethyl-1H-pyrrolo[2,3-b]pyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1,2-dimethyl-1H-pyrrolo[2,3-b]pyridin-3-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-A was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 153 (7.5 mg, 97% purity by UV). LCMS Method A, Rt=4.76 min, m/z=482.2 [M+H]+, exact mass: 481.1726. ¹H-NMR (500 MHz, Acetone-d₆) 510.37 (s, 1H), 9.80 (s, 1H), 8.19 (dd, J=4.7, 1.4 Hz, 1H), 7.93 (dd, J=7.8, 1.4 Hz, 1H), 7.57-7.42 (m, 2H), 7.05 (dd, J=7.8, 4.7 Hz, 1H), 6.76 (dd, J=3.4 2.7 Hz, 1H), 6.29 (dd, J=3.5, 2.9 Hz, 1H), 4.05-3.77 (m, 3H), 3.26 (s, 3H), 3.21-3.14 (m, 2H), 2.58 (s, 3H), 2.17 (d, J=2.5 Hz, 1H), 2.14 (dq, J=4.4, 2.2 Hz, 1H).

### Example 154. (S)-1-(5-(1-methyl-1H-pyrrolo[3,2-c]pyridin-3-yl)-1H-pyrrole-2-carbonyl)-N-(3,4,5-trifluorophenyl)pyrrolidine-3-carboxamide

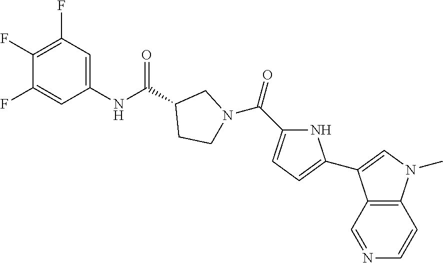 [SMILES: Cn1cc(-c2ccc(C(=O)N3CC[C@H](C(=O)Nc4cc(F)c(F)c(F)c4)C3)[nH]2)c2cnccc21]

### Step 1: Synthesis of methyl 5-(1-methyl-1H-pyrrolo[3,2-c]pyridin-3-yl)-1H-pyrrole-2-carboxylate via Suzuki cross coupling

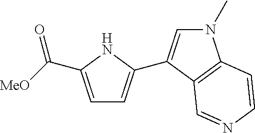 [SMILES: COC(=O)c1ccc(-c2cn(C)c3ccncc23)[nH]1]

1 eq. of 3-bromo-1H-pyrrolo[3,2-c]pyridine was dissolved in DMF at 0° C. under N₂. Then, 2.0 eq. of sodium hydride in DMF was added to the previous solution. The reaction mixture was stirred for 1 hour. In next step, 1.05 eq. of methyl iodide was added. The reaction was left to reach room temperature and stirred for 1 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was purified by liquid column chromatography with EtOAc/hexanes.

1.0 eq. of 3-bromo-1-methyl-1H-pyrrolo[3,2-c]pyridine was dissolved in H₂O:1,4-dioxane (1:5). Then, 1.0 eq. of Intermediate V-A and 3.0 eq. of K₂CO₃ were added to the flask. The reaction mixture was bubbled by argon gas for 10 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1-methyl-1H-pyrrolo[3,2-c]pyridin-3-yl)-1H-pyrrole-2-carboxylate

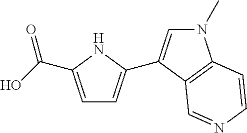 [SMILES: Cn1cc(-c2ccc(C(=O)O)[nH]2)c2cnccc21]

1 eq. of methyl 5-(1-methyl-1H-pyrrolo[3,2-c]pyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1-methyl-1H-pyrrolo[3,2-c]pyridin-3-yl)-1H-pyrrole-2-carboxylic acid and Intermediate III-A (1 eq.) was dissolved in DMF 3 mL. Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 1-5% MeOH/EtOAc to give Example 154 (10 mg, 96% purity by UV). LCMS Method A, Rt=4.23 min, m/z=468.2 [M+H]+, exact mass; 467.1569.

### Example 155. (S)-1-(5-(1-methyl-1H-pyrrolo[2,3-b]pyridin-3-yl)-1H-pyrrole-2-carbonyl)-N-(3,4,5-trifluorophenyl)pyrrolidine-3-carboxamide

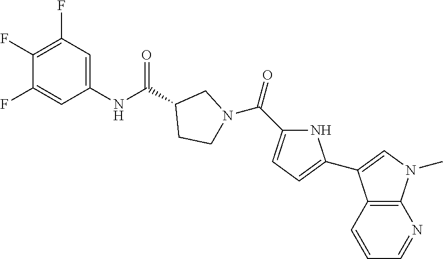 [SMILES: Cn1cc(-c2ccc(C(=O)N3CC[C@H](C(=O)Nc4cc(F)c(F)c(F)c4)C3)[nH]2)c2cccnc21]

### Step 1: Synthesis of methyl 5-(1-methyl-1H-pyrrolo[2,3-b]pyridin-3-yl)-1H-pyrrole-2-carboxylate via Suzuki cross coupling

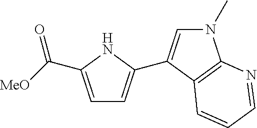 [SMILES: COC(=O)c1ccc(-c2cn(C)c3ncccc23)[nH]1]

1 eq. of 3-bromo-1H-pyrrolo[2,3-b]pyridine was dissolved in DMF at 0° C. under N₂. Then, 2.0 eq. of sodium hydride in DMF was added to the previous solution. The reaction mixture was stirred for 1 hour. In next step, 1.05 eq. of methyl iodide was added. The reaction was left to reach room temperature and stirred for 1 hour. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was purified by liquid column chromatography with EtOAc/hexanes.

1.0 eq. of 3-bromo-1-methyl-1H-pyrrolo[2,3-b]pyridine was dissolved in H₂O:1,4-dioxane (1:5). Then, 1.0 eq. of Intermediate V-A and 3.0 eq. of K₂CO₃ were added to the flask. The reaction mixture was bubbled by argon gas for 10 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1-methyl-1H-pyrrolo[2,3-b]pyridin-3-yl)-1H-pyrrole-2-carboxylate

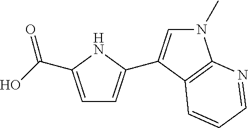 [SMILES: Cn1cc(-c2ccc(C(=O)O)[nH]2)c2cccnc21]

1 eq. of methyl 5-(1-methyl-1H-pyrrolo[2,3-b]pyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1-methyl-1H-pyrrolo[2,3-b]pyridin-3-yl)-1H-pyrrole-2-carboxylic acid and Intermediate III-A (1 eq.) was dissolved in DMF 3 mL. Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 1-5% MeOH/EtOAc to give Example 155 (15 mg, 98% purity by UV). LCMS Method A, Rt=4.71 min, m/z=468.2 [M+H]+, exact mass: 467.1569.

### Example 156. (S)—N-(3-chloro-4-fluorophenyl)-1-(5-(3,5-dimethylpyridin-4-yl)-1H-pyrrole-2-carbonyl)pyrrolidine-3-carboxamide

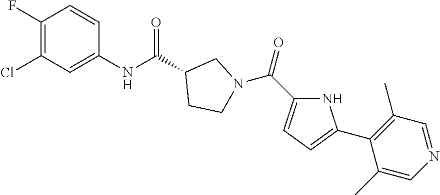 [SMILES: Cc1cncc(C)c1-c1ccc(C(=O)N2CC[C@H](C(=O)Nc3ccc(F)c(Cl)c3)C2)[nH]1]

### Step 1: Synthesis of methyl 5-(3,5-dimethylpyridin-4-yl)-1H-pyrrole-2-carboxylate via Suzuki cross coupling

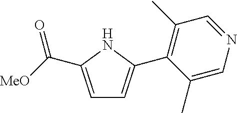 [SMILES: COC(=O)c1ccc(-c2c(C)cncc2C)[nH]1]

1.0 eq. of 3,5-Lutidine-N-Oxide was dissolved in dry carbon tetrachloride (0.015M) Then, 2.0 eq. of bromine and 2.0 eq. anhydrous potassium carbonate were added to the flask. This mixture was refluxed for 3 hours and then filtered. The solvent was removed under vacuum. To achieve pure product, crude product was purified by liquid column chromatography with EtOAc/hexanes.

Next, 0.7 eq. of LiAlH₄ was added to TiCl₄ (1.0 eq.) that was suspended in anhydrous THF under Ar. The reaction mixture was stirred for 15 mins at room temperature. Then, 1.0 eq. of 3-bromo-3,5-Lutidine-N-Oxide was added to the mixture at 0° C. and stirred for 30 mins at room temperature. The reaction was hydrolyzed by adding 1120 (2.5 mL per mmol) and then NH₄₀-1 (33% in water. 2.5 mL per mmol). The mixture was extracted by Et₂O for 3 times and dried by anhydrous Na₂SO₄. The solvent was removed and the crude product was used in the next step.

1.0 eq. of 4-bromo-3,5-dimethylpyridine was dissolved in H₂O:1,4-dioxane (1:5). Then, 1.0 eq. of Intermediate V-A and 3.0 eq. of K₂CO₃ were added to the flask. The reaction mixture was bubbled by argon gas for 10 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(3,5-dimethylpyridin-4-yl)-1H-pyrrole-2-carboxylate

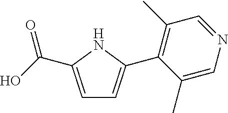 [SMILES: Cc1cncc(C)c1-c1ccc(C(=O)O)[nH]1]

1 eq. of methyl 5-(3,5-dimethylpyridin-4-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and transferred the solution into another flask and dried. Dried crude product was used in further synthesis.

### Step 3: Amide Formation

1 eq. of 5-(3,5-dimethylpyridin-4-yl)-1H-pyrrole-2-carboxylic acid and Intermediate III-I (1 eq.) was dissolved in DMF 3 mL. Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 100% EtOAc to give Example 156 (9 mg, 98% purity by UV). LCMS Method A, Rt=4.24 min, m/z=441.1 [M+H]+, exact mass: 440.1415.

### Example 157. (S)-1-(5-(7-methyl-1H-indazol-5-yl)-1H-pyrrole-2-carbonyl)-N-(3,4,5-trifluorophenyl)pyrrolidine-3-carboxamide

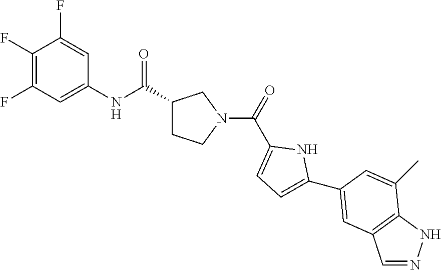 [SMILES: Cc1cc(-c2ccc(C(=O)N3CC[C@H](C(=O)Nc4cc(F)c(F)c(F)c4)C3)[nH]2)cc2cn[nH]c12]

### Step 1: Synthesis of methyl 5-(7-methyl-1H-indazol-5-yl)-1H-pyrrole-2-carboxylatevia Suzuki cross coupling

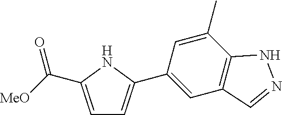 [SMILES: COC(=O)c1ccc(-c2cc(C)c3[nH]ncc3c2)[nH]1]

1.2 eq. of 5-bromo-7-methyl-1H-indazole was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(7-methyl-1H-indazol-5-yl)-1H-pyrrole-2-carboxylate

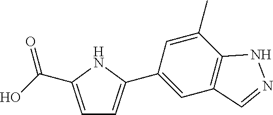 [SMILES: Cc1cc(-c2ccc(C(=O)O)[nH]2)cc2cn[nH]c12]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was stirred at room temperature for overnight. The reaction was concentrated in vacuo. The reaction was acidified by 2N HCl until pH of solution adjusted approximately to 3. During this acidification, a white precipitate formed. The solid was collected via filtration and washed with water, providing the product as a white solid.

### Step 3: Amide Formation

1 eq. of 5-(7-methyl-1H-indazol-5-yl)-1H-pyrrole-2-carboxylic acid and Intermediate III-A (1 eq.) was dissolved in DMF 3 mL Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 1-5% MeOH/EtOAc to give Example 157 (13 mg, 98% purity by UV). LCMS Method A, Rt=4.59 min, m/z=468.2 [M+H]⁺, exact mass: 467.1569.

### Example 158. (S)-1-(5-(1-methyl-1H-pyrrolo[2,3-c]pyridin-3-yl)-1H-pyrrole-2-carbonyl)-N-(3,4,5-trifluorophenyl)pyrrolidine-3-carboxamide

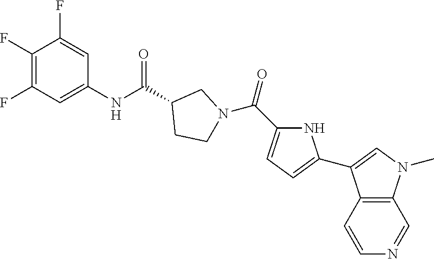 [SMILES: Cn1cc(-c2ccc(C(=O)N3CC[C@H](C(=O)Nc4cc(F)c(F)c(F)c4)C3)[nH]2)c2ccncc21]

### Step 1: Synthesis f methyl 5-(1-methyl-1H-pyrrolo[2,3-c]pyridin-3-yl)-1H-pyrrole-2-carboxylate via Suzuki cross coupling

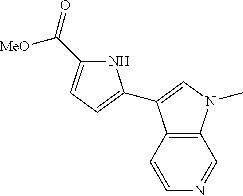 [SMILES: COC(=O)c1ccc(-c2cn(C)c3cnccc23)[nH]1]

1 eq. of 3-bromo-1H-pyrrolo[2,3-c]pyridine was dissolved in DMF at 0° C. under N₂. Then, 1.6 eq. of sodium hydride in DMF was added to the previous solution. The reaction mixture was stirred for 10 mins. In next step, 1.05 eq. of methyl iodide was added. The reaction was left to reach room temperature and stirred for 2 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was used in the next step.

1.2 eq. of 3-bromo-1-methyl-1H-pyrrolo[2,3-c]pyridine was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1-methyl-1H-pyrrolo[2,3-c]pyridin-3-yl)-1H-pyrrole-2-carboxylate

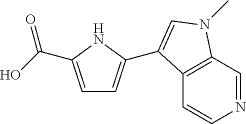 [SMILES: Cn1cc(-c2ccc(C(=O)O)[nH]2)c2ccncc21]

1 eq. of methyl 5-(1-methyl-1H-pyrrolo[2,3-c]pyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1-methyl-1H-pyrrolo[2,3-c]pyridin-3-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-A was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 158 (5 mg, 90% purity by UV). LCMS Method A, Rt=4.19 min, m/z=468.2 [M+H]*, exact mass: 467.1569.

### Example 159. (S)-1-(5-(6-methyl-1H-pyrrolo[2,3-b]pyridin-5-yl)-1H-pyrrole-2-carbonyl)-N-(3,4,5-trifluorophenyl)pyrrolidine-3-carboxamide

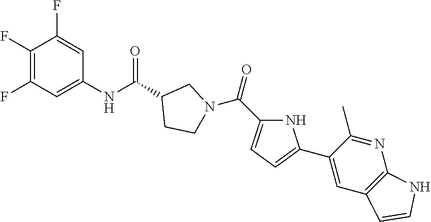

### Step 1: Synthesis of methyl 5-(6-methyl-1H-pyrrolo[2,3-b]pyridin-5-yl)-1H-pyrrole-2-carboxylate via Suzuki cross coupling

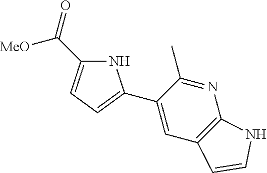 [SMILES: COC(=O)c1ccc(-c2cc3cc[nH]c3nc2C)[nH]1]

1.2 eq. of 5-bromo-6-methyl-1H-pyrrolo[2,3-b]pyridine was dissolved in H₂O:1,4-dioxane) 1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(6-methyl-1H-pyrrolo[2,3-b]pyridin-5-yl)-1H-pyrrole-2-carboxylate

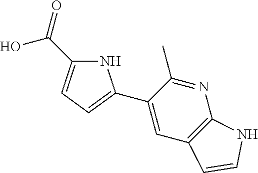 [SMILES: Cc1nc2[nH]ccc2cc1-c1ccc(C(=O)O)[nH]1]

1 eq. of methyl 5-(6-methyl-1H-pyrrolo[2,3-b]pyridin-5-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(6-methyl-1H-pyrrolo[2,3-b]pyridin-5-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-A was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 159 (6 mg, 97% purity by UV). LCMS Method A, Rt=4.47 min, m/z=468.2 [M+H]*, exact mass: 467.1569.

### Example 160. (S)-1-(5-(1-cyclopropyl-6-methyl-1H-indazol-5-yl)-1H-pyrrole-2-carbonyl)-N-(3,4,5-trifluorophenyl)pyrrolidine-3-carboxamide

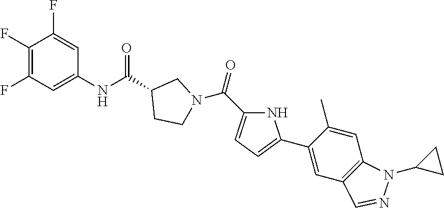 [SMILES: Cc1cc2c(cnn2C2CC2)cc1-c1ccc(C(=O)N2CC[C@H](C(=O)Nc3cc(F)c(F)c(F)c3)C2)[nH]1]

### Step 1: Synthesis of methyl 5-(1-cyclopropyl-6-methyl-1H-indazol-5-yl)-1H-pyrrole-2-carboxylate via Suzuki cross coupling

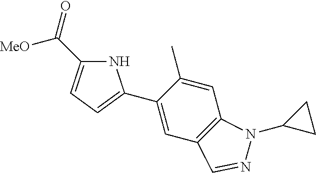 [SMILES: COC(=O)c1ccc(-c2cc3cnn(C4CC4)c3cc2C)[nH]1]

1 eq. of 5-bromo-6-methyl-1H-indazole was dissolved in DCE. The solution was mixed with 1.1 eq. of cyclopropylboronic acid and 2 eq. of Na₂CO₃ under N₂. Then, 1 eq. of pyridine was added to the reaction and followed by 1 eq. of copper (II) acetate. The reaction was refluxed for overnight. The reaction was worked up by adding ammonia solution. The organic layer was washed by brine and purified by column chromatography with EtOAc/hexanes.

1.2 eq. of 5-bromo-1-cyclopropyl-6-methyl-1H-indazole was dissolved in H₂O:1,4-dioxane) 1:5. (Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1-cyclopropyl-6-methyl-1H-indazol-5-yl)-1H-pyrrole-2-carboxylate

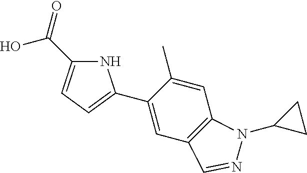 [SMILES: Cc1cc2c(cnn2C2CC2)cc1-c1ccc(C(=O)O)[nH]1]

1 eq. of methyl 5-(1-cyclopropyl-6-methyl-1H-indazol-5-yl)-1H-pyrrole-2-carboxylate was dissolved in THE. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1-cyclopropyl-6-methyl-1H-indazol-5-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-A was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 160 (12.5 mg, 99% purity by UV). LCMS Method A, Rt=5.00 min, m/z=508.2 [M+H]+, 530.2 [M+Na]+, exact mass: 507.1882. ¹H-NMR (500 MHz, Acetone-d₆) δ 10.61 (s, 1H), 9.82 (s, 1H), 7.89 (s, 1H), 7.77 (s, 1H), 7.59-7.52 (m, 3H), 6.74 (t, J=5.0 Hz, 1H), 6.31 (t, J=3.1 Hz, 1H), 4.18-3.82 (m), 3.73-3.61 (m), 3.39 (s), 3.32-3.15 (m), 2.86 (s), 2.56 (d, J=14.8 Hz, 3H), 2.41-2.24 (m, 1H), 2.07-2.04 (overlap, 2H) 1.19-1.09 (m, 4H).

### Example 161. (S)—N-(3-cyano-4-fluorophenyl)-1-(5-(2-cyanopyridin-3-yl)-1H-pyrrole-2-carbonyl)pyrrolidine-3-carboxamide

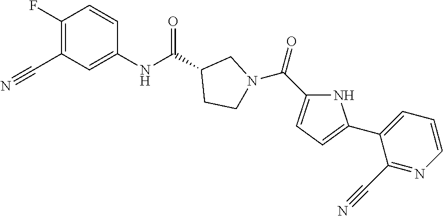 [SMILES: N#Cc1cc(NC(=O)[C@H]2CCN(C(=O)c3ccc(-c4cccnc4C#N)[nH]3)C2)ccc1F]

### Step 1: Synthesis of methyl 5-(2-cyanopyridin-3-yl)-1H-pyrrole-2-carboxylate via Suzuki cross coupling

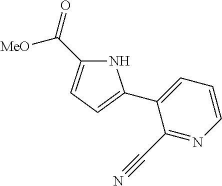 [SMILES: COC(=O)c1ccc(-c2cccnc2C#N)[nH]1]

1.2 eq. of 3-bromopicolinonitrile was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes

### Step 2: Hydrolysis of methyl 5-(2-cyanopyridin-3-yl)-1H-pyrrole-2-carboxylate

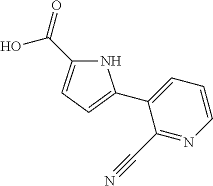 [SMILES: N#Cc1ncccc1-c1ccc(C(=O)O)[nH]1]

1 eq. of methyl 5-(2-cyanopyridin-3-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2 The reaction mixture was concentrated by rotavapor. The solid was washed with 15% MeOH/EtOAc and the solution was transferred into another flask and dried. Dried crude product was used in further synthesis.

### Step 3: Amide formation

1 eq. of 5-(2-cyanopyridin-3-yl)-1H-pyrrole-2-carboxylic acid was dissolved in DMF. Then, 2 eq. of EDCI-HCl, HOBt, and 10 eq of DIPEA were added. The reaction mixture was stirred at room temperature for 10 mins, then Intermediate III-E was added. The mixture was stirred for overnight at room temperature. Then, the mixture was diluted with water and extracted the product with EtOAc for 3 times. The organic layer was washed with brine. The crude reaction was purified by column chromatography with MeOH/EtOAc to afford Example 161 (2.1 mg, 90% purity by UV) LCMS Method B, Rt=4.57 min, m/z=427.1] M−H[⁺, exact mass: 428.1397. ¹H-NMR (500 MHz, Acetone-d₆) δ 10.61 (s, 1H), 9.82 (s, 1H), 7.89 (s, 1H), 7.77 (s, 1H), 7.59-7.52 (m, 3H), 6.74 (t, J=5.0 Hz, 1H), 6.31 (t, J=3.1 Hz, 1H), 4.18-3.82 (m), 3.73-3.61 (m), 3.39 (s), 3.32-3.15 (m), 2.86 (s), 2.56 (d, J=14.8 Hz, 3H), 2.41-2.24 (m, 1H), 2.07-2.04 (overlap, 2H) 1.19-1.09 (m, 4H).

### Example 162. (S)-1-(5-(1H-benzo[d][1,2,3]triazol-5-yl)-1H-pyrrole-2-carbonyl)-N-(3,4,5-trifluorophenyl)pyrrolidine-3-carboxamide

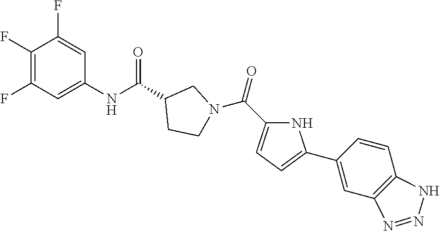

### Step 1: Synthesis of methyl 5-(1H-benzo[d][1,2,3]triazol-5-yl)-1H-pyrrole-2-carboxylate via Suzuki cross coupling

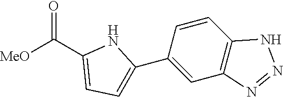 [SMILES: COC(=O)c1ccc(-c2ccc3[nH]nnc3c2)[nH]1]

1.2 eq. of 5-bromo-1H-benzo[d][1,2,3]triazole was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1H-benzo[d][1,2,3]triazol-5-yl)-1H-pyrrole-2-carboxylate

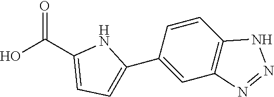 [SMILES: O=C(O)c1ccc(-c2ccc3[nH]nnc3c2)[nH]1]

1 eq. of methyl 5-(1H-benzo[d][1,2,3]triazol-5-yl)-1H-pyrrole-2-carboxylate was dissolved in THF. Then, 5 eq. of LiOH was dissolved in H₂O and added to the THF solution. The reaction mixture was stirred at room temperature for overnight. The reaction was concentrated in vacuo. The reaction was acidified by 2N HCl until pH of solution adjusted approximately to 3. During this acidification, a white precipitate formed. The solid was collected via filtration and washed with water, providing the product as a white solid.

### Step 3: Amide Formation

1 eq. of 5-(1H-benzo[d][1,2,3]triazol-5-yl)-1H-pyrrole-2-carboxylic acid and Intermediate III-A (1 eq.) was dissolved in DMF 3 mL. Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 1% TFA in 5-10% MeOH/EtOAc to give Example 162 (2.1 mg, 91% purity by UV). LCMS Method A, Rt=4.45 min, m/z=455.2 [M+H]+, exact mass: 454.1365.

### Example 163. (S)-1-(5-(1,6-dimethyl-1H-indazol-5-yl)-1H-pyrrole-2-carbonyl)-N-(3,4,5-trifluorophenyl)pyrrolidine-3-carboxamide

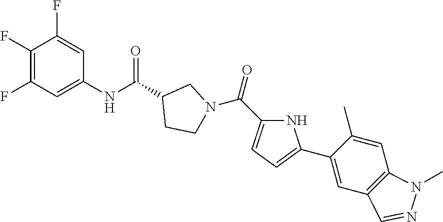

Step 1: Synthesis of methyl 5-(1,6-dimethyl-H-indazol-5-yl)-1H-pyrrole-2-carboxylate via Suzuki cross coupling

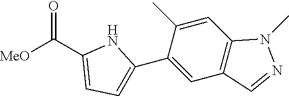 [SMILES: COC(=O)c1ccc(-c2cc3cnn(C)c3cc2C)[nH]1]

1 eq. of 5-bromo-6-methyl-1H-indazole was dissolved in DMF at 0° C. under N₂. Then, 2.0 eq. of sodium hydride in DMF was added to the previous solution. The reaction mixture was stirred for 10 mins. In next step, 1.05 eq. of methyl iodide was added. The reaction was left to reach room temperature and stirred for 1 hours. The reaction was quenched by cooled saturated ammonium chloride solution. The desired product was extracted by EtOAc for 3 times. Next, the organic layer was washed by saturated NaCl solution and dried by anhydrous Na₂SO₄. The solvent was removed and the crude product was purified by liquid column chromatography with EtOAc/hexanes.

1.2 eq. of 5-bromo-1,6-dimethyl-1H-indazole was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(1,6-dimethyl-1H-indazol-5-yl)-1H-pyrrole-2-carboxylate

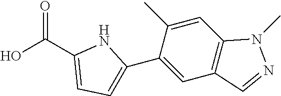 [SMILES: Cc1cc2c(cnn2C)cc1-c1ccc(C(=O)O)[nH]1]

1 eq. of methyl 5-(1,6-dimethyl-1H-indazol-5-yl)-1H-pyrrole-2-carboxylate was dissolved in THF. Then, 5 eq. of LiOH was dissolved in H₂O and added to the THF solution. The reaction mixture was stirred for overnight at room temperature. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(1,6-dimethyl-1H-indazol-5-yl)-1H-pyrrole-2-carboxylic acid and Intermediate III-A (1 eq.) was dissolved in DMF 3 mL. Then, 2 eq. of EDCI-HCl, HOBt, and 1 mL of DIPEA were added. The reaction mixture was stirred at room temperature. The mixture was stirred for overnight at room temperature. Then, the mixture was diluted with water and extracted the product with EtOAc for 3 times. The organic layer was washed with brine. The crude reaction was purified by column chromatography with 1-5% MeOH/EtOAc to give Example 163 (8.6 mg, 99% purity by UV). LCMS Method A, Rt=4.83 min, m/z=482.2 [M+H]+, exact mass: 481.1726.

### Example 164. (3S,4R)-1-(5-(3,5-dimethyl-1H-pyrazol-4-yl)-1H-pyrrole-2-carbonyl)-N-(4-fluoro-3-methylphenyl)-4-methylpyrrolidine-3-carboxamide

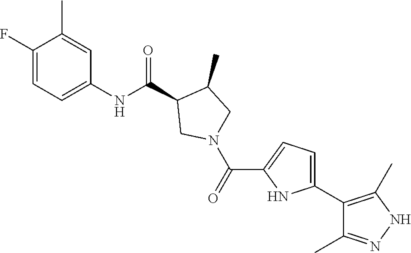 [SMILES: Cc1cc(NC(=O)[C@@H]2CN(C(=O)c3ccc(-c4c(C)n[nH]c4C)[nH]3)C[C@@H]2C)ccc1F]

### Step 1: Synthesis of methyl-5-(3,5-dimethyl-1H-pyrazol-4-yl)-1H-pyrrole-2-carboxylate via Suzuki cross coupling

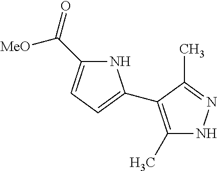 [SMILES: COC(=O)c1ccc(-c2c(C)n[nH]c2C)[nH]1]

1.2 eq. of 4-iodo-3,5-dimethyl-1H-pyrazole was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(3,5-dimethyl-1H-pyrazol-4-yl)-1H-pyrrole-2-carboxylate

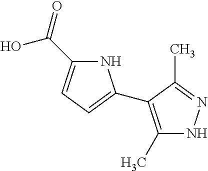

1 eq. of methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(3,5-dimethyl-1H-pyrazol-4-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-Q was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 164 (4.11 mg, 98% purity by UV). LCMS Method B, Rt=4.34 min, m/z=424.2 [M+H]⁺, exact mass: 423.2071.

### Example 165. (3S,4R)-1-(5-(4,6-dimethylpyrimidin-5-yl)-1H-pyrrole-2-carbonyl)-N-(4-fluoro-3-methylphenyl)-4-methylpyrrolidine-3-carboxamide

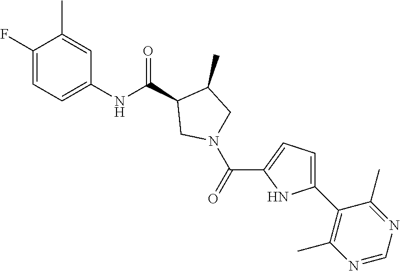 [SMILES: Cc1cc(NC(=O)[C@@H]2CN(C(=O)c3ccc(-c4c(C)ncnc4C)[nH]3)C[C@@H]2C)ccc1F]

### Step 1: Synthesis of methyl 5-(4,6-dimethylpyrimidin-5-yl)-1H-pyrrole-2-carboxylate via Suzuki cross coupling

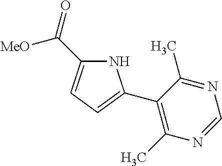 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hours. To achieve pure product, crude product was purified by liquid column chromatography with EtOAc/hexanes.

### Step 2: Hydrolysis of methyl 5-(4,6-dimethylpyrimidin-5-yl)-1H-pyrrole-2-carboxylate

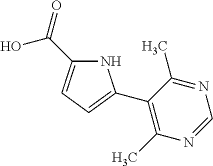 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(4,6-dimethylpyrimidin-5-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-Q was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 165 (6.2 mg, 97% purity by UV). LCMS Method B, Rt=4.49 min, m/z=436.2 [M+H]⁺, exact mass: 435.2071.

### Example 166. (3S,4R)—N-(4-fluoro-3-methylphenyl)-4-methyl-1-(5-(5-methyl-3-(trifluoromethyl)-1H-pyrazol-4-yl)-1H-pyrrole-2-carbonyl)pyrrolidine-3-carboxamide

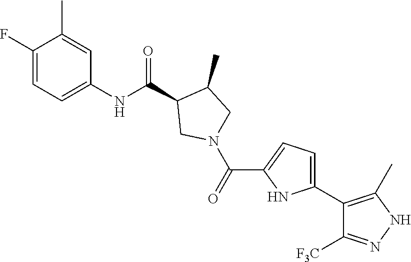 [SMILES: Cc1cc(NC(=O)[C@@H]2CN(C(=O)c3ccc(-c4c(C(F)(F)F)n[nH]c4C)[nH]3)C[C@@H]2C)ccc1F]

### Step 1: Synthesis of methyl 5-(5-methyl-3-(trifluoromethyl)-1H-pyrazol-4-yl)-1H-pyrrole-2-carboxylate via Suzuki cross coupling

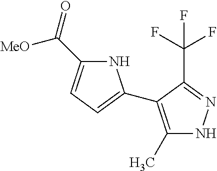 [SMILES: COC(=O)c1ccc(-c2c(C(F)(F)F)n[nH]c2C)[nH]1]

1.2 eq. of 4-bromo-5-methyl-3-(trifluoromethyl)-1H-pyrazole was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(5-methyl-3-(trifluoromethyl)-1H-pyrazol-4-yl)-1H-pyrrole-2-carboxylate

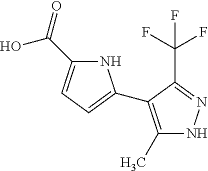 [SMILES: Cc1[nH]nc(C(F)(F)F)c1-c1ccc(C(=O)O)[nH]1]

1 eq. of methyl 5-(5-methyl-3-(trifluoromethyl)-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(5-methyl-3-(trifluoromethyl)-1H-pyrazol-4-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-Q was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 166 (2.79 mg, 97% purity by UV). LCMS Method A, Rt=4.62 min, m/z=478.2 [M+H]+, exact mass: 477.1788.

### Example 167. (3S,4S)-1-(3-chloro-1H-indole-2-carbonyl)-N-(4-fluoro-3-methylphenyl)-4-methylpyrrolidine-3-carboxamide

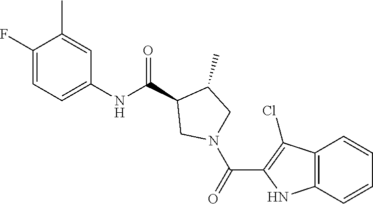 [SMILES: Cc1cc(NC(=O)[C@@H]2CN(C(=O)c3[nH]c4ccccc4c3Cl)C[C@H]2C)ccc1F]

The mixture of Intermediate III-R (1 eq.) and 3-chloro-1H-indole-2-carboxylic acid (1.2 eq.) was dissolved in DMF 3 mL. Then, 1.5 eq. of HATU, and 1 mL of DIPEA were added. The mixture was stirred for overnight at room temperature. Then, the mixture was diluted with water and extracted the product with EtOAc for 3 times. The organic layer was washed with brine. The crude reaction was purified by column chromatography with 70% EtOAc/hexanes to afford Example 167 (2.91 mg, 97% purity by UV) as a white solid. LCMS Method B, Rt=4.90 min, m/z=414.1 [M+H]*, 436.1 [M+Na]⁺, exact mass: 413.1306.

### Example 168. (3S,4S)-1-(5-(4,6-dimethylpyrimidin-5-yl)-1H-pyrrole-2-carbonyl)-N-(4-fluoro-3-methylphenyl)-4-methylpyrrolidine-3-carboxamide

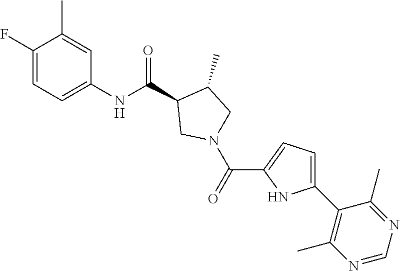 [SMILES: Cc1cc(NC(=O)[C@@H]2CN(C(=O)c3ccc(-c4c(C)ncnc4C)[nH]3)C[C@H]2C)ccc1F]

### Step 1: Synthesis of methyl 5-(4,6-dimethylpyrimidin-5-yl)-1H-pyrrole-2-carboxylate via Suzuki

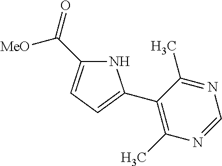 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hours. To achieve pure product, crude product was purified by liquid column chromatography with EtOAc/hexanes.

### Step 2: Hydrolysis of methyl 5-(4,6-dimethylpyrimidin-5-yl)-1H-pyrrole-2-carboxylate

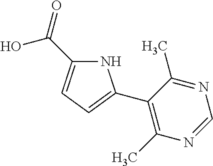 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(4,6-dimethylpyrimidin-5-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-R was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 168 (5.51 mg, 91% purity by UV). LCMS Method B, Rt=4.51 min, m/z=436.2 [M+H]⁺, exact mass: 435.2071.

### Example 169. (3S,4S)-1-(5-(3,5-dimethyl-1H-pyrazol-4-yl)-1H-pyrrole-2-carbonyl)-N-(4-fluoro-3-methylphenyl)-4-methylpyrrolidine-3-carboxamide

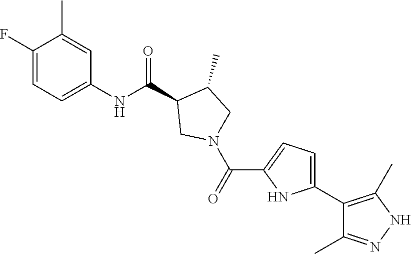 [SMILES: Cc1cc(NC(=O)[C@@H]2CN(C(=O)c3ccc(-c4c(C)n[nH]c4C)[nH]3)C[C@H]2C)ccc1F]

### Step 1: Synthesis of methyl 5-(3,5-dimethyl-1H-pyrazol-4-yl)-1H-pyrrole-2-carboxylate via Suzuki cross coupling

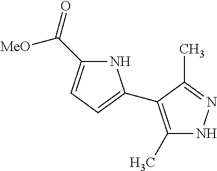 [SMILES: COC(=O)c1ccc(-c2c(C)n[nH]c2C)[nH]1]

1.2 eq. of 4-iodo-3,5-dimethyl-1H-pyrazole was dissolved in H₂O:1,4-dioxane (1:5). Then, 1 eq. of Intermediate V-A and 3 eq. of K₂CO₃ were added to the flask. The reaction mixture was bubbled by argon gas for 5-15 mins. Then, 10 mol % Pd(PPh₃)₄ was added to the reaction. Reaction was heated under argon at 110° C. for 1 hour. To achieve pure product, crude product was purified by liquid column chromatography with EtOAc/hexanes.

### Step 2: Hydrolysis of methyl 5-(3,5-dimethyl-1H-pyrazol-4-yl)-1H-pyrrole-2-carboxylate

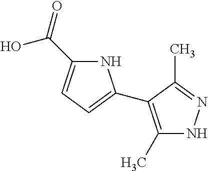

1 eq. of methyl 5-(3,5-dimethyl-1H-pyrazol-4-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(3,5-dimethyl-1H-pyrazol-4-yl)-1H-pyrrole-2-carboxylic acid was dissolved in DMF. Then, added 1.5 eq. of HATU and 3 eq. of DIPEA. The reaction mixture was stirred at room temperature for 10 mins. Then, 1.1 eq. of Intermediate III-R was added to the solution. The mixture was stirred for overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 169 (2.56 mg, 87% purity by UV). LCMS Method B, Rt=4.43 min, m/z=424.2 [M+H]⁺, exact mass: 423.2071.

### Example 170. (2S,3S)—N-(3-cyclopropyl-4-fluorophenyl)-1-(5-(4,6-dimethylpyrimidin-5-yl)-1H-pyrrole-2-carbonyl)-2-methylpyrrolidine-3-carboxamide

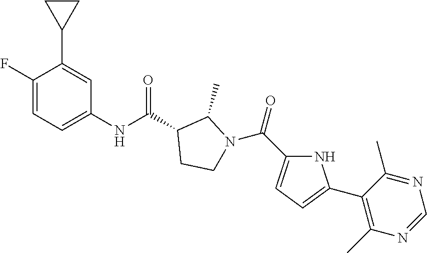 [SMILES: Cc1ncnc(C)c1-c1ccc(C(=O)N2CC[C@H](C(=O)Nc3ccc(F)c(C4CC4)c3)[C@@H]2C)[nH]1]

Step 1: Synthesis of methyl 5-(4,6-dimethylpyrimidin-5-yl)-1H-pyrrole-2-carboxylate via Suzuki cross coupling

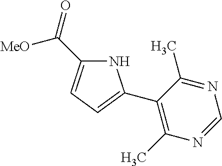 [SMILES: COC(=O)c1ccc(-c2c(C)ncnc2C)[nH]1]

1.2 eq. of 5-bromo-4,6-dimethylpyrimidine was dissolved in H₂O:1,4-dioxane (1:5). Then, 1 eq. of Intermediate V-A and 3 eq. of K₂COM were added to the flask. The reaction mixture was bubbled by argon gas for 5-15 mins. Then, 10 mol % Pd(PPh₃)₄ was added to the reaction. Reaction was heated under argon at 110° C. for 1 hours. To achieve pure product, crude product was purified by liquid column chromatography with EtOAc/hexanes.

### Step 2: Hydrolysis of methyl 5-(4,6-dimethylpyrimidin-5-yl)-1H-pyrrole-2-carboxylate

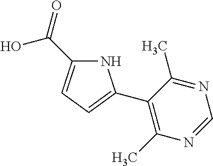 [SMILES: Cc1ncnc(C)c1-c1ccc(C(=O)O)[nH]1]

1 eq. of methyl 5-(4,6-dimethylpyrimidin-5-yl)-1H-pyrrole-2-carboxylate was dissolved in THF. Then, 5 eq. of LiOH was dissolved in H₂O and added to the THF solution. The reaction mixture was heated until it reached to 90° C. and kept heating for an hour. The reaction was acidified by 2N HCl until pH of solution adjusted approximately to 2. The reaction mixture was concentrated by rotavapor. The solid was washed with 15% MeOH/EtOAc and the solution was transferred into another flask and dried. Dried crude product was used in further synthesis.

### Step 3: Amide Formation

1 eq. of 5-(4,6-dimethylpyrimidin-5-yl)-1H-pyrrole-2-carboxylic acid was dissolved in DMF. Then, 1.5 eq. of HATU and 3 eq. of DIPEA were added. The reaction mixture was stirred at room temperature for 10 mins. Then, 1.1 eq. of Intermediate III-P was added to the solution. The mixture was stirred overnight at room temperature. Then, the mixture was diluted with water. The product was extracted with EtOAc for 3 times. The organic layer was washed by brine and purified by column chromatography with MeOH/EtOAc. The desired fractions were pooled and the solvent was removed under reduced pressure to afford Example 170 (2.79 mg, 93% purity by UV). LCMS Method B, Rt=4.65 min, m/z=462.2 [M+H]+, exact mass: 461.2227.

### Biological Activity of the Compounds of the Present Disclosure

The biological activity of the compounds of the present disclosure was determined utilising the assay described herein.

### Example 171. In Vitro Anti-HBV Activity in HepG2.2.15 Cell Line

The stable transfected HBV cell line, called HepG2.2.15 cells was a valuable model with high reproducibility and commonly used for drug screening. To identify the anti-HBV activity, HepG2.2.15 cells were seeded at 5×10⁵ cells/well with high glucose Dulbecco's modified Eagle medium (DMEM) containing 10% heat inactivated fetal bovine serum (FBS), 300 mg/L G418, non-essential amino acids (NEAA) 100 units/ml Antibiotic-Antimycotic, and allowed to stand for an overnight in an incubator set at 37° C. and 5% C₀₂. HepG2.2.15 cells in each well were treated with the medium containing a compound (1 μM with 0.5% dimethyl sulfoxide (DMSO final concentration) for three days in the incubator. After that the medium was removed and fresh medium containing the compound was added to the cells for another 3 days. After 6 days of treatment, intracellular HBV DNA was extracted and determined by using quantitative real-time PCR (qPCR) technique with specific primers. Intrahepatic HBV DNA viral load was reported as percentage compared to vehicle control (0.5% DMSO) as shown in Table A.

For % HBV DNA at 1 μM values shown in Table A, “A” means %>90. “B” means % ranging between 80 and 90; “C” means % ranging between 70 and 80; “D” means % ranging between 60 and 70; “E” means % ranging between 45 and 60, “F” means %<45.

TABLE A
Compounds and biological activity

| Compound | % HBV DNA | Compound | % HBV DNA |
|---|---|---|---|
| No. | at 1 μM | No. | at 1 μM |
| 1 | E | 24 | D |
| 2 | F | 25 | D |
| 3 | C | 26 | B |
| 4 | F | 27 | A |
| 5 | B | 28 | A |
| 6 | A | 29 | A |
| 7 | D | 30 | A |
| 8 | B | 31 | A |
| 9 | B | 32 | B |
| 10 | C | 33 | B |
| 11 | C | 34 | A |
| 12 | B | 35 | C |
| 13 | D | 36 | C |
| 14 | C | 37 | A |
| 15 | D | 38 | A |
| 16 | A | 39 | C |
| 17 | D | 40 | A |
| 18 | C | 41 | F |
| 19 | F | 42 | F |
| 20 | F | 43 | E |
| 21 | A | 44 | C |
| 22 | D | 45 | D |
| 23 | C | 46 | D |
| 47 | B | 83 | F |
| 48 | B | 84 | A |
| 49 | A | 85 | F |
| 50 | C | 86 | F |
| 51 | E | 87 | F |
| 52 | C | 88 | F |
| 53 | C | 89 | F |
| 54 | C | 90 | C |
| 55 | A | 91 | F |
| 56 | A | 92 | E |
| 57 | A | 93 | F |
| 58 | C | 94 | D |
| 59 | E | 95 | E |
| 60 | F | 96 | E |
| 61 | D | 97 | F |
| 62 | E | 98 | C |
| 63 | C | 99 | F |
| 64 | C | 100 | F |
| 65 | F | 101 | F |
| 66 | E | 102 | F |
| 67 | E | 103 | F |
| 68 | F | 104 | F |
| 69 | C | 105 | F |
| 70 | E | 106 | F |
| 71 | C | 107 | C |
| 72 | D | 108 | E |
| 73 | E | 109 | D |
| 74 | F | 110 | F |
| 75 | D | 111 | F |
| 76 | E | 112 | D |
| 77 | F | 113 | F |
| 78 | C | 114 | F |
| 79 | F | 115 | F |
| 80 | F | 116 | E |
| 81 | E | 117 | E |
| 82 | E | 118 | D |
| 119 | F | 145 | C |
| 120 | F | 146 | C |
| 121 | B | 147 | E |
| 122 | F | 148 | E |
| 123 | F | 149 | F |
| 124 | E | 150 | F |
| 125 | F | 151 | C |
| 126 | F | 152 | A |
| 127 | F | 153 | F |
| 128 | F | 154 | E |
| 129 | F | 155 | C |
| 130 | F | 156 | F |
| 131 | E | 157 | E |
| 132 | E | 158 | D |
| 133 | E | 159 | F |
| 134 | F | 160 | A |
| 135 | F | 161 | C |
| 136 | A | 162 | C |
| 137 | E | 163 | F |
| 138 | F | 164 | F |
| 139 | F | 165 | F |
| 140 | A | 166 | F |
| 141 | F | 167 | E |
| 142 | A | 168 | E |
| 143 | A | 169 | F |
| 144 | A | 170 | F |

### Example 172. Effect on Intrahepatic HBV DNA Levels and Cytotoxicity

HepG2.2.15 cells were treated with compounds, at various concentrations in 10% FCS containing medium, at 37° C. for three days. After that the medium was removed and fresh medium containing the compound was added to the cells for another 3 days. After 6 days of treatment, intrahepatic HBV DNA from cell lysates were isolated. Then qPCR reaction of DNA was performed to measure total HBV DNA levels using specific primer set. The fifty-percent effective concentrations (EC₅₀) for HBV DNA inhibition, relative to no drug controls, was determined using nonlinear fitting curve model. EC₅₀ ranges for HBV DNA inhibition were as follows: A>0.3 μM, B=0.05-0.3 μM, C<0.05 μM (Table B).

### Cytotoxicity in HepG2 Cells

HepG2.2.15 cells were seeded onto 96-well culture plate and allowed to adhere for 24 hours. The cells were treated with various concentrations of the compounds for 6 days. After treatment, the culture media were discarded and further incubated with serum-free media containing 0.5 μg/ml of 3-(4,5-dimethylthiazol-2-yl)-2,5-diphenyl tetrazolium bromide (MTT) for 2 hour. The resulting formazan crystals were completely dissolved in dimethyl sulfoxide (DMSO), and then measured absorbance at 570 nm using microplate reader. The concentrations of compounds producing 50% cell death (CC₅₀) were determined from a fitting of concentration-response curve (% cell viability versus concentration) to a four-parameter equation. CC₅₀ ranges were as follows: A>25 μM, B=10-25 μM and C<10 μM (Table B).

TABLE B

| | Anti- | | | Anti- | |
|---|---|---|---|---|---|
| | HBV | | | HBV | |
| | DNA | HepG2.1.15 | | DNA | HepG2.1.15 |
| Compound | (EC50, | cytotoxicity | Compound | (EC50, | cytotoxicity |
| No. | μM) | (CC₅₀, μM) | No. | μM) | (CC₅₀, μM) |
| 2 | C | A | 111 | C | C |
| 4 | A | A | 113 | C | B |
| 19 | B | A | 114 | C | B |
| 20 | A | B | 115 | B | A |
| 41 | B | — | 116 | B | A |
| 42 | B | A | 117 | C | A |
| 60 | B | A | 119 | B | A |
| 65 | B | A | 120 | B | A |
| 70 | B | A | 122 | B | B |
| 74 | B | B | 123 | B | B |
| 83 | C | A | 125 | C | A |
| 85 | C | B | 127 | B | — |
| 99 | C | B | 128 | B | B |
| 100 | C | B | 135 | B | — |
| 101 | B | A | 138 | B | B |
| 102 | C | A | 149 | C | B |
| 103 | C | B | 150 | B | A |
| 104 | C | B | 164 | C | A |
| 105 | C | C | 165 | B | A |
| 110 | B | A | 166 | C | A |

### ANTI-HBV Activity in the Presence of Human Serum

HepG2.2.15 cells were treated with test compounds, at various concentrations in cell culture media containing either 2% or 40% human serum (HS), at 37° C. for 6 days. After treatment, intrahepatic HBV DNA from cell lysates were isolated, and quantified by qPCR using specific primers. The fifty-percent effective concentrations (EC₅₀) for HBV DNA inhibition, relative to no drug controls, was determined using nonlinear fitting curve model. Fold change in EC₅₀ was calculated as the ratio of EC₅₀ in 40% HS to that in 2% HS for each compound.

The representative compounds (e.g. Examples 42, 83, 85, 99, 110, 149) showed low fold change in EC₅₀ (≤6 folds) in the presence of 40% HS.

### Example 173. Capsid Formation Assay for Use in Determining HBV Capsid Assembly by Size Exclusion Chromatography (SEC) and Electron Microscopy (EM)

Hepatitis B virus (HBV) capsid is a step in virus propagation and is mediated by the core protein. HBV core C-terminally truncated protein (HBV Cp149) may assemble into a capsid. Capsid formation was analyzed for the instant compounds as compared to a class I or class U compound. The class I compound induces the formation of morphologically aberrant empty structures (i.e., BAY 41-4109) and the class II compound induces the formation of intact empty capsids (i.e., NVR 3-778).

HBV C₁₄₉ protein was prepared based on a published method [Zlotnick, A et al; Nat Protoc 2007, 2(3), 490-498] with slight modifications. A compound (50 μM) was incubated with 15 μM of HBV core protein (Cp149) in 50 mM HEPES buffer, pH 7.5 and 150 mM NaCl. The mixture was incubated for 16 hours at 21° C. After incubation, an aliquot of the reaction mixture was analyzed by SEC using a size exclusion column (Yarra™ SEC-3000, 3 μm, 150×7.8 mm) with a running buffer (50 mM HEPES, 150 mM NaCl, adjusted to pH 7.5). The UV absorbance was monitored at 280 nm. Compound-induced capsid assembly was determined from the ratio of the area under the curve of the Cp149 dimer to that of the capsid fraction.

For EM study, another aliquot of the reaction mixture was negatively stained with 1% uranyl acetate and visualized on a JEOL JEM-1400 100 kV electron microscope. Images were acquired at a magnification of 100,000× to 300,000×.

### HBV Capsid Assembly

The SEC showed an absorbance peak in early fractions (capsids formation) and in the later fractions (dimers formation) with solvent control (2% DMSO). BAY 41-4109 (class I compound), NVR 3-778 (class II), and all active compounds tested increased the formation of capsids. Based on retention times, the capsids induced by BAY 41-4109 were larger in size, as compared to NVR 3-778. Group 1 compounds (e.g., Examples 4, 19, 20, 45, 52, 60, 74, 83, 85, 99, 101, 102, 103, 104, 110, 111, 113, 114, 115, 120, 122, 123, 125, 128, 129, 132, 149, 150, 160, 162, 164, 165, 166) induced capsids similar in size to NVR 3-778, with Group 2 compounds (e.g., Examples 2, 42, 133, 135, 138, 139, 141) resulting in capsid retention times between BAY 41-4109 and NVR 3-778.

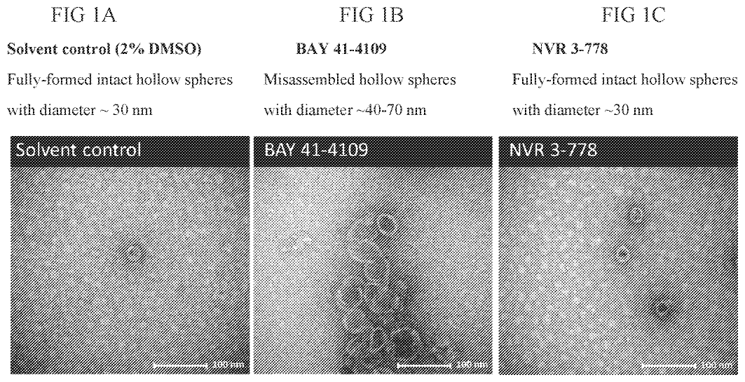

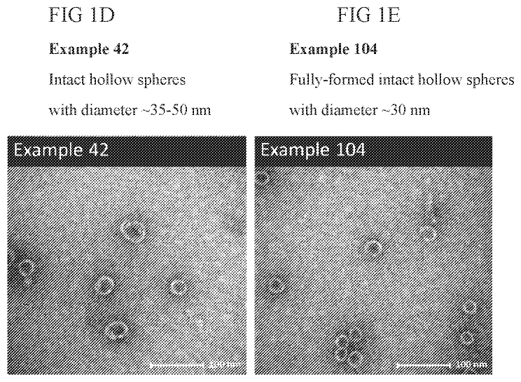

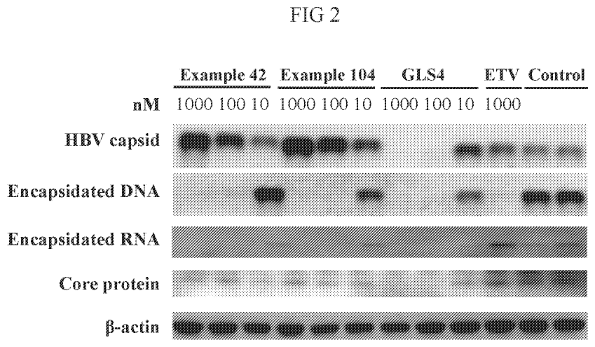

EM results (FIGS. 1A-1E) following incubation of core proteins with solvent control showed capsids as intact hollow spheres. Upon treated with NVR 3-778, formation of the hollow spheres with a similar size as with solvent control was increased. When treated with BAY 41-4109, the capsids appeared larger and misassembled with an irregular shape. Incubation with Example 104, a representative for group 1 compounds, showed similar results as for NVR 3-778, whereas Example 42, a group 2 representative, resulted in larger but normal shape spheres (FIG. 1).

### Example 174. Mechanistic Studies in Stably HBV Expressed Cells and HBV-Infected Primary Human Hepatocytes

The stably HBV expressed HepG2.2.15 cells were treated with test compounds, at various concentrations in culture medium, at 37° C. for three days. After that the medium was removed and fresh medium containing the compound was added to the cells for another 3 days. After 6 days of treatment, cellular lysates from the treated cells were assessed by non-denatured agarose gel electrophoresis. HBV capsid was detected by western blot with monoclonal anti-HBV core. Encapsidated HBV DNA and RNA were detected by southern blot and northern blot. Intracellular HBV core protein was assessed by western Blot with a monoclonal mouse anti-HBV core. β-actin protein assessed by western blot was used as an internal control.

In HepG2.2.15, the Example compounds 42 and 104 dose-dependently accelerated formation of DNA- and RNA-devoid capsid but had no effect on intracellular HBV core protein level (FIG. 2), consistent with the cellular mechanism of class II compounds. In contrast, GLS4 (class I compound) induced formation of DNA- and RNA-devoid capsid, but reduced intracellular HBV core protein level in the concentration-dependent manner. Entecavir (ETV) had no effects on HBV capsid and intracellular core protein levels.

Primary human hepatocytes (PHH) were infected with HBV genotype D produced from HepG2.2.15 cell line on day 1 after plating. Subsequently, the PHH were treated with various concentrations of compounds into 2 different treatment schemes. For treatment scheme 1, the compounds were added into PHH simultaneously with infection media, and incubated for additional 6 days. For treatment scheme 2, the compounds were added into PHH at day 3 after infection, and further incubated for 6 days. After treatment, the cell culture supernatants were collected for the determinations of HBV DNA by qPCR, pgRNA by RT-PCR, HBsAg and HBeAg by ELISA, and cell viability by CCK-8. The cells were harvested for detection of intracellular cccDNA by southern blot.

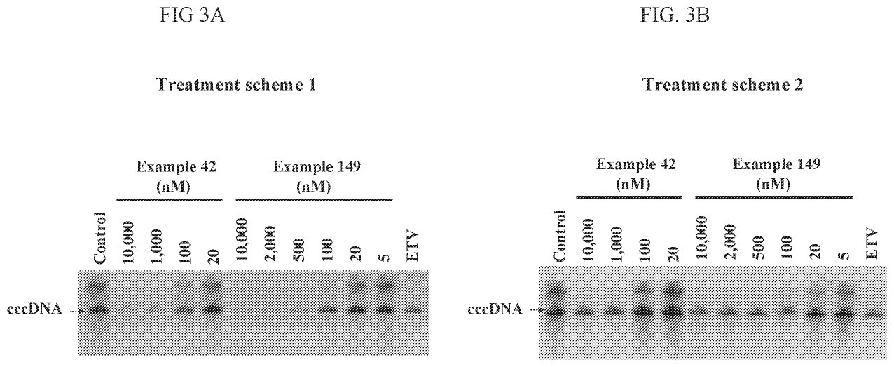

In the treatment scheme 1, but not the treatment scheme 2, the Example compounds 42 and 149 inhibited HBV DNA, surrogate biomarkers of cccDNA (HBsAg and HBeAg), and cccDNA establishment (FIG. 3) in the concentration-dependent manners. In the same experiment, entecavir (ETV) had no effect on cccDNA establishment.

### Example 175. Anti-HBV Activity in Various HBV Genotypes and Core Protein Variants

HBV genomic sequences of HBV genotypes A to D (Genebank ID: HE974381, HE974371, JN406371, AB033554, AB246345, AB246346, U95551, and HE815465) were obtained from NCBI were synthesized and cloned into pcDNA 3.1 construct as a 1.1 mer. Seven core protein mutants (i.e. F23Y, D29G, T33N, 1105T, T109I, T109M, and Y118F) were generated by site-directed mutagenesis of the wild type HBV construct (Genebank ID: U95551).

HepG2 cells were plated and transiently transfected with plasmid DNA carrying HBV genotype or core protein variants. At 24 h after transfection, the cells were treated with various concentrations of test compounds for 3 days. After that the medium was removed and fresh medium containing the compound was added to the cells for another 3 days. After treatment, the cells were harvested and quantified for intracellular HBV DNA level by qPCR. The fifty-percent effective concentrations (EC₅₀) for HBV DNA inhibition, relative to no drug controls, was determined using nonlinear fitting curve model. The EC₅₀ values were compared across HBV genotypes, or calculated as fold changes of EC₅₀ for each core protein variant relative to the wild type construct.

The representative compound (Example 149) was effective across all HBV genotypes tested (A to D) and in almost core protein variants tested (Table C). Fold changes in ECo ranges were as follows: A<1, B=1-5, C=5-15, and D>15 (Table C).

TABLE C
Activities (fold change in EC₅₀) in various
HBV core protein variants

| HBV | | NVR | Example |
|---|---|---|---|
| constructs | GLS4 | 3-778 | 149 |
| U95551 | B | B | B |
| (wild-type) | | | |
| F23Y | C | B | C |
| D29G | B | B | B |
| T33N | D | D | D |
| I105T | B | C | B |
| T109I | C | A | A |
| T109M | B | A | A |
| Y118F | B | B | A |

### Example 176. Pharmacokinetic Study

Test compounds were administered to male Sprague-Dawley rats and male Beagle dogs by oral (PO) and intravenous (TV) administration. For TV dosing, test compounds were dosed at 0.2 to 0.5 mg/kg using a formulation of 10% dimethyl sulfoxide in 20% hydroxypropyl beta-cyclodextrin solution via IV bolus. For PO dosing, test compounds were dosed at 0.3 to 2.0 mg/kg using a formulation of 40% polyethylene glycol 400 in water via oral gavage. Blood samples were collected at pre-dose and various time points up to 24 hours post-dose, and centrifuged to obtain plasma samples. The plasma concentrations of test compounds were quantified by LC-MS/MS methods. The relevant pharmacokinetic parameters (Table D) were calculated via non-compartmental analysis using WinNonlin (Phoenix™, version 8.3).

TABLE D
Pharmacokinetic properties of test compounds
in rats and dogs

| Ex- | CL | | | | | | | |
|---|---|---|---|---|---|---|---|---|
| am- | (mL/ | | V_{d,ss} | | | | | |
| ple | min/kg) | | (L/kg) | | t_{1/2} (h) | | F (%) | |
| No. | Rat | Dog | Rat | Dog | Rat | Dog | Rat | Dog |
| 74 | 3.0 | — | 0.72 | — | 3.4 | — | ~100 | — |
| 83 | 0.3 | — | 0.13 | — | 5.5 | — | 82 | — |
| 85 | 15.2 | 4.4 | 2.88 | 1.64 | 3.6 | 6.2 | 79 | ~100 |
| 99 | 38.4 | | 2.34 | — | 1.0 | — | 36 | — |
| 104 | 12.3 | 1.5 | 1.93 | 1.67 | 1.9 | 14.2 | 66 | 53 |
| 149 | 20.0 | 5.1 | 2.92 | 1.30 | 6.5 | 4.5 | 44 | 63 |

### Example 177. Other Studies

Test compounds were tested for their inhibitory effects on several off-target activities, including major cytochrome P450 enzymes and human Ether-à-go-go-Related Gene (hERG) potassium channel.

The representative compounds had minimal effects on the off-targets tested (IC₅₀≥10 μm).

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (I′): 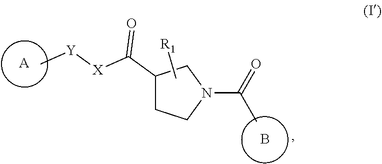
or a prodrug, solvate, or pharmaceutically acceptable salt thereof, wherein:
X is —N(Rₓ)— or —O—;
Y is absent or —C(R_{Y})₂—
R₁ is H, C₁-C₆ alkyl, C₂-C₆ alkenyl, C₂-C₆ alkynyl, or C₁-C₆ haloalkyl;
each R_{Y} independently is H, C₁-C₅ alkyl, C₂-C₆ alkenyl, C₂-C₆ alkynyl, or C₁-C₆ haloalkyl, or
two R_{Y} together with the atom to which they are attached form a 3- to 7-membered heterocycloalkyl or C₃-C₇ cycloalkyl;
Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{A};
Ring B is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B};
R₁ is H, C₁-C₆ alkyl, C₂-C₆ alkenyl, C₂-C₆ alkynyl, or C₁-C₆ haloalkyl;
each R_{A} independently is halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B} independently is halogen, —CN, —(CH₂)ₙ—OR_{B1}, —(CH₂)ₙ—N(R_{B1})(R_{B2}), —(CH₂)ₙ—S(R_{B1}), C₁-C₆ alkyl, C₂-C₆ alkenyl, C₁-C₆ haloalkyl, C₁-C₆ alkoxy, —(CH₂)ₙ—(C₆-C₁₀ aryl), (5- to 10-membered heteroaryl), (C₃-C₇ cycloalkyl), —(CH₂)ₙ-(3- to 7-membered heterocycloalkyl), —C(O)R_{B1}, —C(O)OR_{B1}, or —C(O)N(R_{B1})(R_{B2}), wherein the alkyl, alkenyl, alkynyl, aryl, heteroaryl, cycloalkyl, and heterocycloalkyl are optionally substituted with one or more R_{B4};
each R_{B1} and R_{B2} is independently H, halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}, or
R_{B1} and R_{B2}, together with the atom to which they are attached, form a 3- to 7-membered heterocycloalkyl, optionally substituted with halogen, —CN, —OH, —NH₂, —NH(C₁-C₆ alkyl), —N(C₁-C₆ alkyl)₂, C₁-C₆ alkyl, C₂-C₆ alkenyl, C₂-C₆ alkynyl, C₁-C₆ haloalkyl, C₁-C₆ alkoxy, 3- to 7-membered heterocycloalkyl, or C₃-C₇ cycloalkyl;
each R_{B3} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3′};
each R_{B3′} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, or C₁-C₆ alkoxy;
each R_{B4} is independently oxo, halogen, —CN, —OH, —NH₂, —NH(C₁-C₆ alkyl), —NH(C₁-C₆ alkyl)-OH, —N(C₁-C₆ alkyl)₂, —(CH₂)ₘ—C(O)R_{B4}, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4}, or —N(R_{B4′})(R_{B4″});
each R_{B4′} and R_{B4″} is independently H, —OH, —NH₂, C₁-C₆ alkyl, C₂-C₆ alkenyl, C₂-C₆ alkynyl, C₁-C₆ haloalkyl, C₁-C₆ alkoxy, C₃-C₇ cycloalkyl, or 3- to 7-membered heterocycloalkyl, wherein the alkyl, alkenyl, alkynyl, cycloalkyl, and heterocycloalkyl are optionally substituted with one or more oxo or —OH;
n is 0, 1, 2, 3, 4, or 5; and
m is 0, 1, 2, 3, 4, or 5,
provided that when R_{B} is a substituted or unsubstituted alkyl, Ring A is substituted by at least one R_{A}.

2. The compound of claim 1, wherein:
X is —N(Rₓ)—;
Y is absent;
Rₓ is H or C₁-C₆ alkyl;
Ring A is C₆-C₁₀ aryl or 5- to 10-membered heteroaryl, wherein the aryl and heteroaryl are optionally substituted with one or more R_{A};
Ring B is 5- to 10-membered heteroaryl optionally substituted with one or more R_{B}; and
R₁ is H or C₁-C₆ alkyl;
provided that when R_{B} is a substituted or unsubstituted alkyl, Ring A is substituted by at least on R_{A}.

3. The compound of claim 1, wherein X is —N(Rₓ)—.

4. The compound of claim 1, wherein Y is absent or —CH₂—.

5. The compound of claim 1, wherein Rₓ is H.

6. The compound of claim 1, wherein Rₓ is C₁-C₆ alkyl, C₂-C₆ alkenyl, or C₂-C₆ alkynyl.

7. The compound of claim 1, wherein Ring A is C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are substituted with one or more R_{A}.

8. The compound of claim 1, wherein Ring A is phenyl substituted with two R_{A}.

9. The compound of claim 1, wherein Ring A is phenyl substituted with three R_{A}.

10. The compound of claim 1, wherein Ring B is 5- to 10-membered heteroaryl optionally substituted with one or more R_{B}.

11. The compound of claim 1, wherein R₁ is H.

12. The compound of claim 1, wherein R₁ is C₁-C₆ alkyl, C₂-C₆ alkenyl, C₂-C₆ alkynyl, or C₁-C₆ haloalkyl.

13. The compound of claim 1, wherein each R_{A} independently is halogen, —CN, C₁-C₆ alkyl, or C₃-C₇ cycloalkyl.

14. The compound of claim 1, wherein each R_{B} independently is halogen, —CN, —(CH₂)ₙ—OR_{B1}, —(CH₂)ₙ—N(R_{B1})(R_{B2}), —(CH₂)ₙ—S(R_{B1}), —C(O)R_{B1}, —C(O)OR_{B1}, or —C(O)N(R_{B1})(R_{B2}).

15. The compound of claim 1, wherein each R_{B} independently is C₁-C₆ alkyl, C₂-C₆ alkenyl, C₂-C₆ alkynyl, C₁-C₆ haloalkyl, C₁-C₆ alkoxy, —(CH₂)ₙ—(C₆-C₁₀ aryl), —(CH₂)ₙ-(5- to 10-membered heteroaryl), —(CH₂)ₙ—(C₃-C₇ cycloalkyl), or —(CH₂)-(3- to 7-membered heterocycloalkyl), wherein the alkyl, alkenyl, alkynyl, aryl, heteroaryl, cycloalkyl, and heterocycloalkyl are optionally substituted with one or more R_{B4}.

16. The compound of claim 1, wherein each R_{B1} and R_{B2} is independently H.

17. The compound of claim 1, wherein each R_{B1} and R_{B2} is independently halogen, —CN, —OH, —NH₂, —NH(C₁-C₆ alkyl), —NH(C₁-C₆ alkyl)-OH, —N(C₁-C₆ alkyl)₂, C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₃-C₇ cycloalkyl, or 3- to 7-membered heterocycloalkyl, wherein the alkyl, alkenyl, alkynyl, aryl, heteroaryl, cycloalkyl, and heterocycloalkyl are optionally substituted with one or more R_{B3}.

18. The compound of claim 1, wherein each R_{B3} is independently C₁-C₆ alkyl, C₂-C₆ alkenyl, C₂-C₆ alkynyl, C₁-C₆ haloalkyl, or C₁-C₆ alkoxy, wherein the alkyl, alkenyl, or alkynyl are optionally substituted with one or more R_{B3′}.

19. The compound of claim 1, wherein each R_{B3} is independently C₆-C₁₀ aryl, 5- to 10-membered heteroaryl, C₃-C₇ cycloalkyl, or 3- to 7-membered heterocycloalkyl, wherein the aryl, heteroaryl, cycloalkyl, and heterocycloalkyl are optionally substituted with one or more R_{B3′}.

20. The compound of claim 1, wherein each R_{B4} is independently oxo, halogen, —CN, —OH, —NH₂, —NH(C₁-C₆ alkyl), —NH(C₁-C₆ alkyl)-OH, —N(C₁-C₆ alkyl)₂, or, —(CH₂)ₘ—C(O)R_{B4} wherein the alkyl is optionally substituted with one or more halogen, —CN, —OR_{B4′}, or —N(R_{B4′})(R_{B4″}).

21. The compound of claim 1, wherein each R_{B4} is independently C₁-C₆ alkyl, C₂-C₆ alkenyl, C₂-C₆ alkynyl, C₁-C₆ haloalkyl, C₁-C₆ alkoxy, C₆-C₁₀ aryl, 5- to 10-membered heteroaryl, C₁-C₇ cycloalkyl, or 3- to 7-membered heterocycloalkyl, wherein the alkyl, alkenyl, alkynyl, aryl, heteroaryl, cycloalkyl, or heterocycloalkyl is optionally substituted with one or more halogen, —CN, —OR_{B4′}, or —N(R_{B4′})(R_{B4″}).

22. The compound of claim 1, wherein n is 0, 1, or 2.

23. The compound of claim 1, wherein the compound is of Formula (I′-c), (I′-d), (I′-c1), (I-a′), (I-b′), (I-c′), (I-d′), or (I-c1′): 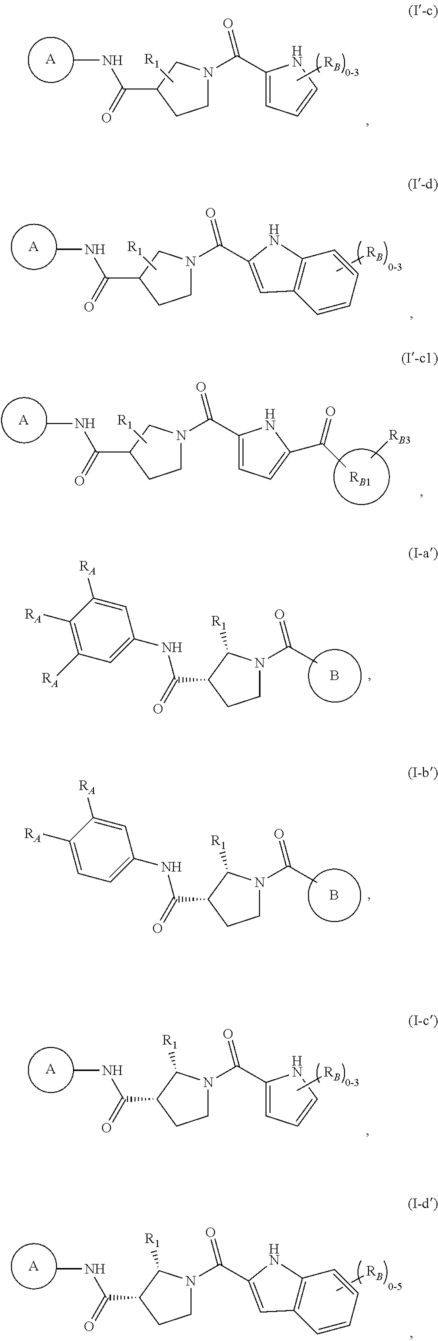 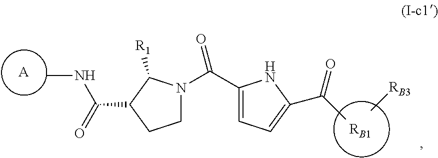
or a prodrug, solvate, or pharmaceutically acceptable salt thereof.

24. The compound of any one of the preceding claims, being selected from Compound Nos. 1-175 and prodrugs and pharmaceutically acceptable salts thereof.

25. A compound obtainable by, or obtained by, a method described herein;
optionally, the method comprises one or more steps described in Schemes I-V.

26. A pharmaceutical composition comprising the compound ofany one of claims 1-25 or a pharmaceutically acceptable salt thereof, and a pharmaceutically acceptable diluent or carrier.

27. The pharmaceutical composition of claim 26, wherein the compound is selected from Compound Nos. 1-175.

28. A method of treating or preventing a disease or disorder in a subject in need thereof, comprising administering to the subject a therapeutically effective amount of the compound of any one of claims 1-25 or a pharmaceutically acceptable salt thereof, or the pharmaceutical composition of claim 26 or claim 27.

29. The compound of any one of claims 1-25, or the pharmaceutical composition of claim 26 or claim 27, for use in treating or preventing a disease or disorder.

30. Use of the compound of any one of claims 1-25 or a pharmaceutically acceptable salt thereof in the manufacture of a medicament for treating or preventing a disease or disorder.

31. The method, compound, pharmaceutical composition, or use of any one of the preceding claims, wherein the disease or disorder is a viral infection.

32. The method, compound, pharmaceutical composition, or use of claim 31, wherein the viral infection is hepatitis B virus.

33. A method of modulating the HBV replication cycle in a subject in need thereof, comprising administering to the subject a therapeutically effective amount of the compound of any one of claims 1-25 or a pharmaceutically acceptable salt thereof, or the pharmaceutical composition of claim 26 or claim 27.

34. The compound of any one of claims 1-25, or the pharmaceutical composition of claim 26 or claim 27, for use modulating the HBV replication cycle.

35. Use of the compound of any one of claims 1-25 or a pharmaceutically acceptable salt thereof in the manufacture of a medicament for modulating the HBV replication cycle.

36. The method, compound, pharmaceutical composition, or use of any one of claims 28-35, in combination with one or more additional therapeutic agent.

37. The method, compound, pharmaceutical composition, or use of claim 36, wherein the one or more additional therapeutic agent is useful for treating virus infection.

38. The method, compound, pharmaceutical composition, or use of claim 36 or 37, wherein the at least one or more additional therapeutic agent comprises a medicament for treatment of HBV.

39. The method, compound, pharmaceutical composition, or use of any one of claims 36-39, wherein the one or more additional therapeutic agent is administered simultaneously, separately, or sequentially.
